(19) United States
(12) Patent Application Publication
(10) Pub. No.: US 2026/0174882 A1
(43) Pub. Date: Jun. 25, 2026

(54) PREPARATION OF LIPID NANOPARTICLE (LNP) CONJUGATES

(71) Applicants: Lei LIU, Kildeer, IL (US); Tessera Therapeutics, Inc., Somerville, MA (US)

(72) Inventors: Zhan WANG, Somerville, MA (US); Rui ZHANG, Somerville, MA (US); Han GU, Somerville, MA (US); Lei LIU, Kildeer, IL (US)

(21) Appl. No.: 19/127,711

(22) PCT Filed: Nov. 7, 2023
(86) PCT No.: PCT/US2023/079005

§ 371 Date: May 6, 2025

Related U.S. Application Data
(60) Provisional application No. 63/423,188, filed on Nov. 7, 2022.
(60) Provisional application No. 63/478,974, filed on Jan. 8, 2023.

(51) Int. Cl. A61K 47/60; A61K 39/395; A61K 47/54; A61K 47/58; A61K 47/64; A61K 47/68

(52) U.S. Cl. CPC A61K 47/60; A61K 39/3955; A61K 47/545; A61K 47/58; A61K 47/645; A61K 47/6849; A61K 47/6851

(57) ABSTRACT

The disclosure provides conjugates comprising a targeting moiety, e.g., an antibody or functional fragment thereof, and a lipid nanoparticle (LNP) encapsulating a therapeutic agent, wherein the targeting moiety is conjugated to the LNP through a linker comprising a thiol moiety and a click product formed via a click reaction between a first click handle and a second click handle.

### CROSS-REFERENCE TO RELATED APPLICATIONS

This application claims priority to U.S. Provisional Patent Application No. 63/423,188 filed Nov. 7, 2022, and U.S. Provisional Application No. 63/478,974 filed Jan. 8, 2023, the disclosure of each which is hereby incorporated by reference in its entirety for all purposes.

### REFERENCE TO AN ELECTRONIC SEQUENCE LISTING

The contents of the electronic sequence listing (252052001940SEQLIST.xml; Size: 147,736 bytes; and Date of Creation: Nov. 6, 2023) is herein incorporated by reference in its entirety.

### BACKGROUND

The development of lipid nanoparticles (LNPs) has recently made significant advances towards intracellular delivery of payloads such as nucleic acids (e.g., mRNA or siRNA). LNPs are generally comprised of multiple components including an ionizable lipid, a PEGylated lipid, a helper lipid and cholesterol, all of which play important roles in effectively delivering the payload to diseased tissue. Nonetheless, substantial safety issues still remain. For instance, LNPs may accumulate and deliver payloads to cells other than the intended target, which results in potential toxicity. Accordingly, an important goal is to develop LNPs that target diseased tissue and that can be administered at nontoxic doses.

One approach to improve the toxicological profile and increase the efficacy of LNPs is to modify the surface of the LNP with an antibody or functional fragment thereof that targets specific cells, such as diseased cells. For instance, LNPs can be coated with targeting moieties, such as antibodies or antigen binding portions thereof, that bind to particular cellular receptors on target cells, resulting in accumulation of the payload in the targeted tissue relative to other tissue in the body. Different approaches have been used to introduce an antibody or functional fragment thereof onto the surface of an LNP. For example, one approach relies on functionalizing a preformed LNP with an antibody or functional fragment thereof. The LNP generally includes a lipid that has polyethylene glycol (PEG) spacer functionalized with a reactive moiety such as a thiol, amine, maleimide or carboxylic acid group. The functionalized lipid of the LNP reacts with a complementary group that is covalently bonded to an antibody or functional fragment thereof, hence generating a conjugate of the LNP and the antibody or functional fragment thereof.

More recent conjugation approaches using milder reactions conditions are based on biorthogonal chemistry reactions such as click chemistry. The so-called click product formed from a click handle on the LNP and a click handle bonded to the antibody or functional fragment thereof links the LNP to the antibody or functional fragment thereof. One biorthogonal approach that has been used to generate LNP/antibody or functional fragment thereof conjugates include copper-catalyzed click reactions such as Huisgen 1,3-dipolar cycloaddition (CuAAC) between an azide and an alkyne. Other biorthogonal approaches rely on using copper-free click chemistry, for example, the click product can be formed between an azide and dibenzocyclooctene (DBCO), the click product formed using an inverse electron demand Diels-alder cycloaddition (IEDDA) between a trans-cyclooctene (TCO) moiety and a tetrazine ring, or a click product formed in a Staudinger reaction between an azide and a phosphine.

Generally, the conjugation approaches described above, including the biorthogonal approaches, when applied to antibodies or antigen binding fragments, produce LNPs that are conjugated to the antibodies in a nonhomogeneous manner. Generally, when an antibody or antigen binding fragment is functionalized with a reactive group or click handle, such functionalization occurs in a random manner, resulting in a heterogeneous population of antibodies that shows significant batch-to-batch variability. The randomly modified antibodies or antigen binding fragments produce LNPs that have their surfaces modified in a random manner. The reaction between the randomly modified antibodies or antigen binding fragments with the LNPs may not occur with optimal efficiency. Moreover, LNPs decorated in this manner may contain a proportion of antibodies or antigen binding fragments that are incapable of efficiently binding to their target receptors on cells because of the ineffective way they were orientated on the surface of the LNP following conjugation. Furthermore, antibodies that conjugate at specific amino acid residues to the antibody or antigen binding fragment may do so in a non-optimized manner that may impair antibody binding capacity, resulting in a sub-optimal therapeutic effect.

Therefore, there exists a need to develop LNPs that have surfaces modified with antibodies of fragments thereof, specifically antibody or antigen binding fragments, where the antibody or antigen binding fragment is linked to the LNP in a highly site-specific manner.

### BRIEF SUMMARY OF INVENTION

In one aspect, the disclosure provides a method of conjugating a lipid nanoparticle (LNP) to an antibody or functional fragment thereof, said method comprising:
(i) contacting the antibody or functional fragment thereof comprising a free cysteine residue with a crosslinker molecule comprising a first click handle and a thiol-reactive functional group, whereby the thiol-reactive functional group of the crosslinker molecule reacts with the free cysteine residue of the antibody or functional fragment thereof; and
(ii) contacting the product of step (i) with an LNP comprising a second click handle covalently bonded to the surface of the LNP, whereby the first click handle reacts with the second click handle via a click chemistry reaction, thereby forming a conjugate between the LNP and the antibody or functional fragment thereof.

In some embodiments, the free cysteine is in the hinge region of the antibody or functional fragment thereof. In some embodiments, the method further comprises reducing disulfide bonds in the hinge region prior to step (i). In some embodiments, the methods further comprise removing an interchain disulfide bond near the C-terminus of the antibody or functional fragment thereof prior to step (i) and introducing a new disulfide bond buried within the CL-CH1 interface of the antibody or functional fragment thereof. In some embodiments, the new interchain disulfide bond is more stable under reducing conditions than the original interchain disulfide bond.

In some embodiments, the crosslinker further comprises a spacer between the first click handle and a thiol-reactive functional group. In some embodiments, the spacer comprises a polyethylene glycol (PEG) and wherein the PEG comprises n ethylene glycol units, wherein n is between 4 and 200 (e.g., between 4 and 100, 25 and 100 or 50 and 100).

In some embodiments, the thiol-reactive functional group is a maleimide group, a parafluoro group, an ene group, an yne group, a vinylsulfone group, a pyridyl disulfide group, a thiosulfonate group, and a thiol-bisulfone group. In some embodiments, the thiol-reactive functional group is a maleimide group. In some such embodiments, the maleimide group is substituted with one or more substituents selected from the group consisting of C₁₋₃ alkyl, halo, and C₁₋₃Oalkyl.

In some embodiments, the reaction between the first click handle and the second click handle forms a click product, and, wherein the click product is formed through a copper-catalyzed click chemistry reaction, a Huisgen 1,3-dipolar cycloaddition between an azide and an alkyne, or through a copper free click chemistry reaction. In some embodiments where the click product is formed through a copper-free click chemistry reaction, and wherein the copper-free click chemistry reaction is selected from the group consisting of (a) a strain-promoted cycloaddition between an azide and a cyclic alkyne; (b) a Staudinger ligation between an azide and a phosphine; (c) an inverse electron demand Diels-Alder reaction between a trans-cyclooctene (TCO) and a tetrazine; (d) an inverse electron demand Diels-Alder reaction between a tetrazine and a norbonene; (e) a photoinducible 1,3-dipolar cycloaddition reaction between a tetrazole and an alkene; (f) an oxime ligation between an aldehyde or ketone and an α effect amine; and (g) a hydrazone ligation between an aldehyde or ketone and an α effect amine. In some embodiments the click product is formed via an inverse electron demand Diels-Alder reaction between a TCO moiety and a tetrazine moiety. In some such embodiments, the tetrazine moiety is unsubstituted. In other such embodiments, the tetrazine moiety is methyltetrazine.

In another aspect, the disclosure provides a conjugate comprising an antibody or functional fragment thereof and a lipid nanoparticle (LNP) encapsulating a therapeutic agent. In some embodiments, the conjugate is produced by a method as disclosed herein. In some embodiments, the conjugate comprises an antibody or functional fragment thereof and a lipid nanoparticle (LNP) encapsulating a therapeutic agent, wherein the antibody or functional fragment thereof is conjugated to the LNP through a linker comprising a thiol moiety and a click product formed via a click reaction between a first click handle and a second click handle. In some embodiments, the thiol moiety is covalently bonded directly to the antibody or functional fragment thereof and the second click handle is covalently bonded directly to the LNP. In some embodiments, the thiol moiety is a thioether moiety. In some embodiments, the linker of the conjugate further comprises a spacer between the thiol moiety and the click product. In some embodiments, the spacer is a polyethylene glycol (PEG). In some such embodiments, the PEG comprises n ethylene glycol units, wherein n is between 4 and 200 (e.g., between 4 and 100, 25 and 100 or 50 and 100). In some embodiments, the thiol moiety of the conjugate is synthesized from the group consisting of a thiol-maleimide reaction, a thiol-parafluoro reaction, a thiol-ene reaction, a thiol-yne reaction, a thiol-vinylsulfone reaction, a thiol-pyridyl disulfide reaction, a thiol-thiosulfonate reaction, and a thiol-bisulfone reaction.

In another aspect, the disclosure provides a conjugate comprising an antibody or functional fragment thereof and a lipid nanoparticle (LNP) encapsulating a therapeutic agent, wherein the antibody or functional fragment thereof is conjugated to the LNP through a linker comprising a thiol moiety and a second moiety selected from the group consisting of a triazole moiety, dihydropyridazine moiety, aza-ylide moiety, hydrazone moiety and an oxime moiety. In some embodiments, the thiol moiety is covalently bonded directly to the antibody or functional fragment thereof. In some embodiments, the thiol moiety is a thioether moiety. In some embodiments, the linker of the conjugate further comprises a spacer between the thiol moiety and the click product. In some embodiments, the spacer is a PEG. In some such embodiments, the PEG comprises n ethylene glycol units, wherein n is between 4 and 200 (e.g., between 4 and 100, 25 and 100 or 50 and 100). In some embodiments, the thiol moiety of the conjugate is synthesized from the group consisting of a thiol-maleimide reaction, a thiol-parafluoro reaction, a thiol-ene reaction, a thiol-yne reaction, a thiol-vinylsulfone reaction, a thiol-pyridyl disulfide reaction, a thiol-thiosulfonate reaction, and a thiol-bisulfone reaction.

In any of the foregoing embodiments, the antibody functional fragment can be a Fab fragment. A Fab fragment, as used herein, refers to a univalent fragment that has at least one free cysteine residue that is capable of reacting with a thiol-reactive functional group, as described herein. In some embodiments, the univalent Fab fragment can be produced by proteolytic cleavage of a bivalent F(ab′)₂ followed by reduction of the two disulfide bridges in the hinge region, hence generating two monovalent F(ab′) fragments, each with two reactive free cysteine residues capable of conjugating to a thiol-reactive functional group. In alternative embodiments, the Fab fragment can be prepared using recombinant procedures such as a procedure described in Example 1. Recombinant production of the Fab fragments allows for introduction of a single free reactive cysteine residue at the C-terminal end of either the heavy or light chain of the Fab fragment. For instance, in certain embodiments, the free cysteine residue is located at the C-terminus of the constant domain of the heavy chain (referred to as C_{H}1) in the region normally occupied by the hinge region of an antibody. A schematic of such a construct in Fab fragment is depicted in FIG. 7A. In some embodiments, the amino acid sequence introduced at the C-terminus of the Fab fragment (e.g., the C-terminus of the heavy chain of the Fab fragment) is part of a sequence that is present in the hinge region of naturally occurring antibodies or Fab fragments (i.e., a conserved sequence), albeit with one of the two cysteine residues not present. For instance, in some embodiments, the sequence at the C-terminus of the Fab fragment is a conserved sequence in a human IgG1 hinge region such as DKTHTC (SEQ ID NO: 97). In these embodiments, the cysteine (C) residue of DKTHTC (SEQ ID NO: 97), is reactive with the thiol-reactive functional group. In some embodiments, additional amino acid residues can be added after the cysteine residue (i.e., C-terminal of the free cysteine residue). For instance, in some embodiments, between 1 and 5 amino acid residues can be added at the C-terminal end of the cysteine residue. In some such embodiments, the amino acid residues added to the C-terminal end of the free cysteine residues are alanine residues. Specific examples of sequences added to constant region (e.g., C_{H}1) of the heavy chain are DKTHTCA (SEQ ID NO: 87), and DKTHTCAA (SEQ ID NO: 99). Other examples are provided in the Detailed Description section below.

It will be understood that sequences other than conserved hinge sequences can be introduced at the C-terminus of the Fab fragment (e.g., the C-terminus of the heavy chain of the Fab fragment), as long as at least one cysteine residue is present.

Following recombinant production of the antibody functional fragment (e.g., Fab fragment), the antibody functional fragment may include a disulfide bond between cysteine groups in the hinge region as a result of an oxidation reaction between the univalent fragments, hence producing a bivalent fragment without a reactive free cysteine residue. In some embodiments, the disulfide bond is reduced prior to reacting with the thiol-reactive functional group, hence generating the free reactive cysteine residue. In some embodiments, the reduction of the disulfide bond in the hinge region of the antigen binding fragment (e.g., Fab fragment) can be accomplished with minimal or no reduction of the interchain disulfide bond at the CL-CH1 interface. In some embodiments, tris(2-carboxyethyl)phosphine (TCEP) or TCEP agarose (i.e., TCEP immobilized on agarose) is used as a reducing agent to minimize reduction of the interchain disulfide bond at the CL-CH1 interface of the antibody functional fragment (e.g., Fab fragment). Following reduction of the disulfide bond in the hinge region, the free cysteine group can be coupled with a reaction partner such as a thiol-reactive functional group, as set forth herein. The procedure allows for site-specific introduction of the antibody functional fragment (e.g., Fab fragment) onto the surface of the LNP.

To maximize site-specific introduction of the antibody functional fragment (e.g., Fab fragment) onto the surface of the LNP, the interchain disulfide bond at the CL-CH1 interface of the antibody functional fragment (e.g., Fab fragment) can be engineered away from the C-terminus and buried within the CL-CH1 interface. Recombinant engineering of the antibody functional fragment (e.g., Fab fragment) to remove the interchain disulfide bond at the CL-CH1 interface and place the interchain disulfide bond within the interior of the CL-CH1 interface of the antibody functional fragment (e.g., Fab fragment) can potentially reduce or eliminate the reduction of the interchain disulfide bridge outside the hinge region, hence allowing for selective reduction of the disulfide bridge in the hinge region. Accordingly, the methodology enables maximum site-specific introduction of the antibody functional fragment (e.g., Fab fragment) onto the surface of the LNP following the reduction of the disulfide bridge in the hinge region of the antibody functional fragment (e.g., Fab fragment) Specific examples of recombinant engineering of the disulfide bond in the hinge region are provided in Example 2. In some embodiments, the conjugate has a sequence set forth in Table 12.

In any of the foregoing embodiments, the antibody or functional fragment thereof of the conjugate binds to a T cell or to a hematopoietic stem cell (HSC). In some embodiments, the antibody or functional fragment thereof binds to CD2, CD3, CD4, CD5, CD6, CD7, CD8, CD90 or CD117.

In any of the foregoing embodiments, the therapeutic agent delivered by the conjugate can be a nucleic acid molecule. In some embodiments, the nucleic acid molecule is a DNA plasmid, closed-ended DNA (ceDNA), or a small circular DNA. In some embodiments, the nucleic acid molecule is a DNA plasmid, closed-ended DNA (ceDNA), or a small circular DNA. In some embodiments, the nucleic acid molecule is an mRNA molecule. In some embodiments, the mRNA molecule encodes an enzyme. In some such embodiments, the enzyme is nuclease, recombinase, integrase, transposase, retrotransposase, helicase, transcriptase, polymerase, reverse transcriptase, deaminase, methylase, demethylase, or ligase, or a combination thereof. In some embodiments, the enzyme is a CRISPR-Cas nuclease (e.g., a nickase). In some such embodiments, the CRISPR-Cas nuclease is a Cas9 or Cas12a. In some embodiments, the conjugate further comprises a guide RNA (gRNA) molecule.

In any of the foregoing embodiments, the therapeutic agent delivered by the conjugate can be a gene modifying polypeptide. In some embodiments, the gene modifying polypeptide is a retrotransposon. In some embodiments, the conjugate further comprises a template RNA that binds to the gene modifying polypeptide. In some such embodiments, the template RNA encodes a chimeric antigen receptor (CAR).

In any of the foregoing embodiments, the therapeutic agent delivered by the conjugate can be a gene modifying system. In some embodiments, the gene modifying system comprises a gene modifying polypeptide and a template RNA. In some embodiments, the gene modifying polypeptide comprises a retrotransposon. In some embodiments, the template RNA encodes a CAR.

In any of the foregoing embodiments, the therapeutic agent delivered by the conjugate can be a heterologous gene modifying system, or a component thereof.

In some embodiments, the conjugates of the disclosure can be used to deliver systems that are capable of inserting a heterologous object sequence into the genome of a cell. In some embodiments, the system comprises: (A) a gene modifying polypeptide or a nucleic acid encoding the gene modifying polypeptide, wherein the gene modifying polypeptide comprises: (i) an endonuclease and/or DNA binding domain; and (ii) a reverse transcriptase (RT) domain, where (i) and (ii) are both derived from a retrotransposon (e.g., from the same retrotransposon or different retrotransposons); and (B) a template RNA (or DNA encoding the template RNA) comprising (i) a sequence that binds the polypeptide and (ii) a heterologous object sequence. A gene modifying polypeptide, in some embodiments, acts as a substantially autonomous protein machine capable of integrating a template nucleic acid sequence into a target DNA molecule (e.g., in a mammalian host cell, such as a genomic DNA molecule in the host cell), substantially without relying on host machinery. The heterologous object sequence may include, e.g., a coding sequence, a regulatory sequence, or a gene expression unit. In some embodiments, the gene modifying polypeptide can be a retrotransposon, e.g., selected from the retrotransposons of Table 7. In some embodiments, the gene modifying polypeptide can be a retrotransposon selected, without limitation, from the following retrotransposon classes: RTE (e.g., RTE-1_MD, RTE-3_BF, and RTE-25_LMi), CR1 (e.g., CR1-1_PH), Crack (e.g., Crack-28_RF), L2 (e.g., L2-2_Dre and L2-5_GA), and Vingi (e.g., Vingi-1_Acar).

### BRIEF DESCRIPTION OF DRAWINGS

FIG. 1 shows an exemplary conjugate comprising a Fab fragment and a lipid nanoparticle (LNP). The Fab fragment is conjugated to the surface of the LNP through a linker.

FIGS. 2A-2B show exemplary conjugates comprising a Fab fragment and an LNP. The Fab fragment is conjugated to the surface of the LNP through a linker. FIG. 2A shows an embodiment where the linker comprises a thioether moiety and a click product formed via a click reaction. FIG. 2B shows an embodiment wherein the linker further comprises a spacer between the thioether moiety and the click product.

FIG. 3A shows an exemplary conjugate comprising a Fab fragment and an LNP. The Fab fragment is conjugated to the surface of the LNP through a linker. FIG. 3B shows a conjugate comprising a thiosuccinimide moiety and a dihydropyridazine moiety.

FIG. 4A shows a schematic of a crosslinker molecule of the disclosure. FIG. 4B shows an embodiment wherein the crosslinker molecule comprises a spacer.

FIG. 5 is a schematic of assembly of an LNP with a click handle (i.e., second click handle) to be reacted with a first click handle linked to the targeting moiety, e.g., antibody, ScFv or Fab fragment.

FIG. 6 shows the chemical structure of an exemplary crosslinker molecule comprising a methyltetrazine as the first click handle, a maleimide as the thiol-reactive functional group, and PEG as the spacer.

FIG. 7A shows an exemplary Fab fragment comprising a hinge region and a free cysteine residue located within the hinge region. The free cysteine residue is available for reaction with a thiol-reactive functional group of a crosslinker molecule. FIG. 7B shows an embodiment of a Fab fragment comprising a hinge region and a free cysteine residue, wherein an inter-chain disulfide bond has been engineered away from the C-terminus of the Fab fragment.

FIG. 8 shows a Fab fragment with a fully reduced free cysteine on the hinge region can react directly with a thiol-reactive functional group in a single-step process, thereby generating a conjugate.

FIGS. 9A-9B show an exemplary schematic of conjugate formation. In FIG. 9A, a Fab fragment comprising a free cysteine residue is contacted with a crosslinker molecule, wherein the thiol-reactive functional group of the crosslinker molecule reacts with the free cysteine residue of the Fab fragment to produce the product of step (i). In FIG. 9B, the product of step (i) is contacted with an LNP comprising a second click handle covalently bonded to the surface of the LNP, wherein the first click handle of the Fab fragment reacts with the second click handle via a click chemistry reaction to produce the conjugate (product of step (ii)).

FIG. 10 shows examples of a pegylated lipid bonded to a second click handle (TCO).

FIG. 11 shows examples of non-pegylated lipid bonded to a second click handle (TCO).

FIG. 12 shows examples of ionizable lipid bonded to a second click handle (TCO).

FIG. 13 shows some examples of sterols bonded to a second click handle (TCO).

FIGS. 14A-14B shows FACs analysis results for CD34+ cells transfected with targeted LNPs or base LNPs showing the percentage of cells expressing GFP (% GFP+) (FIG. 14A) and GFP expression levels (MFI) (FIG. 14B).

FIG. 15 shows SDS-PAGE analysis data for an engineered anti-CD117 Fab fragment for selective reduction with TCEP agarose.

### DETAILED DESCRIPTION OF INVENTION

### I. Definitions

Antigen binding domain: The term “antigen binding domain” as used herein refers to that portion of a targeting moiety, e.g., an antibody or a chimeric antigen receptor which binds an antigen. In some embodiments, an antigen binding domain binds to a cell surface antigen of a cell. In some embodiments an antigen binding domain binds an antigen characteristic of a cancer, e.g., a tumor associated antigen in a neoplastic cell. In some embodiments, an antigen binding domain binds an antigen characteristic of an infectious disease, e.g. a virus associated antigen in a virus infected cell. In some embodiments, an antigen binding domain binds an antigen characteristic of a cell targeted by a subject's immune system in an autoimmune disease, e.g., a self-antigen. In some embodiments, an antigen binding domain is or comprises an antibody or antigen-binding portion thereof. In some embodiments, an antigen binding domain is or comprises an scFv or Fab.

Domain: The term “domain” as used herein refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, an endonuclease domain, a DNA binding domain, a reverse transcriptase domain; an example of a domain of a nucleic acid is a regulatory domain, such as a transcription factor binding domain.

Exogenous: As used herein, the term “exogenous,” when used with reference to a biomolecule (such as a nucleic acid sequence or polypeptide) means that the biomolecule was introduced into a host genome, cell, or organism by the hand of man. For example, a nucleic acid that is as added into an existing genome, cell, tissue, or subject using recombinant DNA techniques or other methods is exogenous to the existing nucleic acid sequence, cell, tissue or subject.

Expression cassette: The term “expression cassette,” as used herein, refers to a nucleic acid construct comprising nucleic acid elements sufficient for the expression of the nucleic acid molecule of the instant invention.

gRNA spacer: A “gRNA spacer”, as used herein, refers to a portion of a nucleic acid that has complementarity to a target nucleic acid and can, together with a gRNA scaffold, target a Cas protein to the target nucleic acid.

gRNA scaffold: A “gRNA scaffold”, as used herein, refers to a portion of a nucleic acid that can bind a Cas protein and can, together with a gRNA spacer, target the Cas protein to the target nucleic acid. In some embodiments, the gRNA scaffold comprises a crRNA sequence, tetraloop, and tracrRNA sequence.

Gene modifying polypeptide: A “gene modifying polypeptide,” and “retrotransposon gene modifying polypeptide” as used herein interchangeably to refer to a polypeptide comprising a retrotransposase reverse transcriptase domain and a retrotransposase endonuclease domain, or a polypeptide comprising an amino acid sequence having at least 75%, 80%, 85%, 90%, 95%, 96%, 97%, 98%, or 99% amino acid sequence identity to said domains, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the endonuclease domain is a catalytically inactive endonuclease domain. In some embodiments, the retrotransposase reverse transcriptase domain and a retrotransposase endonuclease domain are derived from the same retrotransposase. In some embodiments, the gene modifying polypeptide is capable of integrating the sequence substantially without relying on host machinery. In some embodiments, the gene modifying polypeptide integrates a sequence into a random position in a genome, and in some embodiments, the gene modifying polypeptide integrates a sequence into a specific target site. In some embodiments, a gene modifying polypeptide includes one or more domains that, collectively, facilitate 1) binding the template nucleic acid, 2) binding the target DNA molecule, and 3) facilitate integration of the at least a portion of the template nucleic acid into the target DNA. Gene modifying polypeptides include both naturally occurring polypeptides as well as engineered variants of the foregoing, e.g., having one or more amino acid substitutions to the naturally occurring sequence. Gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary gene modifying polypeptides, and systems comprising them and methods of using them, that can be used in the methods provided herein are described, e.g., in WO/2021/178717, which is incorporated herein by reference, including Tables 10, 11, X, 3A, 3B, and Z1 therein. In some embodiments, a gene modifying polypeptide integrates a sequence into a gene. In some embodiments, a gene modifying polypeptide integrates a sequence into a sequence outside of a gene. A “gene modifying system,” as used herein, refers to a system comprising a gene modifying polypeptide and a template nucleic acid.

Gene modifying system: A “gene modifying system,” as used herein, refers to a system comprising a gene modifying polypeptide, or a nucleic acid (e.g., an mRNA) encoding the gene modifying polypeptide, and a template nucleic acid.

Heterologous: The term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector). In some embodiments, a domain is heterologous relative to another domain, if the first domain is not naturally comprised in the same polypeptide as the other domain (e.g., a fusion between two domains of different proteins from the same organism).

Heterologous gene modifying polypeptide: As used herein, the term “heterologous gene modifying polypeptide” refers to a polypeptide comprising a retroviral reverse transcriptase, or a polypeptide comprising an amino acid sequence having at least 75%, 80%, 85%, 90%, 95%, 96%, 97%, 98%, or 99% amino acid sequence identity to a retroviral reverse transcriptase, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the heterologous gene modifying polypeptide is capable of integrating the sequence substantially without relying on host machinery. In some embodiments, the heterologous gene modifying polypeptide integrates a sequence into a random position in a genome, and in some embodiments, the heterologous gene modifying polypeptide integrates a sequence into a specific target site. In some embodiments, the sequence that is integrated comprises a deletion, substitution, or insertion relative to the target DNA molecule. In some embodiments, a heterologous gene modifying polypeptide includes one or more domains that, collectively, facilitate 1) binding the template nucleic acid, 2) binding the target DNA molecule, and 3) facilitate integration of the at least a portion of the template nucleic acid into the target DNA. Heterologous gene modifying polypeptides include both naturally occurring polypeptides as well as engineered variants of the foregoing, e.g., having one or more amino acid substitutions to the naturally occurring sequence. Heterologous gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary heterologous gene modifying polypeptides, and systems comprising them and methods of using them, that can be used in the methods provided herein are described, e.g., in PCT/US2021/020948, which is incorporated herein by reference with respect to heterologous gene modifying polypeptides that comprise a retroviral reverse transcriptase domain. In some embodiments, a heterologous gene modifying polypeptide integrates a sequence into a gene. In some embodiments, a heterologous gene modifying polypeptide integrates a sequence into a sequence outside of a gene. A “heterologous gene modifying system,” as used herein, refers to a system comprising a heterologous gene modifying polypeptide and a template nucleic acid.

Mutation or Mutated: The term “mutated” when applied to nucleic acid sequences means that nucleotides in a nucleic acid sequence may be inserted, deleted or changed compared to a reference (e.g., native) nucleic acid sequence. A single alteration may be made at a locus (a point mutation) or multiple nucleotides may be inserted, deleted, or changed at a single locus. In addition, one or more alterations may be made at any number of loci within a nucleic acid sequence. A nucleic acid sequence may be mutated by any method known in the art. In some embodiments a mutation occurs naturally. In some embodiments a desired mutation can be produced by a system described herein.

Nucleic acid molecule: “Nucleic acid molecule” refers to both RNA and DNA molecules including, without limitation, complementary DNA (“cDNA”), genomic DNA (“gDNA”), and messenger RNA (“mRNA”), and also includes synthetic nucleic acid molecules, such as those that are chemically synthesized or recombinantly produced, such as RNA templates, as described herein. The nucleic acid molecule can be double-stranded or single-stranded, circular, or linear. If single-stranded, the nucleic acid molecule can be the sense strand or the antisense strand. Unless otherwise indicated, and as an example for all sequences described herein under the general format “SEQ ID NO:,” or “nucleic acid comprising SEQ ID NO:1” refers to a nucleic acid, at least a portion which has either (i) the sequence of SEQ ID NO:1, or (ii) a sequence complimentary to SEQ ID NO:1. The choice between the two is dictated by the context in which SEQ ID NO: 1 is used. For instance, if the nucleic acid is used as a probe, the choice between the two is dictated by the requirement that the probe be complementary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are chemically modified bases, backbone, and modified caps. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule, e.g., peptide nucleic acids (PNAs). Other modifications can include, for example, analogs in which the ribose ring contains a bridging moiety or other structure such as modifications found in “locked” nucleic acids (LNAs). In various embodiments, the nucleic acids are in operative association with additional genetic elements, such as tissue-specific expression-control sequence(s) (e.g., tissue-specific promoters and tissue-specific microRNA recognition sequences), as well as additional elements, such as inverted repeats (e.g., inverted terminal repeats, such as elements from or derived from viruses, e.g., AAV ITRs) and tandem repeats, inverted repeats/direct repeats, homology regions (segments with various degrees of homology to a target DNA), untranslated regions (UTRs) (5′, 3′, or both 5′ and 3′ UTRs), and various combinations of the foregoing. The nucleic acid elements of the systems provided by the invention can be provided in a variety of topologies, including single-stranded, double-stranded, circular, linear, linear with open ends, linear with closed ends, and particular versions of these, such as doggybone DNA (dbDNA), closed-ended DNA (ceDNA).

Primer Binding Sequence: The term “primer binding site sequence” or “PBS sequence,” as used herein, refers to a portion of a template RNA capable of binding to a region comprised in a target nucleic acid sequence. In some instances, a PBS sequence is a nucleic acid sequence comprising at least 3, 4, 5, 6, 7, or 8 bases with 100% identity to the region comprised in the target nucleic acid sequence. In some embodiments the primer region comprises at least 5, 6, 7, 8 bases with 100% identity to the region comprised in the target nucleic acid sequence. Without wishing to be bound by theory, in some embodiments when a template RNA comprises a PBS sequence and a heterologous object sequence, the PBS sequence binds to a region comprised in a target nucleic acid sequence, allowing a reverse transcriptase domain to use that region as a primer for reverse transcription, and to use the heterologous object sequence as a template for reverse transcription.

It is understood that aspects and embodiments described herein as “comprising” include “consisting of” and “consisting essentially of” embodiments.

### II. Conjugates

In one aspect, the disclosure provides a conjugate comprising a targeting moiety, e.g., an antibody or functional fragment thereof, and a lipid nanoparticle (LNP) encapsulating a therapeutic agent, wherein the targeting moiety, e.g., antibody or functional fragment thereof, is conjugated to the LNP through a linker comprising a thiol moiety (e.g., a thioether moiety) and a click product formed via a click reaction between a first click handle and a second click handle. In some embodiments, the thiol moiety is generated by reaction of a free thiol group (—SH) of a cysteine residue on the antibody or functional fragment thereof and a thiol-reactive functional group. Particular thiol-based reactions that can be used to generate thiol moieties include, but are not limited to, thiol-maleimide reactions, thiol-parafluoro reactions, thiol-ene reactions, thiol-yne reactions, thiol-vinylsulfone reactions, thiol-pyridyl disulfide reactions, thiol-thiosulfonate reactions, and thiol-bisulfone reactions. Examples of such thiol-based reactions are described in M. H. Stenzel, ACS Macro Lett. 2013, 2, 14-18.

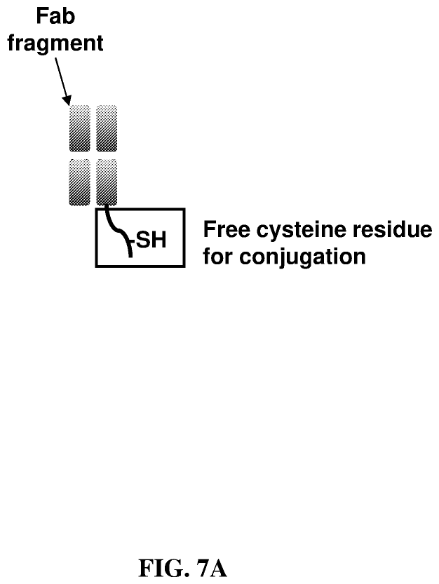

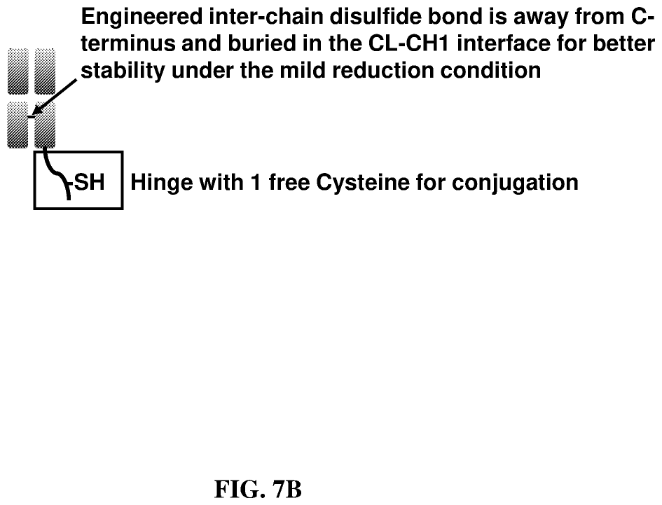

In another aspect, the disclosure provides methods of making a conjugate via a multi-step process (e.g., two-step process), wherein the conjugate comprises a targeting moiety, e.g., an antibody or functional fragment thereof, and a lipid nanoparticle (LNP) encapsulating a therapeutic agent, wherein the targeting moiety, e.g., antibody or functional fragment thereof, is conjugated to the LNP through a linker comprising a thiol moiety (e.g., a thioether moiety) and a click product formed via a click reaction between a first click handle and a second click handle. A Fab fragment, as used herein, refers to a univalent fragment that has at least one free cysteine residue that is capable of reacting with a thiol-reactive functional group, as described herein. In some embodiments, the functional fragment of an antibody is a Fab fragment. In some embodiments, the univalent Fab fragment can be produced by proteolytic cleavage of a bivalent F(ab′)₂ followed by reduction of the two disulfide bridges in the hinge region, hence generating two monovalent F(ab′) fragments, each with two reactive free cysteine residues capable of conjugating to a thiol-reactive functional group. In alternative embodiments, the Fab fragment can be prepared using recombinant procedures such as a procedure described in Example 1. Recombinant production of the Fab fragments allows for introduction of a single free reactive cysteine residue at the C-terminal end of either the heavy or light chain of the Fab fragment. For instance, in certain embodiments, the free cysteine residue is located at the C-terminus of the constant domain of the heavy chain (referred to as C_{H}1) in the region normally occupied by the hinge region of an antibody. A schematic of such a construct in Fab fragment is depicted in FIG. 7A. In some embodiments, the functional fragment of an antibody is a single-chain variable fragment (ScFv). In some embodiments, the functional fragment of an antibody is a VHH domain antibody. In some embodiments, the targeting moiety is an FN3 domain, a nanobody, a single domain antibody or a Centyrin. In some embodiments, the targeting moiety is a ligand that binds to a receptor on the surface of a cell. In some embodiments, the ligand can be a natural ligand for the receptor. In some embodiments, the ligand can be a synthetic ligand for the receptor. In some embodiments, the targeting moiety is a peptide or polypeptide, e.g., a peptide or polypeptide ligand for a receptor on the surface of a cell. In some embodiments, the targeting moiety is a cytokine, e.g., such that the cytokine targeting moiety binds to a cytokine receptor on the surface of a cell. Conjugation of a targeting moiety to the LNP creates a targeted LNP (tLNP).

In one embodiment, the targeting moiety, e.g., antibody or functional fragment, thereof is linked to the LNP via a lipid on the surface of the LNP.

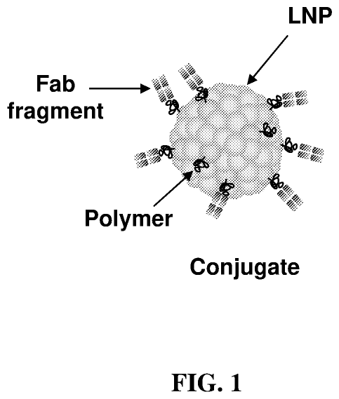

In one embodiment, the targeting moiety, e.g., antibody or functional fragment thereof, is linked to the LNP via a polymer on the surface of the LNP. In some such embodiments, the polymer is covalently attached to a lipid on the surface of the LNP. A particular embodiment is shown in FIG. 1. In FIG. 1, a polymer in the surface of an LNP is covalently attached to a Fab fragment. As set forth herein, attachment of the Fab fragment to the polymer can be achieved via a click reaction between a first click handle that is linked (e.g., covalently bonded) directly or indirectly to the Fab fragment and a second click handle that is linked (e.g., covalently bonded) to the LNP. In some embodiments, the polymer on the surface of the LNP comprises a polyethylene glycol (PEG).

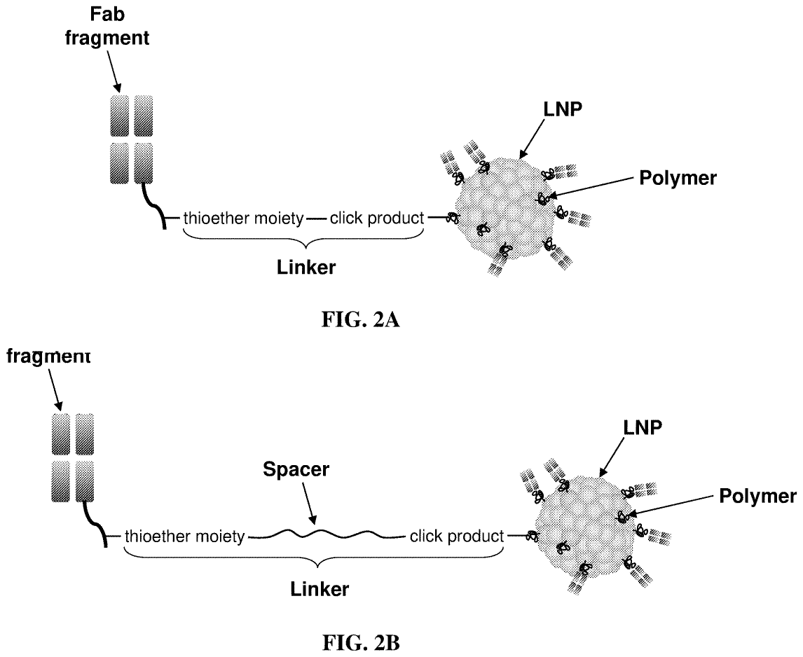

As shown in FIG. 2A, the linker of the conjugate comprises a thioether moiety and a click product formed via a click reaction between a first click handle and a second click handle. The LNP can be as described in any of the embodiments in the Lipid Nanoparticle section below. In some embodiments, the linker of the conjugate further comprises a spacer between the thioether moiety and the click product. (FIG. 2B)

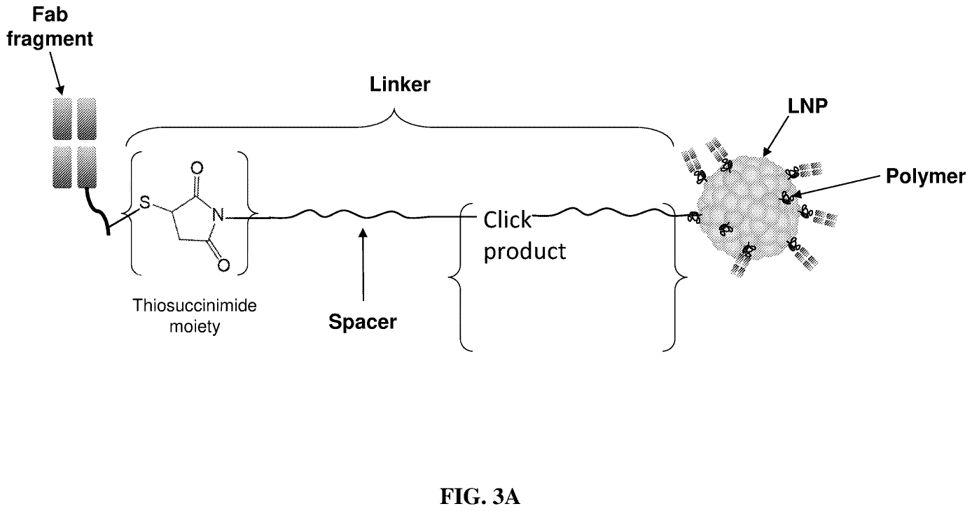

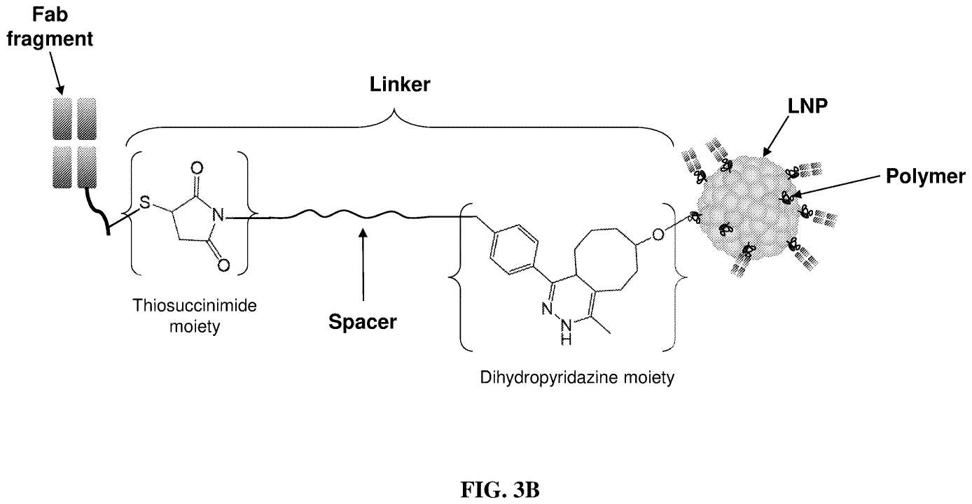

In another embodiment, the conjugate comprising a targeting moiety, e.g., an antibody or functional fragment thereof, and a lipid nanoparticle (LNP) encapsulating a therapeutic agent, wherein the targeting moiety, e.g., antibody of functional fragment thereof, is conjugated to the LNP through a linker comprising a thiol moiety (e.g., a thioether moiety) and a second moiety, wherein the second moiety is a click product formed via a click reaction between a first click handle and a second click handle. In some embodiments, the thioester moiety is a thiosuccinimide. FIG. 3A shows one such embodiment, wherein a Fab fragment is linked to an LNP, wherein the LNP comprises a thiosuccinimide moiety, a spacer and a click product. As set forth herein, the thiosuccinimide moiety can be formed via a reaction between a free cysteine in the hinge region of the Fab fragment and a maleimide moiety on a crosslinker molecule.

In some embodiments, the click product on the linker is selected from a triazole moiety, dihydropyridazine moiety, aza-ylide moiety, hydrazone moiety and oxime moiety. As shown in FIG. 3B, in some embodiments, the linker comprises a thiosuccinimide moiety and a dihydropyridazine moiety.

Another aspect of the disclosure provides a method of conjugating an LNP to a targeting moiety, e.g., an antibody or functional fragment, thereof comprising: (i) contacting the targeting moiety, e.g., antibody or functional fragment thereof, comprising a free cysteine residue with a crosslinker molecule comprising a first click handle and a thiol-reactive functional group, whereby the thiol-reactive functional group of the crosslinker molecule reacts with the free cysteine residue of the targeting moiety, e.g., antibody or functional fragment thereof; and (ii) contacting the product of step (i) with an LNP comprising a second click handle covalently bonded to the surface of the LNP, whereby the first click handle reacts with the second click handle via a click chemistry reaction, thereby conjugating the targeting moiety, e.g., antibody or functional fragment thereof, to the surface of the LNP. In some embodiments, the functional fragment of an antibody is a Fab fragment. In some embodiments, the functional fragment of an antibody is an ScFv. In some embodiments, the functional fragment of an antibody is a VHH domain antibody. In some embodiments, the targeting moiety is an FN3 domain, a nanobody, a single domain antibody or a Centyrin. In some embodiments, the targeting moiety is a ligand that binds to a receptor on the surface of a cell. In some embodiments, the ligand can be a natural ligand for the receptor. In some embodiments, the ligand can be a synthetic ligand for the receptor. In some embodiments, the targeting moiety is a peptide or polypeptide, e.g., a peptide or polypeptide ligand for a receptor on the surface of a cell. In some embodiments, the targeting moiety is a cytokine, e.g., such that the cytokine targeting moiety binds to a cytokine receptor on the surface of a cell. Conjugation of a targeting moiety to the LNP creates a targeted LNP (tLNP).

Another aspect of the disclosure provides a conjugate comprising a targeting moiety, e.g., an antibody or functional fragment thereof, and a lipid nanoparticle (LNP) encapsulating a therapeutic agent (e.g, payload) made by a process, the process comprising: (i) contacting the targeting moiety, e.g., antibody or functional fragment, thereof comprising a free cysteine residue with a crosslinker molecule comprising a first click handle and a thiol-reactive functional group, whereby the thiol-reactive functional group of the crosslinker molecule reacts with the free cysteine residue of the targeting moiety, e.g., antibody or functional fragment thereof; and (ii) contacting the product of step (i) with an LNP comprising a second click handle covalently bonded to the surface of the LNP, whereby the first click handle reacts with the second click handle via a click chemistry reaction, thereby conjugating the targeting moiety, e.g., antibody or functional fragment thereof, to the surface of the LNP.

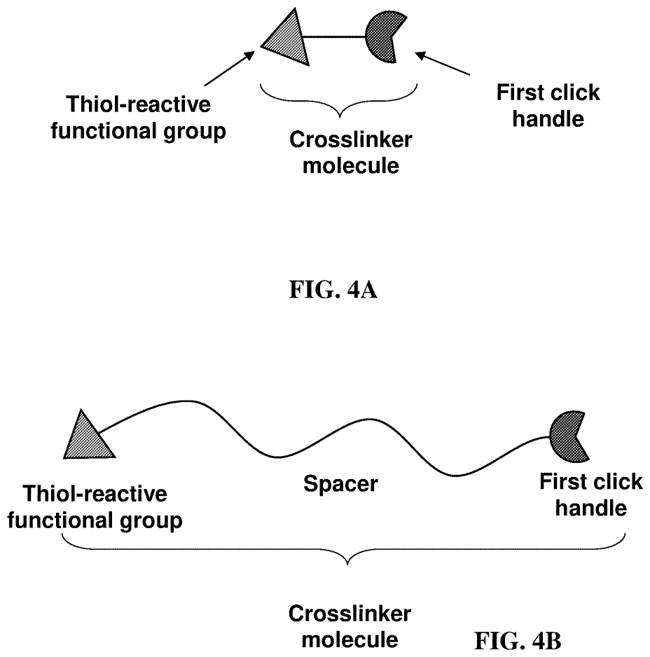

FIG. 4A shows a schematic of a crosslinker molecule of the disclosure. In accordance with the disclosure, the thiol-reactive functional group reacts with a free cysteine on the targeting moiety, e.g., antibody or functional fragment thereof, in a first reaction, thereby attaching the first click handle to the targeting moiety, e.g., antibody or functional fragment thereof. Next, the first click handle on the crosslinker molecule reacts with a complementary second click handle on the LNP, thereby conjugating the targeting moiety, e.g., antibody or functional fragment thereof, to the LNP.

In some embodiments, following reaction between the targeting moiety, e.g., antibody or functional variant thereof, with the crosslinker molecule, the linker is covalently linked to the targeting moiety, e.g., antibody or functional fragment thereof. In some such embodiments, the targeting moiety, e.g., antibody or functional fragment thereof, is attached to the linker through a thiol moiety (e.g., a thioether moiety). In some embodiments, the linker is covalently linked to the targeting moiety, e.g., antibody or functional fragment thereof, through a reaction between a free cysteine residue located on the antibody or functional fragment thereof and a thiol-reactive functional group. The thiol-reactive functional group can be any suitable reactive group, including but not limited to, maleimide, pyridyl disulfide, and haloacetyl. In some embodiments, the thiol-reactive functional group is maleimide, wherein maleimide reacts with the free cysteine residue of the targeting moiety, e.g., antibody or functional fragment thereof, to form a thiosuccinimide moiety (FIG. 3A and FIG. 3B). In some embodiments, the thiosuccinimide moiety is substituted with one or more substituents selected from C₁₋₃ alkyl, halo, and C₁₋₃Oalkyl.

In some embodiments, the reaction between the free cysteine residue of the targeting moiety, e.g., antibody or functional fragment thereof, and the thiol-reactive functional group forms a covalent bond. In some embodiments, the formation of a covalent bond between the free cysteine residue and the thiol-reactive functional group is reversible (e.g., the antibody or functional fragment thereof can be released from the linker). In some embodiments, the formation of a covalent bond between the free cysteine residue and the thiol-reactive functional group is irreversible. In some embodiments, the reaction efficiency (i.e., percent of thiol-reactive group conjugated to antibody or functional fragment thereof) between the free cysteine residue of the targeting moiety, e.g., antibody or functional fragment thereof, and the thiol-reactive functional group is greater than 5%, greater than 10%, greater than 25%, greater than 50%, greater than 60%, greater than 70%, greater than 80%, or greater than 90%. In some embodiments, the reaction efficiency between the free cysteine residue of the targeting moiety, e.g., antibody or functional fragment thereof, and the thiol-reactive functional group is from about 5% to about 30%, about 10% to about 20%, about 25% to about 50%, about 30% to about 40%, about 50% to about 80%, about 60% to about 70%, about 70% to about 95%, or about 80% to about 90%.

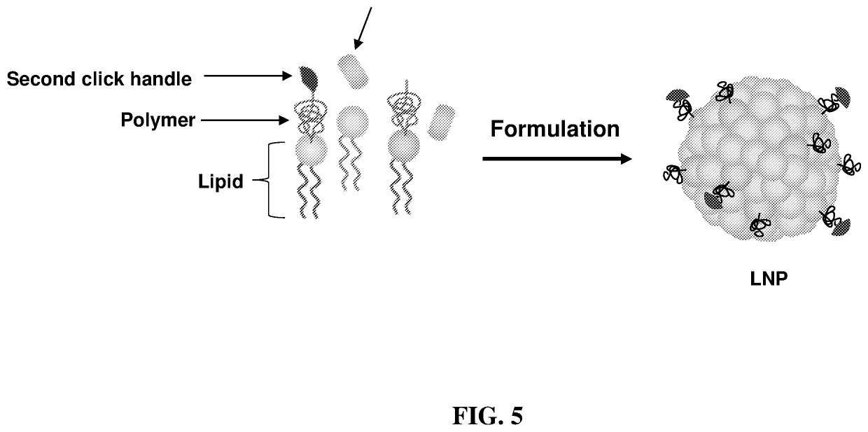

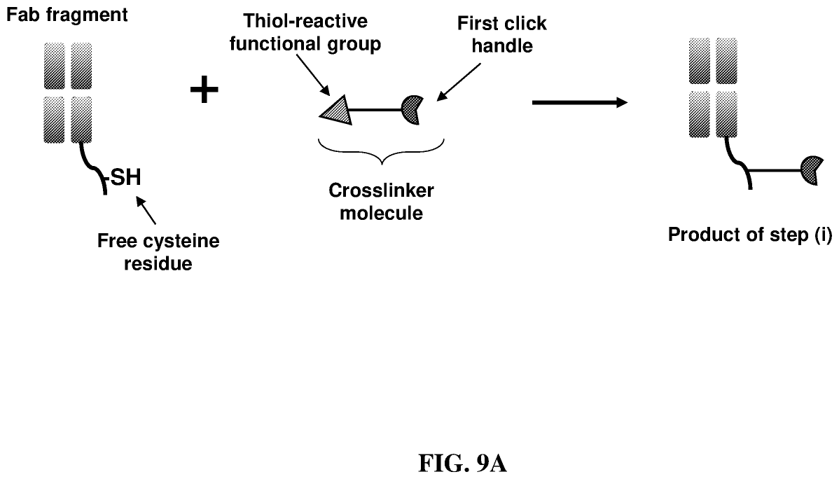

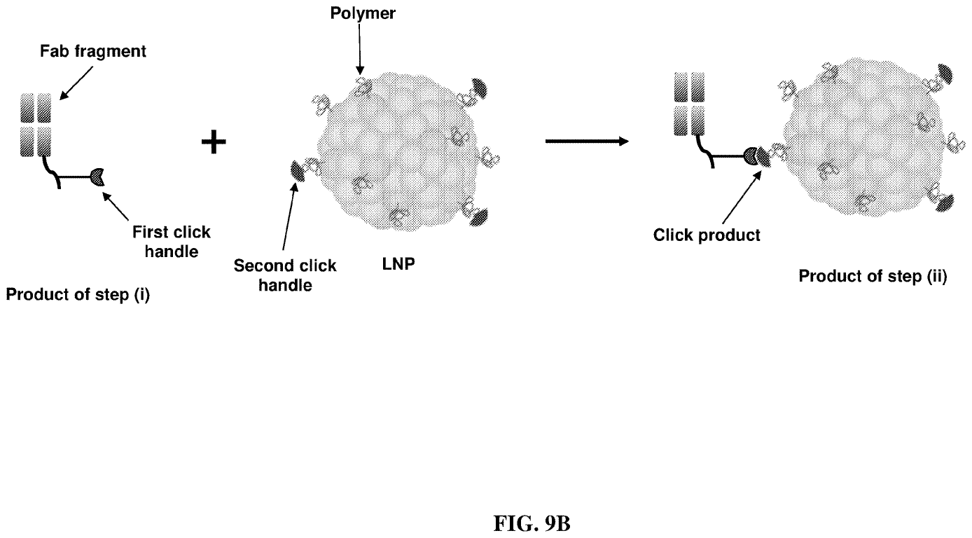

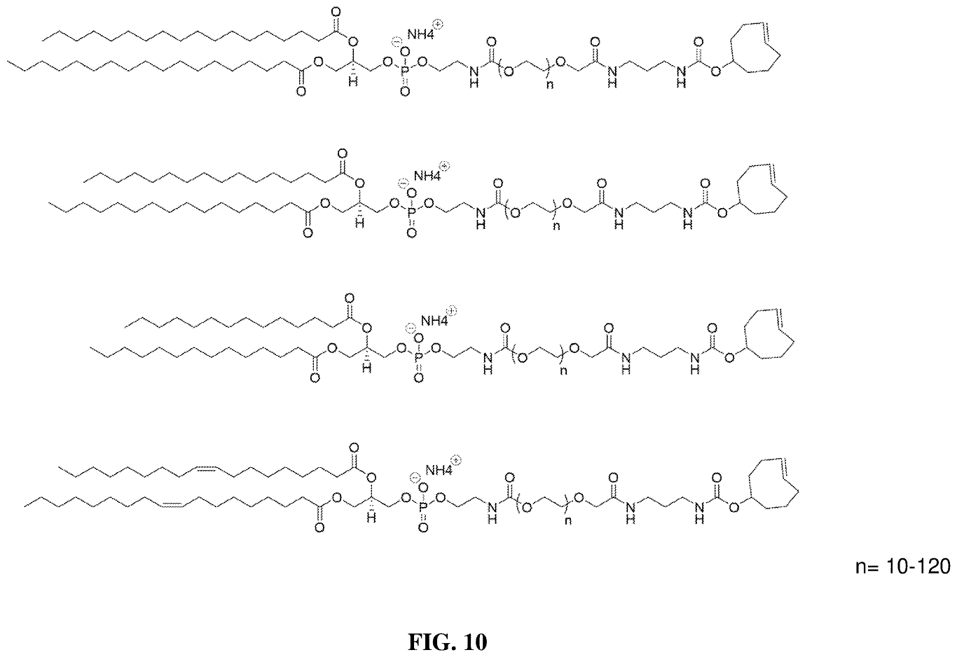

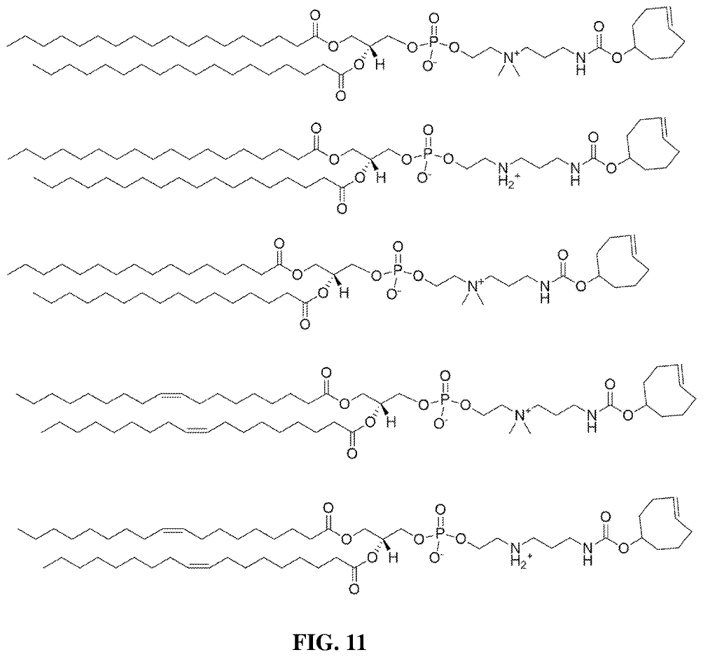

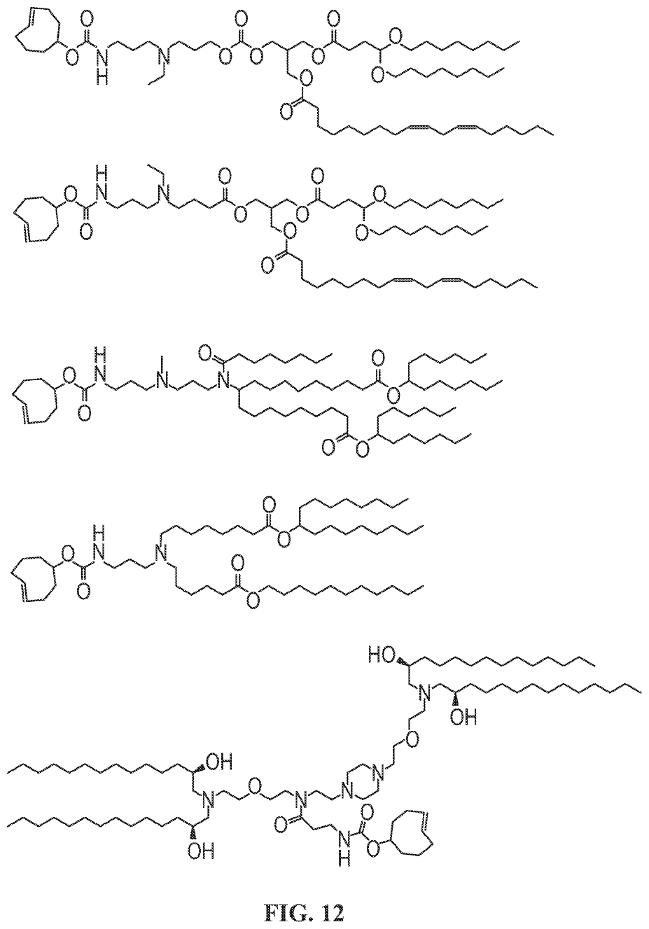

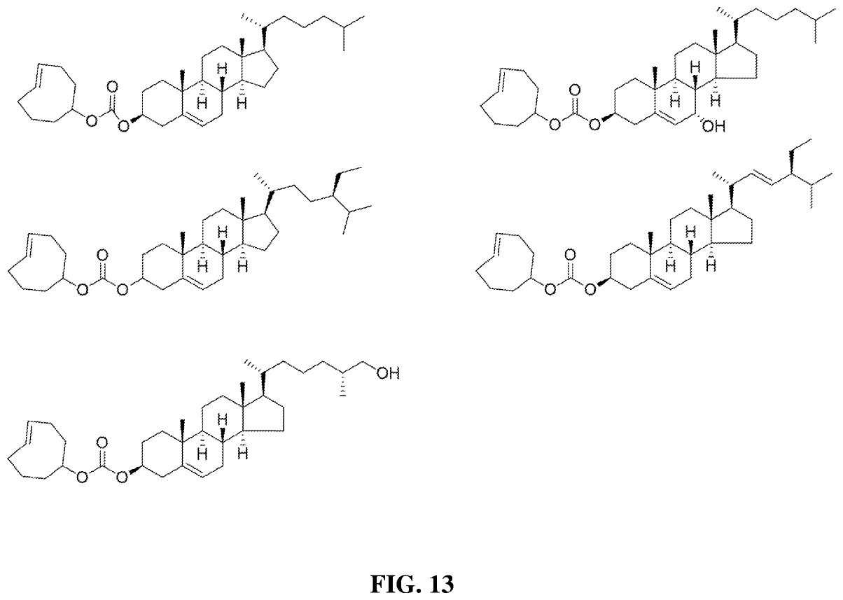

In some embodiments, the linker is attached to the surface of the LNP through a click product formed via a click reaction between a first click handle on the crosslinker molecule and a second click handle of the LNP. In some embodiments, LNPs are formulated with lipids comprising a second click handle. In some embodiments, the second click handle is covalently bonded to at least one of the lipid molecules. In some embodiments, the lipid molecule bonded to the second click handle is a pegylated lipid. In some embodiments, after LNP formation, the second click handle is accessible to the first click handle for conjugation of a targeting moiety, e.g., an antibody or a functional fragment thereof, to the LNP surface. FIG. 5 shows an embodiment where the second click handle is covalently bonded to a pegylated lipid prior to generation of the LNP. Following mixing of the lipid components and payload, an LNP is formed with the second click handle bound to the surface of the LNP. The second click handle is capable of reacting with a first click handle bound to a targeting moiety, e.g., an antibody or functional fragment thereof. See FIG. 9B. The individual lipids comprising the LNP including the lipids bound to click handles can be as described in any of the embodiments in the Lipid Nanoparticle section below.

In some embodiments, the first click handle and the second click handle used to form the click product can be any suitable click chemistry pair (e.g., Azide-BCN, Azide-DBCO, Tz-TCO, meTz-TCO, etc.). The click product can be formed using a biorthogonal chemistry approach. It can be appreciated from the nonlimiting examples disclosed herein that the reaction of the first click handle and the second click handle occur with high specificity such that each click handle is inert towards other components in the reaction system (e.g., PEG, lipids of the LNP, amino acids of the targeting moiety, e.g., antibody or functional fragment thereof).

In one embodiment, the click product can be formed using a copper-catalyzed click reaction. One such copper-catalyzed click reaction is a Huisgen 1,3-dipolar cycloaddition (CuAAC) between an azide and an alkyne (see, e.g., Tornøe et al., J. Org. Chem. 2002, 67 (9), 3057-3064 and Rostovtsev et al., Angew. Chem. Int. Ed. 2002, 41 (14), 2596-2599). In some embodiments, the click product is a triazole moiety.

In some embodiments, the first or second click handle comprises a cyclic derivative of the alkynyl group. In some embodiments, the cyclic derivative of the alkynyl group is selected from dibenzocyclooctyne (DBCO), bicyclononynes (BCN), cyclooctyne, and difluorinated cyclooctyne. In some embodiments, the click chemistry involves a strain-promoted cycloaddition between an azide and a cyclic alkyne (see, e.g., Agard et al., J. Am. Chem. Soc. 2004, 126 (46), 15046-15047 and Dommerholt et al., Angew. Chem., Int. Ed. 2010, 49 (49), 9422-9425). In some embodiments, the click chemistry is based upon a reaction using strained alkynes.

In another embodiment, the click product can be formed using copper-free click chemistry. For example, the click product can be formed between an azide and dibenzocyclooctene (DBCO). Alternatively, the click product can be formed using a Staudinger reaction between an azide and a phosphine, hence producing an aza-ylide (see, e.g., Saxon et al., Science 2000, 287 (5460), 2007-2010).

In some embodiments, the click product can be formed using any suitable photo-induced click chemistry reaction. In some embodiments, the click product can be formed using photoinducible 1,3-dipolar cycloaddition reaction between a tetrazole and an alkene (see, e.g., Song et al., Angew. Chem., Int. Ed. 2008, 47 (15), 2832-2835).

In some embodiments, the click product can be formed using oxime and hydrazone ligations. In some embodiments, a ketone or aldehyde can react with α effect amine, such as hydroxylamine, hydrazine and hydrazide (see, e.g., Agten et al., ChemBioChem 2013, 14 (18), 2431-2434 and Dirksen et al., J. Am. Chem. Soc. 2006, 128 (49), 15602-15603).

In some embodiments, the click product can be formed from any suitable inverse electron demand Diels-Alder reaction. In some embodiments, the click product can be formed from an inverse electron demand Diels-Alder reaction between a trans-cyclooctene (TCO) moiety on the first or second click handle and a tetrazine (Tz) ring on the first or second click handle (see, e.g., Selvaraj et al., Curr. Opin. Chem. Biol. 2013, 17 (5), 753-760 and Rossin et al., Bioconjugate Chem. 2013, 24 (7), 1210-1217). In some embodiments, the first click handle comprises a tetrazine ring and the second click handle comprises a TCO moiety. In some embodiments, the tetrazine ring is unsubstituted. In some such embodiments, the tetrazine ring is methyltetrazine. In some embodiments, the tetrazine ring is a 6-methyl substituted tetrazine. In some embodiments, the click product is a dihydropyridazine moiety.

In some embodiments, the first or second click handle is a tetrazine derivative having one of the following structures:

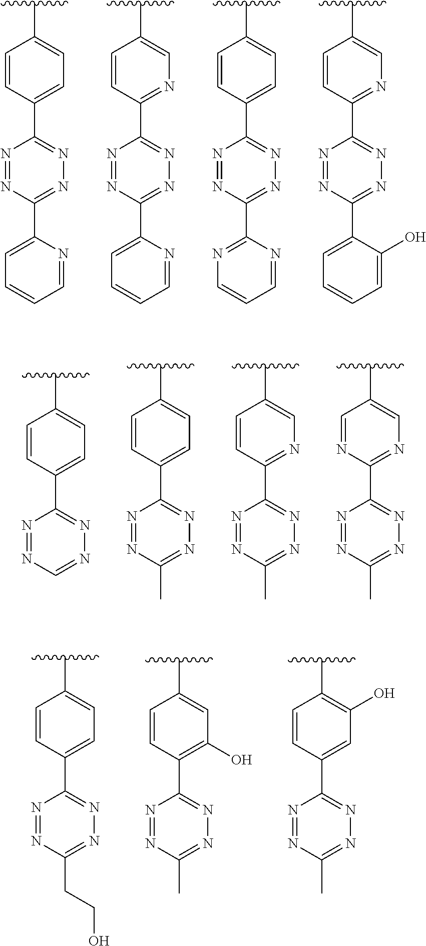

wherein [symbol] represents the point of attachment, directly or indirectly, of the first or second click handle to the targeting moiety, e.g., antibody or a functional fragment thereof, or the LNP, respectively.

In some embodiments, the first or second click handle is a TCO derivative having one of the following structures:

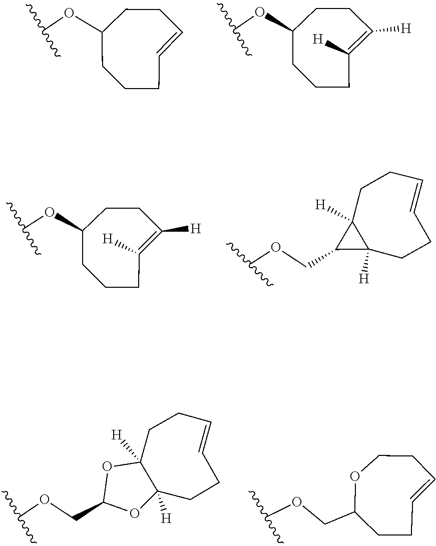

wherein [symbol] represents the point of attachment, directly or indirectly, of the first or second click handle to the targeting moiety, e.g., antibody or a functional fragment thereof, or the LNP, respectively.

In some embodiments, the conjugation efficiency between the first click handle and the second click handle achieved by the disclosed method is greater than 5%, greater than 10%, greater than 25%, greater than 50%, greater than 60%, greater than 70%, greater than 80%, or greater than 90%. In some embodiments, the conjugation efficiency between the first click handle and the second click handle achieved by the disclosed method is from about 5% to about 30%, about 10% to about 20%, about 25% to about 50%, about 30% to about 40%, about 50% to about 80%, about 60% to about 70%, about 70% to about 95%, or about 80% to about 90%. In some embodiments, the conjugate product of the disclosed method can be purified from remaining intermediate product (e.g., the targeting moiety, e.g., antibody or functional fragment thereof, functionalized with a first click handle) using any suitable technique such as, but not limited to, ultrafiltration and diafiltration.

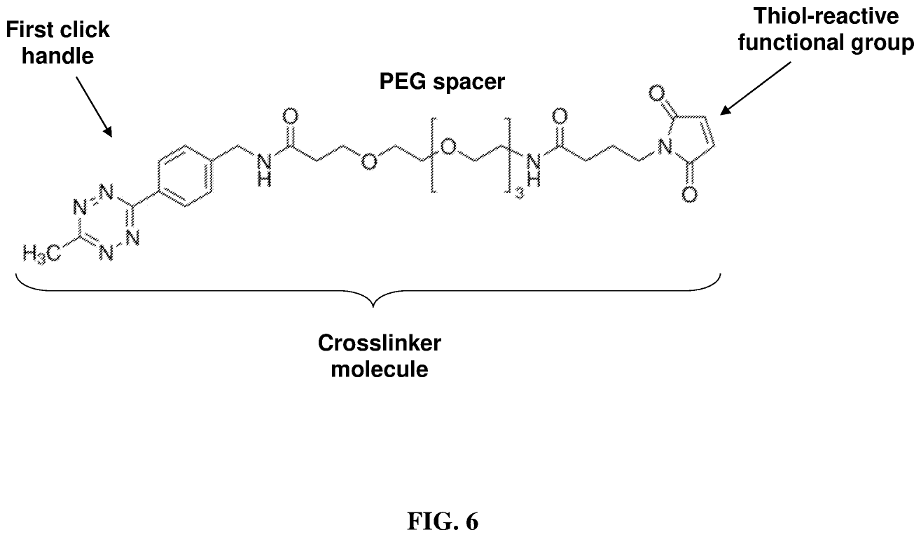

In some embodiments, the crosslinker molecule that conjugates the targeting moiety, e.g., antibody or functional fragment, to the LNP comprises a spacer between the thiol-reactive functional group and the first click handle (see FIG. 4B) In some embodiments, the spacer is a polymer. In some embodiments, the polymer is polyethylene glycol (PEG). In some embodiments, the PEG spacer between the thiol moiety (e.g., a thioether moiety) and the click product comprises n ethylene glycol units. In some embodiments, the PEG spacer comprises at least about 4, 5, 10, 20, 30, 50, 50, 60, 70, 80, 90, 110, or 200 ethylene glycol units. In some embodiments, the PEG spacer comprises about 2 to about 10, about 8 to about 15, about 15 to about 25, about 25 to about 35, about 35 to about 55, about 55 to about 75, about 75 to about 95, about 95 to about 115, about 115 to about 150, or about 150 to about 220. In some embodiments, the PEG spacer comprises about 4 ethylene glycol units. In some embodiments, the PEG spacer comprises more than about 120 ethylene glycol units. FIG. 6 shows the chemical structure of an exemplary crosslinker molecule comprising a methyltetrazine as the first click handle, a maleimide as the thiol-reactive functional group, and PEG as the spacer. In some embodiments, the spacer is polysarcosine (pSar), poly(glycerol) (PGs), poly(2-Oxazoline) and/or poly(peptide).

In some embodiments, the antibody or functional fragment thereof is of the IgG class, the IgM class, or the IgA class. In some embodiments, the antibody or functional fragment thereof is of the IgG class and has an IgG1, IgG2, IgG3, or IgG4 isotype. In some embodiments, the antibody or functional fragment thereof the antibody is a human antibody, a humanized antibody, a bispecific antibody, a monoclonal antibody, a multivalent antibody, or a conjugate antibody. In some embodiments, the antibody or functional fragment thereof is a native protein. In some embodiments, the antibody or functional fragment thereof is an engineered protein.

In some embodiments, a full-length antibody (also referred to as intact antibody or whole antibody used interchangeably herein). In some embodiments, the antibody is a functional antibody fragment, including but not limited to, Fab, Fab′, F(ab′)₂, and Fv fragments; diabodies; linear antibodies; single-chain antibody molecules; and multispecific antibodies formed from antibody fragments. In some embodiments, the antibody fragment is a Single-chain Fv (scFv) antibody fragment which comprises the VH and VL domains of antibody, and wherein these domains are present in a single polypeptide chain. In some embodiments, the antibody is a functional fragment attached to an antigen-binding peptide (e.g., a liner or cyclic peptide) to create a multispecific conjugate. In some embodiments, the antibody is a functional fragment attached to an antigen-binding small molecule to create a multispecific conjugate.

In some embodiments, the linker is attached to the antibody or functional fragment thereof through the C- or N-terminus of the light chain or the C- or N-terminus of the heavy chain. In some embodiments, the linker is attached to the antibody or functional fragment thereof through the C-terminus of the heavy chain. In some embodiments, the linker is attached to the antibody or functional fragment thereof, through both the heavy and light chains. In some embodiments, the antibody or functional fragment thereof comprises a first and second heavy chain and a first and second light chain, each comprising a C- and N-terminus. In some embodiments, the linker is attached to the antibody or functional fragment thereof through the C- or N-terminus of any of the heavy or light chains. In some embodiments, the linker is attached to the antibody or functional fragment thereof through the C-terminus of the first or second heavy chains. In some embodiments, the linker is attached to the antibody or functional fragment thereof through both the first and second heavy chain. In some embodiments, the linker is attached to the antibody or functional fragment thereof through both the first light and heavy chain. In some embodiments, the linker is attached to the antibody or functional fragment thereof through both the second light and heavy chain. In some embodiments, the linker is attached to the antibody or functional fragment thereof site-specifically.

In some embodiments, the targeting moiety is an antibody or fragment thereof, such as a Fab fragment. In some embodiments, the targeting moiety, e.g., antibody or fragment thereof, is engineered to comprise a cysteine at or near (e.g., within 10, 9, 8, 7, 6, 5, 4, 3, 2, or 1 amino acids of) the C-terminus. In some embodiments, the targeting moiety, e.g., antibody or fragment thereof, is engineered to comprise a sequence (e.g., a polypeptide or peptide sequence) at or near the C-terminus, wherein the sequence (e.g., polypeptide or peptide sequence) comprises a free cysteine. In some embodiments, the polypeptide sequence comprises between 1 and 100 amino acids, e.g., 1 to 50 amino acids, 1 to 25 amino acids, 1 to 10 amino acids, 20 to 80 amino acids, or 25 to 100 amino acids. In some embodiments, the polypeptide sequence comprises 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, or 100 amino acids. In some embodiments, the cysteine is located at the C-terminus of the polypeptide sequence. In some embodiments, the cysteine is located within 10 amino acids of the C-terminus of the polypeptide sequence, e.g., within 1, 2, 3, 4, 5, 6, 7, 8, 9, or 10 amino acids of the C-terminus of the polypeptide sequence. In some embodiments, the polypeptide or peptide sequence comprising the free cysteine sequence comprises a flexible spacer sequence, e.g., the polypeptide or peptide sequence comprises a G4S (GGGS) sequence or a series of G4S (GGGS) repeats.

In some embodiments the targeting moiety, e.g., antibody or fragment thereof, such as a Fab fragment, is engineered to comprise a hinge region sequence at or near the C-terminus, wherein the hinge region sequence comprises a cysteine. In some embodiments, the hinge region sequence is engineered to the C or N-terminus of the heavy chain or light chain. In some embodiments, the hinge region sequence is engineered to the C-terminus of the heavy chain or light chain. In some embodiments, the hinge region sequence is engineered to the C-terminus of the heavy chain. In some embodiments, a free cysteine residue is located within the hinge region sequence (FIG. 7A). In some embodiments, the hinge region sequence can comprise a portion of a human IgG1, IgG2, IgG3 or IgG4 hinge region sequence. In some embodiments, the hinge region sequence can comprise a portion of a murine IgG1, IgG2a, IgG2b, or IgG3 hinge region sequence.

In some embodiments, the amino acid sequence introduced at the C-terminus of the Fab fragment (e.g., the C-terminus of the heavy chain of the Fab fragment) is part of a sequence that is present in the hinge region of naturally occurring antibodies or Fab fragments (i.e., a conserved sequence), albeit with one of the two cysteine residues not present. For instance, in some embodiments, the sequence at the C-terminus of the Fab fragment is a conserved sequence in a human IgG1 hinge region such as DKTHTC. In these embodiments, the cysteine (C) residue of DKTHTC is reactive with the thiol-reactive functional group. In some embodiments, additional amino acid residues can be added after the cysteine residue (i.e., C-terminal of the free cysteine residue). For instance, in some embodiments, between 1 and 5 amino acid residues can be added at the C-terminal end of the cysteine residue. In some such embodiments, the amino acid residues added to the C-terminal end of the free cysteine residues are alanine residues.

In some embodiments, the hinge region sequence can comprise or consist of the sequence DKTHTC (SEQ ID NO: 97), DKTHTCA (SEQ ID NO: 98) or DKTHTCAA (SEQ ID NO: 99). In some embodiments, the hinge region sequence can consist or comprise of the sequence EPKSCDKTHTCPPCP (SEQ ID NO: 100), EPKCCVECPPCP (SEQ ID NO: 101), ELKTPLGDTTHTCPRCP(EPKSCDTPPPCPRCP)₃ (SEQ ID NO: 102), ESKYGPPCPSCP (SEQ ID NO: 103), VPRDCGCKPCICT (SEQ ID NO: 104), EPRGPTIKPCPPCK (SEQ ID NO: 105), EPSGPISTINPCPPCK (SEQ ID NO: 106), or EPRIPKPSTPPGSSCP (SEQ ID NO: 107).

It will be understood that sequences other than conserved hinge sequences can be introduced at the C-terminus of the Fab fragment (e.g., the C-terminus of the heavy chain of the Fab fragment), as long as at least one cysteine residue is present.

Following recombinant production of the antibody functional fragment (e.g., Fab fragment), the antibody functional fragment may include a disulfide bond between cysteine groups in the hinge region as a result of an oxidation reaction between the univalent fragments, hence producing a bivalent fragment without a reactive free cysteine residue. In some embodiments, the disulfide bond is reduced prior to reacting with the thiol-reactive functional group, hence generating the free reactive cysteine residue (see FIG. 7A). In some embodiments, the reduction of the disulfide bond in the hinge region of the antigen binding fragment (e.g., Fab fragment) can be accomplished with minimal or no reduction of the interchain disulfide bond at the CL-CH1 interface. It has been discovered that particular sterically hindered reducing agents can reduce the disulfide bonds of the bivalent Fab fragment without substantially effecting interchain disulfide bonds, for instance disulfide bonds near the C-terminus of the Fab fragment. For instance, in some embodiments, tris(2-carboxyethyl)phosphine (TCEP) or TCEP agarose (i.e., TCEP immobilized on agarose) is used as a reducing agent to minimize reduction of the interchain disulfide bond at the CL-CH1 interface of the antibody functional fragment (e.g., Fab fragment). Following reduction of the disulfide bond in the hinge region, the free cysteine group can be coupled with a reaction partner such as a thiol-reactive functional group, as set forth herein. The procedure allows for site-specific introduction of the antibody functional fragment (e.g., Fab fragment) onto the surface of the LNP.

To maximize site-specific introduction of the antibody functional fragment (e.g., Fab fragment) onto the surface of the LNP, the interchain disulfide bond at the CL-CH1 interface of the antibody functional fragment (e.g., Fab fragment) can, is some embodiments, be engineered away from the C-terminus and buried within the CL-CH1 interface (see FIG. 7B). Recombinant engineering of the antibody functional fragment (e.g., Fab fragment) to remove the interchain disulfide bond at the CL-CH1 interface and place the interchain disulfide bond within the interior of the CL-CH1 interface of the antibody functional fragment (e.g., Fab fragment) can potentially reduce or eliminate the reduction of the interchain disulfide bridge outside the hinge region, hence allowing for selective reduction of the disulfide bridge in the hinge region. Accordingly, the methodology enables maximum site-specific introduction of the antibody functional fragment (e.g., Fab fragment) onto the surface of the LNP following the reduction of the disulfide bridge in the hinge region of the antibody functional fragment (e.g., Fab fragment) Specific examples of recombinant engineering of the disulfide bond in the hinge region are provided in Example 2. In some embodiments, the conjugate has a sequence set forth in Table 12.

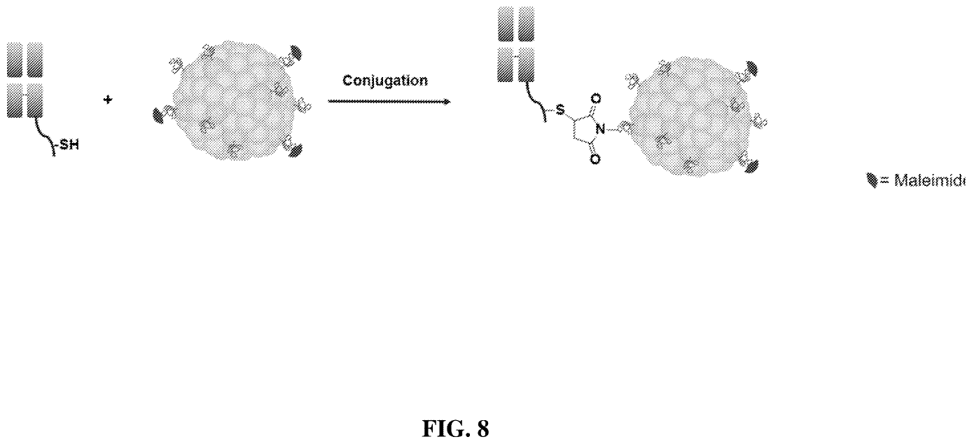

In some embodiments, the Fab fragment with the fully reduced cysteine on the hinge region can react directly with a thiol-reactive functional group in a single-step process, thereby generating a conjugate. FIG. 8 shows one such embodiment, wherein the thiol-reactive functional group is a maleimide.

In some embodiments, the Fab fragment with the fully reduced cysteine in the hinge region can react directly with a thiol-reactive functional group in a two-step process. FIGS. 9A and 9B show an exemplary schematic of conjugate formation. In FIG. 9A, a Fab fragment comprising a free cysteine residue prepared by methods disclosed herein is contacted with a crosslinker molecule. The thiol-reactive functional group of the crosslinker molecule reacts with the free cysteine residue of the Fab fragment to produce the product of step (i). In FIG. 9B, the product of step (i) is contacted with an LNP comprising a second click handle covalently bonded to the surface of the LNP, wherein the first click handle of the Fab fragment reacts with the second click handle via a click chemistry reaction to produce the conjugate (product of step (ii)).

Conjugates prepared by the methods disclosed herein have a high density of the targeting moieties on the surface of the LNP. For instance, the conjugate can comprise more than one targeting moiety, e.g., antibody or functional fragment thereof, per LNP. In some embodiments, the conjugate can comprise more than 10 targeting moieties, e.g., antibodies or functional fragments thereof, per LNP. In some embodiments, the conjugate can comprise more than 20 targeting moieties, e.g., antibodies or functional fragments thereof, per LNP. In some embodiments, the conjugate can comprise more than 30 targeting moieties, e.g., antibodies or functional fragments thereof. In some embodiments, the conjugate can comprise more than 50 targeting moieties, e.g., antibodies or functional fragments thereof, per LNP. In some embodiments, the conjugate can comprise more than 75 targeting moieties, e.g., antibodies or functional fragments thereof, per LNP. In some embodiments, the conjugate can comprise more than 100 targeting moieties, e.g., antibodies or functional fragments thereof, per LNP. In some embodiments, the conjugate can comprise from about 50 to about 200 targeting moieties, e.g., antibodies or functional fragments thereof, per LNP. In some embodiments, the conjugate can comprise from about 100 to about 200 targeting moieties, e.g., antibodies or functional fragments thereof, per LNP. In some embodiments, the conjugate can comprise from about 100 to about 230 targeting moieties, e.g., antibodies or functional fragments thereof, per LNP. In some embodiments, the conjugate can comprise from about 10 to about 150 targeting moieties, e.g., antibodies or functional fragments thereof, per LNP. In some embodiments, the conjugate can comprise from about 10 to about 30 targeting moieties, e.g., antibodies or functional fragments thereof, per LNP.

In any of the foregoing embodies, the targeting moiety, e.g., antibody or functional fragment thereof, can target a cell surface antigen or receptor. In some embodiments, the targeting moiety component of a conjugate, by targeting a cell surface antigen or receptor on a cell, enhances delivery of a payload, e.g., a therapeutic payload, formulated in the LNP component of the conjugate. In some embodiments, the conjugates described herein deliver a payload to more target cells and/or deliver greater amounts of payload to target cells relative to a conjugate lacking a targeting moiety or with fewer targeting moieties per LNP. Enhanced delivery of a therapeutic payload to a target cell, e.g., to an immune cell or a diseased or malfunctional cell, using a targeted conjugate (LNP) as described herein can improve treatment of a disease or ailment in a patient such as a human patient.

### III. Methods of Use

In any of the foregoing embodiments, the targeting moiety component of a conjugate of the disclosure can target a cell surface antigen or receptor. In some embodiments, the targeting moiety component of a conjugate, by targeting a cell surface antigen or receptor on a cell, enhances delivery of a payload, e.g., a therapeutic payload, formulated in the LNP component of the conjugate. In some embodiments, the conjugates described herein deliver a payload to more target cells and/or deliver greater amounts of payload to target cells relative to a conjugate lacking a targeting moiety. Enhanced delivery of a therapeutic payload to a target cell, e.g., to an immune cell or a diseased or malfunctional cell, using a targeted conjugate (LNP) as described herein can improve treatment of a disease or ailment in a patient.

In some embodiments, the targeting moiety component of a conjugate of the disclosure targets T cell receptors including, but not limited to, CD2, CD3, CD4, CD5, CD6, CD7 or CD8. In other embodiments, the targeting moiety component of a conjugate of the disclosure targets hematopoietic stem cells (HSCs). In some embodiments, the targeting moiety component of a conjugate of the disclosure targets HSC receptors including, but not limited to, CD90 or CD117. In other embodiments, the targeting moiety component of a conjugate binds to CD8, TCR alpha, TCR beta, CD10, CD33, CD34, CD68, CD19, CD62L, CD25, CXCR3, CCR2, CCR3, CCR4, CCR5, CCR,6 or CCR7, or combinations thereof.

In certain embodiments, the targeting moiety binds to CD4+ or CD8+ T cell. In other embodiments, the targeting moiety binds to a natural killer (NK) cell. In other embodiments, the targeting moiety binds to a hematopoietic stem cell. In other embodiments, the targeting moiety binds to a lymphoid progenitor cell. In other embodiments, the targeting moiety binds to a myeloid cell. In other embodiments, the targeting moiety binds to a macrophage.

The conjugates of the disclosure can be used to deliver specific payloads to cells, particularly cells expressing cell-surface receptors targeted by the targeting moiety, e.g., antibody, Fab fragment or ScFv, component of the conjugates. In some embodiments the payload is an RNA. In some embodiments, the payload is an mRNA. In other embodiments the payload is a siRNA or a microRNA (miRNA). In other embodiments, the payload is an antisense oligonucleotide (ASO). In other embodiments, the payload is a tRNA. In other embodiments, the payload is a DNA vector, for example, a DNA plasmid, closed-ended DNA (ceDNA), or a small circular DNA (e.g., a nanoplasmid). In other embodiments, the payload is a small molecule. In other embodiments, the payload is a guide RNA. In some embodiments, the payload is a peptide or protein. In some embodiments, the conjugates disclosed herein can include two or more payloads of the same or different payload class, for example, selected from any two or more mRNA, guide RNA, siRNA, miRNA, ASO, DNA vector, small molecule, peptide, and protein.

The conjugates disclosed herein can be used to deliver a therapeutic of interest to a cell. For instance, in some embodiments, the conjugates disclosed herein can be used to deliver a nucleic acid (e.g., mRNA) encoding a vaccine. In other embodiments, the conjugates disclosed herein can be used to deliver a nucleic acid (e.g., mRNA) encoding an enzyme. In other embodiments, the conjugates disclosed herein can be used to deliver a nucleic acid (e.g., a DNA or RNA molecule) encoding a chimeric antigen receptor (CAR) to T cells.

The conjugates described herein may be used to target and modify immune cells. In some embodiments, the conjugates may be used to modify T cells. In some embodiments, T-cells may include any subpopulation of T-cells, e.g., CD4+, CD8+, gamma-delta, naïve T cells, stem cell memory T cells, central memory T cells, or a mixture of subpopulations. In some embodiments, the conjugates may be used to deliver or modify a T-cell receptor (TCR) in a T cell. In some embodiments, the conjugates may be used to deliver at least one chimeric antigen receptor (CAR) to T-cells. For instance, in specific embodiments, the conjugates can be used to deliver a CAR or a nucleic acid (e.g., a DNA or RNA, such as mRNA) encoding a CAR to T-cells. In some embodiments, the conjugates may be used to deliver at least one CAR, a nucleic acid (e.g., a DNA or RNA, such as mRNA) encoding a CAR to natural killer (NK) cells. In some embodiments, the conjugates can be used to deliver at least one CAR or a nucleic acid (e.g., a DNA or RNA, such as mRNA) encoding a CAR to natural killer T (NKT) cells. In some embodiments, the conjugates may be used to deliver at least one CAR or a nucleic acid (e.g., a DNA or RNA, such as mRNA) encoding a CAR to a progenitor cell, e.g., a progenitor cell of T, NK, or NKT cells. In some embodiments, cells modified with at least one CAR (e.g., CAR-T cells, CAR-NK cells, CAR-NKT cells), or a combination of cells modified with at least one CAR (e.g., a mixture of CAR-NK/T cells) are used to treat a condition as identified in the targetable landscape of CAR therapies in MacKay, et al. Nat Biotechnol 38, 233-244 (2020). In some embodiments, the immune cells comprise a CAR specific to a tumor or a pathogen antigen selected from a group consisting of AChR (fetal acetylcholine receptor), ADGRE2, AFP (alpha fetoprotein), BAFF-R, BCMA, CAIX (carbonic anhydrase IX), CCR1, CCR4, CEA (carcinoembryonic antigen), CD3, CD5, CD8, CD7, CD10, CD13, CD14, CD15, CD19, CD20, CD22, CD30, CD33, CLLI, CD34, CD38, CD41, CD44, CD49f, CD56, CD61, CD64, CD68, CD70, CD74, CD99, CD117, CD123, CD133, CD138, CD44v6, CD267, CD269, CDS, CLEC12A, CS1, EGP-2 (epithelial glycoprotein-2), EGP-40 (epithelial glycoprotein-40), EGFR (HER1), EGFR-VIII, EpCAM (epithelial cell adhesion molecule), EphA2, ERBB2 (HER2, human epidermal growth factor receptor 2), ERBB3, ERBB4, FBP (folate-binding protein), Flt3 receptor, folate receptor-a, GD2 (ganglioside G2), GD3 (ganglioside G3), GPC3 (glypican-3), GPI00, hTERT (human telomerase reverse transcriptase), ICAM-1, integrin B7, interleukin 6 receptor, IL13Ra2 (interleukin-13 receptor 30 subunit alpha-2), kappa-light chain, KDR (kinase insert domain receptor), LeY (Lewis Y), LICAM (LI cell adhesion molecule), LILRB2 (leukocyte immunoglobulin like receptor B2), MARTI, MAGE-A1 (melanoma associated antigen Al), MAGE-A3, MSLN (mesothelin), MUC16 (mucin 16), MUCI (mucin I), KG2D ligands, NY-ESO-1 (cancer-testis antigen), PRI (proteinase 3), TRBCI, TRBC2, TFM-3, TACI, tyrosinase, survivin, hTERT, oncofetal antigen (h5T4), p53, PSCA (prostate stem cell antigen), PSMA (prostate-specific membrane antigen), hROR1, TAG-72 (tumor-associated glycoprotein 72), VEGF-R2 (vascular endothelial growth factor R2), WT-1 (Wilms tumor protein), and antigens of HIV (human immunodeficiency virus), hepatitis B, hepatitis C, CMV (cytomegalovirus), EBV (Epstein-Barr virus), HPV (human papilloma virus).

In some embodiments, a conjugate as described herein is administered to an immune cell, e.g., a T-cell, NK cell, NKT cell, or progenitor cell ex vivo or in vitro to deliver a therapeutic payload (e.g., a gene modifying system) and then the cells are delivered to a patient. In some embodiments, immune cells, e.g., T-cells, NK cells, NKT cells, or progenitor cells are modified ex vivo or in vitro and then delivered to a patient. In some embodiments, a nucleic acid (e.g., DNA or RNA, such as mRNA) is delivered by one of the methods mentioned herein, and immune cells, e.g., T-cells, NK cells, NKT cells, or progenitor cells are modified in vivo in the patient. In some embodiments the patient is a human patient such as a human patient in need of such treatment.

In certain embodiments, the targeting moiety is a T-cell targeting moiety, for example, an antibody, Fab fragment or ScFv, that binds to a T-cell antigen selected from the group consisting of CD2, CD3, CD4, CD5, CD7, CD8, CD28, CD137, CD45, T-cell receptor (TCR)β, TCR-α, TCR-α/β, TCR-γ/δ, PD1, CTLA4, TIM3, LAG3, CD18, IL-2 receptor, CD11a, TLR2, TLR4, TLR5, IL-7 receptor, or IL-15 receptor.

In some embodiments, a conjugate as described herein is administered to an HSC (e.g., a LT-HSC) or a HSC progenitor ex vivo or in vitro to deliver a therapeutic payload (e.g., a gene modifying system) and then the cells are delivered to a patient. In some embodiments, a conjugate as described herein is administered to an HSC (e.g., a LT-HSC) or a HSC progenitor in vivo to deliver a therapeutic payload (e.g., a gene modifying system). In some embodiments, HSCs (e.g., LT-HSCs) or HSC progenitor cells are modified ex vivo or in vitro and then delivered to a patient. In some embodiments, HSCs (e.g., LT-HSCs) or HSC progenitor cells are modified in vivo in the patient. In some embodiments the patient is a human patient such as a human patient in need of such treatment.

In certain embodiments, the targeting moiety is a HSC targeting moiety, for example, an antibody, Fab fragment or ScFv, that binds to an HSC antigen selected from CD90 and CD117.

A conjugate as disclosed herein can be introduced into cells, tissues and multicellular organisms. In some embodiments the system or components of the system are delivered to the cells via mechanical means or physical means. In some embodiments the cells are human cells.

In some embodiments, a conjugate described herein is delivered to a tissue or cell from or in the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type. In some embodiments, a conjugate described herein is used to treat a disease, such as a cancer, inflammatory disease, infectious disease, genetic defect, or other disease. A cancer can be cancer of the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type, and can include multiple cancers.

In some embodiments, a conjugate described herein is administered by enteral administration (e.g. oral, rectal, gastrointestinal, sublingual, sublabial, or buccal administration). In some embodiments, a conjugate system described herein is administered by parenteral administration (e.g., intravenous, intramuscular, subcutaneous, intradermal, epidural, intracerebral, intracerebroventricular, epicutaneous, nasal, intra-arterial, intra-articular, intracavernous, intraocular, intraosseous infusion, intraperitoneal, intrathecal, intrauterine, intravaginal, intravesical, perivascular, or transmucosal administration). In some embodiments, a conjugate described herein is administered by topical administration (e.g., transdermal administration).

In some embodiments, a conjugate described herein is used to treat a disease, disorder, or condition. In some embodiments, a conjugate described herein, or component or portion thereof, is used to treat a disease, disorder, or condition listed in any of Tables 1-6. In some such embodiments, the conjugate described herein, or component or portion thereof, is used to treat a disease, disorder, or condition in a human patient. In some embodiments, a conjugate described herein is used to treat a hematopoietic stem cell (HSC) disease, disorder, or condition, e.g., as listed in Table 1. In some embodiments, a conjugate described herein is used to treat a kidney disease, disorder, or condition, e.g., as listed in Table 2. In some embodiments, a conjugate described herein is used to treat a liver disease, disorder, or condition, e.g., as listed in Table 3. In some embodiments, a conjugate described herein is used to treat a lung disease, disorder, or condition, e.g., as listed in Table 4. In some embodiments, a conjugate described herein is used to treat a skeletal muscle disease, disorder, or condition, e.g., as listed in Table 5. In some embodiments, a conjugate described herein is used to treat a skin disease, disorder, or condition, e.g., as listed in Table 6.

### Tables 1-6: Particular Indications

TABLE 1
HSCs

| Disease | Gene Affected |
|---|---|
| Adrenoleukodystrophy (CALD) | ABCD1 |
| Alpha-mannosidosis | MAN2B1 |
| Fanconi anemia | FANCA; FANCC; FANCG |
| Gaucher disease | GBA |
| Globoid cell leukodystrophy | GALC |
| (Krabbe disease) | |
| Hemophagocytic lymphohistiocytosis | PRF1; STX11; STXBP2; UNC13D |
| Malignant infantile osteopetrosis- | TCIRG1; Many genes implicated |
| autosomal recessive osteopetrosis | |
| Metachromatic leukodystrophy | ARSA; PSAP |
| MPS 1S (Scheie syndrome) | IDUA |
| MPS2 | IDS |
| MPS7 | GUSB |
| Mucolipidosis II | GNPTAB |
| Niemann-Pick disease A and B | SMPD1 |
| Niemann-Pick disease C | NPC1 |
| Pompe disease | GAA |
| Sickle cell disease (SCD) | HBB |
| Tay Sachs | HEXA |
| Thalassemia | HBB |

TABLE 2
Kidney

| Disease | Gene Affected |
|---|---|
| Congenital nephrotic syndrome | NPHS2 |
| Cystinosis | CTNS |

TABLE 3
Liver

| Disease | Gene Affected |
|---|---|
| Acute intermittent porphyria | HMBS |
| Alagille syndrome | JAG1 |
| Alpha-1 antitrypsin deficiency | SERPINA1 |
| Carbamoyl phosphate synthetase I deficiency | CPS1 |
| Citrullinemia I | ASS1 |
| Crigler-Najjar | UGT1A1 |
| Fabry | LPL |
| Familial chylomicronemia syndrome | GLA |
| Gaucher | GBE1 |
| GSD1a | G6Pase |
| GSD IV | GBA |
| Heme A | F8 |
| Heme B | F9 |
| HoFH | LDLRAP1 |
| Methylmalonic acidemia | MMUT |
| MPS II | IDS |
| MPS III | Type IIIa: SGSH |
| | Type IIIb: NAGLU |
| | Type IIIc: HGSNAT |
| | Type IIId: GNS S |
| MPS IV | Type IVA: GALNS |
| | Type IVB: GLB1 |
| MPS VI | ARSB |
| MSUD | Type Ia: BCKDHA |
| | Type Ib: BCKDHB |
| | Type II: DBT |
| OTC Deficiency | OTC |
| Polycystic Liver Disease | PRKCSH |
| Pompe | GAA |
| Primary Hyperoxaluria 1 | AGXT (HAO1 or |
| | LDHA for CRISPR) |
| Progressive familial intrahepatic cholestasis type 1 | ATP8B1 |
| Progressive familial intrahepatic cholestasis type 2 | ABCB11 |
| Progressive familial intrahepatic cholestasis type 3 | ABCB4 |
| Propionic acidemia | PCCB; PCCA |
| Wilson's Disease | ATP7B |

TABLE 4
Lung

| Disease | Gene Affected |
|---|---|
| Alpha-1 antitrypsin deficiency | SERPINA1 |
| Cystic fibrosis | CFTR |
| Primary ciliary dyskinesia | DNAI1 |
| Primary ciliary dyskinesia | DNAH5 |
| Primary pulmonary hypertension I | BMPR2 |
| Surfactant Protein B (SP-B) Deficiency | SFTPB |
| (pulmonary surfactant metabolism dysfunction 1) | |

TABLE 5
Skeletal muscle

| Disease | Gene Affected |
|---|---|
| Becker muscular dystrophy | DMD |
| Becker myotonia | CLCN1 |
| Bethlem myopathy | COL6A2 |
| Centronuclear myopathy, X-linked (motubular) | MTM1 |
| Congenital myasthenic syndrome | CHRNE |
| Duchenne muscular dystrophy | DMD |
| Emery-Dreifuss muscular dystrophy, AD | LMNA |
| Limb-girdle muscular dystrophy 2A | CAPN3 |
| Limb-girdle muscular dystrophy, type 2D | SGCA |

TABLE 6
Skin

| Disease | Gene Affected |
|---|---|
| Epidermolysis Bullosa Dystrophica Recessive | COL7A1 |
| (Hallopeau-Siemens) | |
| Epidermolysis Bullosa Junctional | LAMB3 |
| Epidermolytic Ichthyosis | KRT1; KRT10 |
| Hailey-Hailey Disease | ATP2C1 |
| Lamellar Ichthyosis/Nonbullous Congenital | TGM1 |
| Ichthyosiform Erythroderma (ARCI) | |
| Netherton Syndrome | SPINK5 |

### IV. Therapeutic Payloads

The conjugates (targeted LNPs) described herein can be used to deliver payloads (e.g., comprising therapeutic agents) to cells, such as, but not limited to, immune cells (e.g., T cells) or HSCs (e.g., LT-HSCs) or HSC progenitors.

In some embodiments, the payload is one or more nucleic acids. In some embodiments, the payload is one or more RNA molecules. In some embodiments, the payload is an mRNA (e.g., an mRNA encoding an enzyme). In some embodiments, the RNA molecule is a non-coding RNA (ncRNA). In some embodiments, the payload is an RNA template (for example, an RNA template for reverse transcription, e.g., Target Primed Reverse Transcription (TPRT)). In other embodiments the payload is a siRNA or a microRNA (miRNA). In other embodiments, the payload comprises a guide RNA for a CRISPR-Cas system. In other embodiments, the payload is a tRNA. In other embodiments, the payload is an antisense oligonucleotide (ASO). In other embodiments, the payload is a DNA molecule, for example, a DNA plasmid, closed-ended DNA (ceDNA), or a small circular DNA (e.g., a minicircle or nanoplasmid). Nucleic acid payloads can be linear, circular, covalently closed, single-stranded, double-stranded, or hybrid RNA/DNA molecules. In other embodiments, the payload is a small molecule. In some embodiments, the payload is a peptide or protein. In some embodiments, the conjugates disclosed herein can include two or more payloads, for example, selected from RNA (such as mRNA, ncRNA, guide RNA, siRNA, miRNA), an ASO, DNA vector, small molecule, peptide, and protein.

The conjugates (targeted LNPs) described herein can be used to deliver a therapeutic of interest to a cell, such as, but not limited to an immune cell (e.g., a T cell), HSC or HSC progenitor. In some embodiments, the conjugate (targeted LNP) contains a payload that is a therapeutic agent. In some embodiments, the therapeutic agent can be a therapeutic peptide or protein, a nucleic acid comprising a therapeutic agent, or a nucleic acid encoding a therapeutic agent. In some embodiments, the therapeutic agent can be a genetic medicine (e.g., for gene therapy or gene editing), wherein the therapeutic agent is capable of modifying, altering or effecting a change in the genomic DNA of a cell such as, but not limited to, an immune cell, such as a T cell, an HSC (e.g., LT-HSC) or HSC progenitor in the subject). In some embodiments, the therapeutic agent is a gene therapy agent or gene editing agent. In some embodiments, the therapeutic agent is a gene modifying polypeptide, as described herein. In some embodiments, the therapeutic agent is a gene modifying system, as described herein.

In some embodiments, the therapeutic agent can be a peptide or protein, such as an enzyme, or a nucleic acid (e.g., mRNA or DNA) encoding the peptide or protein (e.g., an enzyme). In some embodiments, the enzyme can be or comprise a nuclease, recombinase, integrase, transposase, retrotransposase, helicase, transcriptase, polymerase, reverse transcriptase, deaminase, methylase, demethylase, or ligase, or can have a combination of enzymatic activities thereof. In some embodiments, the therapeutic agent can be a peptide or protein, or a nucleic acid encoding the peptide or protein, for use as a replacement gene therapy. In some embodiments, the therapeutic agent can be a peptide or protein, or a nucleic acid encoding the peptide or protein, for use in modifying or altering the genome or epigenome of a cell, such as, but not limited to, an immune cell, such as a T cell, an HSC or HSC progenitor of a subject. In some embodiments, the therapeutic agent can comprise one or more components of a system for modifying or altering the genome or epigenome of a cell such as, but not limited to, an immune cell such as a T cell, an HSC or HSC progenitor of a subject. In some embodiments, the system for modifying or altering the genome or epigenome of a cell of a subject comprises one or more proteins, one or more nucleic acids (e.g., RNA and/or DNA), or combinations thereof. In some embodiments, the therapeutic agent can be a fusion protein, e.g., a fusion protein comprising a nuclease (e.g., an endonuclease such as Cas9 or a functional portion thereof) and a protein domain comprising recombinase, integrase, transposase, retrotransposase, helicase, transcriptase, polymerase, reverse transcriptase, deaminase, methylase, demethylase, or ligase activity.

In some embodiments, the therapeutic agent can be one or more components of a ribonucleoprotein (RNP) complex for modifying or altering the genome or epigenome of a cell such as, but not limited to, an immune cell, such as a T cell, an HSC or HSC progenitor. For example, in some cases the therapeutic agent can be a protein, or a nucleic acid (e.g., mRNA) encoding the protein, and/or an RNA molecule (e.g., a gRNA or RNA comprising a gRNA) for guiding the protein to a particular location in the genome or epigenome, wherein the protein is capable of modifying or altering the genome or epigenome as a nuclease, recombinase, integrase, transposase, helicase, transcriptase, polymerase, reverse transcriptase, deaminase, methylase, demethylase, or ligase, or combinations thereof.

In certain embodiments, the therapeutic agent comprises a nuclease (e.g., an endonuclease) or a nucleic acid encoding the nuclease (e.g., an endonuclease). In some embodiments, the nuclease cleaves DNA to create a double stranded break, leading to the introduction of insertion and/or deletion (indel) mutations in DNA, e.g., genomic DNA. In certain embodiments, the nuclease is a nickase (i.e., it cleaves a single stand of DNA). In certain embodiments, the nuclease is mutated such that it is inactive or comprises reduced nuclease activity. In some embodiments, the nuclease is a CRISPR-Cas protein. In some embodiments, the nuclease is a recombinant nuclease. In some embodiments, the nuclease is a restriction endonuclease, meganuclease, homing endonuclease, zinc finger nuclease (ZFN), or a transcription activator-like effector nuclease (TALEN).

In some embodiments, the conjugates (targeted LNPs) of the disclosure can be used to deliver gene editing components into cells. In some embodiments, the conjugates described herein can be used to deliver a therapeutic agent comprising a CRISPR-Cas system or a component thereof into a cell. In some embodiments, the therapeutic agent comprises a Class 1 (type I, type III, or type IV) CRISPR-Cas protein or a nucleic acid encoding the CRISPR-Cas protein. In some embodiments, the therapeutic agent comprises a Class 2 (type II, type V, or type VI) CRISPR-Cas protein or a nucleic acid encoding the CRISPR-Cas protein. In some embodiments, the therapeutic agent comprises a CRISPR-Cas9 system, or a nucleic acid encoding one or more components of the CRISPR-Cas9 system. In some embodiments, the therapeutic agent comprises a CRISPR-Cas12 system (e.g., a Cas12a system), or a nucleic acid encoding one or more components of the CRISPR-Cas12 system. In some such embodiments, the conjugates described herein can comprise two RNA molecules, such as an RNA comprising a guide RNA (gRNA) and an mRNA encoding a CRISPR-Cas protein. In some embodiments, the Cas is Cas9 or Cas12a. In some embodiments, the Cas is Cas9. In some embodiments, the Cas is Cas12a. In some embodiments, the gRNA is a single guide RNA (sgRNA). In some embodiments, the LNPs, e.g., targeted LNPs, comprise a payload consisting of or comprising a Cas9 or an mRNA encoding a Cas9.

In some embodiments, the therapeutic agent comprises one of the following CRISPR-Cas proteins or a nucleic acid (e.g., mRNA) encoding one of the following CRISPR-Cas proteins: Cas9 (e.g., dCas9 and nCas9), Cas12a/Cpfl, Cas12b/C2cl, Cas12c/C2c3, Cas12d/CasY, Cas12e/CasX, Cas12g, Cas12h, or Cas12i. In some embodiments, the therapeutic agent comprises an S. pyogenes or an S. thermophilus Cas9, or a functional fragment thereof, or a nucleic acid encoding the Cas9 or functional fragment thereof. In some embodiments, the therapeutic agent comprises a Cas9 sequence, e.g., as described in Chylinski, Rhun, and Charpentier (2013) RNA Biology 10:5, 726-737; incorporated herein by reference.

In embodiments, the therapeutic agent comprises one of the following CRISPR-Cas proteins or a nucleic acid (e.g., mRNA) encoding one of the following CRISPR-Cas proteins: Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpfl, Cas12b/C2cl, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csal, Csa2, Csa3, Csa4, Csa5, Type II Cas effector proteins, Type V Cas effector proteins, Type VI Cas effector proteins, CARF, DinG, Cpfl, Cas12b/C2cl, Cas12c/C2c3, Cas12b/C2cl, Cas12c/C2c3, SpCas9 (K855A), eSpCas9 (1.1), SpCas9-HF1, hyper accurate Cas9 variant (HypaCas9), SpRYCas9, homologues thereof, modified or engineered versions thereof, and/or functional fragments thereof. In embodiments, the Cas9 comprises one or more substitutions, e.g., selected from H840A, D10A, P475A, W476A, N477A, D1125A, W1126A, and D1127A. In embodiments, the Cas9 comprises one or more mutations at positions selected from: D10, G12, G17, E762, H840, N854, N863, H982, H983, A984, D986, and/or A987, e.g., one or more substitutions selected from D10A, G12A, G17A, E762A, H840A, N854A, N863A, H982A, H983A, A984A, and/or D986A. In some embodiments, the therapeutic agent comprises a Cas (e.g., Cas9) or a nucleic acid encoding a Cas from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Campylobacter jejuni, Neisseria meningitidis, Streptococcus pyogenes, or Staphylococcus aureus, or a fragment or variant thereof.

In some embodiments, the therapeutic agent comprises a Cpf1 domain, e.g., comprising one or more substitutions, e.g., at position D917, E1006A, D1255 or any combination thereof, e.g., selected from D917A, E1006A, D1255A, D917A/E1006A, D917A/D1255A, E1006A/D1255A, and D917A/E1006A/D1255A, or a nucleic acid encoding the same.

In some embodiments, the therapeutic agent comprises an spCas9, spCas9-VRQR, spCas9-VRER, xCas9, saCas9, saCas9-KKH, spCas9-MQKSER, spCas9-LRKIQK, or spCas9-LRVSQL, or a nucleic acid encoding the same.

In some embodiments, the therapeutic agent comprises a Cas9 nickase (nCas9), such as an S. pyogenes nCas9, e.g., wherein the Cas9 comprises an amino acid substitution at position D10 or H840, e.g., D10A or H840A, or a nucleic acid (e.g., mRNA) encoding the Cas9 nickase (nCas9). In some embodiments, the therapeutic agent comprises a catalytically inactive or “dead” Cas9 (dCas9), such as an S. pyogenes Cas9, e.g., wherein the Cas9 comprises an amino acid substitution at positions D10 and H840, e.g., D10A and H840A, or a nucleic acid (e.g., an mRNA) encoding the catalytically inactive Cas9 (dCas9).

In certain embodiments, the therapeutic agent comprises a deaminase, such as a cytidine deaminase or an adenine deaminase, or a nucleic acid (e.g., mRNA) encoding a deaminase. In some embodiments, conjugates (targeted LNPs) described herein deliver the deaminase to a target cell to generate a substitution mutation in the DNA, e.g., genomic DNA, of the cell. In some embodiments, the therapeutic agent is a base editor, as described in the art, e.g., a cytidine base editor (CBE) or an adenine nucleobase editor (ABE). Examples of therapeutic agents comprising a deaminase or nucleic acids encoding deaminases can be found in PCT Application Nos. PCT/US2014/038359, PCT/US2017/045381, PCT/US2018/024208, PCT/US2018/056146, and PCT/US2019/050112 incorporated herein by reference in their entirety, including the sequence listing and sequences therein.

In certain embodiments, the therapeutic agent can be used for epigenome editing. In some embodiments, the therapeutic agent comprises a methylase and/or a demethylase, or one or more nucleic acids (e.g., one or more mRNA) encoding a demethylase and/or a methylase. In some embodiments, the therapeutic agent demethylates DNA (e.g., genomic DNA) and/or histones. In some embodiments, the therapeutic agent methylates DNA (e.g., genomic DNA) and/or histones. In some embodiments, the therapeutic agent can be useful in altering the transcription of a gene (e.g., via gene silencing or gene activation using CRISPRoff and/or CRISPRon). In some embodiments, the therapeutic agent can comprise a DNA methyltransferase domain or a nucleic acid encoding a DNA methyltransferase domain. In some embodiments, the therapeutic agent can comprise a KRAB domain, DNMT3A domain, DNMT3B domain, DNMT1 domain, DNMMT3L domain, or SETDB1 domain, or can comprise a nucleic acid encoding a KRAB domain, DNMT3A domain, DNMT3B domain, DNMT1 domain, DNMMT3L domain, SETDB1 domain, VP64 domain, p65 domain, TET1 domain, TET2 domain, or TET3 domain. Examples of therapeutic agents comprising a methylase and//or demethylase or nucleic acids encoding methylases and, or demethylases can be found in PCT Application Nos. PCT/IB2015/058202, PCT/US2021/035244, and PCT/US2021/035937 incorporated herein by reference in their entirety, including the sequence listing and sequences therein.

In certain embodiments, the therapeutic agent can be used to alter or modify a nucleic acid sequence, e.g., to introduce an indel or a substitution into DNA (e.g., genomic DNA) by inducing target-primed reverse transcription (TPRT) to insert a heterologous sequence into the DNA. In certain embodiments, the therapeutic agent (i.e., payload) can be a gene modifying protein, a nucleic acid encoding a gene modifying protein, or a gene modifying system, as described herein. In some embodiments, the therapeutic agent can be a gene modifying polypeptide or nucleic acid encoding a gene modifying polypeptide. In some embodiments, the therapeutic agent can be a template RNA for use with the gene modifying polypeptide. In some embodiments, the therapeutic agent delivered by a conjugate (targeted LNP) described herein is one or more components of a gene modifying system, e.g., a gene modifying polypeptide (or nucleic acid encoding the gene modifying polypeptide) and/or a template RNA for use with the gene modifying polypeptide. In some embodiments, the therapeutic agent comprises or is derived from a retrotransposon or mobile genetic element (MGE). In some embodiments, the therapeutic agent can be a heterologous gene modifying polypeptide or nucleic acid encoding a heterologous gene modifying polypeptide, as described herein. In some embodiments, the therapeutic agent can be an RNA for use with the heterologous gene modifying polypeptide. In some embodiments, the therapeutic agent delivered by a conjugate (targeted LNP) described herein is one or more components of a heterologous gene modifying system, e.g., a heterologous gene modifying polypeptide (or nucleic acid encoding the heterologous gene modifying polypeptide) and/or an RNA for use with the heterologous gene modifying polypeptide. In some embodiments, the therapeutic agent is a fusion protein, e.g., an endonuclease protein fused to a reverse transcriptase, e.g., an endonuclease nickase fused to a reverse transcriptase. In some embodiments, the therapeutic agent is a fusion protein, e.g., an endonuclease protein fused to a polymerase, e.g., an endonuclease nickase fused to a polymerase.

Other examples of therapeutic agents can be found in PCT Application Nos. PCT/US2020/023730, PCT/US2021/031439, PCT/US2020/055156, PCT/US2021/052097, PCT/US2022/012054, PCTUS2022/023175, PCT/US2022/074628, and PCT/US2023/065947 incorporated herein by reference in their entirety, including the sequence listing and sequences therein.

In some embodiments, the mRNA component of a gene modifying system comprises a recombinant nuclease, or a nucleic acid encoding the nuclease, for example a CRISPR-Cas nuclease (such as a nickase), restriction endonuclease, meganuclease, homing endonuclease, zinc finger nuclease (ZFN), or a transcription activator-like effector nuclease (TALEN).

In certain embodiments, the therapeutic agent can be a small molecule. In certain embodiments, the therapeutic agent can be an siRNA or miRNA.

### Gene Modifying Systems

The conjugates (targeted LNPs) described herein can be formulated to comprise one or more components of a gene modifying system or one or more nucleic acids encoding said components. Accordingly, in some embodiments, the payload comprises a gene modifying system, or one or more nucleic acids encoding the components of the gene modifying system. For instance, in some embodiments, the payload comprises a template RNA and an mRNA encoding the gene modifying polypeptide. In some embodiments, conjugates prepared in accordance with this disclosure are used to deliver to target cells systems that are capable of inserting a heterologous object sequence (e.g., a sequence encoding a CAR) into the genome of the cell, e.g., an immune cell, such as a T cell. In some embodiments, the system comprises: (A) a gene modifying polypeptide or a nucleic acid encoding the gene modifying polypeptide, wherein the gene modifying polypeptide comprises: (i) an endonuclease and/or DNA binding domain; and (ii) a reverse transcriptase (RT) domain, where (i) and (ii) are both derived from a retrotransposon (e.g., from the same retrotransposon or different retrotransposons); and (B) a template RNA (or DNA encoding the template RNA) comprising (i) a sequence that binds the polypeptide and (ii) a heterologous object sequence. A gene modifying polypeptide, in some embodiments, acts as a substantially autonomous protein machine capable of integrating a template nucleic acid sequence into a target DNA molecule (e.g., in a mammalian host cell, such as a genomic DNA molecule in the host cell), substantially without relying on host machinery. The heterologous object sequence may include, e.g., a coding sequence, a regulatory sequence, a gene expression unit.

In some embodiments, systems described herein can have a number of advantages relative to various earlier systems. For instance, the disclosure describes retrotransposases capable of inserting long sequences of heterologous nucleic acid into a genome. In addition, retrotransposases described herein can insert heterologous nucleic acid in an endogenous site in the genome, such as the rDNA locus. This is in contrast to Cre/loxP systems, which require a first step of inserting an exogenous loxP site before a second step of inserting a sequence of interest into the loxP site.

### Gene Modifying Polypeptides

Non-long terminal repeat (LTR) retrotransposons are a type of mobile genetic elements that are widespread in eukaryotic genomes. They include, for example, the apurinic/apyrimidinic endonuclease (APE)-type, the restriction enzyme-like endonuclease (RLE)-type, and the Penelope-like element (PLE)-type.

The APE class retrotransposons are comprised of two functional domains: an endonuclease/DNA binding domain, and a reverse transcriptase domain. Examples of APE-class retrotransposons can be found, for example, in Table 1 of PCT Application No. PCT/US2019/048607, US 2023/0235358, US 2023/0242899, and US 2020/0109398, the disclosures of which are incorporated herein by reference in their entireties, including the sequence listing and sequences referred to in Table 1 in PCT/US2019/048607 and US 2020/0109398.

The RLE class are comprised of three functional domains: a DNA binding domain, a reverse transcription domain, and an endonuclease domain. Examples of RLE-class retrotransposons can be found, for example, in Table 2 of PCT Application No. PCT/US2019/048607, US 2023/0235358, US 2023/0242899, and US 2020/0109398, the disclosures of which are incorporated herein by reference in their entireties, including the sequence listing and sequences referred to in Table 2 in in PCT/US2019/048607 and US 2020/0109398.

The reverse transcriptase domain of non-LTR retrotransposon functions by binding an RNA sequence template and reverse transcribing it into the host genome's target DNA. The RNA sequence template has a 3′ untranslated region which is specifically bound to the retrotransposase, and a variable 5′ region generally having Open Reading Frame(s) (“ORF”) encoding retrotransposase proteins. The RNA sequence template may also comprise a 5′ untranslated region which specifically binds the retrotransposase.

Penelope-like elements (PLEs) are distinct from both LTR and non-LTR retrotransposons. PLEs generally comprise a reverse transcriptase domain distinct from that of APE and RLE elements, but similar to that of telomerases and Group II introns, and an optional GIY-YIG endonuclease domain.

Other exemplary classes of retrotransposon include, without limitation, RTE (e.g., RTE-1_MD, RTE-3_BF, and RTE-25_LMi), CR1 (e.g., CR1-1_PH), Crack (e.g., Crack-28_RF), L2 (e.g., L2-2_Dre and L2-5_GA), and Vingi (e.g., Vingi-1_Acar) retrotransposons.

As described herein, the elements of such retrotransposons can be functionally modularized and/or modified to target, edit, modify or manipulate a target DNA sequence, e.g., to insert an object (e.g., heterologous) nucleic acid sequence into a target genome, e.g., a mammalian genome, by reverse transcription. In some embodiments, a gene modifying system comprises: (A) a polypeptide or a nucleic acid encoding a polypeptide, wherein the polypeptide comprises (i) a retrotransposase reverse transcriptase domain, and (ii) a retrotransposase endonuclease domain that contains DNA binding functionality; and (B) a template RNA (or DNA encoding the template RNA) comprising (i) a sequence that binds the polypeptide and (ii) a heterologous object sequence. The RNA template element of a gene modifying system is typically heterologous to the polypeptide element and provides an object sequence to be inserted (reverse transcribed) into the host genome.

In some embodiments, the gene modifying system comprises a retrotransposase sequence of an element listed in any one of Table 10, Table 11, Table X, Table Z1 Table 3A, or 3B of PCT Pub. No.: WO/2021/178717, US 2023/0235358, and US 2023/0242899, which are incorporated herein by reference as they relate to domains from retrotransposons.

In some embodiments, an amino acid sequence encoded by an element of Table 7 is an amino acid sequence encoded by the full length sequence of an element listed in Table 7, or a sequence having at least 75%, 80%, 85%, 90%, 95%, 96%, 97%, 98%, or 99% sequence identity thereto. In some embodiments, the full-length sequence of an element listed in Table 7 may comprise one or more (e.g., all of) of a 5′ UTR, polypeptide-encoding sequence, or 3′ UTR of a retrotransposon as described herein. In some embodiments, an amino acid sequence of Table 7 is an amino acid sequence encoded by the full length sequence of an element listed in Table 7, or a sequence having at least 75%, 80%, 85%, 90%, 95%, 96%, 97%, 98%, or 99% sequence identity thereto. In some embodiments, a 5′ UTR of an element of Table 7 comprises a 5′ UTR of the full length sequence of an element listed in Table 7, or a sequence having at least 75%, 80%, 85%, 90%, 95%, 96%, 97%, 98%, or 99% sequence identity thereto. In some embodiments, a 3′ UTR of an element of Table 7 comprises a 3′ UTR of the full length sequence of an element listed in Table 7, or a sequence having at least 75%, 80%, 85%, 90%, 95%, 96%, 97%, 98%, or 99% sequence identity thereto.

Also indicated in Table 7 are the host organisms from which the nucleic acid sequences were obtained and a listing of domains present within the polypeptide encoded by the open reading frame of the nucleic acid sequence.

In certain embodiments, the gene modifying polypeptide further comprises a heterologous protein domain.

Table 7 provides gene modifying polypeptides comprising retrotransposon elements, altered for improved efficiency of integration into the human genome. Retrotransposase polypeptides were improved through consensus mapping to re-derive the optimal amino acid sequence. Template molecules for use with cognate retrotransposase enzymes were mapped back to their host genomes and flanking genomic DNA used to elucidate target site motifs. When detectable, conserved sequence motifs from the flanking genomic DNA of endogenous occurrences of an element were aligned to the human genome, and new sequences were derived from the human genome as 5′ or 3′ “Human Homology Arms.” In some embodiments, a template RNA described herein comprises one or both of a first homology domain comprising a sequence of a 5′ Human Homology Arm of Table 7 (or a sequence having at least 80%, 85%, 90%, 95%, 96%, 97%, 98%, or 99% identity thereto) and a second homology domain comprising a sequence of a 3′ Human Homology Arm of Table 7 (or a sequence having at least 80%, 85%, 90%, 95%, 96%, 97%, 98%, or 99% identity thereto).

TABLE 7
Retrotransposase systems with improved integration activity

| | | | | | 5′ Human | 3′ Human | | |
|---|---|---|---|---|---|---|---|---|
| | | | Target | Consensus Optimized Protein | Homology | Homology | | |
| Element | Organism | Domains | Motif | Sequence | Arm | Arm | 5′ UTR | 3′ UTR |
| L2-2 DRe | Danio | RT and EN | TGAGC | (SEQ ID NO: 31) | (SEQ ID | A | (SEQ ID NO: 34) | (SEQ ID |
| (1) | rerio | | ACGGT | MCFLIPVVTNTRKTREVRCRRNPHNLR | NO: 32) | | GCAGGAGAAGC | NO: 35) |
| | | | AGCATT | SIHVSTISQLSLSVGLWNCQSAVNKAD | GTTTCGCG | | ACTTTAGCAGC | TAATCTGC |
| | | | AGTCGa | FITSIATYSDYNLMALTETWLRPEDTAT | TCGTCCGC | | ATCTAGAACAG | AATTGCCT |
| | | | (SEQ ID | HATLSANFSFSHTPRQTGRGGGTGLLI | CTCAGTTTC | | CAGCCTGTAAG | CTCTGAAT |
| | | | NO: 30) | SKEWKFTLIPSLPTISSFEFHAVTIIHPFYI | GGCCCTTG | | TACATTTAAGAT | ATCACACT |
| | | | | NVVVIYRPPGKLGHFLDELDVLLSSFSN | TTTTGACTC | | TTGTTTCAGTTG | AACTGTA |
| | | | | FATPLLVLGDFNIYVDKPQAADFQTLLA | GGGAGGC | | TTGTGTATGGTT | CCCAAAA |
| | | | | SFDLKRAPTSATHKSGNQLDLIYTRHCF | GTGTCCAA | | TGAGGACTTGT | AAAAAAA |
| | | | | TDQTIVTPLQISDHFLLSLNIHITPEPPH | ACGCCAGG | | TTCCAGCTGTTT | AAAAAAA |
| | | | | TPTLVTFRRNLRSLSPNRLSTIVSDSLPP | TAACCACT | | GTGTAAAGTTG | AAAAAAA |
| | | | | SRKLTALDSNSATNTLCSTLASCLDRLC | ATATAGTG | | TAGGACATTTA | ATAAAAA |
| | | | | PLASRPARASPPAPWLSDALREHRSKL | AGCACGGT | | AACTTGCTTTCA | WACTACT |
| | | | | RAAERIWRKTKNPAHLLTYQTLLSSFSA | AGCATTAG | | GTTGTGTATAAT | AATACTTC |
| | | | | EVTSAKQTYYRLKINNATNPRLLFKTFS | TCG | | ACTAGAAGTTG | CCTTCTTA |
| | | | | SLLYPPPPPASSTLTTDDFATFFCTKTAK | | | TTTAGCCACTGT | GACTTTAC |
| | | | | ISAQFAAPTTNTQDTTPTPHTLTSFSQL | | | TTCCTTGGTTAC | AGACCTG |
| | | | | SESEVSKLVLSSHATTCPLDPIPSHLLQA | | | TATAAGAGCTT | AAACTTG |
| | | | | ISPAVIPTLTHIINTSLDSGLFPTTFKQAR | | | GTGTAGCGAAC | CCTATAG |
| | | | | VTPLLKKPNLDHTLLENYRPVSLLPFMA | | | GCAGACGCGGT | CACTTATT |
| | | | | KILEKVVFNQVLDFLTQNNLMDNKQS | | | TCGCGTCGTCC | CATTGTT |
| | | | | GFKKGHSTETALLSVVEDLRLAKADSKS | | | GCCTCAGTTTCG | GCTCTTA |
| | | | | SVLILLDLSAAFDTVNHQILLSTLESLGV | | | GCCCTTGTTTTG | GTTGTGT |
| | | | | AGTVIQWFRSYLSDRSFRVSWRGEVS | | | ACTCGGGAGGC | AAATTGC |
| | | | | NLQHLNTGVPQGSVLGPLLFSIYTSSLG | | | GTGTCCAAACG | TTCCTTGT |
| | | | | PVIQRHGFSYHCYADDTQLYLSFHPDD | | | CCAGGTAACCA | CCTCATTT |
| | | | | PSVPARISACLLDISHWMKDHHLQLNL | | | CTATATAGTGA | GTAAGTC |
| | | | | AKTEMLVVSANPTLHHNFSIQMDGAT | | | GCACGGTAGCA | GCTTTGG |
| | | | | ITASKMVKSLGVTIDDQLNFSDHISRTA | | | TTAGTCGGCAG | ATAAAAG |
| | | | | RSCRFALYNIRKIRPFLSEHAAQLLVQAL | | | GAGAAGCACTT | CGTCTGC |
| | | | | VLSKLDYCNSLLAGLPANSIKPLQLLQN | | | TAGCAGCATCT | TAAATGA |
| | | | | AAARVVFNEPKRAHVTPLLVRLHWLP | | | AGAACAGCAGC | CTAAATG |
| | | | | VAARIKFKTLMFAYKVTSGLAPSYLHSL | | | CTGTAAGTACA | TAAATGT |
| | | | | LQIYVPSRNLRSVNERRLVVPSQRGKKS | | | TTTAAGATTTGT | AAATGT |
| | | | | LSRTLTLNLPSWWNELPNCIRTAESLAI | | | TTCAGTTGTTGT | |
| | | | | FKKRLKTQLFSLHFTS | | | GTATGGTTTGA | |
| | | | | | | | GGACTTGTTTCC | |
| | | | | | | | AGCTGTTTGTGT | |
| | | | | | | | AAAGTTGTAGG | |
| | | | | | | | ACATTTAAACTT | |
| | | | | | | | GCTTTCAGTTGT | |
| | | | | | | | GTATAATACTA | |
| | | | | | | | GAAGTTGTTTA | |
| | | | | | | | GCCACTGTTTCC | |
| | | | | | | | TTGGTTACTATA | |
| | | | | | | | AGAGCTTGTGT | |
| | | | | | | | AGCGAACGCAG | |
| | | | | | | | ACGCGGTTCGC | |
| | | | | | | | GTCGTCCGCCTC | |
| | | | | | | | AGTTTCGGCCCT | |
| | | | | | | | TGTTTTGACTCG | |
| | | | | | | | GGAGGCGTGTC | |
| | | | | | | | CAAACGCCAGG | |
| | | | | | | | TAACCACTATAT | |
| | | | | | | | AGTGAGCACGG | |
| | | | | | | | TAGCATTAGTC | |
| | | | | | | | GGCAGGAGAA | |
| | | | | | | | GCACTTTAGCA | |
| | | | | | | | GCATCTAGAAC | |
| | | | | | | | AGCAGCCTGTA | |
| | | | | | | | AGTACATTTAA | |
| | | | | | | | GATTTGTTTCAG | |
| | | | | | | | TTGTTGTGTATG | |
| | | | | | | | GTTTGAGGACT | |
| | | | | | | | TGTTTCCAGCTG | |
| | | | | | | | TTTGTGTAAAGT | |
| | | | | | | | TGTAGGACATT | |
| | | | | | | | TAAACTTGCTTT | |
| | | | | | | | CAGTTGTGTAT | |
| | | | | | | | AATACTAGAAG | |
| | | | | | | | TTGTTTAGCCAC | |
| | | | | | | | TGTTTCCTTGGT | |
| | | | | | | | TACTATAAGAG | |
| | | | | | | | CTTGTGTAGCG | |
| | | | | | | | AACGCAGACGC | |
| | | | | | | | GGTTCGCGTCG | |
| | | | | | | | TCCGCCTCAGTT | |
| | | | | | | | TCGGCCCTTGTT | |
| | | | | | | | TTGACTCTCGA | |
| | | | | | | | GGCGTGTCCAG | |
| | | | | | | | CTGAATTCAATC | |
| | | | | | | | AGCTAGTGCTTT | |
| | | | | | | | GGGGTTATATA | |
| | | | | | | | AACAACTAGTT | |
| | | | | | | | CACCGCGGCAG | |
| | | | | | | | CGGTCGCGGCA | |
| | | | | | | | GCCTCGTGTGA | |
| | | | | | | | AGACCGACGAG | |
| | | | | | | | GGTAAAGACCA | |
| | | | | | | | TCGACTCTACCT | |
| | | | | | | | GCGCGACTCCA | |
| | | | | | | | CCGAGCAAAGA | |
| | | | | | | | CACCGACAAAG | |
| | | | | | | | CACTTGAGTACT | |
| | | | | | | | TTACTGTATTGT | |
| | | | | | | | TTTACTTTACAC | |
| | | | | | | | TTATTTTTTGTT | |
| | | | | | | | GTCAGTGCACT | |
| | | | | | | | TTTATT | |
| L2-2_Dre | Danio | RT and EN | TGAGC | (SEQ ID NO: 36) | (SEQ ID | (SEQ ID | (SEQ ID NO: 37) | (SEQ ID |
| (2) | rerio | | ACGGT | MCFLIPVVTNTRKTREVRCRRNPHNLR | NO: 32) | NO: 33) | GCAGGAGAAGC | NO: 38) |
| | | | AGCATT | SIHVSTISQLSLSVGLWNCQSAVNKAD | GTTTCGCG | a | ACTTTAGCAGC | TAATCTGC |
| | | | AGTCGa | FITSIATYSDYNLMALTETWLRPEDTAT | TCGTCCGC | | ATCTAGAACAG | AATTGCCT |
| | | | (SEQ ID | HATLSANFSFSHTPRQTGRGGGTGLLI | CTCAGTTTC | | CAGCCTGTAAG | CTCTGAAT |
| | | | NO: 30) | SKEWKFTLIPSLPTISSFEFHAVTIIHPFYI | GGCCCTTG | | TACATTTAAGAT | ATCACACT |
| | | | | NVVVIYRPPGKLGHFLDELDVLLSSFSN | TTTTGACTC | | TTGTTTCAGTTG | AACTGTA |
| | | | | FATPLLVLGDFNIYVDKPQAADFQTLLA | GGGAGGC | | TTGTGTATGGTT | CCCAAAA |
| | | | | SFDLKRAPTSATHKSGNQLDLIYTRHCF | GTGTCCAA | | TGAGGACTTGT | AAAAAAA |
| | | | | TDQTIVTPLQISDHFLLSLNIHITPEPPH | ACGCCAGG | | TTCCAGCTGTTT | AAAAAAA |
| | | | | TPTLVTFRRNLRSLSPNRLSTIVSDSLPP | TAACCACT | | GTGTAAAGTTG | AAAAAAA |
| | | | | SRKLTALDSNSATNTLCSTLASCLDRLC | ATATAGTG | | TAGGACATTTA | ATAAAAA |
| | | | | PLASRPARASPPAPWLSDALREHRSKL | AGCACGGT | | AACTTGCTTTCA | WACTACT |
| | | | | RAAERIWRKTKNPAHLLTYQTLLSSFSA | AGCATTAG | | GTTGTGTATAAT | AATACTTC |
| | | | | EVTSAKQTYYRLKINNATNPRLLFKTFS | TCG | | ACTAGAAGTTG | CCTTCTTA |
| | | | | SLLYPPPPPASSTLTTDDFATFFCTKTAK | | | TTTAGCCACTGT | GACTTTAC |
| | | | | ISAQFAAPTTNTQDTTPTPHTLTSFSQL | | | TTCCTTGGTTAC | AGACCTG |
| | | | | SESEVSKLVLSSHATTCPLDPIPSHLLQA | | | TATAAGAGCTT | AAACTTG |
| | | | | ISPAVIPTLTHIINTSLDSGLFPTTFKQAR | | | GTGTAGCGAAC | CCTATAG |
| | | | | VTPLLKKPNLDHTLLENYRPVSLLPFMA | | | GCAGACGCGGT | CACTTATT |
| | | | | KILEKVVFNQVLDFLTQNNLMDNKQS | | | TCGCGTCGTCC | CATTGTT |
| | | | | GFKKGHSTETALLSVVEDLRLAKADSKS | | | GCCTCAGTTTCG | GCTCTTA |
| | | | | SVLILLDLSAAFDTVNHQILLSTLESLGV | | | GCCCTTGTTTTG | GTTGTGT |
| | | | | AGTVIQWFRSYLSDRSFRVSWRGEVS | | | ACTCGGGAGGC | AAATTGC |
| | | | | NLQHLNTGVPQGSVLGPLLFSIYTSSLG | | | GTGTCCAAACG | TTCCTTGT |
| | | | | PVIQRHGFSYHCYADDTQLYLSFHPDD | | | CCAGGTAACCA | CCTCATTT |
| | | | | PSVPARISACLLDISHWMKDHHLQLNL | | | CTATATAGTGA | GTAAGTC |
| | | | | AKTEMLVVSANPTLHHNFSIQMDGAT | | | GCACGGTAGCA | GCTTTGG |
| | | | | ITASKMVKSLGVTIDDQLNFSDHISRTA | | | TTAGTCGGCAG | ATAAAAG |
| | | | | RSCRFALYNIRKIRPFLSEHAAQLLVQAL | | | GAGAAGCACTT | CGTCTGC |
| | | | | VLSKLDYCNSLLAGLPANSIKPLQLLQN | | | TAGCAGCATCT | TAAATGA |
| | | | | AAARVVFNEPKRAHVTPLLVRLHWLP | | | AGAACAGCAGC | CTAAATG |
| | | | | VAARIKFKALMFAYKVTSGLAPSYLLSLL | | | CTGTAAGTACA | TAAATGT |
| | | | | QIYVPSRNLRSVNERRLVVPSQRGKKSL | | | TTTAAGATTTGT | AAATGT |
| | | | | SRTLTLNLPSWWNELPNCIRTAESLAIF | | | TTCAGTTGTTGT | |
| | | | | KKRLKTQLFSLHFTS | | | GTATGGTTTGA | |
| | | | | | | | GGACTTGTTTCC | |
| | | | | | | | AGCTGTTTGTGT | |
| | | | | | | | AAAGTTGTAGG | |
| | | | | | | | ACATTTAAACTT | |
| | | | | | | | GCTTTCAGTTGT | |
| | | | | | | | GTATAATACTA | |
| | | | | | | | GAAGTTGTTTA | |
| | | | | | | | GCCACTGTTTCC | |
| | | | | | | | TTGGTTACTATA | |
| | | | | | | | AGAGCTTGTGT | |
| | | | | | | | AGCGAACGCAG | |
| | | | | | | | ACGCGGTTCGC | |
| | | | | | | | GTCGTCCGCCTC | |
| | | | | | | | AGTTTCGGCCCT | |
| | | | | | | | TGTTTTGACTCG | |
| | | | | | | | GGAGGCGTGTC | |
| | | | | | | | CAAACGCCAGG | |
| | | | | | | | TAACCACTATAT | |
| | | | | | | | AGTGAGCACGG | |
| | | | | | | | TAGCATTAGTC | |
| | | | | | | | GGCAGGAGAA | |
| | | | | | | | GCACTTTAGCA | |
| | | | | | | | GCATCTAGAAC | |
| | | | | | | | AGCAGCCTGTA | |
| | | | | | | | AGTACATTTAA | |
| | | | | | | | GATTTGTTTCAG | |
| | | | | | | | TTGTTGTGTATG | |
| | | | | | | | GTTTGAGGACT | |
| | | | | | | | TGTTTCCAGCTG | |
| | | | | | | | TTTGTGTAAAGT | |
| | | | | | | | TGTAGGACATT | |
| | | | | | | | TAAACTTGCTTT | |
| | | | | | | | CAGTTGTGTAT | |
| | | | | | | | AATACTAGAAG | |
| | | | | | | | TTGTTTAGCCAC | |
| | | | | | | | TGTTTCCTTGGT | |
| | | | | | | | TACTATAAGAG | |
| | | | | | | | CTTGTGTAGCG | |
| | | | | | | | AACGCAGACGC | |
| | | | | | | | GGTTCGCGTCG | |
| | | | | | | | TCCGCCTCAGTT | |
| | | | | | | | TCGGCCCTTGTT | |
| | | | | | | | TTGACTCTCGA | |
| | | | | | | | GGCGTGTCCAG | |
| | | | | | | | CTGAATTCAATC | |
| | | | | | | | AGCTAGTGCTTT | |
| | | | | | | | GGGGTTATATA | |
| | | | | | | | AACAACTAGTT | |
| | | | | | | | CACCGCGGCAG | |
| | | | | | | | CGGTCGCGGCA | |
| | | | | | | | GCCTCGTGTGA | |
| | | | | | | | AGACCGACGAG | |
| | | | | | | | GGTAAAGACCA | |
| | | | | | | | TCGACTCTACCT | |
| | | | | | | | GCGCGACTCCA | |
| | | | | | | | CCGAGCAAAGA | |
| | | | | | | | CACCGACAAAG | |
| | | | | | | | CACTTGAGTACT | |
| | | | | | | | TTACTGTATTGT | |
| | | | | | | | TTTACTTTACAC | |
| | | | | | | | TTATTTTTTGTT | |
| | | | | | | | GTCAGTGCACT | |
| | | | | | | | TTTATT | |
| RTE-1 MD | Mono- | L1-EN, RT, | NA | (SEQ ID NO: 39) | | | (SEQ ID NO: 40) | (SEQ ID |
| (1) | delphis | ZF | | MDSTAHPNQGRGLEKVSQTLPALQTP | | | gggtgtatggggtgc | NO: 41) |
| | domestica | | | GQHTAAGGSSPLSGRNQRKNTKKLLL | | | tcagggaggggtag | tgaaactgc |
| | | | | GAWNIRTLLDRENTPRPERRTALIGKEL | | | tatctctggtatgga | acaaagaca |
| | | | | ARYNIDIAALSETRLPEEGSLSEPTTGYT | | | gggcttgtcgtgccc | atagtcattc |
| | | | | FFWKGRASNEDRIHGVGLAIKTSLLKQ | | | tcctagggcagctct | tcgatcaccg |
| | | | | LPDLPVGISERLMKIRLPLSKDRYATIISA | | | ccagcctctgacccc | agagactac |
| | | | | YAPTLTSTEETIEQFYSDLSAVLHSVPTN | | | cacctgacacccag | cact |
| | | | | DKLILLGDFNARVGQDHERWKGVLGK | | | ctctcacttgtggctc | |
| | | | | HGVGKMNNNGLLLLSKCSEFELTITNT | | | ccagtagctgctagc | |
| | | | | VFRMANKYKTTWMHPRSKQWHLIDY | | | atgtggcagcggcc | |
| | | | | IIVRRRDIQDVKITRAMRGAECWTDHR | | | acaccccgggcaac | |
| | | | | LVRATLQMRIAPRHPKRAQTVRAFYN | | | ggcttcgacaggccg | |
| | | | | VSRLRDPSYLQTFQSCLDDKLSAKGPLT | | | gctaaaccttgtgag | |
| | | | | GSSTEKWNQFRDAVKETSKAVLGPKQ | | | ggtagccatcgggtc | |
| | | | | RNHQDWFDENNTAIEDLLSKKNKAFM | | | atcgacccctggtga | |
| | | | | EWQNNPNSAPKKDRFKSLQATAQREI | | | accagggctttgctc | |
| | | | | RKMQDRWWEKKAEEIQRFADMKNY | | | acccagcatgtgaa | |
| | | | | KQFFSALKTVYGPLKPTTTPLLSSDGDT | | | gactgcttcggctga | |
| | | | | LIKDKKGISNRWKEHFSQLLNRPSSVD | | | acagacggaagaaa | |
| | | | | QSALDQIPQNRTIEQLDVPPSIEEVQKA | | | ccaataagaaggttc | |
| | | | | IKQMSAGKAPGKDGIPTEVYKALNGK | | | aacggctgagaggg | |
| | | | | ALQAFHIVLTSIWEEEDMPPELRDASIV | | | cgacgcagcaaagc | |
| | | | | ALYKNKGSRAACDNYRGISLLSTAGKIL | | | actgtggagtgctta | |
| | | | | ARVILNRLLSSVSEQNLPESQCGFRPDR | | | gggcgtgttggagc | |
| | | | | STIDMVFTVRQMQEKCLEQNLSLYIVFI | | | acaaaggacaacac | |
| | | | | DLTKAFDTVNRDALWVILSKLGCPAKF | | | ggccatccaatgca | |
| | | | | VKLIQLFHVDMTGEVLSGGETSDRFNI | | | gctgaggaagtctcc | |
| | | | | SNGVKQGCVLAPVLFNLFFTQVLRHAV | | | agatgtaacaatttt | |
| | | | | MDLDLGVYIKYRLDGSLFDLRRLTAKTK | | | tcgtgccactggacc | |
| | | | | TTERLILEALFADDCALMAHQENHLQT | | | caggcttccaacgcc | |
| | | | | IVDRFSTATKLFGLTISLSKTEVLFQPAP | | | gagagagtgggact | |
| | | | | GRPTNQPCITIDGTQLSNVNTFKYLGST | | | gtctctgtgcatcgg | |
| | | | | IANDGSLDHEINARIQKASQALGRLRC | | | cttttccacttaaatc | |
| | | | | KVLQHRGVSTATKLKVYNAVVLSSLLY | | | tctttcacgcacaag | |
| | | | | GCETWTLYRKHMKQLEQFHQRSLRSI | | | tatctttgtgcacact | |
| | | | | MRIRWQDRITNQEVLDRANSTSIEVM | | | catctatcctaaccc | |
| | | | | VLKTQLRWSGHVIRMDPQRIPRQVFY | | | cgtccaccctcttca | |
| | | | | GELSAGLRKQGRPKKRFKDQLKSNLK | | | agacctgcggcgat | |
| | | | | WAGITPKQLELAASDRSSWRTHINHA | | | gggggagtggcgac | |
| | | | | ATTFEDERRRRLAAARERRHQATTAPP | | | gcaacaggtggagg | |
| | | | | VTTGVPCPMCHKLCASAFGLQSHMRV | | | tgaccactggcagtt | |
| | | | | HRR | | | gtagtcacgatcctg | |
| | | | | | | | cacgtaggcggccc | |
| | | | | | | | acggaccagtggtc | |
| | | | | | | | gctcggccctgtggg | |
| | | | | | | | cagcagggacgttc | |
| | | | | | | | ggcagcatcctgggc | |
| | | | | | | | gactgagcagccctc | |
| | | | | | | | tctag | |
| RTE-1_MD | Mono- | L1-EN, RT, | NA | (SEQ ID NO: 42) | | | (SEQ ID NO: 43) | (SEQ ID |
| (2) | delphis | ZF | | MDSTAHPNQGRGLEKVSQTLPALQTP | | | gggtgtatggggtgc | NO: 44) |
| | domestica | | | GQHTAAGGSSPLSGRNQRKNTKKLLL | | | tcagggaggggtag | tgaaactgc |
| | | | | GAWNIRTLLDRENTPRPERRTALIGKEL | | | tatctctggtatgga | acaaagaca |
| | | | | ARYNIDIAALSETRLPEEGSLSEPTTGYT | | | gggcttgtcgtgccc | atagtcattc |
| | | | | FFWKGRASNEDRIHGVGLAIKTSLLKQ | | | tcctagggcagctct | tcgatcaccg |
| | | | | LPDLPVGISERLMKIRLPLSKDRYATIISA | | | ccagcctctgacccc | agagactac |
| | | | | YAPTLTSTEETIEQFYSDLSAVLHSVPTN | | | cacctgacacccag | cact |
| | | | | DKLILLGDFNARVGQDHERWKGVLGK | | | ctctcacttgtggctc | |
| | | | | HGVGKMNNNGLLLLSKCSEFELTITNT | | | ccagtagctgctagc | |
| | | | | VFRMANKYKTTWMHPRSKQWHLIDY | | | atgtggcagcggcc | |
| | | | | IIVRRRDIQDVKITRAMRGAECWTDHR | | | acaccccgggcaac | |
| | | | | LVRATLQMRIAPRHPKRAQTVRAFYN | | | ggcttcgacaggccg | |
| | | | | VSRLRDPSYLQTFQSCLDNKLSAKGPLT | | | gctaaaccttgtgag | |
| | | | | GSSTEKWNQFRDAVKETSKAVLGPKQ | | | ggtagccatcgggtc | |
| | | | | RNHQDWFDENNTAIEDLLSKKNKAFM | | | atcgacccctggtga | |
| | | | | EWQNNPNSAPKKDRFKSLQATAQREI | | | accagggctttgctc | |
| | | | | RKMQDRWWEKKAEEIQRFADMKNY | | | acccagcatgtgaa | |
| | | | | KQFFSALKTVYGPLKPTTTPLLSSDGDT | | | gactgcttcggctga | |
| | | | | LIKDKKGISNRWKEHFSQLLNRPSSVD | | | acagacggaagaaa | |
| | | | | QSALDQIPQNRSIEQLDVPPSIEEVQKA | | | ccaataagaaggttc | |
| | | | | IKQMSAGKAPGKDGIPTEVYKALNGK | | | aacggctgagaggg | |
| | | | | ALQAFHIVLTSIWEEEDMPPELRDASIV | | | cgacgcagcaaagc | |
| | | | | ALYKNKGSRAACDNYRGISLLSTAGKIL | | | actgtggagtgctta | |
| | | | | ARVILNRLLSSVSEQNLPESQCGFRPDR | | | gggcgtgttggagc | |
| | | | | STIDMVFTVRQMQEKCLEQNLSLYIVFI | | | acaaaggacaacac | |
| | | | | DLTKAFDTVNRDALWVILSKLGCPAKF | | | ggccatccaatgca | |
| | | | | VKLIQLFHVDMTGEVLSGGETSDRFNI | | | gctgaggaagtctcc | |
| | | | | SNGVKQGCVLAPVLFNLFFTQVLRHAV | | | agatgtaacaatttt | |
| | | | | MDLDLGVYIKYRLDGSLFDLRRLTAKTK | | | tcgtgccactggacc | |
| | | | | TTERLILEALFADDCALMAHQENHLQT | | | caggcttccaacgcc | |
| | | | | IVDRFSTATKLFGLTISLSKTEVLFQPAP | | | gagagagtgggact | |
| | | | | GRPTNQPCITIDGTQLSNVNTFKYLGST | | | gtctctgtgcatcgg | |
| | | | | IANDGSLDHEINARIQKASQALGRLRC | | | cttttccacttaaatc | |
| | | | | KVLQHRGVSTATKLKVYNAVVLSSLLY | | | tctttcacgcacaag | |
| | | | | GCETWTLYRKHMKQLEQFHQRSLRSI | | | tatctttgtgcacact | |
| | | | | MRIRWQDRITNQEVLDRANSTSIEVM | | | catctatcctaaccc | |
| | | | | VLKTQLRWSGHVIRMDPQRIPRQVFY | | | cgtccaccctcttca | |
| | | | | GELSAGLRKQGRPKKRFKDQLKSNLK | | | agacctgcggcgat | |
| | | | | WAGITPKQLELAASDRSSWRTHINHA | | | gggggagtggcgac | |
| | | | | ATTFEDERRRRLAAARERRHQATTAPP | | | gcaacaggtggagg | |
| | | | | VTTGVPCPMCHKLCASAFGLQSHMRV | | | tgaccactggcagtt | |
| | | | | HRR | | | gtagtcacgatcctg | |
| | | | | | | | cacgtaggcggccc | |
| | | | | | | | acggaccagtggtc | |
| | | | | | | | gctcggccctgtggg | |
| | | | | | | | cagcagggacgttc | |
| | | | | | | | ggcagcatcctgggc | |
| | | | | | | | gactgagcagccctc | |
| | | | | | | | tctag | |
| Vingi- | Anolis | DNAse- | NA | (SEQ ID NO: 45) | | | (SEQ ID NO: 46) | (SEQ ID |
| 1_Acar | carol- | like, RT | | MDEYQRSLSRPLLTIMSINIEGLSLAKEE | | | GGGGGACACG | NO: 47) |
| (1) | inensis | | | LLAKMSEDISCDILCIQETHRDITMRRP | | | GAAAGAGCCTC | TAGTTGC |
| | | | | KILGMQLAVERPHRQYGSAIFVRSGVA | | | CCCGAAGATTG | TTGTGATT |
| | | | | ISATSLTEVNNIEILSVELDSCTVSSLYKP | | | AGTGAATTCAG | TCTTTTCT |
| | | | | PGADFYFTPPTSCHNHEAHFVVGDFN | | | TCGGGCGTCCC | TTTTTATT |
| | | | | SHSCVWGYDEDDRNGEAVLTWADNS | | | CTGGGCAACGT | TTATTTCC |
| | | | | RMSLLHDSKLPPSFNSGRWKRGYNPD | | | TTCTTGTAAGCG | ATTATTTG |
| | | | | LIFVKESISHQCTKRVLNPIPNTQHRPIC | | | GCCGATCTTTCC | AAATGTA |
| | | | | CVAYAAVRPKSVPFRRRYNFNKANWT | | | ACCCCAAAAGC | TTTGCTGT |
| | | | | KFTETLEAAISDIEPSIENYDLFVEAVKRS | | | ATTGGATGA | ACCAATG |
| | | | | SRLSIPRGCRTSYLPGLNEESLNQLQEYL | | | | CTTTTGAC |
| | | | | RLFQENPYSDGTIAAGQKLSTALANAK | | | | ACGAAAT |
| | | | | KDRWIELLENLDMSKSSRKAWQLLRRL | | | | AAATAAA |
| | | | | DSDPLVNPGHANVTPDQIAHQLIQNG | | | | |
| | | | | KTNCSRIKMKINRVPELETHQLSSPLNL | | | | |
| | | | | KELREAIKRCKTGKAPGLDDLMMEQIK | | | | |
| | | | | HLGPKAENWLLKFYNQCLAHKQIPRA | | | | |
| | | | | WRKTKIIAILKPGKDASNARNYRPISLLC | | | | |
| | | | | HLYKVYERMLLNRLGPVIEPKLIAQQA | | | | |
| | | | | GFRPGKNCTGQILHLTEHIEEGYEKGCI | | | | |
| | | | | TGTVFVDLTAAYDTVQHRKMLHKVYH | | | | |
| | | | | ITRDFDFTKTVQTLLENRSFYVEFQGQK | | | | |
| | | | | SRWRRQKNGLPQGSVLAPTLFNIFTN | | | | |
| | | | | DQPQPPLTKSFIYADDLGLTTQAKDFE | | | | |
| | | | | TVEKQLTNALKDLSSYYKENHLKPNPA | | | | |
| | | | | KTQVCAFHLRNREANRKLKVTWEGQE | | | | |
| | | | | LEHCFHPKYLGVTLDRTLTYRKHCMNT | | | | |
| | | | | KHKVAARNNILRKLTGSAWGADPQVI | | | | |
| | | | | RTSALALSFSTAEYACPVWHKSAHAKQ | | | | |
| | | | | VDIALNETCRIITGCLKPTPVDKLYKLAG | | | | |
| | | | | IAPPDVRREVAANGERKKVEHCESHPL | | | | |
| | | | | HDYHPPPTRLKSRKGFMRTTTPLDVPP | | | | |
| | | | | ATARVSLWAAKPGNSNWMAPQEGLP | | | | |
| | | | | PGANQEWATWKSLNRLRSGVGRSKD | | | | |
| | | | | NLARWHYLEESSTLCDCGAEQTTQHM | | | | |
| | | | | YACPQCPASCTEEELFKATDNAVAVAR | | | | |
| | | | | FWSKTI | | | | |
| Vingi- | Anolis | DNAse- | NA | (SEQ ID NO: 48) | | | (SEQ ID NO: 49) | (SEQ ID |
| 1_Acar | carol- | like, RT | | MDEYQRSLSRPLLTIMSINIEGLSLAKEE | | | GGGGGACACG | NO: 50) |
| (2) | inensis | | | LLAKMSEDISCDILCIQETHRDITMRRP | | | GAAAGAGCCTC | TAGTTGC |
| | | | | KILGMQLAVERPHRQYGSAIFVRSGVA | | | CCCGAAGATTG | TTGTGATT |
| | | | | ISATSLTEVNNIEILSVELDSCTVSSLYKP | | | AGTGAATTCAG | TCTTTTCT |
| | | | | PGADFYFTPPTSCHNHEAHFVVGDFN | | | TCGGGCGTCCC | TTTTTATT |
| | | | | SHSCVWGYDEDDRNGEAVLTWADNS | | | CTGGGCAACGT | TTATTTCC |
| | | | | RMSLLHDSKLPPSFNSGRWKRGYNPD | | | TTCTTGTAAGCG | ATTATTTG |
| | | | | LIFVKESISHQCTKRVLNPIPNTQHRPIC | | | GCCGATCTTTCC | AAATGTA |
| | | | | CVAYAAVRPKSVPFRRRYNFNKANWT | | | ACCCCAAAAGC | TTTGTTGT |
| | | | | KFTETLEAAISDIEPSIENYDLFVEAVKRS | | | ATTGGATGA | AGCAATG |
| | | | | SRLSIPRGCRTSYLPGLNEESLNQLQEYL | | | | CTTTTGAC |
| | | | | RLFQENPYSDGTIAAGQKLSTALANAK | | | | ACGAAAT |
| | | | | KDRWIELLENLDMSKSSRKAWQLLRRL | | | | AAATAAA |
| | | | | DSDPLVNPGHANVTPDQIAHQLIQNG | | | | |
| | | | | KTNCSRIKMKINRVPELETHQLSSPLNL | | | | |
| | | | | KELREAIKRCKTGKAPGLDDLMMEQIK | | | | |
| | | | | HLGAKAENWLLKFYNQCLAHKQIPRA | | | | |
| | | | | WRKTKIIAILKPGKDASNARNYRPISLLC | | | | |
| | | | | HLYKVYERMLLNRLGPVIEPKLIAQQA | | | | |
| | | | | GFRPGKNCTGQILHLTEHIEEGYEKGCI | | | | |
| | | | | TGTVFVDLTAAYDTVQHRKMLHKVYH | | | | |
| | | | | ITRDFDFTKTVQTLLENRSFYVEFQGQK | | | | |
| | | | | SRWRRQKNGLPQGSVLAPTLFNIFTN | | | | |
| | | | | DQPQPPLTKSFIYADDLGLTTQAKDFE | | | | |
| | | | | TVEKQLTNALKDLSSYYKENHLKPNPA | | | | |
| | | | | KTQVCAFHLRNREANRKLKVTWEGQE | | | | |
| | | | | LEHCFHPKYLGVTLDRTLTYRKHCMNT | | | | |
| | | | | KHKVAARNNILRKLTGSAWGADPQVI | | | | |
| | | | | RTSALALSFSTAEYACPVWHKSAHAKQ | | | | |
| | | | | VDIALNETCRIITGCLKPTPVDKLYKLAG | | | | |
| | | | | IAPPDVRREVAANGERKKVEHCESHPL | | | | |
| | | | | HGYHPPPTRLKSRKGFMRTTTPLDVPP | | | | |
| | | | | AAARVSLWAAKPGNSNWMAPQEGL | | | | |
| | | | | PPGANQEWATWKSLNRLRSGVGRSK | | | | |
| | | | | DNLARWHYLEESSTLCDCGAEQTTQH | | | | |
| | | | | MYACPQCPASCTEEELFKATDNAVAV | | | | |
| | | | | ARFWSKTI | | | | |
| CR1-1_PH | Parhyale | DNAse I- | NA | (SEQ ID NO: 51) | | | (SEQ ID NO: 52) | (SEQ ID |
| | hawaiensis | like, RT | | MLYTIFLVYGILCFFFVYFCIYLYITVYLCF | | | gcgtggcctgcgcgt | NO: 53) |
| | | | | LFFLFLLLCGDVESNPGPGRARGCRLLY | | | tatcaggccgccccc | tagcagcgtt |
| | | | | CNIRGLHANLAELDFVSRGVDVVCCSE | | | attgtccggccaccg | ttgggcttcc |
| | | | | TLVAGRRHDAELALAGFQSPFRRLCGS | | | gacgcctctttgtttg | cggtgttgca |
| | | | | GPGFRGMAVYVRSGFCAYRQSVHECA | | | tatgagctggctttg | agatgcttca |
| | | | | CHEIIVVRVCGRLNNYYLFSLYRSPATD | | | ggtttggggctctgg | tttgggttgt |
| | | | | DSLYDCLLTAMASIQSTDPKAAFVFVG | | | gtagcccctgagttg | attacctgtc |
| | | | | DVNAHHRDWLGSASPTDCHGVAALD | | | gaggaaatgccact | cgtgggagc |
| | | | | FCTLSSCVQLVRGSTHIAGNCLDLVMT | | | ctaaaggcggatga | tcaagcgtg |
| | | | | DVPDLMTVTVGSPIGSSDHSHLVVSLD | | | gacctcttttggtcca | aagttttaat |
| | | | | LNQVVPVVDTRRVVFVKSRANWVAIT | | | acggcatccctgag | aataataat |
| | | | | RAVRTLPWRQIIHSEDPVSELNDLTVSI | | | aggagatgaacagg | aa |
| | | | | LERFVPKRTILVRSRDKPWFDDQCRLA | | | ctggtgccatagctg | |
| | | | | FEAKQAAYRAWRRSRDRTLWQTYVD | | | ggtactcagtcttcc | |
| | | | | RRAEAKRVYEEAQRRLRQRSRESLLSID | | | gcagtctgctcctctc | |
| | | | | HPHRWWSELKGSVFGAEPSLPPLVGP | | | tctcttccatcaggta | |
| | | | | GGGIITDPLARAELLSAHFDGKQSRDVI | | | gtaccacctgatgct | |
| | | | | ALPHGCHPEPRLTSLAFRSGAIKVLLEG | | | cttttcctttcctttct | |
| | | | | LDPYGGVDPVGMFPLFYKQLADVLAP | | | cttcttcttcttttcgg | |
| | | | | KLAVIFRRLIRLGNFPRCWRTGNITPIPK | | | ccaaagcctgtactg | |
| | | | | GPVSPYVANYRPITLTPILSKVFEKLIAG | | | gtgatggcagggtct | |
| | | | | KLGRFAEVTGLLPAGQFAYRIGLGCCD | | | ggggcgcgtagata | |
| | | | | ALLSVSHHLQSALDCRSEARLVQLDFSA | | | gctggccgctttcgt | |
| | | | | AFDRVNHRGLLYKLESFGVGGRVLSIIR | | | agctatcgttactgtc | |
| | | | | DFVSERTQSVSVDGVLSASVGVVSGVP | | | gatttgtttttgcttta | |
| | | | | QGSVLGPLLFVLYTSDMFSSLENTLINY | | | tt | |
| | | | | ADDSTLMAVIPAPRLRDAVAQSLNRDL | | | | |
| | | | | SRISAWCSAWSMKLNASKTKSMIISRS | | | | |
| | | | | RTLVPQHPQLEIDDTLLQESSSLEILGVV | | | | |
| | | | | FDEKLTFEPHIRRLVSRASTKIGLLRKVN | | | | |
| | | | | SVFGDSQVARRCFYAFLLPVLEYCSPV | | | | |
| | | | | WASAADTHLRLLDRLVSSASRLCADND | | | | |
| | | | | LVNLSHRRRVAELCMFYKVYNNERHW | | | | |
| | | | | LYSSLPALKVFGRETRAACGAHSFTLEA | | | | |
| | | | | VRCRTNQFCRCFVPWSAKVWNLLPAS | | | | |
| | | | | AFGRIGLQAFKSAVNGFLLDFL | | | | |
| Crack- | Branchios | RT and EN | NNNNN | (SEQ ID NO: 55) | | | (SEQ ID NO: 56) | (SEQ ID |
| 28_BF | toma | | NNNNN | MWEKATSNVHLQSGTWITQNAYSVKI | | | AGCCCTAGTCC | NO: 57) |
| | floridae | | NNNNN | NPGLSGVRLIGRSTTCTERTERTLNLLV | | | CT | AACTTGA |
| | | | NNNNT | CATLLLAGDVSPNPGPDTGGLPVWRK | | | | TCTGGCT |
| | | | NNNNN | GIVYAFYNVVSLPRHLDEIQQLLLRNTRI | | | | GATGCTC |
| | | | CNNGT | HVLGLNETRLSDSIPDSSVDINGYTLYR | | | | CGTAGTC |
| | | | NNNNA | TDRDRQGGGVGVYVKQTIASQRRCEL | | | | TGGACTT |
| | | | NNNNN | EQEDLEVCCVEIKPEKARKTLLTCVYRP | | | | TGATATT |
| | | | NNNNN | PTSGPDWRNSAESLVHKLNQTAEKEN | | | | GGATAAT |
| | | | NNNNC | ADVAIMGDFNSDLLTSTQAMSSVEFL | | | | TTTATTAT |
| | | | NNNNN | MGLYQLVPVIREPTRITEKTESCIDNIFV | | | | GTATTGT |
| | | | NNNNN | SNPDRYKSSASVAWGPSDHNLILTCAK | | | | ATATGTG |
| | | | NNNNN | AGSEAGAAHRCEYRSYKLYTQQSFIDSL | | | | CTATGTG |
| | | | NTNNN | KSVRWDTVFDCTDVSEAWNAFKDIFL | | | | TACTTTGT |
| | | | NNNNN | NVADEHAPLRTKTARENNRPAPWMT | | | | ACTTTTAT |
| | | | NNNNN | DTVKNMMGRRDAARRKAIRTKDVQD | | | | GTCCAGG |
| | | | NNNNN | WDTYRSLRNQTTSIIRKEKKSHFATAVS | | | | ATTACCTG |
| | | | NNTNN | EAKGDQSLMWKIINSFTGKSKSTKRVQ | | | | AAAAGCA |
| | | | NNNNN | KLLRADNTSMSDPGEMAQEFNDYFTS | | | | GGCCGAT |
| | | | NNNNN | CASRLTDGMPDSEEDPLRHIPDSTTKFS | | | | TTCCGGC |
| | | | nntnnn | FDCVEETEVLNELQKLKTKKATGLDKIP | | | | CTGAGAT |
| | | | nnnnnn | AKLLKDSAPVVAKPLAHIFNLSLASGEV | | | | GTAATTA |
| | | | nnnnnn | PSDWKEAQITPVHKSGSCADVGNYRP | | | | CTGGTAA |
| | | | nnnnnc | VSVLSVTSKVMEKLVCNQVTRYLTRCK | | | | AATAAAT |
| | | | nnnnnn | LLTTHQSGFRRHHSTATAVQKVVEDIT | | | | AAAGTGA |
| | | | nnnnnn | SGYNCSKVTVALFLDLRKAFDSVNHEI | | | | AGTGAA |
| | | | nnnnnn | MLSKLKKFGFDSDAMKWFTSYLSERL | | | | |
| | | | gcnnnn | QCTCLQGQYSSKTRVSCGVPQGSVLG | | | | |
| | | | nnnntn | PLLFCLYVNDLPNVIQKCSIHMYADDT | | | | |
| | | | nnnnnn | VLYYSAVSVKVCEETVSMDMKRVVKW | | | | |
| | | | nnnnnn | LSENRLLLHPDKTKSMLFGLPQKLKHA | | | | |
| | | | nnnnnn | GTTVNITDGVNVYEQVDSFTYLGITLDP | | | | |
| | | | nnnnnn | ALRWAAHVQKITKKLLSGLGAMGRAR | | | | |
| | | | tnnnnn | AFVTNEVLKTMYQTLLLAHLEYCATAW | | | | |
| | | | nnnnnn | LPSLAQGNKTLMLQLDRLVNRAARLIT | | | | |
| | | | annnnn | GHKLRDHVTVDNLRAEAGIDSVRKRTE | | | | |
| | | | nnnn | ITTLVTVFKTIRGKAPAYLASLFKWEAP | | | | |
| | | | (SEQ ID | PTMSVRPTRSEVKRLRDYDPHLLWCP | | | | |
| | | | NO: 54) | PARVIAFRNSLQSYGPFLWNSLPLKQR | | | | |
| | | | | QLLSLRTFKKFIEN | | | | |
| L2-5_GA | Gastero- | RT and | NNNNN | (SEQ ID NO: 58) | | | (SEQ ID NO: 59) | (SEQ ID |
| | steus | EN, signal | NNNNN | MRRLLLLFLIMCLTPSPVPVRISSRRYYR | | | CAGTGTGCATC | NO: 60) |
| | aculeatus | peptide | NNNNN | PRARSALYRNLSSLSYPTRSTHVQHLVT | | | TCTTCTACAAGG | TAAAGAC |
| | | | NNNNN | GGLWNCQSATRKADFISGFAIQQSLDF | | | CCACCAGACAT | TAACAAA |
| | | | NNNNN | LALTETWITPENTSTPAALSSAFSFSHTP | | | CCAGGCGACCT | TTGTAGC |
| | | | NNNNN | RPTGRGGGTGLLISPKWSFSLYPLPPST | | | GAAATCGGACT | ACTTAAAT |
| | | | NNNNN | PLSFEFHAVTITHPVQLTIIVLYRPPGSL | | | AAGTATCTCTTT | TGTACTT |
| | | | NNNNN | GHFLEELDILLSNFPENGPPLILLGDFNI | | | CTTTAAACCAA | GTAACGT |
| | | | NNNNN | QTEKSSDLLHLLSSFALSLSPSPPTHKAG | | | GCTAGTCTGCT | CACTCATC |
| | | | NNNNN | NHLDYIFTRNCSTTNLSVTPLHVSDHFF | | | AAATTTGCTGTT | TATAGCA |
| | | | NNNNN | ISYSLPLSITNKPPSLTNSIPARRNIRSLSP | | | GATTCTAGTGTC | AATTGTA |
| | | | NNNNN | SSLASSVLSALPSTDSFSLLHPNAAAETL | | | TTAAGTTAATTT | AATTGGC |
| | | | NNNNN | LSTLSSSLDSLCPLTTRRTGKSPPAPWLS | | | GATTGCTGATTT | TTATTTGA |
| | | | NNNNN | QPVRAMRATMRASERRWRKYKRPDD | | | GCTTTAGTTTTT | GGAAATT |
| | | | NNNNN | LLEFQSLLSSFSASISAAKSSFYQSKIESSF | | | GTCGTCTCACG | GCACTTTC |
| | | | NNNNN | SNPKKLFSIFSNLLEPPTPPPPSTLLPGD | | | GCCAGTTCTCTT | TTGTTTCT |
| | | | NNNNN | FVNYFTKKIADIRSSFSNPPPTSRVPPTS | | | TGTTTGAAGTTA | TGTTCTCC |
| | | | NNNNN | PLSPSLSSFTALSPNQILTLVTSARPTTC | | | TCTTGGTTGCTA | TGAGTTT |
| | | | NNNNN | PLDPIPSHLLQSIAPDLLPFLTCLINNALS | | | GTTTGCTCAAG | GTACCCT |
| | | | NNNNN | SGCFPNSLKEARVNPLLKKPTLNPSEEN | | | CTCTTTCAGTCT | ATGGTTG |
| | | | nnnnnn | NYRPVSLLPFLSKTLERAIFNQLSSYLHC | | | TTTAGTGCCAGT | AATGCAC |
| | | | nnnnnn | NNLLDPHQSGFKAGHSTETALLAVSEQ | | | TTGTGTTCTAGA | TTATTGTA |
| | | | nnnnnn | LHTARAASLSSVLILLDLSAAFDTVNHQI | | | TTCTACTATTAA | CGTCGCT |
| | | | nnnnnn | LISSLQELGVTGSALSLLSSYLDGRTYRV | | | GTTAACTGCCA | TTGGATA |
| | | | nnnnnn | TWRGSVSEPCPLTTGVPQGSVLGPLLF | | | GTGTGCCCTCCT | AAAGCGT |
| | | | nnnnnn | SLYTNSLGAVIRSHGFSYHSYADDTQLI | | | AACTCTGCTAG | CAGCTAA |
| | | | nnnnnn | LSFPHSDTQVAARISACLTDISQWMSA | | | GTTTTGACCTGA | ATGACAT |
| | | | nnnnnn | HHLKINPDKTELLLFPGKDSLTQDLTVN | | | TCTAAGTCTGTT | GTAATGT |
| | | | nnnnnn | FGNSVLTPTSTAKNLGVTLDSQLSLTPN | | | TTGCCTTGTTTT | AATGTAA |
| | | | nnnnnn | ITATTRSCRYTLYNIRRIRPLLTQKAAQV | | | CTGAAGCAAAT | TGTAATG |
| | | | nnnnnn | LIQALVISRLDYCNSLLAGLPATAIRPLQ | | | AAGACTTGTAT | |
| | | | nnnnnn | LIQNAAARLVFNLPKFSHTTPLLRSLHW | | | CCTTAAACTCTC | |
| | | | nnnnnn | LPVAARIQFKTLVLTYHAVNGSGPAYIQ | | | ATTTTGTCAAAA | |
| | | | nnnnnn | DMVKPYIPTRTLRSASAKLLVPPSLRAK | | | CACCACATGGT | |
| | | | nnnnnn | HSTRSRLFAVLAPKWWNELSEDTRTAE | | | GTTTGTTCACTG | |
| | | | nnnnnn | SLHIFRRKLKTHLFRLYLD | | | ATTAGGGTGTC | |
| | | | nnnn | | | | AAGCTATTGGT | |
| | | | | | | | GCTTTGCATTTT | |
| | | | | | | | GAGGAGTTTCT | |
| | | | | | | | GTCGAGGCCAA | |
| | | | | | | | TCGACCTTTTGT | |
| | | | | | | | CTCCTAAACGA | |
| | | | | | | | CGGGGGAGCG | |
| | | | | | | | GCCAGCGCAGC | |
| | | | | | | | CGCAGCCGACT | |
| | | | | | | | TCCACTAACGA | |
| | | | | | | | GGGAGTATTTA | |
| | | | | | | | ACTAGAAATCG | |
| | | | | | | | TGGGAAGCGTT | |
| | | | | | | | AGCTTATCCTCG | |
| | | | | | | | CAGCACGAAGA | |
| | | | | | | | CGAGCAGAACA | |
| | | | | | | | AAGACCAGGGA | |
| | | | | | | | GTCTTTCGTGG | |
| | | | | | | | AGTCAAGACGA | |
| | | | | | | | GAGACAAAGAC | |
| | | | | | | | CAGGGAGTCTT | |
| | | | | | | | TCGTGGAGTCA | |
| | | | | | | | AGACGAGCAGA | |
| | | | | | | | ACAAAGACAAG | |
| | | | | | | | GGAGTCTTTCG | |
| | | | | | | | TGGCAGTCTTC | |
| | | | | | | | GGGGCGGCTGA | |
| RTE-3_BF | Branchios | RT and EN | NTNNN | (SEQ ID NO: 62) | - | | (SEQ ID NO: 63) | (SEQ ID |
| | toma | | NNNNN | MSGTPRVAFDSGKDLRNPIGQSPPALS | | | TCTGTAAATGG | NO: 64) |
| | floridae | | NNNTN | RAAPGQLGTDPSRSACFIGCLELRVLLD | | | CTGTGTGATGC | TGACCTG |
| | | | NNNNN | KWIVCRAPDKQESKEKRRQKTQPIRIG | | | GTCTAGTGTGT | ATACAGA |
| | | | NNNNN | SWNVRTMRTGLSDDLTVIEDIRKTAAI | | | AGGCGTAGCGG | GCGCTAC |
| | | | NNNNN | DRELYRLNIDIVALQETRLPDSGSLKEDS | | | TAGTGTGCTTG | CATCATCT |
| | | | NNNNN | YTFFWQGKGMEETREHGVGFAVRNT | | | CCACTTCTCGCT | GGAAAGA |
| | | | GNNNN | LLHMIEPPTGGTERIITLRLSTHEGPVNL | | | TTCAGCCCTCAC | TGGAAGG |
| | | | NNNNN | LCVYAPTLQATSEVKDQFYGQLDSAIK | | | CGCTGGCTAGC | ATGCCTA |
| | | | NNNNN | KIPVSEHIFILGDFNARVGTDQESWQT | | | GGAGCTGCATG | CTAC |
| | | | NNNNN | VLGHHGIGKMNENGQRLLELCCYHNL | | | CAGCACTGCGA | |
| | | | NNNNN | CVTNTFFQNKAIHKASWRHPRSQRW | | | AAAGAGAGCCG | |
| | | | NNNNN | HQLDLVITRRTSLNSVCNTRAYHSADC | | | AGTGCGTAAGT | |
| | | | NNNNN | DTDHSLIAARIKLRPKKLHHMKKKGQP | | | CTCTCCTGCCAG | |
| | | | NNTNN | KIDVSKTMLPDRNQKFLECLEGTLNNI | | | TACATAGCCTCT | |
| | | | NTNNN | QPQDAEHRWETLSKTIYSAAAQSYGK | | | CCACAGCAAGT | |
| | | | NNNNN | KERKNTDWFEAYISELEPVMDTKRKAL | | | CCCCATTGCAGT | |
| | | | NNNNA | VSYKQNPSSQNLQALKAARQEAQRAS | | | GCCTCCTCGTG | |
| | | | NNNNN | RRCANNYWLLLSERIQLASATGDIRRM | | | GCTACAGACGG | |
| | | | NNNNN | YEGIKQATGKPIKKSAPLKAKSGEIITDK | | | AAACTGGGCAC | |
| | | | nnnnnn | DKQMARWVEHYLDIYSTENSVSQDAL | | | CGTAAGGCCCC | |
| | | | nnnnnn | DNIEDFSVLAELDADPTIEELSKAIDSM | | | AGGCTAAACTG | |
| | | | tnnnnn | SNGKAPGEDNIPAEIIKSGKSVLLEPLHE | | | CAGGGAGCTCG | |
| | | | nnnnnn | LLRLCWKEGKVPQSMRNSKIVTLYKNK | | | GAGGAGGTCTG | |
| | | | nnngng | GDRTDCNSYRGISLLSIVGKVFAKVVLT | | | GCCCCCAGACG | |
| | | | nnnnnn | RLQVLADRVYPESQCGFRAERSTTDMI | | | CACGGCATGCA | |
| | | | tnnnnnc | FSVRQLQEKCREQQRPLYIAFIDLTKAF | | | TGGCCCACCGG | |
| | | | nnnnnn | DLVSRRGLFQLLRKIGCPPQLLDIIISFHE | | | CGTGTGGACAC | |
| | | | nntnnn | DMKGVVSFDGETSEPFAIRSGVKQGC | | | GCCCTGATGCC | |
| | | | nnnnnn | VLAPTLFGIFFSLLLKSAFGHSTQGVHL | | | TGCGAACCAGA | |
| | | | nnnnnt | HTRSDGKLFNLARLRAKTKVRSVLIRD | | | CCCCCAGCTAT | |
| | | | nnnnnn | MLFADDAALVAHVEDELQQLLNQFAH | | | GGGCAAATAGC | |
| | | | nnnnnn | ACSEFALTISIKKTVVMGQDVPQPPVV | | | ACGGGTAGACG | |
| | | | nannnn | TIGSEVLEVTDHFTYLGSTVTSNLSLDKE | | | GAGCTCGTCAG | |
| | | | nnnnnn | IDRRIARAAGVMTKLGTRVWNNSHLT | | | CCTTGGATGGC | |
| | | | nnnnnn | LNTKLEVYRSCVLSTLLYGSETWTTYAK | | | AGTTCGTCTAG | |
| | | | nnn | QENRLESFHLRCLRRILGISWRDRVPNT | | | GGGAAGGAAA | |
| | | | (SEQ ID | TVLERSCSLSIHLLLCQRRLRWLGHVSR | | | ACCCTGATTCAA | |
| | | | NO: 61 | MKDGRIPKDILFGELATGKRPVGRPAL | | | AAACCTCCGCT | |
| | | | | RFRDVCKRDLKLTDIDPASWEQIAADR | | | GCCTTGCGGCT | |
| | | | | NRWRHTVKDGLAKGQERRTEHLESRR | | | ATACCCAGTCCT | |
| | | | | RKRKEKPQQGNPSAFICPNCGRDCHA | | | GGGAAAGGCTA | |
| | | | | RIGLQSHSRRCQPP | | | CGGGAGTTAAC | |
| | | | | | | | CCAGAGAGAAA | |
| | | | | | | | ATCCGGAGTGG | |
| | | | | | | | AGTACGTGAGG | |
| | | | | | | | CGGTTGGCTGT | |
| | | | | | | | CAAACTCTGTCA | |
| | | | | | | | TCCTTCCGGCAA | |
| | | | | | | | CTCCTGCAGCC | |
| | | | | | | | AAACCAACGCC | |
| | | | | | | | AAGTGTCACGC | |
| | | | | | | | CTCGCGTTCCCT | |
| | | | | | | | TGGACCACGTC | |
| | | | | | | | GGTGAGGTCGA | |
| | | | | | | | GAGGGGGGTCC | |
| | | | | | | | TGTTGTGTTTTT | |
| | | | | | | | GGGCAGCGCAG | |
| | | | | | | | GTCCTCCATAAA | |
| | | | | | | | CCTGCCCAGGC | |
| | | | | | | | TAGCGCTCTGG | |
| | | | | | | | AGAGGCCACTC | |
| | | | | | | | CAGTCGCCCCC | |
| | | | | | | | ATCACTGGGGG | |
| | | | | | | | TGAGAAACAAA | |
| | | | | | | | CCGGGAGACAG | |
| | | | | | | | CAGTTTACGGG | |
| | | | | | | | TTATAAGTCCTT | |
| | | | | | | | GCTAAATTGAC | |
| | | | | | | | GTAA | |
| RTE- | Locusta | RT and EN | NNNNN | (SEQ ID NO: 66) | | | (SEQ ID NO: 67) | (SEQ ID |
| 25_LMi | migratoria | | NNNNN | MPRKNWNCGRRTGDEKRKMMFGC | | | CCCGTGTGGAG | NO: 68) |
| | | | NNNNN | WNVQGISTKIDLLPAELDMFNIDVVVL | | | TTTGCTGGTCTT | TAGTGTA |
| | | | NNNNN | SETKRKGKGEEELDNYVHIWSGVSKAV | | | CCATCGGGCAC | AAACCTT |
| | | | NNANN | RAKAGVSIMIQKKWKKRITNWTFINER | | | CTCCCCAGGTG | ATGTACT |
| | | | NNNNN | IITVEMTLFAREVVIIGVYAPTNDTKDK | | | GCGGATAGGG | AGGTGTA |
| | | | NNNNN | EKDAFWDTLRETIEKIPRRKELIIMGDM | | | GAATGCTCACC | TTCATTTC |
| | | | NNNNA | NGRVGIRESCKIVGKHGEAEYNDNGER | | | AGATATGGTGG | TGGGCGT |
| | | | NNNNN | LIDICAQFDLKITNTFFKHKDIHKYTWQ | | | GTACCGGGGAA | ATTAGTAT |
| | | | CNNNA | QNTKELRSIIDYIIIRQTSSFKAADVRSYR | | | ATAAAATACCC | GTTGGAG |
| | | | NNNNN | GAQCGSDHYLVKMKSFWPWKNATN | | | GGGGTGGACCA | GTAAACC |
| | | | NNNNN | DTSNINKMNCSEKVQNVHFNIDSLQD | | | AAACCAGCAAC | TCTGTAAT |
| | | | NNNNN | ESIRTFFKARMERTLDESFEGSTEEIYEYI | | | TGCTGCCTTGTA | GAGGACA |
| | | | NNNNN | KTKVKNVASEVLGIKENNPKRAAEWW | | | GTATGATATTG | ATCTCAAT |
| | | | NNNNN | SEEIETSVKEKRNAFVQWLNDKSEGTR | | | GCTTATCAAAG | AATAAAA |
| | | | NNNNN | SKYKEKKNEVEKKIRLAKNEAWERTCA | | | GCTAAAGGAAG | TAAAATA |
| | | | NNNNN | NVNSKLGFGRAKEAWSVLKALRQDTK | | | AAAACCTTGAA | AA |
| | | | NNNNN | GKSNLQLVTQKEWEEYFKKLLNEDRDE | | | TACAAATTACCT | |
| | | | NNNNN | YLEEGTVEENEHHDDEILISESEVLQVLR | | | GGTCCTCCAGG | |
| | | | NCNNN | TGKNGKSPGPGNINMEFLKYGGDKIV | | | TTGGGGGTTGT | |
| | | | nnnnnn | KLILQLFNKMLHGDSVPKEMKLGYISTI | | | GCAGTGGGCCA | |
| | | | nnnnna | FKKGDRKICSNYRGICVTNTLMRIFGKII | | | GCTCCTCACTCA | |
| | | | nnnnnn | KNKLEKNFRTQQEQCGFTAGRSCVDHI | | | CATAAAAATAT | |
| | | | nnnnnn | FTLRQILEKHREKSKNVGLIFIDLEKAYD | | | AAAATGCTAAA | |
| | | | nantnnt | TVPRKLLWRALHRANINTSLIKIIEQMY | | | AAACCTAATAA | |
| | | | nnnnnn | KDNICQVKIGNTLSQKFRTSKGLLQGC | | | T | |
| | | | nnnnnn | PMSPTLFKIYIDICLRTWSQKCNSMGLE | | | | |
| | | | nnnnan | IRDGVYLHHLLFADDQVVIAQDGEDA | | | | |
| | | | nnnnnn | NYMCNQLAIAYKNWGLKINYQKTEYL | | | | |
| | | | nnnnnn | TNDPHELRIEGKKIKKVNTFCYLGSILET | | | | |
| | | | gnnnnn | EGKSDSEINKRISSGRKVIGMLNSVLWS | | | | |
| | | | nannnn | RNVMNRTKKIIYKSIFESAVLYGAETWT | | | | |
| | | | ntnnnn | INQKHTKKLQALEMDFWRRSARISRKE | | | | |
| | | | nnannn | KRRNTEVIKRMEIIERIDEVMDRKKLR | | | | |
| | | | nnnnnn | WYGHVRRMEDTRIPKLVLEWQPEGR | | | | |
| | | | nnannn | RRRGRPVTTWIKNVQLTMNRLGAEEE | | | | |
| | | | nnn | DTQDRHTWRNIVNN | | | | |
| | | | (SEQ ID | | | | | |
| | | | NO: 65) | | | | | |

### Retrotransposon Discovery Tools

As the result of repeated mobilization over time, transposable elements in genomic DNA often exist as tandem or interspersed repeats (Jurka Curr Opin Struct Biol 8, 333-337 (1998)). Tools capable of recognizing such repeats can be used to identify new elements from genomic DNA and for populating databases, e.g., Repbase (Jurka et al Cytogenet Genome Res 110, 462-467 (2005)). One such tool for identifying repeats that may comprise transposable elements is RepeatFinder (Volfovsky et al Genome Biol 2 (2001)), which analyzes the repetitive structure of genomic sequences. Repeats can further be collected and analyzed using additional tools, e.g., Censor (Kohany et al BMC Bioinformatics 7, 474 (2006)). The Censor package takes genomic repeats and annotates them using various BLAST approaches against known transposable elements. An all-frames translation can be used to generate the ORF(s) for comparison.

Other exemplary methods for identification of transposable elements include RepeatModeler2, which automates the discovery and annotation of transposable elements in genome sequences (Flynn et al bioRxiv (2019)). In addition to accomplishing this via available packages like Censor, one can perform an all-frames translation of a given genome or sequence and annotate with a protein domain tool like InterProScan, which tags the domains of a given amino acid sequence using the InterPro database (Mitchell et al. Nucleic Acids Res 47, D351-360 (2019)), allowing the identification of potential proteins comprising domains associated with known transposable elements.

Retrotransposons can be further classified according to the reverse transcriptase domain using a tool such as RTclass1 (Kapitonov et al Gene 448, 207-213 (2009)).

### Polypeptide Component of Gene Modifying System

### RT Domain

In certain aspects, the reverse transcriptase domain of the gene modifying system is based on a reverse transcriptase domain of an APE-type or RLE-type non-LTR retrotransposon, or of a PLE-type retrotransposon. A wild-type reverse transcriptase domain of an APE-type, RLE-type, or PLE-type retrotransposon can be used in a gene modifying system or can be modified (e.g., by insertion, deletion, or substitution of one or more residues) to alter the reverse transcriptase activity for target DNA sequences. In some embodiments, the reverse transcriptase is altered from its natural sequence to have altered codon usage, e.g. improved for human cells. In some embodiments, the reverse transcriptase domain is a heterologous reverse transcriptase from a different LTR-retrotransposon, non-LTR retrotransposon, or other source. In certain embodiments, a gene modifying system includes a polypeptide that comprises a reverse transcriptase domain of a RTE (e.g., RTE-1_MD, RTE-3_BF, and RTE-25_LMi), CR1 (e.g., CR1-1_PH), Crack (e.g., Crack-28_RF), L2 (e.g., L2-2_Dre and L2-5_GA), and Vingi (e.g., Vingi-1_Acar) retrotransposon.

In certain embodiments, a gene modifying system includes a polypeptide that comprises a reverse transcriptase domain of a retrotransposon listed in Table 10, Table 11, Table X, Table Z1, Table Z2, or Table 3A or 3B of PCT Pub. No.: WO/2021/178717.

In certain embodiments, a gene modifying system includes a polypeptide that comprises a reverse transcriptase domain of a retrotransposon listed in Table 7. In some embodiments, the amino acid sequence of the reverse transcriptase domain of a gene modifying system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a reverse transcriptase domain of a retrotransposon whose DNA sequence is referenced in Table 7. Reverse transcriptase domains can be identified, for example, based upon homology to other known reverse transcription domains using routine tools as Basic Local Alignment Search Tool (BLAST). In some embodiments, reverse transcriptase domains are modified, for example by site-specific mutation. In some embodiments, the reverse transcriptase domain is engineered to bind a heterologous template RNA.

In some embodiments, a polypeptide (e.g., RT domain) comprises an RNA-binding domain, e.g., that specifically binds to an RNA sequence. In some embodiments, a template RNA comprises an RNA sequence that is specifically bound by the RNA-binding domain.

In some embodiments, the RT domain forms a dimer (e.g., a heterodimer or homodimer). In some embodiments, the RT domain is monomeric. In some embodiments, an RT domain naturally functions as a monomer or as a dimer (e.g., heterodimer or homodimer). In some embodiments, an RT domain naturally functions as a monomer. Naturally heterodimeric RT domains may, in some embodiments, also be functional as homodimers. In some embodiments, dimeric RT domains are expressed as fusion proteins, e.g., as homodimeric fusion proteins or heterodimeric fusion proteins. In some embodiments, the RT function of the system is fulfilled by multiple RT domains (e.g., as described herein). In further embodiments, the multiple RT domains are fused or separate, e.g., may be on the same polypeptide or on different polypeptides.

In some embodiment, a gene modifying polypeptide described herein comprises an RNase H domain, e.g., wherein the RNase H domain may be part of the RT domain. In some embodiments, an RT domain (e.g., as described herein) comprises an RNase H domain, e.g., an endogenous RNAse H domain or a heterologous RNase H domain. In some embodiments, an RT domain (e.g., as described herein) lacks an RNase H domain. In some embodiments, an RT domain (e.g., as described herein) comprises an RNase H domain that has been added, deleted, mutated, or swapped for a heterologous RNase H domain. In some embodiments, mutation of an RNase H domain yields a polypeptide exhibiting lower RNase activity, e.g., as determined by the methods described in Kotewicz et al. Nucleic Acids Res 16(1):265-277 (1988), e.g., lower by at least 10%, 20%, 30%, 40%, 50%, 60%, 70%, 80%, or 90% compared to an otherwise similar domain without the mutation. In some embodiments, RNase H activity is abolished.

In some embodiments, an RT domain is mutated to increase fidelity compared to an otherwise similar domain without the mutation. For instance, in some embodiments, a YADD (SEQ ID NO: 69) or YMDD (SEQ ID NO: 70) motif in an RT domain (e.g., in a reverse transcriptase) is replaced with YVDD. In embodiments, replacement of the YADD (SEQ ID NO: 69) or YMDD (SEQ ID NO: 70) or YVDD (SEQ ID NO: 71) results in higher fidelity in retroviral reverse transcriptase activity (e.g., as described in Jamburuthugoda and Eickbush J Mol Biol 2011.)

### Endonuclease Domain:

In some embodiments, the polypeptide comprises an endonuclease domain (e.g., a heterologous endonuclease domain). In certain embodiments, the endonuclease/DNA binding domain of an APE-type retrotransposon, the endonuclease domain of an RLE-type retrotransposon, or the endonuclease domain of a PLE-type retrotransposon can be used or can be modified (e.g., by insertion, deletion, or substitution of one or more residues) in a gene modifying system described herein. In some embodiments, the endonuclease domain or endonuclease/DNA binding domain is altered from its natural sequence to have altered codon usage, e.g. improved for human cells. In some embodiments, the endonuclease element is a heterologous endonuclease element. The amino acid sequence of an endonuclease domain of a gene modifying system described herein may be at least about 50%, at least about 60%, at least about 70%, at least about 80%, at least about 85%, at least about 90%, at least about 95%, at least about 96%, at least about 97%, at least about 98%, at least about 99% identical to the amino acid sequence of an endonuclease domain of a retrotransposon whose DNA sequence is referenced in Table X, Z1, Z2, 3A, or 3B of PCT Pub. No: WO/2021/178717.

In certain embodiments, a gene modifying system includes a polypeptide that comprises an endonuclease domain of a retrotransposon listed in Table 7. In some embodiments, the amino acid sequence of the endonuclease domain of a gene modifying system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a endonuclease domain of a retrotransposon whose DNA sequence is referenced in Table 7. Endonuclease domains can be identified, for example, based upon homology to other known endonuclease domains using tools as Basic Local Alignment Search Tool (BLAST).

In some embodiments, a gene modifying polypeptide possesses the function of DNA target site cleavage via an endonuclease domain. In some embodiments, the endonuclease domain is also a DNA-binding domain. In some embodiments, the endonuclease domain is also a template nucleic acid (e.g., template RNA) binding domain. In certain embodiments, the endonuclease/DNA binding domain of an APE-type retrotransposon or the endonuclease domain of an RLE-type retrotransposon can be used or can be modified (e.g., by insertion, deletion, or substitution of one or more residues) in a gene modifying system described herein.

### Template Nucleic Acid Binding Domain:

A gene modifying polypeptide typically contains regions capable of associating with the template nucleic acid (e.g., template RNA). In some embodiments, the template nucleic acid binding domain is an RNA binding domain. In some embodiments, the RNA binding domain is a modular domain that can associate with RNA molecules containing specific signatures, e.g., structural motifs, e.g., secondary structures present in the 3′ UTR in non-LTR retrotransposons. In other embodiments, the template nucleic acid binding domain (e.g., RNA binding domain) RNA binding domain is contained within the reverse transcription domain, e.g., the reverse transcriptase-derived component has a known signature for RNA preference, e.g., secondary structures present in the 3′ UTR in non-LTR retrotransposons.

### DNA Binding Domain:

In certain aspects, the DNA-binding domain of a gene modifying polypeptide described herein is selected, designed, or constructed for binding to a desired host DNA target sequence. In certain embodiments, the DNA-binding domain of the engineered retrotransposon is a heterologous DNA-binding protein or domain relative to a native retrotransposon sequence. In certain embodiments, the heterologous DNA-binding domain is a DNA binding domain of a retrotransposon described in Table 7 herein or in Table X, Table Z1, Table Z2, or Table 3A or 3B of PCT Pub. No.: WO/2021/178717. In some embodiments, DNA binding domains can be identified based upon homology to other known DNA binding domains using tools as Basic Local Alignment Search Tool (BLAST). In still other embodiments, DNA-binding domains are modified, for example by site-specific mutation. In some embodiments, the DNA binding domain is altered from its natural sequence to have altered codon usage, e.g. improved for human cells.

In embodiments, the DNA binding domain comprises one or more modifications relative to a wild-type DNA binding domain, e.g., a modification via directed evolution, e.g., phage-assisted continuous evolution (PACE).

In certain aspects of the present invention, the host DNA-binding site integrated into by the gene modifying system can be in a gene, in an intron, in an exon, an ORF, outside of a coding region of any gene, in a regulatory region of a gene, or outside of a regulatory region of a gene. In other aspects, the engineered retrotransposon may bind to one or more than one host DNA sequence. In other aspects, the engineered retrotransposon may have low sequence specificity, e.g., bind to multiple sequences or lack sequence preference.

In some embodiments, a gene modifying system is used to edit a target locus in multiple alleles. In some embodiments, a gene modifying system is designed to edit a specific allele. For example, a gene modifying polypeptide may be directed to a specific sequence that is only present on one allele, e.g., comprises a template RNA with homology to a target allele, e.g., an annealing domain, but not to a second cognate allele. In some embodiments, a gene modifying system can alter a haplotype-specific allele. In some embodiments, a gene modifying system that targets a specific allele preferentially targets that allele, e.g., has at least a 2, 4, 6, 8, or 10-fold preference for a target allele.

### Localization Sequences for Gene Modifying Systems

In certain embodiments, a gene modifying system RNA further comprises an intracellular localization sequence, e.g., a nuclear localization sequence.

The nuclear localization sequence may be an RNA sequence that promotes the import of the RNA into the nucleus. In certain embodiments, the nuclear localization signal is located on the template RNA. In certain embodiments, the retrotransposase polypeptide is encoded on a first RNA, and the template RNA is a second, separate, RNA, and the nuclear localization signal is located on the template RNA and not on an RNA encoding the retrotransposase polypeptide. While not wishing to be bound by theory, in some embodiments, the RNA encoding the retrotransposase is targeted primarily to the cytoplasm to promote its translation, while the template RNA is targeted primarily to the nucleus to promote its retrotransposition into the genome. In some embodiments, the nuclear localization signal is at the 3′ end, 5′ end, or in an internal region of the template RNA. In some embodiments the nuclear localization signal is 3′ of the heterologous sequence (e.g., is directly 3′ of the heterologous sequence) or is 5′ of the heterologous sequence (e.g., is directly 5′ of the heterologous sequence). In some embodiments, the nuclear localization signal is placed outside of the 5′ UTR or outside of the 3′ UTR of the template RNA. In some embodiments the nuclear localization signal is placed between the 5′ UTR and the 3′ UTR, wherein optionally the nuclear localization signal is not transcribed with the transgene (e.g., the nuclear localization signal is an anti-sense orientation or is downstream of a transcriptional termination signal or polyadenylation signal). In some embodiments, the nuclear localization sequence is situated inside of an intron. In some embodiments a plurality of the same or different nuclear localization signals are in the RNA, e.g., in the template RNA. In some embodiments, the nuclear localization signal is less than 5, 10, 25, 50, 75, 100, 150, 200, 250, 300, 350, 400, 450, 500, 600, 700, 800, 900, or 1000 bp in legnth. Various RNA nuclear localization sequences can be used. For example, Lubelsky and Ulitsky, Nature 555 (107-111), 2018 describe RNA sequences, which drive RNA localization into the nucleus. In some embodiments, the nuclear localization signal is a SINE-derived nuclear RNA localization (SIRLOIN) signal. In some embodiments, the nuclear localization signal binds a nuclear-enriched protein. In some embodiments, the nuclear localization signal binds the HNRNPK protein. In some embodiments the nuclear localization signal is rich in pyrimidines, e.g., is a C/T rich, C/U rich, C rich, T rich, or U rich region. In some embodiments, the nuclear localization signal is derived from a long non-coding RNA. In some embodiments, the nuclear localization signal is derived from MALATI long non-coding RNA or is the 600 nucleotide M region of MALAT1 (described in Miyagawa et al., RNA 18, (738-751), 2012). In some embodiments, the nuclear localization signal is derived from BORG long non-coding RNA or is a AGCCC motif (described in Zhang et al., Molecular and Cellular Biology 34, 2318-2329 (2014). In some embodiments, the nuclear localization sequence is described in Shukla et al., The EMBO Journal e98452 (2018). In some embodiments, the nuclear localization signal is derived from a non-LTR retrotransposon, an LTR retrotransposon, retrovirus, or an endogenous retrovirus.

In some embodiments, a polypeptide described herein comprises one or more (e.g., 2, 3, 4, 5) nuclear targeting sequences, for example, a nuclear localization sequence (NLS), e.g., as described herein. In some embodiments, the NLS is a bipartite NLS. In some embodiments, an NLS facilitates the import of a protein comprising an NLS into the cell nucleus. In some embodiments, the NLS is fused to the N-terminus of a gene modifying polypeptide described herein. In some embodiments, the NLS is fused to the C-terminus of the gene modifying polypeptide. In some embodiments, a linker sequence is disposed between the NLS and the neighboring domain of the gene modifying polypeptide. In some embodiments, an NLS comprises the amino acid sequence of an NLS described herein.

In some embodiments, a nucleic acid described herein (e.g., an RNA encoding a gene modifying polypeptide, or a DNA encoding the RNA) comprises a microRNA binding site. In some embodiments, the microRNA binding site is used to increase the target-cell specificity of a gene modifying system. For instance, the microRNA binding site can be chosen on the basis that is recognized by a miRNA that is present in a non-target cell type, but that is not present (or is present at a reduced level relative to the non-target cell) in a target cell type. Thus, when the RNA encoding the gene modifying polypeptide is present in a non-target cell, it would be bound by the miRNA, and when the RNA encoding the gene modifying polypeptide is present in a target cell, it would not be bound by the miRNA (or bound but at reduced levels relative to the non-target cell). While not wishing to be bound by theory, binding of the miRNA to the RNA encoding the gene modifying polypeptide may reduce production of the gene modifying polypeptide, e.g., by degrading the mRNA encoding the polypeptide or by interfering with translation. Accordingly, the heterologous object sequence would be inserted into the genome of target cells more efficiently than into the genome of non-target cells. A system having a microRNA binding site in the RNA encoding the gene modifying polypeptide (or encoded in the DNA encoding the RNA) may also be used in combination with a template RNA that is regulated by a second microRNA binding site.

In some embodiments, a polypeptide for use in any of the systems described herein can be a molecular reconstruction or ancestral reconstruction based upon the aligned polypeptide sequence of multiple retrotransposons. In some embodiments, a 5′ or 3′ untranslated region for use in any of the systems described herein can be a molecular reconstruction based upon the aligned 5′ or 3′ untranslated region of multiple retrotransposons. Based on the Accession numbers, polypeptides or nucleic acid sequences can be aligned, e.g., by using routine sequence analysis tools as Basic Local Alignment Search Tool (BLAST) or CD-Search for conserved domain analysis. Molecular reconstructions can be created based upon sequence consensus, e.g. using approaches described in Ivics et al., Cell 1997, 501-510; Wagstaff et al., Molecular Biology and Evolution 2013, 88-99. In some embodiments, the retrotransposon from which the 5′ or 3′ untranslated region or polypeptide is derived is a young or a recently active mobile element, as assessed via phylogenetic methods such as those described in Boissinot et al., Molecular Biology and Evolution 2000, 915-928.

### Inteins

In some embodiments, the gene modifying system comprises an intein. Generally, an intein comprises a polypeptide that has the capacity to join two polypeptides or polypepide fragments together via a peptide bond. In some embodiments, the intein is a trans-splicing intein that can join two polypeptide fragments, e.g., to form the polypeptide component of a system as described herein. Promoters

In some embodiments, one or more promoter or enhancer elements are operably linked to a nucleic acid encoding a gene modifying protein or a template nucleic acid, e.g., that controls expression of the heterologous object sequence. In certain embodiments, the one or more promoter or enhancer elements comprise cell-type or tissue specific elements. In some embodiments, the promoter or enhancer is the same or derived from the promoter or enhancer that naturally controls expression of the heterologous object sequence.

In some embodiments, a gene modifying system is capable of producing a substitution into the target site of at least 1, 2, 3, 4, 5, 6, 7, 8, 9, 10, 15, 20, 25, 30, 35, 40, 45, 50, 60, 70, 80, 90, or 100 or more nucleotides. In some embodiments, the substitution is a transition mutation. In some embodiments, the substitution is a transversion mutation. In some embodiments, the substitution converts an adenine to a thymine, an adenine to a guanine, an adenine to a cytosine, a guanine to a thymine, a guanine to a cytosine, a guanine to an adenine, a thymine to a cytosine, a thymine to an adenine, a thymine to a guanine, a cytosine to an adenine, a cytosine to a guanine, or a cytosine to a thymine.

In some embodiments, a gene modifying system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases).

In some embodiments, an insertion, deletion, substitution, or combination thereof, increases or decreases expression (e.g. transcription or translation) of a gene. In some embodiments, an insertion, deletion, substitution, or combination thereof, increases or decreases expression (e.g. transcription or translation) of a gene by altering, adding, or deleting sequences in a promoter or enhancer, e.g. sequences that bind transcription factors. In some embodiments, an insertion, deletion, substitution, or combination thereof alters translation of a gene (e.g. alters an amino acid sequence), inserts or deletes a start or stop codon, alters or fixes the translation frame of a gene. In some embodiments, an insertion, deletion, substitution, or combination thereof alters splicing of a gene, e.g. by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g. from the cytoplasm to a mitochondria, from the cytoplasm into the extracellular space (e.g. adds a secretion tag)). In some embodiments, an insertion, deletion, substitution, or combination thereof alters (e.g. improves) protein folding (e.g. to prevent accumulation of misfolded proteins). In some embodiments, an insertion, deletion, substitution, or combination thereof, alters, increases, decreases the activity of a gene, e.g. a protein encoded by the gene.

In some embodiments, a system or method described herein results in “scarless” insertion of the heterologous object sequence, while in some embodiments, the target site can show deletions or duplications of endogenous DNA as a result of insertion of the heterologous sequence. The mechanisms of different retrotransposons could result in different patterns of duplications or deletions in the host genome occurring during retrotransposition at the target site. In some embodiments, the system results in a scarless insertion, with no duplications or deletions in the surrounding genomic DNA. In some embodiments, the system results in a deletion of less than 1, 2, 3, 4, 5, 10, 50, or 100 bp of genomic DNA upstream of the insertion. In some embodiments, the system results in a deletion of less than 1, 2, 3, 4, 5, 10, 50, or 100 bp of genomic DNA downstream of the insertion. In some embodiments, the system results in a duplication of less than 1, 2, 3, 4, 5, 10, 50, or 100 bp of genomic DNA upstream of the insertion. In some embodiments, the system results in a duplication of less than 1, 2, 3, 4, 5, 10, 50, or 100 bp of genomic DNA downstream of the insertion.

In some embodiments, a gene modifying system described herein, or a DNA-binding domain thereof, binds to its target site specifically, e.g., as measured using an assay of Example 21 of PCT Application No. PCT/US2019/048607. In some embodiments, the gene modifying polypeptide or DNA-binding domain thereof binds to its target site more strongly than to any other binding site in the human genome. For example, in some embodiments, in an assay of Example 21 of PCT Application No. PCT/US2019/048607, the target site represents more than 50%, 60%, 70%, 80%, 90%, or 95% of binding events of the gene modifying polypeptide or DNA-binding domain thereof to human genomic DNA. In some embodiments, the DNA binding domain of the gene modifying polypeptide is heterologous to the remainder of the gene modifying polypeptide, e.g., such that the gene modifying polypeptide targets a different target site that the endogenous DNA binding domain associated with the remainder of the gene modifying polypeptide.

### Genetically Engineered, e.g., Dimerized Gene Modifying Polypeptides

Some non-LTR retrotransposons utilize two subunits to complete retrotransposition (Christensen et al PNAS 2006). In some embodiments, a retrotransposase described herein comprises two connected subunits as a single polypeptide. For instance, two wild-type retrotransposases could be joined with a linker to form a covalently “dimerized” protein. In some embodiments, the nucleic acid coding for the retrotransposase codes for two retrotransposase subunits to be expressed as a single polypeptide. In some embodiments, the subunits are connected by a peptide linker. Based on mechanism, not all functions are required from both retrotransposase subunits. In some embodiments, the fusion protein may consist of a fully functional subunit and a second subunit lacking one or more functional domains. In some embodiments, one subunit may lack reverse transcriptase functionality. In some embodiments, one subunit may lack the reverse transcriptase domain. In some embodiments, one subunit may possess only endonuclease activity. In some embodiments, one subunit may possess only an endonuclease domain. In some embodiments, the two subunits comprising the single polypeptide may provide complimentary functions.

In some embodiments, one subunit may lack endonuclease functionality. In some embodiments, one subunit may lack the endonuclease domain. In some embodiments, one subunit may possess only reverse transcriptase activity. In some embodiments, one subunit may possess only a reverse transcriptase domain. In some embodiments, one subunit may possess only DNA-dependent DNA synthesis functionality.

### Evolved Variants of Gene Modifying Polypeptides

In some embodiments, the invention provides evolved variants of gene modifying polypeptides. Evolved variants are described, e.g., at p. 1179-1182 of PCT application WO/2021/178720.

### Template RNA Component of Gene Modifying System

The gene modifying systems described herein can transcribe an RNA sequence template into host target DNA sites by target-primed reverse transcription. By writing DNA sequence(s) via reverse transcription of the RNA sequence template directly into the host genome, the gene modifying system can insert an object sequence into a target genome without the need for exogenous DNA sequences to be introduced into the host cell (unlike, for example, CRISPR systems), as well as eliminate an exogenous DNA insertion step. Therefore, the gene modifying system provides a platform for the use of customized RNA sequence templates containing object sequences, e.g., sequences comprising heterologous gene coding and/or function information.

In some embodiments, the template RNA encodes a gene modifying protein in cis with a heterologous object sequence. Various cis constructs were described, for example, in Kuroki-Kami et al (2019) Mobile DNA 10:23 (incorporated by reference herein in its entirety), and can be used in combination with any of the embodiments described herein. For instance, in some embodiments, the template RNA comprises a heterologous object sequence, a sequence encoding a gene modifying protein (e.g., a protein comprising (i) a reverse transcriptase domain and (ii) an endonuclease domain, e.g., as described herein), a 5′ untranslated region, and a 3′ untranslated region. The components may be included in various orders. In some embodiments, the gene modifying protein and heterologous object sequence are encoded in different directions (sense vs. anti-sense), e.g., using an arrangement shown in FIG. 3A of Kuroki-Kami et al, Id. In some embodiments, the gene modifying protein and heterologous object sequence are encoded in the same direction. In some embodiments, the nucleic acid encoding the polypeptide and the template RNA or the nucleic acid encoding the template RNA are covalently linked, e.g., are part of a fusion nucleic acid, and/or are part of the same transcript. In some embodiments, the fusion nucleic acid comprises RNA or DNA.

The nucleic acid encoding the gene modifying polypeptide may, in some instances, be 5′ of the heterologous object sequence. For example, in some embodiments, the template RNA comprises, from 5′ to 3′, a 5′ untranslated region, a sense-encoded gene modifying polypeptide, a sense-encoded heterologous object sequence, and 3′ untranslated region. In some embodiments, the template RNA comprises, from 5′ to 3′, a 5′ untranslated region, a sense-encoded gene modifying polypeptide, anti-sense-encoded heterologous object sequence, and 3′ untranslated region.

It is understood that, when a template RNA is described as comprising an open reading frame or the reverse complement thereof, in some embodiments the template RNA must be converted into double stranded DNA (e.g., through reverse transcription) before the open reading frame can be transcribed and translated.

In certain embodiments, customized RNA sequence template can be identified, designed, engineered and constructed to contain sequences altering or specifying host genome function, for example by introducing a heterologous coding region into a genome; affecting or causing exon structure/alternative splicing; causing disruption of an endogenous gene; causing transcriptional activation of an endogenous gene; causing epigenetic regulation of an endogenous DNA; causing up- or down-regulation of operably liked genes, etc. In certain embodiments, a customized RNA sequence template can be engineered to contain sequences coding for exons and/or transgenes, provide for binding sites to transcription factor activators, repressors, enhancers, etc., and combinations of thereof. In other embodiments, the coding sequence can be further customized with splice acceptor sites, poly-A tails. In certain embodiments the RNA sequence can contain sequences coding for an RNA sequence template homologous to the retrotransposase, be engineered to contain heterologous coding sequences, or combinations thereof.

The template RNA may have some homology to the target DNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the RNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 175, 180, or 200 or more bases of at least 50%, 60%, 70%, 80%, 85%, 90%, 95%, 97%, 98%, 99% or 100% homology to the target DNA, e.g., at the 5′ end of the template RNA. In some embodiments, the template RNA has a 3′ untranslated region derived from a retrotransposon, e.g. a retrotransposons described herein. In some embodiments the template RNA has a 3′ region of at least 10, 15, 20, 25, 30, 40, 50, 60, 80, 100, 120, 140, 160, 180, 200 or more bases of at least 50%, 60%, 70%, 80%, 85%, 90%, 95%, 97%, 98%, 99% or 100% homology to the 3′ sequence of a retrotransposon, e.g., a retrotransposon described herein, e.g. a retrotransposon in Table 7. In some embodiments, the template RNA has a 5′ untranslated region derived from a retrotransposon, e.g. a retrotransposons described herein. In some embodiments the template RNA has a 5′ region of at least 10, 15, 20, 25, 30, 40, 50, 60, 80, 100, 120, 140, 160, 180, or 200 or more bases of at least 40%, 50%, 60%, 70%, 80%, 90%, 95% or greater homology to the 5′ sequence of a retrotransposon, e.g., a retrotransposon described herein, e.g. a retrotransposon described in Table 7.

The template RNA component of a gene modifying system described herein typically is able to bind the gene modifying protein of the system. In some embodiments, the template RNA has a 3′ region that is capable of binding a gene modifying genome editing protein. The binding region, e.g., 3′ region, may be a structured RNA region, e.g., having at least 1, 2 or 3 hairpin loops, capable of binding the gene modifying protein of the system.

The template RNA component of a gene modifying system described herein typically is able to bind the gene modifying protein of the system. In some embodiments, the template RNA has a 5′ region that is capable of binding a gene modifying protein. The binding region, e.g., 5′ region, may be a structured RNA region, e.g., having at least 1, 2 or 3 hairpin loops, capable of binding the gene modifying protein of the system. In some embodiments, the 5′ untranslated region comprises a pseudoknot, e.g., a pseudoknot that is capable of binding to the gene modifying protein.

In some embodiments, the template RNA (e.g., an untranslated region of the hairpin RNA, e.g., a 5′ untranslated region) comprises a stem-loop sequence. In some embodiments, the template RNA (e.g., an untranslated region of the hairpin RNA, e.g., a 5′ untranslated region) comprises a hairpin. In some embodiments, the template RNA (e.g., an untranslated region of the hairpin RNA, e.g., a 5′ untranslated region) comprises a helix. In some embodiments, the template RNA (e.g., an untranslated region of the hairpin RNA, e.g., a 5′ untranslated region) comprises a psuedoknot. In some embodiments, the template RNA comprises a ribozyme. In some embodiments the ribozyme is similar to a hepatitis delta virus (HDV) ribozyme, e.g., has a secondary structure like that of the HDV ribozyme and/or has one or more activities of the HDV ribozyme, e.g., a self-cleavage activity. See, e.g., Eickbush et al., Molecular and Cellular Biology, 2010, 3142-3150.

In some embodiments, the template RNA (e.g., an untranslated region of the hairpin RNA, e.g., a 3′ untranslated region) comprises one or more stem-loops or helices. Exemplary structures of R2 3′ UTRs are shown, for example, in Ruschak et al. “Secondary structure models of the 3′ untranslated regions of diverse R2 RNAs” RNA. 2004 June; 10 (6): 978-987, e.g., at FIG. 3, therein, and in Eikbush and Eikbush, “R2 and R2/R1 hybrid non-autonomous retrotransposons derived by internal deletions of full-length elements” Mobile DNA (2012) 3:10; e.g., at FIG. 3 therein, which articles are hereby incorporated by reference in their entirety.

In some embodiments, a template RNA described herein comprises a sequence that is capable of binding to a gene modifying protein described herein. For instance, in some embodiments, the template RNA comprises an MS2 RNA sequence capable of binding to an MS2 coat protein sequence in the gene modifying protein. In some embodiments, the template RNA comprises an RNA sequence capable of binding to a B-box sequence. In some embodiments, in addition to or in place of a UTR, the template RNA is linked (e.g., covalently) to a non-RNA UTR, e.g., a protein or small molecule.

In some embodiments, the template RNA has a poly-A tail at the 3′ end. In some embodiments, the template RNA does not have a poly-A tail at the 3′ end.

In some embodiments the template RNA has a 5′ region of at least 10, 15, 20, 25, 30, 40, 50, 60, 80, 100, 120, 140, 160, 180, 200 or more bases of at least 40%, 50%, 60%, 70%, 80%, 90%, 95% or greater homology to the 5′ sequence of a retrotransposon, e.g., a retrotransposon described herein.

The template RNA of the system typically comprises an object sequence for insertion into a target DNA. The object sequence may be coding or non-coding.

In some embodiments, a system or method described herein comprises a single template RNA. In some embodiments, a system or method described herein comprises a plurality of template RNAs.

In some embodiments, the object sequence may contain an open reading frame. In some embodiments, the template RNA has a Kozak sequence. In some embodiments, the template RNA has an internal ribosome entry site. In some embodiments, the template RNA has a self-cleaving peptide such as a T2A or P2A site. In some embodiments, the template RNA has a start codon. In some embodiments, the template RNA has a splice acceptor site. In some embodiments, the template RNA has a splice donor site. Exemplary splice acceptor and splice donor sites are described in U.S. Pat. No. 10,435,677, incorporated herein by reference in its entirety. Exemplary splice acceptor site sequences are known to those of skill in the art and include, by way of example only, CTGACCCTTCTCTCTCTCCCCCAGAG (SEQ ID NO: 72) (from human HBB gene) and TTTCTCTCCCACAAG (SEQ ID NO: 73) (from human immunoglobulin-gamma gene). In some embodiments the template RNA, has a microRNA binding site downstream of the stop codon. In some embodiments, the template RNA has a poly A tail downstream of the stop codon of an open reading frame. In some embodiments, the template RNA comprises one or more exons. In some embodiments, the template RNA comprises one or more introns. In some embodiments, the template RNA comprises a eukaryotic transcriptional terminator. In some embodiments, the template RNA comprises an enhanced translation element or a translation enhancing element. In some embodiments, the RNA comprises the human T-cell leukemia virus (HTLV-1) R region. In some embodiments, the RNA comprises a posttranscriptional regulatory element that enhances nuclear export, such as that of Hepatitis B Virus (HPRE) or Woodchuck Hepatitis Virus (WPRE). In some embodiments, in the template RNA, the heterologous object sequence encodes a polypeptide and is coded in an antisense direction with respect to the 5′ and 3′ UTR. In some embodiments, in the template RNA, the heterologous object sequence encodes a polypeptide and is coded in a sense direction with respect to the 5′ and 3′ UTR.

In some embodiments, a nucleic acid described herein (e.g., a template RNA or a DNA encoding a template RNA) comprises a microRNA binding site. In some embodiments, the microRNA binding site is used to increase the target-cell specificity of a gene modifying system. For instance, the microRNA binding site can be chosen on the basis that is recognized by a miRNA that is present in a non-target cell type, but that is not present (or is present at a reduced level relative to the non-target cell) in a target cell type. Thus, when the template RNA is present in a non-target cell, it would be bound by the miRNA, and when the template RNA is present in a target cell, it would not be bound by the miRNA (or bound but at reduced levels relative to the non-target cell). While not wishing to be bound by theory, binding of the miRNA to the template RNA may interfere with insertion of the heterologous object sequence into the genome. Accordingly, the heterologous object sequence would be inserted into the genome of target cells more efficiently than into the genome of non-target cells. A system having a microRNA binding site in the template RNA (or DNA encoding it) may also be used in combination with a nucleic acid encoding a gene modifying polypeptide, wherein expression of the gene modifying polypeptide is regulated by a second microRNA binding site, e.g., as described herein, e.g., in the section entitled “Polypeptide component of gene modifying system.”

In some embodiments, the object sequence may contain a non-coding sequence. For example, the template RNA may comprise a promoter or enhancer sequence. In some embodiments, the template RNA comprises a tissue specific promoter or enhancer, each of which may be unidirectional or bidirectional. In some embodiments, the promoter is an RNA polymerase I promoter, RNA polymerase II promoter, or RNA polymerase III promoter. In some embodiments, the promoter comprises a TATA element. In some embodiments, the promoter comprises a B recognition element. In some embodiments, the promoter has one or more binding sites for transcription factors. In some embodiments, the non-coding sequence is transcribed in an antisense-direction with respect to the 5′ and 3′ UTR. In some embodiments, the non-coding sequence is transcribed in a sense direction with respect to the 5′ and 3′ UTR.

In some embodiments, a nucleic acid described herein (e.g., a template RNA or a DNA encoding a template RNA) comprises a promoter sequence, e.g., a tissue specific promoter sequence. In some embodiments, the tissue-specific promoter is used to increase the target-cell specificity of a gene modifying system. For instance, the promoter can be chosen on the basis that it is active in a target cell type but not active in (or active at a lower level in) a non-target cell type. Thus, even if the promoter integrated into the genome of a non-target cell, it would not drive expression (or only drive low-level expression) of an integrated gene. A system having a tissue-specific promoter sequence in the template RNA may also be used in combination with a microRNA binding site, e.g., in the template RNA or a nucleic acid encoding a gene modifying protein, e.g., as described herein. A system having a tissue-specific promoter sequence in the template RNA may also be used in combination with a DNA encoding a gene modifying polypeptide, driven by a tissue-specific promoter, e.g., to achieve higher levels of gene modifying protein in target cells than in non-target cells.

In some embodiments, a heterologous object sequence comprised by a template RNA (or DNA encoding the template RNA) is operably linked to at least one regulatory sequence. In some embodiments, the heterologous object sequence is operably linked to a tissue-specific promoter, such that expression of the heterologous object sequence, e.g., a therapeutic protein, is upregulated in target cells, as above. In some embodiments, the heterologous object sequence is operably linked to a miRNA binding site, such that expression of the heterologous object sequence, e.g., a therapeutic protein, is downregulated in cells with higher levels of the corresponding miRNA, e.g., non-target cells, as above.

In some embodiments, the template RNA comprises a microRNA sequence, a siRNA sequence, a guide RNA sequence, a piwi RNA sequence.

In some embodiments, the template RNA comprises a non-coding heterologous object sequence, e.g., a regulatory sequence. In some embodiments, integration of the heterologous object sequence thus alters the expression of an endogenous gene. In some embodiments, integration of the heterologous object sequence upregulates expression of an endogenous gene. In some embodiments, integration of the heterologous object sequence downregulated expression of an endogenous gene.

In some embodiments, the template RNA comprises a site that coordinates epigenetic modification. In some embodiments, the template RNA comprises an element that inhibits, e.g., prevents, epigenetic silencing. In some embodiments, the template RNA comprises a chromatin insulator. For example, the template RNA comprises a CTCF site or a site targeted for DNA methylation.

In order to promote higher level or more stable gene expression, the template RNA may include features that prevent or inhibit gene silencing. In some embodiments, these features prevent or inhibit DNA methylation. In some embodiments, these features promote DNA demethylation. In some embodiments, these features prevent or inhibit histone deacetylation. In some embodiments, these features prevent or inhibit histone methylation. In some embodiments, these features promote histone acetylation. In some embodiments, these features promote histone demethylation. In some embodiments, multiple features may be incorporated into the template RNA to promote one or more of these modifications. CpG dinculeotides are subject to methylation by host methyl transferases. In some embodiments, the template RNA is depleted of CpG dinucleotides, e.g., does not comprise CpG nucleotides or comprises a reduced number of CpG dinucleotides compared to a corresponding unaltered sequence. In some embodiments, the promoter driving transgene expression from integrated DNA is depleted of CpG dinucleotides.

In some embodiments, the template RNA comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA).

In some embodiments, the object sequence of the template RNA is inserted into a target genome in an endogenous intron. In some embodiments, the object sequence of the template RNA is inserted into a target genome and thereby acts as a new exon. In some embodiments, the insertion of the object sequence into the target genome results in replacement of a natural exon or the skipping of a natural exon.

In some embodiments, the object sequence of the template RNA is inserted into the target genome in a genomic safe harbor site, such as AAVSI, CCR5, or ROSA26. In some embodiments, the object sequence of the template RNA is inserted into the albumin locus. In some embodiments, the object sequence of the template RNA is inserted into the TRAC locus. In some embodiments, the object sequence of the template RNA is added to the genome in an intergenic or intragenic region. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments, the object sequence of the template RNA can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp. In some embodiments, the heterologous object sequence is less than 1,000, 1,300, 1500, 2,000, 3,000, 4,000, 5,000, or 7,500 nucleotides in length.

The template nucleic acid (e.g., template RNA) component of a gene modifying system described herein typically is able to bind the gene modifying protein of the system. In some embodiments, the template nucleic acid (e.g., template RNA) has a 3′ region that is capable of binding a gene modifying protein. The binding region, e.g., 3′ region, may be a structured RNA region, e.g., having at least 1, 2 or 3 hairpin loops, capable of binding the gene modifying protein of the system. The binding region may associate the template nucleic acid (e.g., template RNA) with any of the polypeptide modules. In some embodiments, the binding region of the template nucleic acid (e.g., template RNA) may associate with an RNA-binding domain in the polypeptide. In some embodiments, the binding region of the template nucleic acid (e.g., template RNA) may associate with the reverse transcription domain of the polypeptide (e.g., specifically bind to the RT domain). For example, where the reverse transcription domain is derived from a non-LTR retrotransposon, the template nucleic acid (e.g., template RNA) may contain a binding region derived from a non-LTR retrotransposon, e.g., a 3′ UTR from a non-LTR retrotransposon. In some embodiments a system or method described herein comprises a single template nucleic acid (e.g., template RNA). In some embodiments a system or method described herein comprises a plurality of template nucleic acids (e.g., template RNAs). In some embodiments, when the system comprises a plurality of nucleic acids, each nucleic acid comprises a conjugating domain. In some embodiments, a conjugating domain enables association of nucleic acid molecules, e.g., by hybridization of complementary sequences.

In some embodiments, the template nucleic acid may comprise one or more UTRs (e.g., a 5′ UTR or a 3′ UTR, e.g., from an R2-type retrotransposon). In some embodiments, the UTR facilitates interaction of the template with the reverse transcriptase domain of the polypeptide. In some embodiments, the template possesses one or more sequences aiding in association of the template with the gene modifying polypeptide. In some embodiments, these sequences may be derived from retrotransposon UTRs. In some embodiments, the UTRs may be located flanking the desired insertion sequence. In some embodiments, a sequence with target site homology may be located outside of one or both UTRs. In some embodiments, the sequence with target site homology can anneal to the target sequence to prime reverse transcription. In some embodiments, the 5′ and/or 3′ UTR may be located terminal to the target site homology sequence. In some embodiments, the gene modifying system may result in the insertion of a desired payload without any additional sequence (e.g., a gene expression unit without UTRs used to bind the gene modifying protein).

The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous sequence, wherein the reverse transcription will result in insertion of the heterologous sequence into the target DNA. In other embodiments, the RNA template may be designed to write a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to write an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, a gene modifying system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases).

### Methods and Compositions for Modified RNA (e.g., Template RNA)

In some embodiments, an RNA component of the system (e.g., a template RNA, as described herein) comprises one or more nucleotide modifications. In some embodiments, the modification pattern of the template RNA can significantly affect in vivo activity compared to unmodified or end-modified guides. Without wishing to be bound by theory, this process may be due, at least in part, to a stabilization of the RNA conferred by the modifications. Non-limiting examples of such modifications may include 2′-O-methyl(2′-O-Me), 2′-O-(2-methoxyethyl) (2′-O-MOE), 2′-fluoro (2′-F), phosphorothioate (PS) bond between nucleotides, G-C substitutions, and inverted abasic linkages between nucleotides and equivalents thereof.

In some embodiments, the template RNA (e.g., at the portion thereof that binds a target site) comprises a 5′ terminus region. In some embodiments, the template RNA does not comprise a 5′ terminus region. In some embodiments, the 5′ terminus region comprises a 5′ end modification. In some embodiments, the template RNA comprises a 2′-O-methyl(2′-O-Me) modified nucleotide. In some embodiments, the template RNA comprises a 2′-O-(2-methoxy ethyl) (2′-O-moe) modified nucleotide. In some embodiments, the template RNA comprises a 2′-fluoro (2′-F) modified nucleotide. In some embodiments, the template RNA comprises a phosphorothioate (PS) bond between nucleotides. In some embodiments, the template RNA comprises a 5′ end modification, a 3′ end modification, or 5′ and 3′ end modifications. In some embodiments, the 5′ end modification comprises a phosphorothioate (PS) bond between nucleotides. In some embodiments, the 5′ end modification comprises a 2′-O-methyl(2′-O-Me), 2′-O-(2-methoxy ethyl) (2′-O-MOE), and/or 2′-fluoro (2′-F) modified nucleotide. In some embodiments, the 5′ end modification comprises at least one phosphorothioate (PS) bond and one or more of a 2′-O-methyl(2′-O-Me), 2′-O-(2-methoxyethyl) (2′-O-MOE), and/or 2′-fluoro (2′-F) modified nucleotide. The end modification may comprise a phosphorothioate (PS), 2′-O-methyl(2′-O-Me), 2′-O-(2-methoxyethyl) (2′-O-MOE), and/or 2′-fluoro (2′-F) modification. Equivalent end modifications are also encompassed by embodiments described herein. In some embodiments, the template RNA comprises an end modification in combination with a modification of one or more regions of the template RNA. In some embodiments, structure-guided and systematic approaches are used to introduce modifications (e.g., 2′-OMe-RNA, 2′-F-RNA, and PS modifications) to a template RNA, for example, as described in Mir et al. Nat Commun 9:2641 (2018) (incorporated by reference herein in its entirety). In some embodiments, the incorporation of 2′-F-RNAs increases thermal and nuclease stability of RNA: RNA or RNA: DNA duplexes, e.g., while minimally interfering with C3′-endo sugar puckering. In some embodiments, 2′-F may be better tolerated than 2′-OMe at positions where the 2′-OH is important for RNA: DNA duplex stability. In some embodiments, structure-guided and systematic approaches (e.g., as described in Mir et al. Nat Commun 9:2641 (2018); incorporated herein by reference in its entirety) are employed to find modifications for the template RNA. In some embodiments, a structure of polypeptide bound to template RNA is used to determine non-protein-contacted nucleotides of the RNA that may then be selected for modifications, e.g., with lower risk of disrupting the association of the RNA with the polypeptide. Secondary structures in a template RNA can also be predicted in e.g., silico by software tools, the RNAstructure tool available at rna.urmc.rochester.edu/RNAstructureWeb (Bellaousov et al. Nucleic Acids Res 41:W471-W474 (2013); incorporated by reference herein in its entirety), e.g., to determine secondary structures for selecting modifications, e.g., hairpins, stems, and/or bulges.

It is contemplated that it may be useful to employ circular and/or linear RNA states during the formulation, delivery, or gene modifying reaction within the target cell. Thus, in some embodiments of any of the aspects described herein, a gene modifying system comprises one or more circular RNAs (circRNAs). In some embodiments of any of the aspects described herein, a gene modifying system comprises one or more linear RNAs. In some embodiments, a nucleic acid as described herein (e.g., a template nucleic acid, a nucleic acid molecule encoding a gene modifying polypeptide, or both) is a circRNA. In some embodiments, a circular RNA molecule encodes the gene modifying polypeptide. In some embodiments, the circRNA molecule encoding the gene modifying polypeptide is delivered to a host cell. In some embodiments, the circRNA molecule encoding the gene modifying polypeptide is linearized (e.g., in the host cell, e.g., in the nucleus of the host cell) prior to translation. Circular RNAs are described, e.g., at p. 1215-1218 of PCT Pub. No. WO/2021/178720.

Further included here are compositions and methods for the assembly of full or partial template RNA molecules. Methods of making template RNAs are described, e.g., at p. 1150-1155 of PCT Pub. No. WO/2021/178720.

### Additional Template Features

In some embodiments, the template (e.g., template RNA) comprises certain structural features, e.g., determined in silico. In embodiments, the template RNA is predicted to have minimal energy structures between −280 and −480 kcal/mol (e.g., between −280 to −300, −300 to −350, −350 to −400, −400 to −450, or −450 to −480 kcal/mol), e.g., as measured by RNAstructure, e.g., as described in Turner and Mathews Nucleic Acids Res 38: D280-282 (2009) (incrated herein by reference in its entirety).

In some embodiments, the template (e.g., template RNA) comprises certain structural features, e.g., determined in vitro. In embodiments, the template RNA is sequence optimized, e.g., to reduce secondary structure as determined in vitro, for example, by SHAPE-MaP (e.g., as described in Siegfried et al. Nat Methods 11:959-965 (2014); incorporated herein by reference in its entirety). In some embodiments, the template (e.g., template RNA) comprises certain structural features, e.g., determined in cells. In embodiments, the template RNA is sequence optimized, e.g., to reduce secondary structure as measured in cells, for example, by DMS-MaPseq (e.g., as described in Zubradt et al. Nat Methods 14:75-82 (2017); incorporated by reference herein in its entirety).

### Additional Functional Characteristics and Features of Gene Modifying Systems

A gene modifying system as described herein may, in some instances, be characterized by one or more functional measurements or characteristics. In some embodiments, the DNA binding domain has one or more of the functional characteristics described below. In some embodiments, the RNA binding domain has one or more of the functional characteristics described below. In some embodiments, the endonuclease domain has one or more of the functional characteristics described below. In some embodiments, the reverse transcriptase domain has one or more of the functional characteristics described below. In some embodiments, the template (e.g., template RNA) has one or more of the functional characteristics described below. In some embodiments, the target site bound by the gene modifying polypeptide has one or more of the functional characteristics described below.

### Gene Modifying Polypeptide

### DNA Binding Domain

In some embodiments, the DNA binding domain is capable of binding to a target sequence (e.g., a dsDNA target sequence) with greater affinity than a reference DNA binding domain. In some embodiments, the reference DNA binding domain is a DNA binding domain from R2 BM of B. mori. In some embodiments, the DNA binding domain is capable of binding to a target sequence (e.g., a dsDNA target sequence) with an affinity between 100 pM-10 nM (e.g., between 100 pM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 μM-10 nM (e.g., between 100 μM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

In some embodiments, a gene modifying polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, a gene modifying polypeptide comprises a modification to an endonuclease domain, e.g., relative to the wild-type polypeptide. In some embodiments, the endonuclease domain comprises an addition, deletion, replacement, or modification to the amino acid sequence of the original endonuclease domain. In some embodiments, the endonuclease domain is modified to include a heterologous functional domain that binds specifically to and/or induces endonuclease cleavage of a target nucleic acid (e.g., DNA) sequence of interest.

### RNA Binding Domain

In some embodiments, the RNA binding domain is capable of binding to a template RNA with greater affinity than a reference RNA binding domain. In some embodiments, the reference RNA binding domain is an RNA binding domain from R2_BM of B. mori. In some embodiments, the RNA binding domain is capable of binding to a template RNA with an affinity between 100 μM-10 nM (e.g., between 100 μM-1 nM or 1 nM-10 nM). In some embodiments, the affinity of a RNA binding domain for its template RNA is measured in vitro, e.g., by thermophoresis, e.g., as described in Asmari et al. Methods 146:107-119 (2018) (incorporated by reference herein in its entirety). In some embodiments, the affinity of a RNA binding domain for its template RNA is measured in cells (e.g., by FRET or CLIP-Seq).

In some embodiments, the RNA binding domain is associated with the template RNA in vitro at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019) Nucleic Acids Res 47 (11): 5490-5501 (incorporated by reference herein in its entirety). In some embodiments, the RNA binding domain is associated with the template RNA in cells (e.g., in HEK293T cells) at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019), supra.

### Endonuclease Domain

In some embodiments, the endonuclease domain is associated with the target dsDNA in vitro at a frequency at least about 5-fold or 10-fold higher than with a scrambled dsDNA. In some embodiments, the endonuclease domain is associated with the target dsDNA in vitro at a frequency at least about 5-fold or 10-fold higher than with a scrambled dsDNA, e.g., in a cell (e.g., a HEK293T cell). In some embodiments, the frequency of association between the endonuclease domain and the target DNA or scrambled DNA is measured by ChIP-seq, e.g., as described in He and Pu (2010) Curr. Protoc Mol Biol Chapter 21 (incorporated by reference herein in its entirety).

In some embodiments, the endonuclease domain can catalyze the formation of a nick at a target sequence, e.g., to an increase of at least about 5-fold or 10-fold relative to a non-target sequence (e.g., relative to any other genomic sequence in the genome of the target cell). In some embodiments, the level of nick formation is determined using NickSeq, e.g., as described in Elacqua et al. (2019) bioRxiv doi.org/10.1101/867937 (incorporated herein by reference in its entirety).

In some embodiments, the endonuclease domain is capable of nicking DNA in vitro. In embodiments, the nick results in an exposed base. In embodiments, the exposed base can be detected using a nuclease sensitivity assay, e.g., as described in Chaudhry and Weinfeld (1995) Nucleic Acids Res 23(19):3805-3809 (incorporated by reference herein in its entirety). In embodiments, the level of exposed bases (e.g., detected by the nuclease sensitivity assay) is increased by at least 10%, 50%, or more relative to a reference endonuclease domain. In some embodiments, the reference endonuclease domain is an endonuclease domain from R2_BM of B. mori.

In some embodiments, the endonuclease domain is capable of nicking DNA in a cell. In embodiments, the endonuclease domain is capable of nicking DNA in a HEK293T cell. In embodiments, an unrepaired nick that undergoes replication in the absence of Rad51 results in increased NHEJ rates at the site of the nick, which can be detected, e.g., by using a Rad51 inhibition assay, e.g., as described in Bothmer et al. (2017) Nat Commun 8:13905 (incorporated by reference herein in its entirety). In embodiments, NHEJ rates are increased above 0-5%. In embodiments, NHEJ rates are increased to 20-70% (e.g., between 30%-60% or 40-50%), e.g., upon Rad51 inhibition.

In some embodiments, the endonuclease domain releases the target after cleavage. In some embodiments, release of the target is indicated indirectly by assessing for multiple turnovers by the enzyme, e.g., as described in Yourik at al. RNA 25(1):35-44 (2019) (incorporated herein by reference in its entirety) and shown in FIG. 2. In some embodiments, the kexp of an endonuclease domain is 1×10⁻³-1×10⁻⁵ min−1 as measured by such methods.

In some embodiments, the endonuclease domain has a catalytic efficiency (k_{cat}/Kₘ) greater than about 1×10⁸ s⁻¹ M⁻¹ in vitro. In embodiments, the endonuclease domain has a catalytic efficiency greater than about 1×10⁵, 1×10⁶, 1×10⁷, or 1×10⁸, s⁻¹ M⁻¹ in vitro. In embodiments, catalytic efficiency is determined as described in Chen et al. (2018) Science 360(6387):436-439 (incorporated herein by reference in its entirety). In some embodiments, the endonuclease domain has a catalytic efficiency (k_{cat}/Kₘ) greater than about 1×10⁸ s⁻¹ M⁻¹ in cells. In embodiments, the endonuclease domain has a catalytic efficiency greater than about 1×10⁵, 1×10⁶, 1×10⁷, or 1×10⁸ s⁻¹ M⁻¹ in cells.

### Reverse Transcriptase Domain

In some embodiments, the reverse transcriptase domain has a lower probability of premature termination rate (P_{off}) in vitro relative to a reference reverse transcriptase domain. In some embodiments, the reference reverse transcriptase domain is a reverse transcriptase domain from R2_BM of B. mori or a viral reverse transcriptase domain, e.g., the RT domain from M-MLV.

In some embodiments, the reverse transcriptase domain has a lower probability of premature termination rate (Poff) in vitro of less than about 5×10⁻³/nt, 5×10⁻⁴/nt, or 5×10⁻⁶/nt, e.g., as measured on a 1094 nt RNA. In embodiments, the in vitro premature termination rate is determined as described in Bibillo and Eickbush (2002) J Biol Chem 277(38):34836-34845 (incorporated by reference herein its entirety).

In some embodiments, the reverse transcriptase domain is able to complete at least about 30% or 50% of integrations in cells. The percent of complete integrations can be measured by dividing the number of substantially full-length integration events (e.g., genomic sites that comprise at least 98% of the expected integrated sequence) by the number of total (including substantially full-length and partial) integration events in a population of cells. In embodiments, the integrations in cells is determined (e.g., across the integration site) using long-read amplicon sequencing, e.g., as described in Karst et al. (2020) bioRxiv doi.org/10.1101/645903 (incorporated by reference herein in its entirety).

In embodiments, quantifying integrations in cells comprises counting the fraction of integrations that contain at least about 75%, 80%, 85%, 90%, 95%, 96%, 97%, 98%, 99%, or 100% of the DNA sequence corresponding to the template RNA (e.g., a template RNA having a length of at least 0.05, 0.1, 0.5, 0.6, 0.7, 0.8, 0.9, 1, 1.5, 2, 3, 4, or 5 kb, e.g., a length between 0.5-0.6, 0.6-0.7, 0.7-0.8, 0.8-0.9, 1.0-1.2, 1.2-1.4, 1.4-1.6, 1.6-1.8, 1.8-2.0, 2-3, 3-4, or 4-5 kb).

In some embodiments, the reverse transcriptase domain is capable of polymerizing dNTPs in vitro. In embodiments, the reverse transcriptase domain is capable of polymerizing dNTPs in vitro at a rate between 0.1-50 nt/sec (e.g., between 0.1-1, 1-10, or 10-50 nt/sec). In embodiments, polymerization of dNTPs by the reverse transcriptase domain is measured by a single-molecule assay, e.g., as described in Schwartz and Quake (2009) PNAS 106 (48): 20294-20299 (incorporated by reference in its entirety).

In some embodiments, the reverse transcriptase domain has an in vitro error rate (e.g., misincorporation of nucleotides) of between 1×10⁻³-1×10⁻⁴ or 1×10⁻⁴-1×10⁻⁵ substitutions/nt, e.g., as described in Yasukawa et al. (2017) Biochem Biophys Res Commun 492(2):147-153 (incorporated herein by reference in its entirety). In some embodiments, the reverse transcriptase domain has an error rate (e.g., misincorporation of nucleotides) in cells (e.g., HEK293T cells) of between 1×10⁻³-1×10⁻⁴ or 1×10⁻⁴-1×10⁻⁵ substitutions/nt, e.g., by long-read amplicon sequencing, e.g., as described in Karst et al. (2020) bioRxiv doi.org/10.1101/645903 (incorporated by reference herein in its entirety).

In some embodiments, the reverse transcriptase domain is capable of performing reverse transcription of a target RNA in vitro. In some embodiments, the reverse transcriptase requires a primer of at least 3 nt to initiate reverse transcription of a template. In some embodiments, reverse transcription of the target RNA is determined by detection of cDNA from the target RNA (e.g., when provided with a ssDNA primer, e.g., which anneals to the target with at least 3, 4, 5, 6, 7, 8, 9, or 10 nt at the 3′ end), e.g., as described in Bibillo and Eickbush (2002) J Biol Chem 277(38):34836-34845 (incorporated herein by reference in its entirety).

In some embodiments, the reverse transcriptase domain performs reverse transcription at least 5 or 10 times more efficiently (e.g., by cDNA production), e.g., when converting its RNA template to cDNA, for example, as compared to an RNA template lacking the protein binding motif (e.g., a 3′ UTR). In embodiments, efficiency of reverse transcription is measured as described in Yasukawa et al. (2017) Biochem Biophys Res Commun 492(2):147-153 (incorporated by reference herein in its entirety).

In some embodiments, the reverse transcriptase domain specifically binds a specific RNA template with higher frequency (e.g., about 5 or 10-fold higher frequency) than any endogenous cellular RNA, e.g., when expressed in cells (e.g., HEK293T cells). In embodiments, frequency of specific binding between the reverse transcriptase domain and the template RNA are measured by CLIP-seq, e.g., as described in Lin and Miles (2019) Nucleic Acids Res 47(11):5490-5501 (incorporated herein by reference in its entirety).

### Target Site and Integration

In some embodiments, after gene editing, the target site surrounding the integrated sequence contains a limited number of insertions or deletions, for example, in less than about 50% or10% of integration events, e.g., as determined by long-read amplicon sequencing of the target site, e.g., as described in Karst et al. (2020) bioRxiv doi.org/10.1101/645903 (incorporated by reference herein in its entirety). In some embodiments, the target site does not show multiple insertion events, e.g., head-to-tail or head-to-head duplications, e.g., as determined by long-read amplicon sequencing of the target site, e.g., as described in Karst et al. bioRxiv doi.org/10.1101/645903 (2020) (incorporated herein by reference in its entirety). In some embodiments, the target site contains an integrated sequence corresponding to the template RNA. In some embodiments, the target site does not contain insertions resulting from endogenous RNA in more than about 1% or10% of events, e.g., as determined by long-read amplicon sequencing of the target site, e.g., as described in Karst et al. bioRxiv doi.org/10.1101/645903 (2020) (incorporated herein by reference in its entirety). In some embodiments, the target site contains the integrated sequence corresponding to the template RNA.

In some embodiments, the target site contains an integrated sequence corresponding to the template RNA. In embodiments, the target site does not comprise sequence outside of the template, e.g., as determined by long-read amplicon sequencing of the target site (for example, as described in Karst et al. bioRxiv doi.org/10.1101/645903 (2020); incorporated herein by reference in its entirety).

### DNA Damage Response

In some embodiments, modifying a genome of a cell (e.g., a primary cell, e.g., a T cell) using a gene modifying system does not result in activation of the endogenous DNA damage response (DDR) pathway. In some embodiments, modifying a genome of a cell (e.g., a primary cell) using a gene modifying system results in activation of the cell's endogenous DDR pathway less than in an otherwise similar cell treated with Cas9.

In some embodiments, modifying a genome of a cell (e.g., a primary cell, e.g., a T cell) using a gene modifying system does not result in activation of the endogenous interferon response. In some embodiments, modifying a genome of a cell using a gene modifying system results in activation of the cell's interferon response less than in an otherwise similar cell treated with a gene modifying system comprising elements from a LINE-1 retrotransposase.

In some embodiments, the gene modifying polypeptide systems described herein includes a self-inactivating module. The self-inactivating module leads to a decrease of expression of the gene modifying polypeptide, the gene modifying template, or both. Self-inactivating modules are described, e.g., at p. 1200-1201 of PCT Pub. No. WO/2021/178720.

In some embodiments a polypeptide described herein (e.g., a gene modifying polypeptide) is controllable via a small molecule. In some embodiments, the polypeptide is dimerized via a small molecule. Polypeptides of this type are described, e.g., at p. 1201-1203 of WO/2021/178720.

### Heterologous Gene Modifying Systems

The conjugates (targeted LNPs) described herein can be formulated to comprise one or more components of a heterologous gene modifying system, or one or more nucleic acids encoding said components. Accordingly, in some embodiments, the payload comprises a heterologous gene modifying system, or one or more nucleic acids encoding the components of the heterologous gene modifying polypeptide. For instance, in some embodiments, the payload comprises a template RNA and an mRNA encoding the heterologous gene modifying polypeptide.

A heterologous gene modifying system may comprise a heterologous gene modifying polypeptide and a template RNA. The heterologous gene modifying polypeptide may comprise an endonuclease domain, a DNA binding domain, a linker, and a reverse transcriptase domain derived from a retrovirus. The heterologous gene modifying polypeptide may comprise a Cas domain, a linker, and a reverse transcriptase domain derived from a retrovirus. The template RNA compatible with the heterologous gene modifying polypeptide may comprise (e.g., from 5′ to 3′) (i) a gRNA spacer that binds a target site, (ii) a gRNA scaffold that binds the heterologous gene modifying polypeptide, e.g., the Cas domain of the heterologous gene modifying polypeptide, (iii) a heterologous object sequence, and (iv) a primer binding site (PBS) sequence. These components are now described in more detail.

In some aspects, a heterologous gene modifying polypeptide described herein comprises (e.g., a system described herein comprises a gene modifying polypeptide that comprises): 1) a Cas domain (e.g., a Cas nickase domain, e.g., a Cas9 nickase domain); 2) a reverse transcriptase (RT) domain, wherein the RT domain is C-terminal of the Cas domain; and a linker disposed between the RT domain and the Cas domain.

In some embodiments, the heterologous gene modifying polypeptide comprises a sequence of SEQ ID NO: 4000 which comprises the first NLS and the Cas domain, or a sequence having at least 70%, 75%, 80%, 85%, 90%, 95%, 98%, or 99% identity thereto. In some embodiments, the heterologous gene modifying polypeptide comprises a sequence of SEQ ID NO: 4001 which comprises the second NLS, or a sequence having at least 70%, 75%, 80%, 85%, 90%, 95%, 98%, or 99% identity thereto.

Exemplary N-terminal NLS-Cas9 domain:

(SEQ ID NO: 74)
MPAAKRVKLDGGDKKYSIGLDIGTNSVGWAVITDEYKVPSKKFKV
LGNTDRHSIKKNLIGALLFDSGETAEATRLKRTARRRYTRRKNRI
CYLQEIFSNEMAKVDDSFFHRLEESFLVEEDKKHERHPIFGNIVD
EVAYHEKYPTIYHLRKKLVDSTDKADLRLIYLALAHMIKFRGHFL
IEGDLNPDNSDVDKLFIQLVQTYNQLFEENPINASGVDAKAILSA
RLSKSRRLENLIAQLPGEKKNGLFGNLIALSLGLTPNFKSNFDLA
EDAKLQLSKDTYDDDLDNLLAQIGDQYADLFLAAKNLSDAILLSD
ILRVNTEITKAPLSASMIKRYDEHHQDLTLLKALVRQQLPEKYKE
IFFDQSKNGYAGYIDGGASQEEFYKFIKPILEKMDGTEELLVKLN
REDLLRKQRTFDNGSIPHQIHLGELHAILRRQEDFYPFLKDNREK
IEKILTFRIPYYVGPLARGNSRFAWMTRKSEETITPWNFEEVVDK
GASAQSFIERMTNFDKNLPNEKVLPKHSLLYEYFTVYNELTKVKY
VTEGMRKPAFLSGEQKKAIVDLLFKTNRKVTVKQLKEDYFKKIEC
FDSVEISGVEDRFNASLGTYHDLLKIIKDKDFLDNEENEDILEDI
VLTLTLFEDREMIEERLKTYAHLFDDKVMKQLKRRRYTGWGRLSR
KLINGIRDKQSGKTILDFLKSDGFANRNFMQLIHDDSLTFKEDIQ
KAQVSGQGDSLHEHIANLAGSPAIKKGILQTVKVVDELVKVMGRH
KPENIVIEMARENQTTQKGQKNSRERMKRIEEGIKELGSQILKEH
PVENTQLQNEKLYLYYLQNGRDMYVDQELDINRLSDYDVDHIVPQ
SFLKDDSIDNKVLTRSDKARGKSDNVPSEEVVKKMKNYWRQLLNA
KLITQRKFDNLTKAERGGLSELDKAGFIKRQLVETRQITKHVAQI
LDSRMNTKYDENDKLIREVKVITLKSKLVSDFRKDFQFYKVREIN
NYHHAHDAYLNAVVGTALIKKYPKLESEFVYGDYKVYDVRKMIAK
SEQEIGKATAKYFFYSNIMNFFKTEITLANGEIRKRPLIETNGET
GEIVWDKGRDFATVRKVLSMPQVNIVKKTEVQTGGFSKESILPKR
NSDKLIARKKDWDPKKYGGFDSPTVAYSVLVVAKVEKGKSKKLKS
VKELLGITIMERSSFEKNPIDFLEAKGYKEVKKDLIIKLPKYSLF
ELENGRKRMLASAGELQKGNELALPSKYVNFLYLASHYEKLKGSP
EDNEQKQLFVEQHKHYLDEIIEQISEFSKRVILADANLDKVLSAY
NKHRDKPIREQAENIIHLFTLTNLGAPAAFKYFDTTIDRKRYTST
KEVLDATLIHQSITGLYETRIDLSQLGGDGG

### Exemplary C-Terminal Sequence Comprising an NLS:

(SEQ ID NO: 75)
AGKRTADGSEFEKRTADGSEFESPKKKAKVE

In some embodiments, a heterologous gene modifying polypeptide described herein comprises an RT domain having an amino acid sequence according to Table 6 of International Application WO/2023/039440 (which Table is incorporated herein by reference in its entirety), or a sequence having at least 70%, 80%, 85%, 90%, 95%, 97%, 98%, or 99% identity thereto.

In some embodiments, a heterologous gene modifying polypeptide comprises: (i) a linker comprising a linker sequence as listed in a row of Table 8, or an amino acid sequence having at least 70%, 75%, 80%, 85%, 90%, 95%, or 99% identity thereto; and (ii) an RT domain comprising an RT domain sequence as listed in the same row of Table 8, or an amino acid sequence having at least 70%, 75%, 80%, 85%, 90%, 95%, or 99% identity thereto. In some embodiments, a heterologous gene modifying polypeptide comprises an amino acid sequence according to Table 9, or an amino acid sequence having at least 70%, 75%, 80%, 85%, 90%, 95%, or 99% identity thereto.

TABLE 8
Selection of exemplary gene modifying polypeptides

| SEQ ID NO: | | |
|---|---|---|
| for Full | | |
| Polypeptide | Linker | |
| Sequence | Sequence | RT name and sequence |
| 81 | AEAAAKEAAAK | AVIRE_P03360_3mutA |
| | EAAAKEAAAKA | TAPLEEEYRLFLEAPIQNVTLLEQWKREIPKVWAEINPPGLASTQAPIHVQ |
| | LEAEAAAKEAA | LLSTALPVRVRQYPITLEAKRSLRETIRKFRAAGILRPVHSPWNTPLLPVRKS |
| | AKEAAAKEAAA | GTSEYRMVQDLREVNKRVETIHPTVPNPYTLLSLLPPDRIWYSVLDLKDAF |
| | KA (SEQ ID | FCIPLAPESQLIFAFEWADAEEGESGQLTWTRLPQGFKNSPTLFNEALNR |
| | NO: 76) | DLQGFRLDHPSVSLLQYVDDLLIAADTQAACLSATRDLLMTLAELGYRVS |
| | | GKKAQLCQEEVTYLGFKIHKGSRSLSNSRTQAILQIPVPKTKRQVREFLGKI |
| | | GYCRLFIPGFAELAQPLYAATRPGNDPLVWGEKEEEAFQSLKLALTQPPAL |
| | | ALPSLDKPFQLFVEETSGAAKGVLTQALGPWKRPVAYLSKRLDPVAAGW |
| | | PRCLRAIAAAALLTREASKLTFGQDIEITSSHNLESLLRSPPDKWLTNARITQ |
| | | YQVLLLDPPRVRFKQTAALNPATLLPETDDTLPIHHCLDTLDSLTSTRPDLT |
| | | DQPLAQAEATLFTDGSSYIRDGKRYAGAAVVTLDSVIWAEPLPIGTSAQK |
| | | AELIALTKALEWSKDKSVNIYTDSRYAFATLHVHGMIYRERGWLTAGGKAI |
| | | KNAPEILALLTAVWLPKRVAVMHCKGHQKDDAPTSTGNRRADEVAREV |
| | | AIRPLSTQATIS (SEQ ID NO: 77) |
| 82 | AEAAAKEAAAK | FLV_P10273_3mutA |
| | EAAAKEAAAKA | TLQLEEEYRLFEPESTQKQEMDIWLKNFPQAWAETGGMGTAHCQAPVL |
| | LEAEAAAKEAA | IQLKATATPISIRQYPMPHEAYQGIKPHIRRMLDQGILKPCQSPWNTPLLP |
| | AKEAAAKEAAA | VKKPGTEDYRPVQDLREVNKRVEDIHPTVPNPYNLLSTLPPSHPWYTVLD |
| | KA | LKDAFFCLRLHSESQLLFAFEWRDPEIGLSGQLTWTRLPQGFKNSPTLFNE |
| | (SEQ ID NO: | ALHSDLADFRVRYPALVLLQYVDDLLLAAATRTECLEGTKALLETLGNKGY |
| | 76) | RASAKKAQICLQEVTYLGYSLKDGQRWLTKARKEAILSIPVPKNSRQVREF |
| | | LGKAGYCRLFIPGFAELAAPLYPLTRPGTLFQWGTEQQLAFEDIKKALLSSP |
| | | ALGLPDITKPFELFIDENSGFAKGVLVQKLGPWKRPVAYLSKKLDTVASG |
| | | WPPCLRMVAAIAILVKDAGKLTLGQPLTILTSHPVEALVRQPPNKWLSNA |
| | | RMTHYQAMLLDAERVHFGPTVSLNPATLLPLPSGGNHHDCLQILAETHG |
| | | TRPDLTDQPLPDADLTWYTDGSSFIRNGEREAGAAVTTESEVIWAAPLPP |
| | | GTSAQRAELIALTQALKMAEGKKLTVYTDSRYAFATTHVHGEIYRRRGWL |
| | | TSEGKEIKNKNEILALLEALFLPKRLSIIHCPGHQKGDSPQAKGNRLADDTA |
| | | KKAATETHSSLTVLP (SEQ ID NO: 78) |
| 83 | AEAAAKEAAAK | MLVMS_P03355_3mutA_WS |
| | EAAAKEAAAKA | TLNIEDEHRLHETSKEPDVSLGSTWLSDFPQAWAETGGMGLAVRQAPLII |
| | LEAEAAAKEAA | PLKATSTPVSIKQYPMSQEARLGIKPHIQRLLDQGILVPCQSPWNTPLLPV |
| | AKEAAAKEAAA | KKPGTNDYRPVQDLREVNKRVEDIHPTVPNPYNLLSGLPPSHQWYTVLD |
| | KA (SEQ ID | LKDAFFCLRLHPTSQPLFAFEWRDPEMGISGQLTWTRLPQGFKNSPTLFN |
| | NO: 76) | EALHRDLADFRIQHPDLILLQYVDDLLLAATSELDCQQGTRALLQTLGNLG |
| | | YRASAKKAQICQKQVKYLGYLLKEGQRWLTEARKETVMGQPTPKTPRQL |
| | | REFLGKAGFCRLFIPGFAEMAAPLYPLTKPGTLFNWGPDQQKAYQEIKQA |
| | | LLTAPALGLPDLTKPFELFVDEKQGYAKGVLTQKLGPWRRPVAYLSKKLDP |
| | | VAAGWPPCLRMVAAIAVLTKDAGKLTMGQPLVILAPHAVEALVKQPPD |
| | | RWLSNARMTHYQALLLDTDRVQFGPVVALNPATLLPLPEEGLQHNCLDI |
| | | LAEAHGTRPDLTDQPLPDADHTWYTDGSSLLQEGQRKAGAAVTTETEVI |
| | | WAKALPAGTSAQRAELIALTQALKMAEGKKLNVYTDSRYAFATAHIHGEI |
| | | YRRRGWLTSEGKEIKNKDEILALLKALFLPKRLSIIHCPGHQKGHSAEARGN |
| | | RMADQAARKAAITETPDTSTLL (SEQ ID NO: 79) |
| 84 | AEAAAKEAAAK | SFV3L_P27401_2mutA |
| | EAAAKEAAAKA | MDPLQLLQPLEAEIKGTKLKAHWNSGATITCVPQAFLEEEVPIKNIWIKTI |
| | LEAEAAAKEAA | HGEKEQPVYYLTFKIQGRKVEAEVISSPYDYILVSPSDIPWLMKKPLQLTTL |
| | AKEAAAKEAAA | VPLQEYEERLLKQTMLTGSYKEKLQSLFLKYDALWQHWENQVGHRRIKP |
| | KA (SEQ ID | HHIATGTVNPRPQKQYPINPKAKASIQTVINDLLKQGVLIQQNSIMNTPV |
| | NO: 76) | YPVPKPDGKWRMVLDYREVNKTIPLIAAQNQHSAGILSSIFRGKYKTTLDL |
| | | SNGFWAHSITPESYWLTAFTWLGQQYCWTRLPQGFLNSPALFNADVVD |
| | | LLKEVPNVQVYVDDIYISHDDPREHLEQLEKVFSLLLNAGYVVSLKKSEIAQ |
| | | HEVEFLGFNITKEGRGLTETFKQKLLNITPPRDLKQLQSILGKLNFARNFIPN |
| | | FSELVKPLYNIIATAPGKYITWTTDNSQQLQNIISMLNSAENLEERNPEVRL |
| | | IMKVNTSPSAGYIRFYNEFAKRPIMYLNYVYTKAEVKFTNTEKLLTTIHKGLI |
| | | KALDLGMGQEILVYSPIVSMTKIQKTPLPERKALPIRWITWMSYLEDPRIQ |
| | | FHYDKTLPELQQVPTVTDDIIAKIKHPSEFSMVFYTDGSAIKHPNVNKSHN |
| | | AGMGIAQVQFKPEFTVINTWSIPLGDHTAQLAEVAAVEFACKKALKIDGP |
| | | VLIVTDSFYVAESVNKELPYWQSNGFFNNKKKPLKHVSKWKSIADCIQLKP |
| | | DIIIIHEKGHQPTASTFHTEGNNLADKLATQGSYVVN (SEQ ID NO: 80) |

TABLE 9
Full length amino acid sequence corresponding to Table 8

| SEQ | |
|---|---|
| ID | |
| NO: | Amino acid sequence |
| 81 | MPAAKRVKLDGGDKKYSIGLDIGTNSVGWAVITDEYKVPSKKFKVLGNTDRHSIKKNLIGA |
| | LLEDSGETAEATRLKRTARRRYTRRKNRICYLQEIFSNEMAKVDDSFFHRLEESELVEEDK |
| | KHERHPIFGNIVDEVAYHEKYPTIYHLRKKLVDSTDKADLRLIYLALAHMIKERGHFLIEG |
| | DLNPDNSDVDKLFIQLVQTYNQLFEENPINASGVDAKAILSARLSKSRRLENLIAQLPGEK |
| | KNGLFGNLIALSLGLTPNFKSNFDLAEDAKLQLSKDTYDDDLDNLLAQIGDQYADLFLAAK |
| | NLSDAILLSDILRVNTEITKAPLSASMIKRYDEHHQDLTLLKALVRQQLPEKYKEIFFDQS |
| | KNGYAGYIDGGASQEEFYKFIKPILEKMDGTEELLVKLNREDLLRKQRTFDNGSIPHQIHL |
| | GELHAILRRQEDFYPFLKDNREKIEKILTFRIPYYVGPLARGNSRFAWMTRKSEETITPWN |
| | FEEVVDKGASAQSFIERMTNEDKNLPNEKVLPKHSLLYEYFTVYNELTKVKYVTEGMRKPA |
| | FLSGEQKKAIVDLLFKTNRKVTVKQLKEDYFKKIECFDSVEISGVEDRENASLGTYHDLLK |
| | IIKDKDELDNEENEDILEDIVLTLTLFEDREMIEERLKTYAHLEDDKVMKQLKRRRYTGWG |
| | RLSRKLINGIRDKQSGKTILDFLKSDGFANRNEMQLIHDDSLTFKEDIQKAQVSGQGDSLH |
| | EHIANLAGSPAIKKGILQTVKVVDELVKVMGRHKPENIVIEMARENQTTQKGQKNSRERMK |
| | RIEEGIKELGSQILKEHPVENTQLQNEKLYLYYLQNGRDMYVDQELDINRLSDYDVDHIVP |
| | QSFLKDDSIDNKVLTRSDKARGKSDNVPSEEVVKKMKNYWRQLLNAKLITQRKEDNLTKAE |
| | RGGLSELDKAGFIKRQLVETRQITKHVAQILDSRMNTKYDENDKLIREVKVITLKSKLVSD |
| | FRKDFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVAYSVLVVAK |
| | VEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGAPAAFKYEDTTI |
| | DRKRYTSTKEVLDATLIHQSITGLYETRIDLSQLGGDGGAEAAAKEAAAKEAAAKEAAAKA |
| | LEAEAAAKEAAAKEAAAKEAAAKAGGTAPLEEEYRLFLEAPIQNVTLLEQWKREIPKVWAE |
| | INPPGLASTQAPIHVQLLSTALPVRVRQYPITLEAKRSLRETIRKFRAAGILRPVHSPWNT |
| | PLLPVRKSGTSEYRMVQDLREVNKRVETIHPTVPNPYTLLSLLPPDRIWYSVLDLKDAFFC |
| | IPLAPESQLIFAFEWADAEEGESGQLTWTRLPQGFKNSPTLFNEALNRDLQGERLDHPSVS |
| | LLQYVDDLLIAADTQAACLSATRDLLMTLAELGYRVSGKKAQLCQEEVTYLGFKIHKGSRS |
| | LSNSRTQAILQIPVPKTKRQVREFLGKIGYCRLFIPGFAELAQPLYAATRPGNDPLVWGEK |
| | EEEAFQSLKLALTQPPALALPSLDKPFQLFVEETSGAAKGVLTQALGPWKRPVAYLSKRLD |
| | PVAAGWPRCLRAIAAAALLTREASKLTFGQDIEITSSHNLESLLRSPPDKWLTNARITQYQ |
| | VLLLDPPRVRFKQTAALNPATLLPETDDTLPIHHCLDTLDSLTSTRPDLTDQPLAQAEATL |
| | FTDGSSYIRDGKRYAGAAVVTLDSVIWAEPLPIGTSAQKAELIALTKALEWSKDKSVNIYT |
| | DSRYAFATLHVHGMIYRERGWLTAGGKAIKNAPEILALLTAVWLPKRVAVMHCKGHQKDDA |
| | PTSTGNRRADEVAREVAIRPLSTQATISAGKRTADGSEFEKRTADGSEFESPKKKAKVE |
| 82 | MPAAKRVKLDGGDKKYSIGLDIGTNSVGWAVITDEYKVPSKKFKVLGNTDRHSIKKNLIGA |
| | LLFDSGETAEATRLKRTARRRYTRRKNRICYLQEIFSNEMAKVDDSFFHRLEESELVEEDK |
| | KHERHPIFGNIVDEVAYHEKYPTIYHLRKKLVDSTDKADLRLIYLALAHMIKERGHFLIEG |
| | DLNPDNSDVDKLFIQLVQTYNQLFEENPINASGVDAKAILSARLSKSRRLENLIAQLPGEK |
| | KNGLFGNLIALSLGLTPNFKSNEDLAEDAKLQLSKDTYDDDLDNLLAQIGDQYADLELAAK |
| | NLSDAILLSDILRVNTEITKAPLSASMIKRYDEHHQDLTLLKALVRQQLPEKYKEIFFDQS |
| | KNGYAGYIDGGASQEEFYKFIKPILEKMDGTEELLVKLNREDLLRKORTEDNGSIPHQIHL |
| | GELHAILRRQEDFYPELKDNREKIEKILTFRIPYYVGPLARGNSRFAWMTRKSEETITPWN |
| | FEEVVDKGASAQSFIERMTNFDKNLPNEKVLPKHSLLYEYFTVYNELTKVKYVTEGMRKPA |
| | FLSGEQKKAIVDLLEKTNRKVTVKOLKEDYFKKIECFDSVEISGVEDRENASLGTYHDLLK |
| | IIKDKDFLDNEENEDILEDIVLTLTLFEDREMIEERLKTYAHLEDDKVMKQLKRRRYTGWG |
| | RLSRKLINGIRDKQSGKTILDELKSDGFANRNEMQLIHDDSLTFKEDIQKAQVSGQGDSLH |
| | EHIANLAGSPAIKKGILQTVKVVDELVKVMGRHKPENIVIEMARENQTTQKGQKNSRERMK |
| | RIEEGIKELGSQILKEHPVENTQLQNEKLYLYYLQNGRDMYVDQELDINRLSDYDVDHIVP |
| | QSFLKDDSIDNKVLTRSDKARGKSDNVPSEEVVKKMKNYWRQLLNAKLITQRKEDNLTKAE |
| | RGGLSELDKAGFIKRQLVETRQITKHVAQILDSRMNTKYDENDKLIREVKVITLKSKLVSD |
| | FRKDFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVAYSVLVVAK |
| | VEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKOLFVEQHKHYLDEII |
| | EQISEFSKRVILADANLDKVLSAYNKHRDKPIREQAENIIHLFTLTNLGAPAAFKYEDTTI |
| | DRKRYTSTKEVLDATLIHQSITGLYETRIDLSQLGGDGGAEAAAKEAAAKEAAAKEAAAKA |
| | LEAEAAAKEAAAKEAAAKEAAAKAGGTLQLEEEYRLFEPESTQKQEMDIWLKNFPQAWAET |
| | GGMGTAHCQAPVLIQLKATATPISIRQYPMPHEAYQGIKPHIRRMLDQGILKPCQSPWNTP |
| | LLPVKKPGTEDYRPVQDLREVNKRVEDIHPTVPNPYNLLSTLPPSHPWYTVLDLKDAFFCL |
| | RLHSESQLLFAFEWRDPEIGLSGQLTWTRLPQGEKNSPTLFNEALHSDLADERVRYPALVL |
| | LQYVDDLLLAAATRTECLEGTKALLETLGNKGYRASAKKAQICLQEVTYLGYSLKDGQRWL |
| | TKARKEAILSIPVPKNSRQVREFLGKAGYCRLFIPGFAELAAPLYPLTRPGTLFQWGTEQQ |
| | LAFEDIKKALLSSPALGLPDITKPFELFIDENSGFAKGVLVQKLGPWKRPVAYLSKKLDTV |
| | ASGWPPCLRMVAAIAILVKDAGKLTLGQPLTILTSHPVEALVRQPPNKWLSNARMTHYQAM |
| | LLDAERVHFGPTVSLNPATLLPLPSGGNHHDCLQILAETHGTRPDLTDQPLPDADLTWYTD |
| | GSSFIRNGEREAGAAVTTESEVIWAAPLPPGTSAQRAELIALTQALKMAEGKKLTVYTDSR |
| | YAFATTHVHGEIYRRRGWLTSEGKEIKNKNEILALLEALFLPKRLSIIHCPGHQKGDSPQA |
| | KGNRLADDTAKKAATETHSSLTVLPAGKRTADGSEFEKRTADGSEFESPKKKAKVE |
| 83 | MPAAKRVKLDGGDKKYSIGLDIGTNSVGWAVITDEYKVPSKKFKVLGNTDRHSIKKNLIGA |
| | LLFDSGETAEATRLKRTARRRYTRRKNRICYLQEIFSNEMAKVDDSFFHRLEESELVEEDK |
| | KHERHPIFGNIVDEVAYHEKYPTIYHLRKKLVDSTDKADLRLIYLALAHMIKERGHFLIEG |
| | DLNPDNSDVDKLFIQLVQTYNQLFEENPINASGVDAKAILSARLSKSRRLENLIAQLPGEK |
| | KNGLFGNLIALSLGLTPNFKSNFDLAEDAKLQLSKDTYDDDLDNLLAQIGDQYADLELAAK |
| | NLSDAILLSDILRVNTEITKAPLSASMIKRYDEHHQDLTLLKALVRQQLPEKYKEIFFDQS |
| | KNGYAGYIDGGASQEEFYKFIKPILEKMDGTEELLVKLNREDLLRKQRTFDNGSIPHQIHL |
| | GELHAILRRQEDFYPELKDNREKIEKILTFRIPYYVGPLARGNSRFAWMTRKSEETITPWN |
| | FEEVVDKGASAQSFIERMTNFDKNLPNEKVLPKHSLLYEYFTVYNELTKVKYVTEGMRKPA |
| | FLSGEQKKAIVDLLFKTNRKVTVKQLKEDYFKKIECFDSVEISGVEDRENASLGTYHDLLK |
| | IIKDKDFLDNEENEDILEDIVLTLTLFEDREMIEERLKTYAHLEDDKVMKQLKRRRYTGWG |
| | RLSRKLINGIRDKQSGKTILDELKSDGFANRNEMQLIHDDSLTFKEDIQKAQVSGQGDSLH |
| | EHIANLAGSPAIKKGILQTVKVVDELVKVMGRHKPENIVIEMARENQTTQKGQKNSRERMK |
| | RIEEGIKELGSQILKEHPVENTQLQNEKLYLYYLQNGRDMYVDQELDINRLSDYDVDHIVP |
| | QSFLKDDSIDNKVLTRSDKARGKSDNVPSEEVVKKMKNYWRQLLNAKLITQRKEDNLTKAE |
| | RGGLSELDKAGFIKRQLVETRQITKHVAQILDSRMNTKYDENDKLIREVKVITLKSKLVSD |
| | FRKDFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGESKESILPKRNSDKLIARKKDWDPKKYGGFDSPTVAYSVLVVAK |
| | VEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGAPAAFKYEDTTI |
| | DRKRYTSTKEVLDATLIHQSITGLYETRIDLSQLGGDGGAEAAAKEAAAKEAAAKEAAAKA |
| | LEAEAAAKEAAAKEAAAKEAAAKAGGTLNIEDEHRLHETSKEPDVSLGSTWLSDEPQAWAE |
| | TGGMGLAVRQAPLIIPLKATSTPVSIKQYPMSQEARLGIKPHIQRLLDQGILVPCQSPWNT |
| | PLLPVKKPGTNDYRPVQDLREVNKRVEDIHPTVPNPYNLLSGLPPSHQWYTVLDLKDAFFC |
| | LRLHPTSQPLFAFEWRDPEMGISGQLTWTRLPQGFKNSPTLFNEALHRDLADFRIQHPDLI |
| | LLQYVDDLLLAATSELDCQQGTRALLQTLGNLGYRASAKKAQICQKQVKYLGYLLKEGQRW |
| | LTEARKETVMGQPTPKTPRQLREFLGKAGFCRLFIPGFAEMAAPLYPLTKPGTLENWGPDQ |
| | QKAYQEIKQALLTAPALGLPDLTKPFELFVDEKQGYAKGVLTQKLGPWRRPVAYLSKKLDP |
| | VAAGWPPCLRMVAAIAVLTKDAGKLTMGQPLVILAPHAVEALVKQPPDRWLSNARMTHYQA |
| | LLLDTDRVQFGPVVALNPATLLPLPEEGLQHNCLDILAEAHGTRPDLTDQPLPDADHTWYT |
| | DGSSLLQEGQRKAGAAVTTETEVIWAKALPAGTSAQRAELIALTQALKMAEGKKLNVYTDS |
| | RYAFATAHIHGEIYRRRGWLTSEGKEIKNKDEILALLKALFLPKRLSIIHCPGHQKGHSAE |
| | ARGNRMADQAARKAAITETPDTSTLLAGKRTADGSEFEKRTADGSEFESPKKKAKVE |
| 84 | MPAAKRVKLDGGDKKYSIGLDIGTNSVGWAVITDEYKVPSKKFKVLGNTDRHSIKKNLIGA |
| | LLFDSGETAEATRLKRTARRRYTRRKNRICYLQEIFSNEMAKVDDSFFHRLEESELVEEDK |
| | KHERHPIFGNIVDEVAYHEKYPTIYHLRKKLVDSTDKADLRLIYLALAHMIKERGHFLIEG |
| | DLNPDNSDVDKLFIQLVQTYNQLFEENPINASGVDAKAILSARLSKSRRLENLIAQLPGEK |
| | KNGLFGNLIALSLGLTPNEKSNEDLAEDAKLQLSKDTYDDDLDNLLAQIGDQYADLELAAK |
| | NLSDAILLSDILRVNTEITKAPLSASMIKRYDEHHQDLTLLKALVRQQLPEKYKEIFFDQS |
| | KNGYAGYIDGGASQEEFYKFIKPILEKMDGTEELLVKLNREDLLRKQRTEDNGSIPHQIHL |
| | GELHAILRRQEDFYPELKDNREKIEKILTFRIPYYVGPLARGNSRFAWMTRKSEETITPWN |
| | FEEVVDKGASAQSFIERMTNEDKNLPNEKVLPKHSLLYEYFTVYNELTKVKYVTEGMRKPA |
| | FLSGEQKKAIVDLLFKTNRKVTVKQLKEDYFKKIECFDSVEISGVEDRENASLGTYHDLLK |
| | IIKDKDELDNEENEDILEDIVLTLTLFEDREMIEERLKTYAHLEDDKVMKQLKRRRYTGWG |
| | RLSRKLINGIRDKQSGKTILDFLKSDGFANRNEMQLIHDDSLTFKEDIQKAQVSGQGDSLH |
| | EHIANLAGSPAIKKGILQTVKVVDELVKVMGRHKPENIVIEMARENQTTQKGQKNSRERMK |
| | RIEEGIKELGSQILKEHPVENTQLQNEKLYLYYLONGRDMYVDQELDINRLSDYDVDHIVP |
| | QSFLKDDSIDNKVLTRSDKARGKSDNVPSEEVVKKMKNYWRQLLNAKLITQRKEDNLTKAE |
| | RGGLSELDKAGFIKRQLVETRQITKHVAQILDSRMNTKYDENDKLIREVKVITLKSKLVSD |
| | FRKDFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGEDSPTVAYSVLVVAK |
| | VEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKOLFVEQHKHYLDEII |
| | EQISEFSKRVILADANLDKVLSAYNKHRDKPIREQAENIIHLFTLTNLGAPAAFKYEDTTI |
| | DRKRYTSTKEVLDATLIHQSITGLYETRIDLSQLGGDGGAEAAAKEAAAKEAAAKEAAAKA |
| | LEAEAAAKEAAAKEAAAKEAAAKAGGMDPLQLLQPLEAEIKGTKLKAHWNSGATITCVPQA |
| | FLEEEVPIKNIWIKTIHGEKEQPVYYLTFKIQGRKVEAEVISSPYDYILVSPSDIPWLMKK |
| | PLQLTTLVPLQEYEERLLKQTMLTGSYKEKLQSLFLKYDALWQHWENQVGHRRIKPHHIAT |
| | GTVNPRPQKQYPINPKAKASIQTVINDLLKQGVLIQONSIMNTPVYPVPKPDGKWRMVLDY |
| | REVNKTIPLIAAQNQHSAGILSSIFRGKYKTTLDLSNGEWAHSITPESYWLTAFTWLGQQY |
| | CWTRLPQGELNSPALFNADVVDLLKEVPNVQVYVDDIYISHDDPREHLEQLEKVESLLLNA |
| | GYVVSLKKSEIAQHEVEFLGENITKEGRGLTETFKQKLLNITPPRDLKQLQSILGKLNFAR |
| | NFIPNESELVKPLYNIIATAPGKYITWTTDNSQQLQNIISMLNSAENLEERNPEVRLIMKV |
| | NTSPSAGYIRFYNEFAKRPIMYLNYVYTKAEVKFTNTEKLLTTIHKGLIKALDLGMGQEIL |
| | VYSPIVSMTKIQKTPLPERKALPIRWITWMSYLEDPRIQFHYDKTLPELQQVPTVTDDIIA |
| | KIKHPSEFSMVFYTDGSAIKHPNVNKSHNAGMGIAQVQFKPEFTVINTWSIPLGDHTAQLA |
| | EVAAVEFACKKALKIDGPVLIVTDSFYVAESVNKELPYWQSNGFENNKKKPLKHVSKWKSI |
| | ADCIQLKPDIIIIHEKGHQPTASTFHTEGNNLADKLATQGSYVVNAGKRTADGSEFEKRTA |
| | DGSEFESPKKKAKVE |

In some embodiments, the heterologous gene modifying polypeptide has a sequence disclosed in International Application WO/2023/039424 (which is incorporated by reference herein in its entirety), or a sequence having at least 70%, 80%, 85%, 90%, 95%, 97%, 98%, or 99% identity thereto. For instance, in some embodiments, the heterologous gene modifying polypeptide has an RT domain as described in Table 6 of International Application WO/2023/039424 (which table is incorporated by reference herein in its entirety), or a sequence having at least 70%, 80%, 85%, 90%, 95%, 97%, 98%, or 99% identity thereto. In some embodiments, a heterologous gene modifying polypeptide may comprise a linker, e.g., a peptide linker, e.g., a linker as described in Table 10 of International Application WO/2023/039424 (which Table is incorporated herein by reference in its entirety). In some embodiments, the heterologous gene modifying polypeptide has a sequence according to Table T2 of International Application WO/2023/039424 (which table is incorporated by reference herein in its entirety), or a sequence having at least 70%, 80%, 85%, 90%, 95%, 97%, 98%, or 99% identity thereto. In some embodiments, the heterologous gene modifying polypeptide has a sequence according to Table A1 of International Application WO/2023/039424 (which table is incorporated by reference herein in its entirety), or a sequence having at least 70%, 80%, 85%, 90%, 95%, 97%, 98%, or 99% identity thereto.

In some embodiments, a gene modifying polypeptide comprises the RT domain from a retroviral reverse transcriptase, e.g., an M-MLVRT, e.g., comprising the following sequence:

(SEQ ID NO: 85)
TLNIEDEHRLHETSKEPDVSLGSTWLSDFPQAWAETGGMGLAVRQAPLI
IPLKATSTPVSIKQYPMSQEARLGIKPHIQRLLDQGILVPCQSPWNTPLL
PVKKPGTNDYRPVQDLREVNKRVEDIHPTVPNPYNLLSGLPPSHQWYTV
LDLKDAFFCLRLHPTSQPLFAFEWRDPEMGISGQLTWTRLPQGFKNSPTL
FDEALHRDLADFRIQHPDLILLQYVDDLLLAATSELDCQQGTRALLQTLG
NLGYRASAKKAQICQKQVKYLGYLLKEGQRWLTEARKETVMGQPTPKTPR
QLREFLGTAGFCRLWIPGFAEMAAPLYPLTKTGTLFNWGPDQQKAYQEIK
QALLTAPALGLPDLTKPFELFVDEKQGYAKGVLTQKLGPWRRPVAYLSKK
LDPVAAGWPPCLRMVAAIAVLTKDAGKLTMGQPLVILAPHAVEALVKQPP
DRWLSNARMTHYQALLLDTDRVQFGPVVALNPATLLPLPEEGLQHNCLDI
LAEAHGTRPDLTDQPLPDADHTWYTDGSSLLQEGQRKAGAAVTTETEVIW
AKALPAGTSAQRAELIALTQALKMAEGKKLNVYTDSRYAFATAHIHGEIY
RRRGLLTSEGKEIKNKDEILALLKALFLPKRLSIIHCPGHQKGHSAEARG
NRMADQAARKAAITETPDTSTLL,

ID NO: 85), or a sequence having at least 70%, 80%, 85%, 90%, 95%, 97%, 98%, or 99% identity thereto.

In some embodiments, an M-MLV RT domain comprises, relative to the M-MLV (WT) sequence above, one or more mutations, e.g., selected from D200N, L603W, T330P, T306K, W313F, D524G, E562Q, D583N, P51L, S67R, E67K, T197A, H204R, E302K, F309N, L435G, N454K, H594Q, D653N, R110S, K103L, e.g., a combination of mutations, such as D200N, L603W, and T330P, optionally further including T306K and W313F. In some embodiments, an M-MLV RT used herein comprises the mutations D200N, L603W, T330P, T306K and W313F. In embodiments, the mutant M-MLV RT comprises the following amino acid sequence:

(SEQ ID NO: 86)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVL
DLKDAFFCLRLHPTSQPLFAFEWRDPEMGISGQLTWTRLPQGFKNSPTLF
NEALHRDLADFRIQHPDLILLQYVDDLLLAATSELDCQQGTRALLQTLGN
LGYRASAKKAQICQKQVKYLGYLLKEGQRWLTEARKETVMGQPTPKTPRQ
LREFLGKAGFCRLFIPGFAEMAAPLYPLTKPGTLFNWGPDQQKAYQEIKQ
ALLTAPALGLPDLTKPFELFVDEKQGYAKGVLTQKLGPWRRPVAYLSKKL
DPVAAGWPPCLRMVAAIAVLTKDAGKLTMGQPLVILAPHAVEALVKQPPD
RWLSNARMTHYQALLLDTDRVQFGPVVALNPATLLPLPEEGLQHNCLDIL
AEAHGTRPDLTDQPLPDADHTWYTDGSSLLQEGQRKAGAAVTTETEVIWA
KALPAGTSAQRAELIALTQALKMAEGKKLNVYTDSRYAFATAHIHGEIYR
RRGWLTSEGKEIKNKDEILALLKALFLPKRLSIIHCPGHQKGHSAEARGN
RMADQAARKAAITETPDTSTLLI

### Exemplary Gene Modifying System Comprises Mutant M-MLV RT Region:

(SEQ ID NO: 87)
MPAAKRVKLDGGDKKYSIGLDIGTNSVGWAVITDEYKVPSKKFKVLGNTD
RHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASA
QSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGI
KELGSQILKEHPVENTQLQNEKLYLYYLQNGRDMYVDQELDINRLSDYDV
DHIVPQSFLKDDSIDNKVLTRSDKARGKSDNVPSEEVVKKMKNYWRQLLN
AKLITQRKFDNLTKAERGGLSELDKAGFIKRQLVETRQITKHVAQILDSR
MNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYL
ASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTS
TKEVLDATLIHQSITGL YETRIDLSQLGGDGGSGGSSGGSSGSETPGTS
ESATPESSGGSSGGSSGGTLNIEDEYRLHETSKEPDVSLGSTWLSDFPQA
WAETGGMGLAVRQAPLIIPLKATSTPVSIKQYPMSQEARLGIKPHIQRLL
DQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFNEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGKAGFCRLFIPGFAEMAAPLYPLTKPG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLOHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGWLTSEGKEIKNKDEILALLKALFLPKRLS
IIHCPGHQKGHSAEARGNRMADQAARKAAITETPDTSTLLIENSSPSGGS
KRTADGSEFEAGKRTADGSEFEKRTADGSEFESPKKKAKVE

In some embodiments, the heterologous gene modifying polypeptide comprises an amino acid sequence according to SEQ ID NO: 4002, or a sequence having at least 70%, 80%, 85%, 90%, 95%, 97%, 98%, or 99% identity thereto.

In some embodiments, a template RNA molecule for use in the system comprises, from 5′ to 3′ (1) a gRNA spacer; (2) a gRNA scaffold; (3) heterologous object sequence (4) a primer binding site (PBS) sequence. In some embodiments:
(1) Is a gRNA spacer of ˜18-22 nt, e.g., is 20 nt
(2) Is a gRNA scaffold comprising one or more hairpin loops, e.g., 1, 2, of 3 loops for associating the template with a Cas domain, e.g., a nickase Cas9 domain. In some embodiments, the gRNA scaffold comprises the sequence, from 5′ to 3′,

(SEQ ID NO: 88)
GTTTTAGAGCTAGAAATAGCAAGTTAAAATAAGGCTAGTCCGTTATCAA
CTTGAAAAAGTGGGACCGAGTCGGTCC.

(3) In some embodiments, the heterologous object sequence is, e.g., 7-74, e.g., 10-20, 20-30, 30-40, 40-50, 50-60, 60-70, or 70-80 nt or, 80-90 nt in length.
(4) In some embodiments, the PBS sequence that binds the target priming sequence after nicking occurs is e.g., 3-20 nt, e.g., 7-15 nt, e.g., 12-14 nt. In some embodiments, the PBS sequence has 40-60% GC content.

### V. Lipid Nanoparticles

The disclosure provides lipid nanoparticles (LNPs) that are conjugated to targeting moieties in a site-specific manner through specific enzyme-recognized linkers, as disclosed herein. Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); or combinations of the foregoing. The conjugation methods described herein can be used to site-specifically conjugate a targeting moiety (or a plurality of targeting moieties) to the surfaces of LNPs comprising various formulations and specific lipid compositions.

Lipids that can be used in nanoparticle formations (e.g., lipid nanoparticles) include, for example those described in Table 4 of US20210371858, which is incorporated by reference—e.g., a lipid-containing nanoparticle can comprise one or more of the lipids in Table 4 of US20210371858. Lipid nanoparticles can include additional elements, such as polymers, such as the polymers described in Table 5 of US20210371858, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-l-0-(w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of US20210059953 (incorporated by reference), and combinations of the foregoing.

In some embodiments, sterols that can be incorporated into lipid nanoparticles include one or more of cholesterol or cholesterol derivatives, such as those in U.S. Pat. No. 11,141,378 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the conjugates described herein, include a lipid of any one of formulas (i)-(ix).

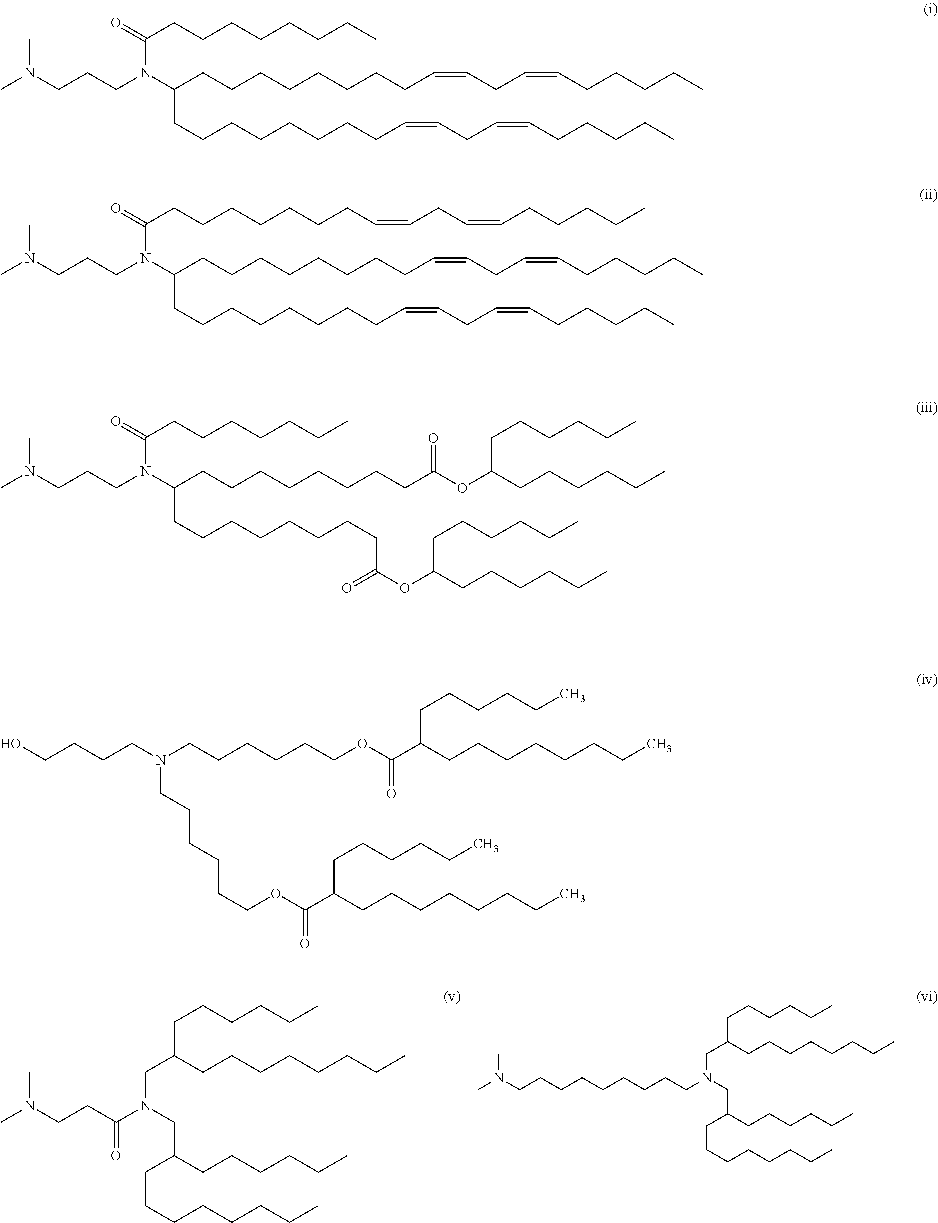

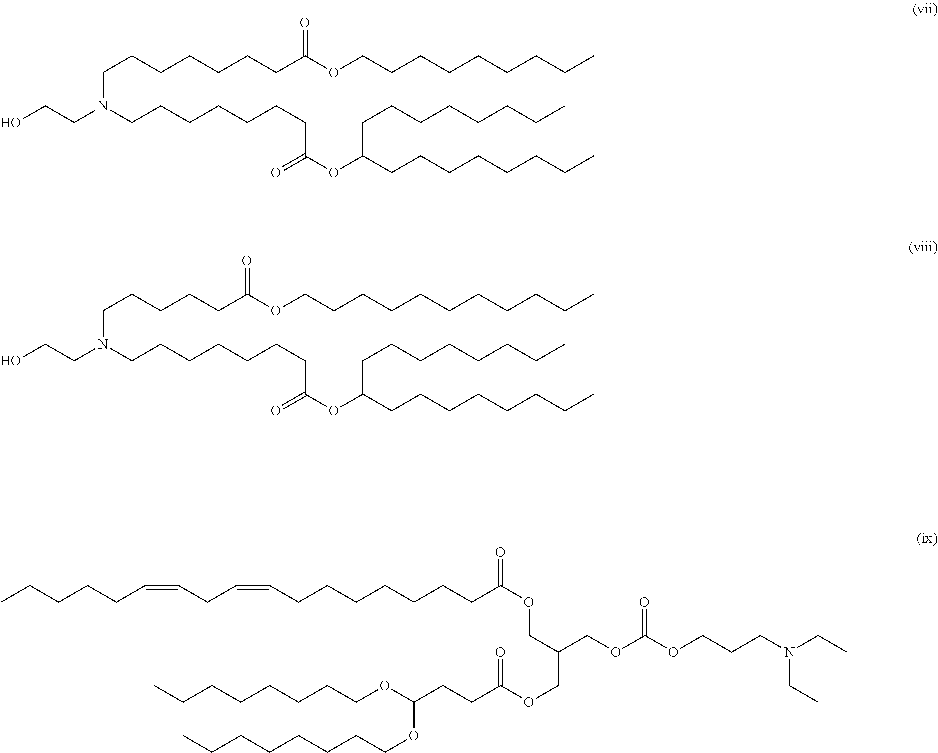

Another non-limiting example of lipid compounds that may be used (e.g., in combination with other lipid components) to form lipid nanoparticles for the conjugates described herein, include a lipid of formula (x):

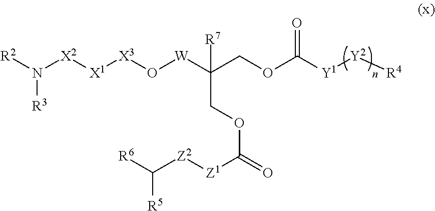

wherein
X¹ is O, NR¹, or a direct bond, X² is C₂₋₅ alkylene, X³ is C(═O) or a direct bond, R¹ is H or Me, R³ is C₁₋₃ alkyl, R² is C₁₋₃ alkyl, or R² is taken together with the nitrogen atom to which it is attached and 1-3 carbon atoms of X² to form a 4-, 5-, or 6-membered ring, or X¹ is NR¹, R¹ and R² are taken together with the nitrogen atoms to which they are attached form a 5- or 6-membered ring, or R² is taken together with R³ and the nitrogen atom to which they are attached form a 5-, 6-, or 7-membered ring, Y¹ is C2-12 alkylene, Y² is selected from

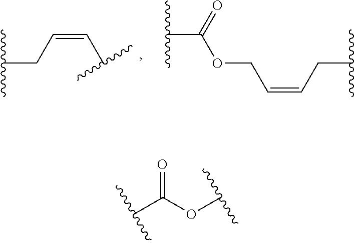 [SMILES: */C=C\C* ; *C/C=C\COC(*)=O ; *OC(*)=O]

(in either orientation), (in either orientation), (in either orientation),
n is 0 to 3, R⁴ is Ci-15 alkyl, Z¹ is Ci-6 alkylene or a direct bond,
Z² is

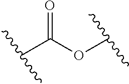 [SMILES: *OC(*)=O]

(in either orientation) or absent, provided that if Z1 is a direct bond, Z2 is absent;
R⁵ is C5-9 alkyl or C6-10 alkoxy, R⁶ is C₅₋₉ alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

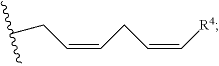

R⁴ is linear C5 alkyl, Z1 is C2 alkylene, Z² is absent, W is methylene, and R⁷ is H, then R⁵ and R⁶ are not Cx alkoxy.

Another non-limiting example of lipid compounds that may be used (e.g., in combination with other lipid components) to form lipid nanoparticles for the conjugates described herein, include a lipid of formula (xi):

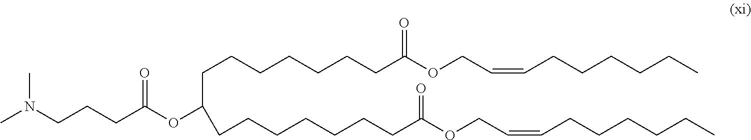 [SMILES: CCCCCC/C=C\COC(=O)CCCCCCCC(CCCCCCCC(=O)OC/C=C\CCCCCC)OC(=O)CCCN(C)C]

Another non-limiting example of lipid compounds that may be used (e.g., in combination with other lipid components) to form lipid nanoparticles for the conjugates described herein, include a lipid of any one of the formulas (xii)-(xiv):

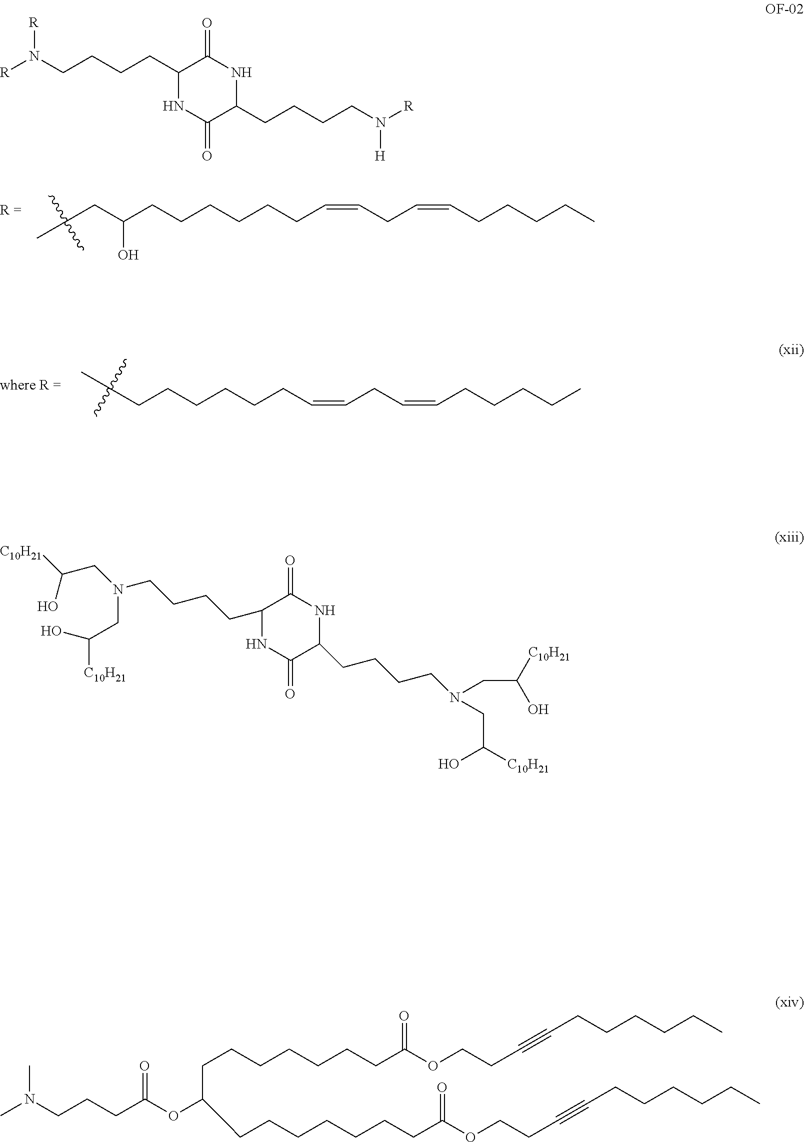

Another non-limiting example of lipid compounds that may be used (e.g., in combination with other lipid components) to form lipid nanoparticles for the conjugates described herein, include a lipid of formula (xv):

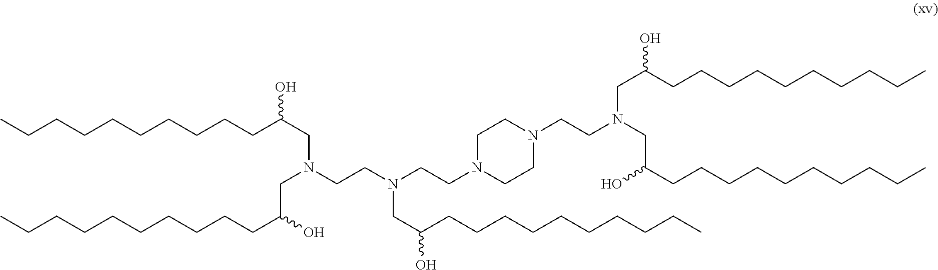 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

Another non-limiting example of lipid compounds that may be used (e.g., in combination with other lipid components) to form lipid nanoparticles for the conjugates described herein, include a lipid of formula (xvi):

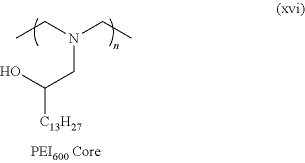

Another non-limiting example of lipid compounds that may be used (e.g., in combination with other lipid components) to form lipid nanoparticles for the conjugates described herein, include a lipid of any one of the formulas (xvii)-(xix):

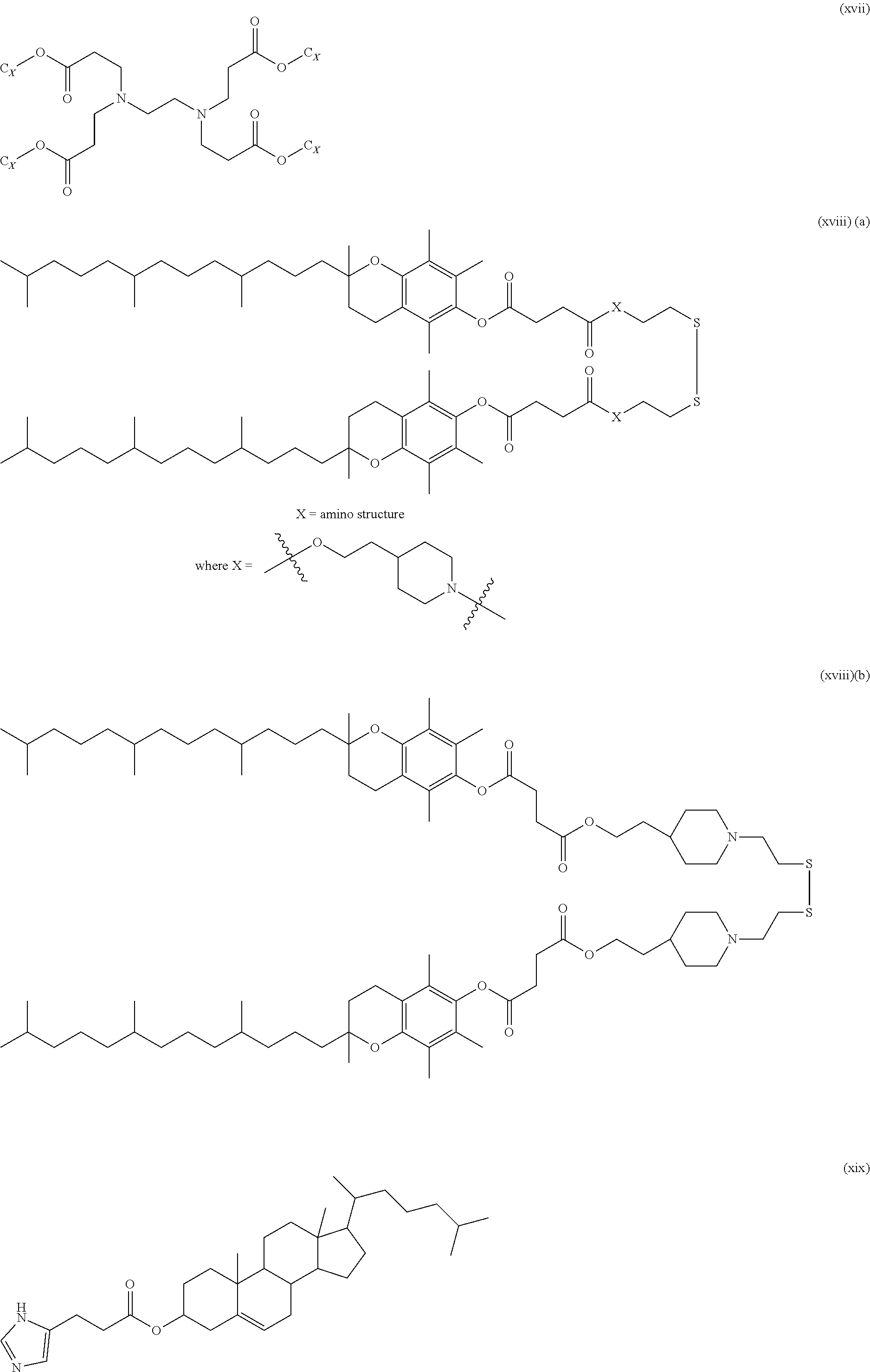

Another non-limiting example of lipid compounds that may be used (e.g., in combination with other lipid components) to form lipid nanoparticles for the conjugates described herein, include a lipid of any one of the formulas (xx)(a) or (xx)(b):

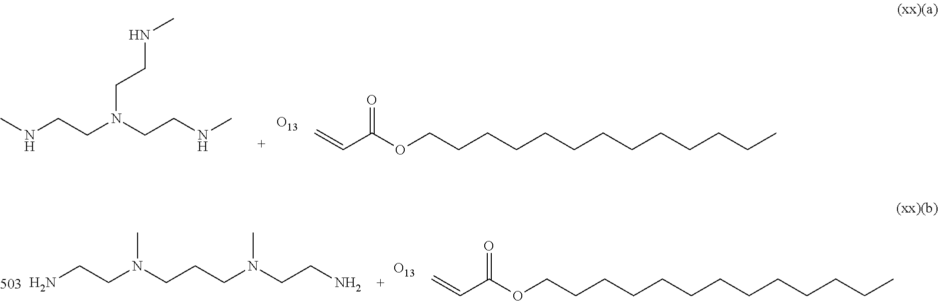

In some embodiments, a conjugate described herein comprises an LNP that comprises an ionizable lipid. In some embodiments, the ionizable lipid is heptadecan-9-yl 8-((2-hydroxyethyl)(6-oxo-6-(undecyloxy)hexyl)amino) octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of U.S. Pat. No. 11,420,933 (incorporated by reference herein in its entirety). In some embodiments, ionizable the lipid is Di((Z)-non-2-en-1-yl) 9-((4-dimethylamino)butanoyl)oxy) heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of U.S. Pat. No. 8,450,298 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl) propanoate, e.g., Structure (I) from US 2022/0324926 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 30 and 60 mol percent of a cationic lipid, a neutral lipid, a steroid, a polymer conjugated lipid, and a therapeutic agent, e.g., a nucleic acid (e.g., RNA) described herein (e.g., a template nucleic acid or a nucleic acid encoding a desired polypeptide), encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., template RNA and/or a mRNA encoding a desired polypeptide.

Exemplary ionizable lipids that can be used in the disclosed conjugates include, without limitation, those listed in Table 1 of US20210059953, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; Iincorpo-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,31Z)-heptatriaconta-6,9,28,31-tetraen-19-yl-4-(dimethylamino)butanoate (DLin-MC3-DMA or MC3), e.g., as described in Example 9 of US2021/0059953 (incorporated by reference herein in its entirety). In some embodiments, the ionizable lipid is the lipid ATX-002, e.g., as described in Example 10 of US2021/0059953 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of US2021/0059953 (incorporated by reference herein in its entirety). In some embodiments, the ionizable lipid is Compound 6 or Compound 22, e.g., as described in Example 12 of US2021/0059953 (incorporated by reference herein in its entirety).

Specific ionizable lipids are shown in Table 10.

TABLE 10
Ionizable Lipids

| | | Molec- | |
|---|---|---|---|
| | | ular | |
| LIPID ID | Chemical Name | Weight | Structure |
| LIPIDV003 | (9Z,12Z)-3-((4,4- bis(octyloxy)butanoyl)oxy)- 2-((((3-(diethyl- amino)propoxy)carbo- nyl)oxy)methyl)propyl octadeca-9,12-dienoate | 852.29 | 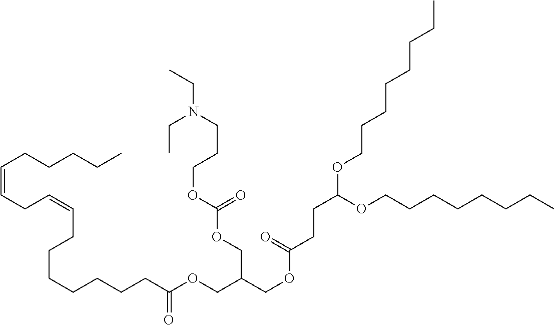 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| LIPIDV004 | Heptadecan-9-yl 8-((2- hydroxyethyl)(8- (nonyloxy)-8- oxooctyl)amino)octanoate | 710.18 | 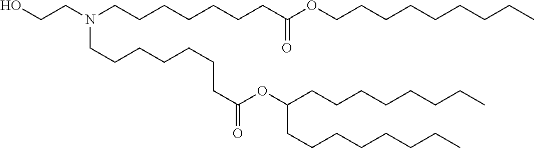 [SMILES: CCCCCCCCCOC(=O)CCCCCCCN(CCO)CCCCCCCC(=O)OC(CCCCCCCC)CCCCCCCC] |
| LIPIDV005 | | 919.56 | 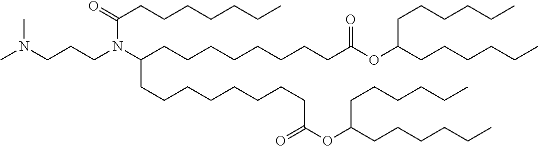 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which are incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., dcholesterol-(4′-hydroxy)-butyl ether. Exemplary cholesterol derivatives are described in PCT publication WO2009/127060 and US patent publication US2010/0130588, which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-l-0-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy (polyethylene glycol)-2000]. In some embodiments, the PEG-lipid comprises PEG-DMG, 1,2-dimyristoyl-sn-glycero-3-phosphoethanolamine-N-[methoxy (polyethylene glycol)-2000]. In some embodiments, the PEG-lipid comprises a structure selected from:

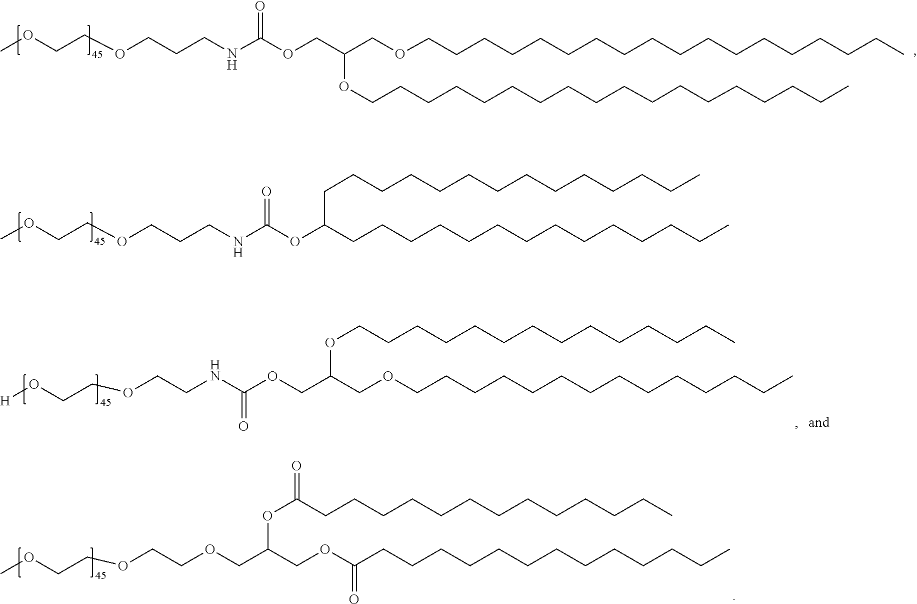

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of US2021/0059953, the contents of all of which are incorporated herein by reference in its entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a pegylated lipid in a molar ratio of 50:10:38.5:1.5. In some other embodiments, the lipid particle formulation comprises ionizable lipid, cholesterol and a pegylated lipid in a molar ratio of 60:38.5:1.5.

In some embodiments, the lipid particle comprises ionizable lipid, non-cationic lipid (e.g. phospholipid), a sterol (e.g., cholesterol) and a pegylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of pegylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, LNPs are directed to specific cell types or tissues by the addition of targeting domains (other than the targeting moieties of the disclosed conjugates). For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl) propyl octadeca-9,12-dienoate, also called 3-((4,4-bis(octyloxy) butanoyl)oxy)-2-((((3-(diethylamino) propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10 nm and 150 nm, e.g., measured by dynamic light scattering (DLS). In some embodiments, the average LNP diameter of the LNP formulation may be between 10 nm and 100 nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 70 nm to about 150 nm, from about 80 nm to about 120 nm, from about 80 nm to about 110 nm, from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid (e.g., DNA or RNA, such as mRNA) encoding the polypeptide, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

In some embodiments, a lipid nanoparticle (or a formulation comprising lipid nanoparticles) lacks reactive impurities (e.g., aldehydes or ketones), or comprises less than a preselected level of reactive impurities (e.g., aldehydes or ketones). While not wishing to be bound by theory, in some embodiments, a lipid reagent is used to make a lipid nanoparticle formulation, and the lipid reagent may comprise a contaminating reactive impurity (e.g., an aldehyde or ketone). A lipid regent may be selected for manufacturing based on having less than a preselected level of reactive impurities (e.g., aldehydes or ketones). Without wishing to be bound by theory, in some embodiments, aldehydes can cause modification and damage of RNA, e.g., cross-linking between bases and/or covalently conjugating lipid to RNA (e.g., forming lipid-RNA adducts). This may, in some instances, lead to failure of a reverse transcriptase reaction and/or incorporation of inappropriate bases, e.g., at the site(s) of lesion(s), e.g., a mutation in a newly synthesized target DNA.

In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, a lipid nanoparticle formulation is produced using a lipid reagent comprising: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation is produced using a plurality of lipid reagents, and each lipid reagent of the plurality independently meets one or more criterion described in this paragraph. In some embodiments, each lipid reagent of the plurality meets the same criterion, e.g., a criterion of this paragraph.

In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation comprises: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, one or more, or optionally all, of the lipid reagents used for a lipid nanoparticle as described herein or a formulation thereof comprise: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, total aldehyde content and/or quantity of any single reactive impurity (e.g., aldehyde) species is determined by liquid chromatography (LC), e.g., coupled with tandem mass spectrometry (MS/MS), e.g., as described herein. In some embodiments, reactive impurity (e.g., aldehyde) content and/or quantity of reactive impurity (e.g., aldehyde) species is determined by detecting one or more chemical modifications of a nucleic acid molecule (e.g., an RNA molecule, e.g., as described herein) associated with the presence of reactive impurities (e.g., aldehydes), e.g., in the lipid reagents. In some embodiments, reactive impurity (e.g., aldehyde) content and/or quantity of reactive impurity (e.g., aldehyde) species is determined by detecting one or more chemical modifications of a nucleotide or nucleoside (e.g., a ribonucleotide or ribonucleoside, e.g., comprised in or isolated from a template nucleic acid, e.g., as described herein) associated with the presence of reactive impurities (e.g., aldehydes), e.g., in the lipid reagents, e.g., as described herein. In embodiments, chemical modifications of a nucleic acid molecule, nucleotide, or nucleoside are detected by determining the presence of one or more modified nucleotides or nucleosides, e.g., using LC-MS/MS analysis, e.g., as described herein.

In some embodiments, the lipid nanoparticle are liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

It has been surprisingly discovered that increased amounts of the non-pegylated phospholipid (e.g., DSPC) in the conjugates disclosed herein improves the delivery of payloads (e.g., mRNA) to cells of interest (e.g., T cells or HSCs). In some embodiments, the ratio between the ionizable lipid and the non-pegylated phospholipid (e.g., DSPC) is from about 1:1 to about 7:1. In some embodiments, the ratio between the ionizable lipid and the non-pegylated phospholipid (e.g., DSPC) is from about 1:1 to about 4:1. In some embodiments, the ratio between the ionizable lipid and the non-pegylated phospholipid (e.g., DSPC) is from about 1:1 to about 3:1. In some embodiments, the ratio between the ionizable lipid and the non-pegylated phospholipid (e.g., DSPC) is from about 1:1 to about 2.5:1. In some embodiments, the ratio between the ionizable lipid and the non-pegylated phospholipid (e.g., DSPC) is from about 1:1 to about 2:1. In some embodiments, the ratio between the ionizable lipid and the non-pegylated phospholipid (e.g., DSPC) is from about 1.5:1 to about 2.5:1. In some embodiments, the ratio between the ionizable lipid and the non-pegylated phospholipid (e.g., DSPC) is from about 2:1 to about 2.5:1.

Additionally, increased ratios non-pegylated phospholipid (e.g., DSPC) to the cholesterol molecule in the conjugates disclosed herein can result in increased delivery of payloads (e.g., mRNA) to cells of interest (e.g., T cells or HSCs). In some embodiments, the ratio between the cholesterol molecule and the non-pegylated phospholipid (e.g., DSPC) is from about 6:1 to about 0.5:1. In some embodiments, the ratio between the cholesterol molecule and the non-pegylated phospholipid (e.g., DSPC) is from about 3:1 to about 0.5:1. In some embodiments, the ratio between the cholesterol molecule and the non-pegylated phospholipid (e.g., DSPC) is from about 2:1 to about 0.5:1. In some embodiments, the ratio between the cholesterol molecule and the non-pegylated phospholipid (e.g., DSPC) is from about 1.5:1 to about 0.5:1. In some embodiments, the ratio between the cholesterol molecule and the non-pegylated phospholipid (e.g., DSPC) is from about 1:1 to about 0.5:1. In some embodiments, the ratio between the cholesterol molecule and the non-pegylated phospholipid (e.g., DSPC) is from about 1:2 to about 0.8:1.

### EXAMPLES

The following examples further illustrate the invention but should not be construed as in any way limiting its scope. In light of the present disclosure and the general level of skill in the art, those of skill will appreciate that the following Examples are intended to be exemplary only and that numerous changes, modifications, and alterations can be employed without departing from the scope of the presently disclosed subject matter. The attached figures are meant to be considered as integral parts of the specification and description of the disclosure.

### Example 1: Conjugate Synthesis

The methods disclosed herein are suitable to conjugate an antibody or an antigen binding fragment thereof, such as a Fab fragment, scFv or a VHH (nanobody), to the surface of a lipid nanoparticle (LNP) to generate a targeted LNP (tLNP). The antibody or antigen binding fragment thereof is engineered to include a sequence at its C terminal end, such as a hinge region sequence, wherein the sequence is engineered to include a single free cysteine at or near its C terminus. The antibody or antigen binding fragment is subsequently exposed to a reduction reaction to remove the disulfide linkage, hence generating the free cysteine. The free cysteine group is than coupled with a reaction partner such as a maleimide, as set forth herein. For instance, conjugating the reduced cysteine-terminated antibody or antigen binding fragment thereof to the surface of the LNP can be accomplished using a two-step process, as described herein.

### Engineered Fab Comprising a Single Free C-Terminal Cysteine

An anti-CD117 Fab sequence was engineered to include a hinge region sequence comprising the sequence DKTHTCAA (SEQ ID NO: 99) at the C-terminal end of the heavy chain. This sequence comprises a portion of the human IgG1 hinge region with a cysteine near the C-terminus of the sequence followed by two additional alanine residues. Table 11 provides the heavy and light chain sequences of the engineered Fab fragment with engineered hinge region sequence (DKTHTCAA) (SEQ ID NO: 99). The engineered Fab sequence was cloned into a pcDNA3.1 mammalian expression plasmid, expressed in ExpiCHO cells, and then purified with CH1XL resin.

TABLE 11
An engineered anti-CD117 Fab fragment
comprising a free C-terminal cysteine

| Fab | | |
|---|---|---|
| Construct | HC | LC |
| Anti- | EVQLVESGGGLVQPG | DIQMTQSPSSLSASV |
| CD117 | GSLRLSCAASGFTFS | GDRVTITCRASQSIS |
| Fab with a | DADMDWVRQAPGKGL | SYLNWYQQKPGKAPK |
| C-term | EWVGRTRNKAGSYTT | LLIYAASSLQSGVPS |
| cysteine | EYAASVKGRFTISRD | RFSGSGSGTDFTLTI |
| (Wt) | DSKNSLYLQMNSLKT | SSLQPEDFATYYCQQ |
| | EDTAVYYCAREPKYW | SYIAPYTFGGGTKVE |
| | IDFDLWGRGTLVTVS | IKRTVAAPSVFIFPP |
| | SASTKGPSVFPLAPS | SDEQLKSGTASVVCL |
| | SKSTSGGTAALGCLV | LNNFYPREAKVQWKV |
| | KDYFPEPVTVSWNSG | DNALQSGNSQESVTE |
| | ALTSGVHTFPAVLQS | QDSKDSTYSLSSTLT |
| | SGLYSLSSVVTVPSS | LSKADYEKHKVYACE |
| | SLGTQTYICNVNHKP | VTHQGLSSPVTKSFN |
| | SNTKVDKKVEPKSCD | RGEC |
| | KTHTCAA | (SEQ ID NO: 109) |
| | (SEQ ID NO: 108) | |

The purified Fab fragment was reduced before conjugation to an LNP. For the reduction of the disulfide bond, the Fab solution was buffer exchanged with PBS containing 10 mM EDTA by a desalting column. 10 equivalents of Tris (2-carboxyethyl) phosphine (TCEP) was added to the solution and the reduction was carried out for 1 hour at room temperature. After the reaction, TCEP was removed using a desalting column. Complete reduction was confirmed by SDS PAGE gels.

### A Two-Step Process for Conjugating Fab Fragment Comprising a Reduced Free Cysteine to an LNP

Step 1: Site-Specific Modification of a Cysteine Terminated Fab with a Click Ligand

Following reduction of the disulfide bond, as described above, the engineered Fab (Table 11) was reacted with a bifunctional linker containing maleimide and the click ligand, methyltetrazine (MeTz). Specifically, three equivalents of Mal-PEG-MeTz was added to the solution of the freshly reduced anti-CD117 Fab, hence generating Fab-MeTz used in step 2 of the process, described below. The reaction was carried out at room temperature for 2 hours. After the reaction, the excess maleimide linker was removed using a zeba spin desalting column (7 KDa cut off).

### Step 2: Conjugation Between the Fab Fragment and a Base LNP

LNP-TCO was formulated similarly as described herein. Briefly, an ethanol phase was prepared with five lipids, containing ionizable lipid, DSPC, cholesterol, PEG-DMG and DSPE-PEG2K-TCO at a molar ratio of 47%:8%:42.5%:2%:0.5%, respectively. An aqueous phase was composed of GFP mRNA dissolved in 25 mM acetate buffer. The two phases were mixed using a microfluidic device to formulate LNP-TCO at a total flow rate of 18 ml/min (aqueous phase: ethanol phase=3:1). 3× volume of CBS was added to LNP solution and the resulting solution was neutralized by 1 vol % of 1M Tris-HCl, pH 8 after 15 minutes.

The Fab-MeTz made in the step 1 was added to the solution of LNP-TCO to produce an anti-CD117 Fab-LNP conjugate. The solution was incubated at room temperature for 2 hours and then at 4° C. overnight. The targeted LNP was concentrated and any unbound Fab was removed by a centrifugal filter (100K MWCO). The solution was exchanged with Tris buffer containing 9% sucrose during the process. The resulting tLNP was characterized by Zetasizer for size and PDI and by Ribogreen assay for mRNA concentration.

Delivery of GFP mRNA to Primary CD34+ Cells with Targeted LNPs (tLNPs) Manufactured Using the Two-Step Processes

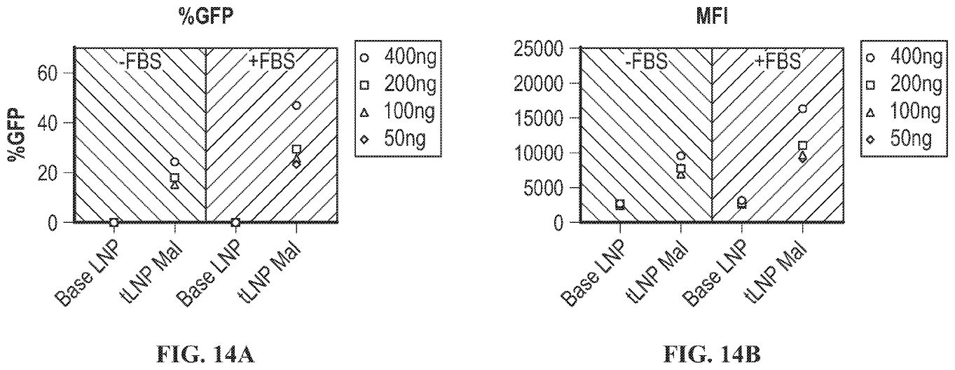

Anti-CD117 targeted LNPs generated using the two-step process described above were administered to primary CD34+ cells to determine whether they could effectively deliver an mRNA payload to the cells. Primary CD34+ cells were also transfected with a base LNP that was not conjugated to an anti-CD117 Fab as a baseline control for GFP mRNA delivery. FIGS. 14A and 14B show that administration of the targeted LNPs resulted in significantly higher numbers of cells expressing GFP and higher GFP expression levels (MFI), respectively, relative to cells transfected with the base LNP in culture conditions both with and without serum.

### Example 2: Engineering Anti-CD117 Fab Fragments with Alternative Disulfide Bonds to Enhance Stability

An anti-CD117 Fab was engineered to have a hinge region sequence with one free cysteine suitable for reduction and then conjugation to an LNP, as described in Example 1. This Fab was further engineered to create a series of mutants (m1, m2, m5, m6, m9, m10; see Table 12) wherein the natural inter-chain disulfide bond (between the heavy and light chains, see FIG. 7B) was removed and a new interchain disulfide bond was added elsewhere. Generally, each mutant was engineered such that a new disulfide bond was added further away from the C-terminus, such that it was buried in the interface between the light chain and heavy chain to attempt to increase stability of the Fab during the mild reduction conditions imposed during the two-step conjugation process. To create each mutant, the wild-type Fab was modeled using the human IgG1 crystal structure (1HZH) as a template. The amino acid positions at the interface of the heavy chain and the light chain having a distance within 10 Angstroms (A) was determined. Cysteine mutations were introduced at these sites within the heavy and light chains with the aim of creating a new disulfide bond (bolded cysteine residues in the heavy and light chain sequences in Table 12). The nascent cysteine residues in the light and heavy chains of human IgG1 that create the natural disulfide bond were mutated to serine residues (also bolded in the heavy and light chain sequences of Table 12), thereby disrupting the bond.

The wild-type (wt) and mutant Fabs were buffer exchanged into reaction buffer (PBS, pH 6.8) and incubated with 10% or 50% TCEP agarose in 2:1 ratio (vol/vol) for 2 hours at room temperature followed by overnight incubation at 4° C. Samples were aliquoted for 1 hour, 2 hour and overnight (O/N) incubations for SDS-PAGE analysis.

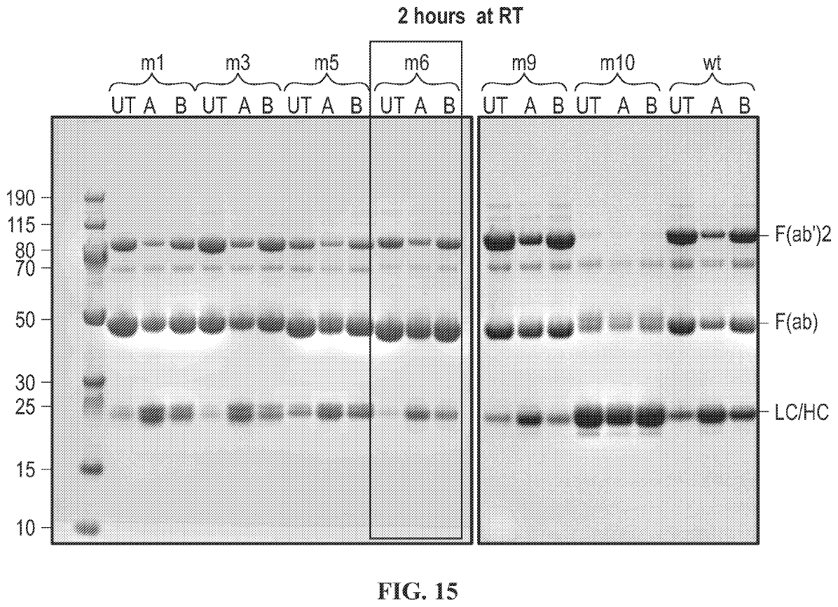

As shown in FIG. 15, mutant m6 produced the highest yield of Fab fragment after incubation with TCEP-agarose for 2 hours at room temperature and at 4° C. overnight. Mutant m5 also produced higher yield levels of Fab fragment after incubation with TCEP agarose. The m6 mutant was subsequently labeled with 2 equivalents of Alexa-488 maleimide in PBS, pH 6.8 and mass spectrometry results showed site-specific modification with a dye-to-antibody ratio (DAR) of 1.

Modification of Mutant m6 with a Maleimide-PEG-Methyltetrazine Linker:

Anti-CD117 Fab mutant m6 was incubated with 50% TCEP-agaorse in a 2:1 ratio (vol/vol) for 2 hours at room temperature followed by an overnight incubation at 4° C. TCEP-agarose was removed by spin column and the reduced Fab was incubated with 2 equivalents of maleimide-PEG-methyltetrazine crosslinker at 25° C. for 2 hours in the reaction buffer. Subsequently, linker-modified Fab was purified using a desalting column (7 KDa cut off) into PBS, pH 7.4. The DOL of the modified Fab was estimated to be 0.73 using a TCO-594 reaction. A LC-MS characterization of the linker-modified anti-CD117 Fab Cys m6 mutant showed that the maleimide=PEG-methyltetrazine crosslinker was attached to the hinge cysteine in a site-specific manner (data not shown).

TABLE 12
Anti-CD117 Fab + Free C-terminal Cysteine Mutants

| Fab Construct | HC | LC |
|---|---|---|
| Anti-CD117 | EVQLVESGGGLVQPGGSLRLS | DIQMTQSPSSLSASVGDRVTITCRA |
| Fab + C-term | CAASGFTFSDADMDWVRQAP | SQSISSYLNWYQQKPGKAPKLLIYA |
| Cysteine (Wt) | GKGLEWVGRTRNKAGSYTTE | ASSLQSGVPSRFSGSGSGTDFTLTIS |
| | YAASVKGRFTISRDDSKNSLY | SLQPEDFATYYCQQSYIAPYTFGGG |
| | LQMNSLKTEDTAVYYCAREP | TKVEIKRTVAAPSVFIFPPSDEQLKS |
| | KYWIDFDLWGRGTLVTVSSAS | GTASVVCLLNNFYPREAKVQWKV |
| | TKGPSVFPLAPSSKSTSGGTAA | DNALQSGNSQESVTEQDSKDSTYS |
| | LGCLVKDYFPEPVTVSWNSGA | LSSTLTLSKADYEKHKVYACEVTH |
| | LTSGVHTFPAVLQSSGLYSLSS | QGLSSPVTKSFNRGEC (SEQ ID NO: |
| | VVTVPSSSLGTQTYICNVNHK | 109) |
| | PSNTKVDKKVEPKSCDKTHTC | |
| | AA (SEQ ID NO: 108) | |
| Anti-CD117 | EVQLVESGGGLVQPGGSLRLS | DIQMTQSPSSLSASVGDRVTITCRA |
| Fab + C-term | CAASGFTFSDADMDWVRQAP | SQSISSYLNWYQQKPGKAPKLLIYA |
| Cysteine (m1) | GKGLEWVGRTRNKAGSYTTE | ASSLQSGVPSRFSGSGSGTDFTLTIS |
| | YAASVKGRFTISRDDSKNSLY | SLQPEDFATYYCQQSYIAPYTFGGG |
| | LQMNSLKTEDTAVYYCAREP | TKVEIKRTVAAPSVFIFPPCDEQLKS |
| | KYWIDFDLWGRGTLVTVSSAS | GTASVVCLLNNFYPREAKVQWKV |
| | TKGPSVCPLAPSSKSTSGGTA | DNALQSGNSQESVTEQDSKDSTYS |
| | ALGCLVKDYFPEPVTVSWNSG | LSSTLTLSKADYEKHKVYACEVTH |
| | ALTSGVHTFPAVLQSSGLYSLS | QGLSSPVTKSFNRGES (SEQ ID NO: |
| | SVVTVPSSSLGTQTYICNVNH | 111) |
| | KPSNTKVDKKVEPKSSDKTHT | |
| | CAA (SEQ ID NO: 110) | |
| Anti-CD117 | EVQLVESGGGLVQPGGSLRLS | DIQMTQSPSSLSASVGDRVTITCRA |
| Fab + C-term | CAASGFTFSDADMDWVRQAP | SQSISSYLNWYQQKPGKAPKLLIYA |
| Cysteine (m3) | GKGLEWVGRTRNKAGSYTTE | ASSLQSGVPSRFSGSGSGTDFTLTIS |
| | YAASVKGRFTISRDDSKNSLY | SLQPEDFATYYCQQSYIAPYTFGGG |
| | LQMNSLKTEDTAVYYCAREP | TKVEIKRTVAAPSVFICPPSDEQLKS |
| | KYWIDFDLWGRGTLVTVSSAS | GTASVVCLLNNFYPREAKVQWKV |
| | TKGPSVFPCAPSSKSTSGGTAA | DNALQSGNSQESVTEQDSKDSTYS |
| | LGCLVKDYFPEPVTVSWNSGA | LSSTLTLSKADYEKHKVYACEVTH |
| | LTSGVHTFPAVLQSSGLYSLSS | QGLSSPVTKSFNRGES (SEQ ID NO: |
| | VVTVPSSSLGTQTYICNVNHK | 113) |
| | PSNTKVDKKVEPKSSDKTHTC | |
| | AA (SEQ ID NO: 112) | |
| Anti-CD117 | EVQLVESGGGLVQPGGSLRLS | DIQMTQSPSSLSASVGDRVTITCRA |
| Fab + C-term | CAASGFTFSDADMDWVRQAP | SQSISSYLNWYQQKPGKAPKLLIYA |
| Cysteine (m5) | GKGLEWVGRTRNKAGSYTTE | ASSLQSGVPSRFSGSGSGTDFTLTIS |
| | YAASVKGRFTISRDDSKNSLY | SLQPEDFATYYCQQSYIAPYTFGGG |
| | LQMNSLKTEDTAVYYCAREP | TKVEIKRTVAAPSVCIFPPSDEQLKS |
| | KYWIDFDLWGRGTLVTVSSAS | GTASVVCLLNNFYPREAKVQWKV |
| | TKGPSVFPLAPSSKSTSGGTAC | DNALQSGNSQESVTEQDSKDSTYS |
| | LGCLVKDYFPEPVTVSWNSGA | LSSTLTLSKADYEKHKVYACEVTH |
| | LTSGVHTFPAVLQSSGLYSLSS | QGLSSPVTKSFNRGES (SEQ ID NO: |
| | VVTVPSSSLGTQTYICNVNHK | 115) |
| | PSNTKVDKKVEPKSSDKTHTC | |
| | AA (SEQ ID NO: 114) | |
| Anti-CD117 | EVQLVESGGGLVQPGGSLRLS | DIQMTQSPSSLSASVGDRVTITCRA |
| Fab + C-term | CAASGFTFSDADMDWVRQAP | SQSISSYLNWYQQKPGKAPKLLIYA |
| Cysteine (m6) | GKGLEWVGRTRNKAGSYTTE | ASSLQSGVPSRFSGSGSGTDFTLTIS |
| | YAASVKGRFTISRDDSKNSLY | SLQPEDFATYYCQQSYIAPYTFGGG |
| | LQMNSLKTEDTAVYYCAREP | TKVEIKRTVAAPSVFIFPPSDEQLKS |
| | KYWIDFDLWGRGTLVTVSSAS | GTASVVCLLNNFYPREAKVQWKV |
| | TKGPSVFPLAPSSKSTSGGTAA | DNALQSGNSQESVTEQDSKDSTYC |
| | LGCLVKDYFPEPVTVSWNSGA | LSSTLTLSKADYEKHKVYACEVTH |
| | LTSGVCTFPAVLQSSGLYSLSS | QGLSSPVTKSFNRGES (SEQ ID NO: |
| | VVTVPSSSLGTQTYICNVNHK | 117) |
| | PSNTKVDKKVEPKSSDKTHTC | |
| | AA (SEQ ID NO: 116) | |
| Anti-CD117 | EVQLVESGGGLVQPGGSLRLS | DIQMTQSPSSLSASVGDRVTITCRA |
| Fab + C-term | CAASGFTFSDADMDWVRQAP | SQSISSYLNWYQQKPGKAPKLLIYA |
| Cysteine (m9) | GKGLEWVGRTRNKAGSYTTE | ASSLQSGVPSRFSGSGSGTDFTLTIS |
| | YAASVKGRFTISRDDSKNSLY | SLQPEDFATYYCQQSYIAPYTFGGG |
| | LQMNSLKTEDTAVYYCAREP | TKVEIKRTVAAPSVFIFPPSDEQLKS |
| | KYWIDFDLWGRGTLVTVSSAS | GTASVVCLLNNFYPREAKVQWKV |
| | TKGPSVFPLAPSSKSTSGGTAA | DNALQSGNSCESVTEQDSKDSTYS |
| | LGCLVKDYFPEPVTVSWNSGA | LSSTLTLSKADYEKHKVYACEVTH |
| | LTSGVHTFPACLQSSGLYSLSS | QGLSSPVTKSFNRGES (SEQ ID NO: |
| | VVTVPSSSLGTQTYICNVNHK | 119) |
| | PSNTKVDKKVEPKSSDKTHTC | |
| | AA (SEQ ID NO: 118) | |
| Anti-CD117 | EVQLVESGGGLVQPGGSLRLS | DIQMTQSPSSLSASVGDRVTITCRA |
| Fab + C-term | CAASGFTFSDADMDWVRQAP | SQSISSYLNWYQQKPGKAPKLLIYA |
| Cysteine (m10) | GKGLEWVGRTRNKAGSYTTE | ASSLQSGVPSRFSGSGSGTDFTLTIS |
| | YAASVKGRFTISRDDSKNSLY | SLQPEDFATYYCQQSYIAPYTFGGG |
| | LQMNSLKTEDTAVYYCAREP | TKVEIKRTVAAPSVFIFPPSDEQLKS |
| | KYWIDFDLWGRGTLVTVSSAS | GTASVVCLLNNFYPREAKVQWKV |
| | TKGPSVFPLAPSSKSTSGGTAA | DNALQSGNSQESVTEQDSKDSTYS |
| | LGCLVKDYFPEPVTVSWNSGA | LCSTLTLSKADYEKHKVYACEVTH |
| | LTSGVHTFPAVLQSSGLYSLCS | QGLSSPVTKSFNRGES (SEQ ID NO: |
| | VVTVPSSSLGTQTYICNVNHK | 121) |
| | PSNTKVDKKVEPKSSDKTHTC | |
| | AA (SEQ ID NO: 120) | |

1. A conjugate comprising an antibody or functional fragment thereof and a lipid nanoparticle (LNP) encapsulating a therapeutic agent, wherein the antibody or functional fragment thereof is conjugated to the LNP through a linker comprising a thiol moiety and a click product formed via a click reaction between a first click handle and a second click handle.

2. The conjugate of claim 1, wherein the thiol moiety is covalently bonded directly to the antibody or functional fragment, and wherein the second click handle is covalently bonded directly to the LNP.

3. The conjugate of claim 1 or claim 2, wherein the linker further comprises a spacer between the thiol moiety and the click product.

4. The conjugate of claim 3, wherein the spacer comprises a polyethylene glycol (PEG) and wherein the PEG comprises n ethylene glycol units, wherein n is between 4 and 200.

5. The conjugate of any one of claims 1-4, wherein the antibody or functional fragment thereof is conjugated to the LNP through a free cysteine residue located within a hinge region sequence of the antibody or functional fragment thereof.

6. The conjugate of claim 5, wherein the hinge region sequence of the antibody or functional fragment thereof comprises the sequence DKTHTC (SEQ ID NO: 97).

7. The conjugate of claim 5, wherein the hinge region sequence of the antibody or functional fragment thereof comprises the sequence DKTHTCA (SEQ ID NO: 98).

8. The conjugate of claim 5, wherein the hinge region sequence of the antibody or functional fragment thereof comprises the sequence DKTHTCAA (SEQ ID NO: 99).

9. The conjugate of claim 5, wherein the hinge region sequence of the antibody or functional fragment thereof comprises the sequence selected from the group consisting of EPKSCDKTHTCPPCP (SEQ ID NO: 100), EPKCCVECPPCP (SEQ ID NO: 101), ELKTPLGDTTHTCPRCP(EPKSCDTPPPCPRCP)₃ (SEQ ID NO: 102), ESKYGPPCPSCP (SEQ ID NO: 103), VPRDCGCKPCICT (SEQ ID NO: 104), EPRGPTIKPCPPCK (SEQ ID NO: 105), EPSGPISTINPCPPCK (SEQ ID NO: 106), and EPRIPKPSTPPGSSCP (SEQ ID NO: 107).

10. The conjugate of any one of claims 1-9, wherein the thiol moiety is synthesized from the group consisting of a thiol-maleimide reaction, a thiol-parafluoro reaction, a thiol-ene reaction, a thiol-yne reaction, a thiol-vinylsulfone reaction, a thiol-pyridyl disulfide reaction, a thiol-thiosulfonate reaction, and a thiol-bisulfone reaction.

11. The conjugate of any one of claims 1-9, wherein the thiol moiety is a thioether moiety.

12. The conjugate of claim 11, wherein the thioether moiety is a thiosuccinimide moiety.

13. The conjugate of claim 12, wherein the thiosuccinimide moiety is substituted with one or more substituents selected from the group consisting of C₁₋₃ alkyl, halo, and C₁₋₃Oalkyl.

14. The conjugate of claim 12 or claim 13, wherein the thiosuccinimide moiety is formed from a reaction between a free cysteine located within the hinge region sequence of the targeting moiety, e.g., antibody, Fab fragment or ScFv, and a maleimide moiety.

15. The conjugate of any one of claims 1-14, wherein the click product is formed through a copper-catalyzed click chemistry reaction.

16. The conjugate of any one of claims 1-14, wherein the click product is formed via a Huisgen 1,3-dipolar cycloaddition between an azide and an alkyne.

17. The conjugate of any one of claims 1-14, wherein the click product is formed through a copper free click chemistry reaction.

18. The conjugate of claim 17, wherein the copper-free click chemistry reaction is selected from the group consisting of (a) a strain-promoted cycloaddition between an azide and a cyclic alkyne; (b) a Staudinger ligation between an azide and a phosphine; (c) an inverse electron demand Diels-Alder reaction between a trans-cyclooctene (TCO) and a tetrazine; (d) an inverse electron demand Diels-Alder reaction between a tetrazine and a norbonene; (e) a photoinducible 1,3-dipolar cycloaddition reaction between a tetrazole and an alkene; (f) an oxime ligation between an aldehyde or ketone and an α effect amine; and (g) a hydrazone ligation between an aldehyde or ketone and an α effect amine.

19. The conjugate of claim 18, wherein the click product is formed via an inverse electron demand Diels-Alder reaction between a TCO moiety and a tetrazine moiety.

20. The conjugate of claim 19, wherein the TCO moiety is covalently bonded directly to the LNP.

21. The conjugate of claim 19 or claim 20, wherein the tetrazine is unsubstituted.

22. The conjugate of claim 19 or claim 20, wherein the tetrazine is methyltetrazine.

23. The conjugate of any one of claims 1-22, wherein the antibody functional fragment is a Fab fragment.

24. A conjugate comprising an antibody or functional fragment thereof and a lipid nanoparticle (LNP) encapsulating a therapeutic agent, wherein the antibody or functional fragment thereof is conjugated to the LNP through a linker comprising a thiol moiety and a second moiety selected from the group consisting of a triazole moiety, dihydropyridazine moiety, aza-ylide moiety, hydrazone moiety and an oxime moiety.

25. The conjugate of claim 24, wherein the thiol moiety is covalently bonded directly to the antibody or functional fragment.

26. The conjugate of claim 24 or claim 25, wherein the linker further comprises a spacer between the thiol moiety and the click product.

27. The conjugate of claim 26, wherein the spacer comprises a polyethylene glycol (PEG) and wherein the PEG comprises n ethylene glycol units, wherein n is between 4 and 200.

28. The conjugate of any one of claims 24-27, wherein the antibody or functional fragment thereof is conjugated to the LNP through a free cysteine residue located within a hinge region sequence of the antibody or functional fragment thereof.

29. The conjugate of claim 28, wherein the hinge region sequence of the antibody or functional fragment thereof comprises the sequence DKTHTC (SEQ ID NO: 97).

30. The conjugate of claim 28, wherein the hinge region sequence of the antibody or functional fragment thereof comprises the sequence DKTHTCA. (SEQ ID NO: 98).

31. The conjugate of claim 28, wherein the hinge region sequence of the antibody or functional fragment thereof comprises the sequence DKTHTCAA (SEQ ID NO: 99).

32. The conjugate of claim 28, wherein the hinge region sequence of the antibody or functional fragment thereof comprises the sequence selected from the group consisting of EPKSCDKTHTCPPCP (SEQ ID NO: 100), EPKCCVECPPCP (SEQ ID NO: 101), ELKTPLGDTTHTCPRCP(EPKSCDTPPPCPRCP)₃ (SEQ ID NO: 102), ESKYGPPCPSCP (SEQ ID NO: 103), VPRDCGCKPCICT (SEQ ID NO: 104), EPRGPTIKPCPPCK (SEQ ID NO: 105), EPSGPISTINPCPPCK (SEQ ID NO: 106), and EPRIPKPSTPPGSSCP (SEQ ID NO: 107).

33. The conjugate of any one of claims 24-32, wherein the thiol moiety is synthesized from a thiol-maleimide reaction, a thiol-parafluoro reaction, a thiol-ene reaction, a thiol-yne reaction, a thiol-vinylsulfone reaction, a thiol-pyridyl disulfide reaction, a thiol-thiosulfonate reaction, and a thiol-bisulfone reaction.

34. The conjugate of any one of claims 24-32, wherein the thiol moiety is a thioether moiety.

35. The conjugate of claim 34, wherein the thioether moiety is a thiosuccinimide moiety.

36. The conjugate of claim 35, wherein the thiosuccinimide moiety is substituted with one or more substituents selected from the group consisting of C₁₋₃ alkyl, halo, and C₁₋₃Oalkyl.

37. The conjugate of claim 35 or claim 36, wherein the thiosuccinimide moiety is formed from a reaction between a free cysteine located within the hinge region sequence of the targeting moiety, e.g., antibody, Fab fragment or ScFv, and a maleimide moiety.

38. The conjugate of any one of claims 24-37, wherein the antibody functional fragment is a Fab fragment.

39. The conjugate of any one of claims 1-38, wherein the LNP comprises one or more lipid molecules.

40. The conjugate of claims 1-23, wherein the second click handle is covalently bonded to at least one of the lipid molecules.

41. The conjugate of claim 40, wherein the lipid molecule bonded to the second click handle is a pegylated lipid.

42. The conjugate of any one of claims 1-41, wherein the LNP comprises at least one ionizable lipid molecule.

43. The conjugate of claim 42, wherein the ionizable lipid is selected from V003, V004, V005, and V040.

44. The conjugate of any one of claims 1-43, wherein the antibody or functional fragment thereof binds to a T cell.

45. The conjugate of claim 44, wherein the antibody or functional fragment thereof binds to CD2, CD3, CD4, CD5, CD6, CD7 or CD8.

46. The conjugate of any one of claims 1-43, wherein the antibody or functional fragment thereof binds to hematopoietic stem cells (HSCs).

47. The conjugate of claim 46, wherein the antibody or functional fragment thereof binds to CD90 or CD117.

48. The conjugate of any one of claims 1-47, wherein the number of antibodies or functional fragments thereof conjugated to each LNP is greater than 10.

49. The conjugate of any one of claims 1-47, wherein the number of antibodies or functional fragments thereof conjugated to each LNP is greater than 20.

50. A conjugate of any one of claims 1-49, wherein the therapeutic agent is a nucleic acid molecule.

51. The conjugate of claim 50, wherein the nucleic acid molecule is a DNA molecule, e.g., a DNA plasmid, closed-ended DNA (ceDNA), or a small circular DNA.

52. The conjugate of claim 50, wherein the nucleic acid molecule is a DNA molecule, e.g., a DNA plasmid, closed-ended DNA (ceDNA), or a small circular DNA.

53. The conjugate of claim 50, wherein the nucleic acid molecule is an mRNA molecule.

54. The conjugate of claim 53, wherein the mRNA encodes a chimeric antigen receptor (CAR) or a protein with enzymatic activity.

55. The conjugate of claim 53, wherein the mRNA encodes an enzyme.

56. The conjugate of claim 55, wherein enzyme comprises a nuclease, recombinase, integrase, transposase, retrotransposase, helicase, transcriptase, polymerase, reverse transcriptase, deaminase, methylase, demethylase, or ligase, or a combination thereof.

57. The conjugate of claim 56, wherein the enzyme comprises a nuclease.

58. The conjugate of claim 57, wherein the nuclease comprises a CRISPR-Cas nuclease.

59. The conjugate of claim 58, wherein the CRISPR-Cas nuclease is a nickase.

60. The conjugate of claim 58 or claim 59, wherein the CRISPR-Cas nuclease is a Cas9.

61. The conjugate of claim 58, wherein the CRISPR-Cas nuclease is a Cas12a.

62. The conjugate of any of claims 58-61, wherein the conjugate further comprises a gRNA molecule.

63. The conjugate of any one of claims 1-49, wherein the therapeutic agent comprises a gene modifying polypeptide.

64. The conjugate of claim 63, wherein the gene modifying polypeptide comprises a retrotransposon.

65. The conjugate of claim 63 or claim 64, wherein the conjugate further comprises a template RNA that binds to the gene modifying polypeptide.

66. The conjugate of claim 65, wherein the template RNA encodes a CAR.

67. The conjugate of claim 65, wherein the therapeutic agent comprises a template RNA molecule, e.g., a template RNA that binds to a gene modifying polypeptide.

68. The conjugate of claim 67, wherein the template RNA molecule encodes a CAR.

69. The conjugate of any one of claims 1-49, wherein the therapeutic agent comprises a gene modifying system.

70. The conjugate of claim 69, wherein the gene modifying system comprises a gene modifying polypeptide and a template RNA.

71. The conjugate of claim 70, wherein the gene modifying polypeptide comprises a retrotransposon.

72. The conjugate of claim 71, wherein the template RNA encodes a CAR.

73. The conjugate of any one of claims 1-49, wherein the therapeutic agent comprises a heterologous gene modifying system, or a component thereof.

74. The conjugate of any one of claims 1-73, wherein the antibody or functional fragment thereof is of the IgG class, the IgM class, or the IgA class.

75. The conjugate of claim 74, wherein the antibody or functional fragment thereof is of the IgG class and has an IgG1, IgG2, IgG3, or IgG4 isotype.

76. The conjugate of any one of claims 1-75, wherein the antibody functional fragment is a Fab fragment, wherein the Fab fragment comprises an interchain disulfide bond and a CL-CH1 interface, and wherein the interchain disulfide bond is engineered away from the C-terminus and buried within the CL-CH1 interface.

77. The conjugate of claim 76, whereby the interchain disulfide bond is stable under mild reducing conditions.

78. The conjugate of claim 76 or claim 77, wherein the conjugate has a sequence set forth in Table 12.

79. A pharmaceutical composition comprising the conjugate of any of claims 1-78

80. A cell comprising a conjugate of any of claims 1-78.

81. A method of conjugating a lipid nanoparticle (LNP) to an antibody or functional fragment thereof, said method comprising:
(i) contacting the antibody or functional fragment thereof comprising a free cysteine residue with a crosslinker molecule comprising a first click handle and a thiol-reactive functional group, whereby the thiol-reactive functional group of the crosslinker molecule reacts with the free cysteine residue of the antibody or functional fragment thereof; and
(ii) contacting the product of step (i) with an LNP comprising a second click handle covalently bonded to the surface of the LNP, whereby the first click handle reacts with the second click handle via a click chemistry reaction, thereby forming a conjugate between the LNP and the antibody or functional fragment thereof.

82. The method of claim 81, wherein the free cysteine is in a hinge region of the antibody or functional fragment thereof.

83. The method of claim 82, wherein the method further comprises reducing disulfide bonds in the hinge region prior to step (i).

84. The method of any one of claims 81-83, further comprising removing an interchain disulfide bond near the C-terminus of the antibody or functional fragment thereof and introducing a new disulfide bond buried within the CL-CH1 interface of the antibody or functional fragment thereof.

85. The method of claim 84, wherein the antibody functional fragment is a Fab fragment.

86. The method of any one of claims 81-84, wherein the crosslinker further comprises a spacer between the first click handle and a thiol-reactive functional group.

87. The method of claim 86, wherein the spacer comprises a polyethylene glycol (PEG) and wherein the PEG comprises n ethylene glycol units, wherein n is between 4 and 200.

88. The method of any one of claims 82-87, wherein the hinge region sequence of the antibody or functional fragment thereof comprises the sequence DKTHTC (SEQ ID NO: 97).

89. The method of any one of claims 82-87, wherein the hinge region sequence of the antibody or functional fragment thereof comprises the sequence DKTHTCA (SEQ ID NO: 98).

90. The method of any one of claims 82-87, wherein the hinge region sequence of the antibody or functional fragment thereof comprises the sequence DKTHTCAA (SEQ ID NO: 99).

91. The method of any one of claims 82-87, wherein the hinge region sequence of the antibody or functional fragment thereof comprises the sequence selected from the group consisting of EPKSCDKTHTCPPCP (SEQ ID NO: 100), EPKCCVECPPCP (SEQ ID NO: 101), ELKTPLGDTTHTCPRCP(EPKSCDTPPPCPRCP)₃ (SEQ ID NO: 102), ESKYGPPCPSCP (SEQ ID NO: 103), VPRDCGCKPCICT (SEQ ID NO: 104), EPRGPTIKPCPPCK (SEQ ID NO: 105), EPSGPISTINPCPPCK (SEQ ID NO: 106), and EPRIPKPSTPPGSSCP (SEQ ID NO: 107).

92. The method of any one of claims 81-91, wherein the thiol-reactive functional group is a maleimide group, a parafluoro group, an ene group, an yne group, a vinylsulfone group, a pyridyl disulfide group, a thiosulfonate group, and a thiol-bisulfone group.

93. The method of claim 92, wherein the thiol-reactive group is a maleimide group.

94. The method of claim 93, wherein the maleimide group is substituted with one or more substituents selected from the group consisting of C₁₋₃ alkyl, halo, and C₁₋₃Oalkyl.

95. The method of any one of claim 81-94, wherein the reaction between the first click handle and the second click handle forms a click product, and, wherein the click product is formed through a copper-catalyzed click chemistry reaction, a Huisgen 1,3-dipolar cycloaddition between an azide and an alkyne, or through a copper free click chemistry reaction.

96. The method of claim 95, wherein the click product is formed through a copper-free click chemistry reaction, and wherein the copper-free click chemistry reaction is selected from the group consisting of (a) a strain-promoted cycloaddition between an azide and a cyclic alkyne; (b) a Staudinger ligation between an azide and a phosphine; (c) an inverse electron demand Diels-Alder reaction between a trans-cyclooctene (TCO) and a tetrazine; (d) an inverse electron demand Diels-Alder reaction between a tetrazine and a norbonene; (e) a photoinducible 1,3-dipolar cycloaddition reaction between a tetrazole and an alkene; (f) an oxime ligation between an aldehyde or ketone and an α effect amine; and (g) a hydrazone ligation between an aldehyde or ketone and an α effect amine.

97. The method of claim 96, wherein the click product is formed via an inverse electron demand Diels-Alder reaction between a TCO moiety and a tetrazine moiety.

98. The method of claim 97, wherein the TCO moiety is covalently bonded directly to the LNP.

99. The method of claim 97 or claim 98, wherein the tetrazine is unsubstituted.

100. The method of claim 97 or claim 98, wherein the tetrazine is methyltetrazine.


SEQUENCE LISTING (121 sequences)

SEQ ID NO: 1 - protein, 1110 aa, synthetic construct
MDSTAHPNQGRGLEKVSQTLPALQTPGQHTAAGGSSPLSGRNQRKNTKKLLLGAWNIRTLLDRENTPRPERRTALIGKELARYNIDIAALSETRLPEEGSLSEPTTGYTFFWKGRASNEDRIHGVGLAIKTSLLKQLPDLPVGISERLMKIRLPLSKDRYATIISAYAPTLTSTEETIEQFYSDLSAVLHSVPTNDKLILLGDFNARVGQDHERWKGVLGKHGVGKMNNNGLLLLSKCSEFELTITNTVFRMANKYKTTWMHPRSKQWHLIDYIIVRRRDIQDVKITRAMRGAECWTDHRLVRATLQMRIAPRHPKRAQTVRAFYNVSRLRDPSYLQTFQSCLDDKLSAKGPLTGSSTEKWNQFRDAVKETSKAVLGPKQRNHQDWFDENNTAIEDLLSKKNKAFMEWQNNPNSAPKKDRFKSLQATAQREIRKMQDRWWEKKAEEIQRFADMKNYKQFFSALKTVYGPLKPTTTPLLSSDGDTLIKDKKGISNRWKEHFSQLLNRPSSVDQSALDQIPQNRTIEQLDVPPSIEEVQKAIKQMSAGKAPGKDGIPTEVYKALNGKALQAFHIVLTSIWEEEDMPPELRDASIVALYKNKGSRAACDNYRGISLLSTAGKILARVILNRLLSSVSEQNLPESQCGFRPDRSTIDMVFTVRQMQEKCLEQNLSLYIVFIDLTKAFDTVNRDALWVILSKLGCPAKFVKLIQLFHVDMTGEVLSGGETSDRFNISNGVKQGCVLAPVLFNLFFTQVLRHAVMDLDLGVYIKYRLDGSLFDLRRLTAKTKTTERLILEALFADDCALMAHQENHLQTIVDRFSTATKLFGLTISLSKTEVLFQPAPGRPTNQPCITIDGTQLSNVNTFKYLGSTIANDGSLDHEINARIQKASQALGRLRCKVLQHRGVSTATKLKVYNAVVLSSLLYGCETWTLYRKHMKQLEQFHQRSLRSIMRIRWQDRITNQEVLDRANSTSIEVMVLKTQLRWSGHVIRMDPQRIPRQVFYGELSAGLRKQGRPKKRFKDQLKSNLKWAGITPKQLELAASDRSSWRTHINHAATTFEDERRRRLAAARERRHQATTAPPVTTGVPCPMCHKLCASAFGLQSHMRVHRR

SEQ ID NO: 2 - protein, 4 aa, synthetic construct
GGGS

SEQ ID NO: 3 - protein, 7 aa, synthetic construct
PKKKRKV

SEQ ID NO: 4 - protein, 16 aa, synthetic construct
SGSETPGTSESATPES

SEQ ID NO: 5 - protein, 11 aa, synthetic construct
VSGWRLFKKIS

SEQ ID NO: 6 - RNA, 1467 nt, synthetic construct
  source 1..1467: mol_type = other RNA
atggctctgccggtgaccgccctgcttctgcctcttgccctgctcttgcatgccgctcgcccgcaggtgcagcttgttcagtccggcgcggaggtaaaaaagcctggcgccagcgtgaaagtgagctgtaaggcttccggctacagcttcccagattactatatcaattgggtgcggcaggctcccgggcagggactcgagtggatgggttggatctattttgcgagcggaaactctgagtacaaccagaagttcaccggcagggttaccatgacccgcgacacgtcaagttctacggcatacatggagctgtctagtctcaggtccgaagataccgccgtgtatttctgcgcctccctgtatgattatgactggtacttcgacgtctggggccagggcaccatggtgacagtgtcttccggtggcggtggctccggcggaggtggctctggagggggtggcagcgatatcgtgatgacccagactccactgtctctgtctgtgacccctggtgagccggcgagcatctcctgcaagagttcccagagtctggtccactctaacggcaacacctacctgcactggtatctgcagaagccgggccaaagtccacagctcctgatctacaaggtttccaaccgtttcagcggggtcccggatcgcttttccggctcaggtagcggtgccgatttcaccctgaagattagtcgggtcgaggctgaggacgtcggcgtctactattgtgcagagaccagtcacgtcccctggactttcggtcaggggaccaaactggagatcaagaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaggagtaagaggagcaggctcctgcacagtgactacatgaacatgactccccgccgccccgggcccacccgcaagcattaccagccctatgccccaccacgcgacttcgcagcctatcgctccagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 7 - protein, 489 aa, synthetic construct
MALPVTALLLPLALLLHAARPQVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 4 aa, synthetic construct
LPET

SEQ ID NO: 9 - protein, 5 aa, synthetic construct
  VARIANT 1..5: note = Can be present in repeats of up to 10
GGGGS

SEQ ID NO: 10 - protein, 10 aa, synthetic construct
  VARIANT 2..10: note = Can be absent
GGGGGGGGGG

SEQ ID NO: 11 - protein, 5 aa, synthetic construct
  VARIANT 1..5: note = Can be present in repeats of up to 10
EAAAK

SEQ ID NO: 12 - protein, 5 aa, synthetic construct
  VARIANT 1..5: note = Can be present in repeats of up to 10
PAPAP

SEQ ID NO: 13 - protein, 4 aa, synthetic construct
  VARIANT 3..4: note = Can be absent, or present in repeats of up to 9
APAP

SEQ ID NO: 14 - protein, 16 aa, synthetic construct
  VARIANT 2..6: note = Can be present in repeats of up to 10
  VARIANT 11..15: note = Can be present in repeats of up to 10
AEAAAKALEAEAAAKA

SEQ ID NO: 15 - protein, 12 aa, synthetic construct
GFEDKVWYDLDA

SEQ ID NO: 16 - protein, 4 aa, synthetic construct
LLQG

SEQ ID NO: 17 - protein, 5 aa, synthetic construct
LPXTG

SEQ ID NO: 18 - protein, 5 aa, synthetic construct
LPXTA

SEQ ID NO: 19 - protein, 5 aa, synthetic construct
LPXTG

SEQ ID NO: 20 - protein, 7 aa, synthetic construct
LPETGGG

SEQ ID NO: 21 - protein, 8 aa, synthetic construct
LPETGGGG

SEQ ID NO: 22 - protein, 9 aa, synthetic construct
LPETGGGGG

SEQ ID NO: 23 - protein, 8 aa, synthetic construct
LPETAGGG

SEQ ID NO: 24 - protein, 10 aa, synthetic construct
LPETGGGGGG

SEQ ID NO: 25 - protein, 4 aa, synthetic construct
GGGG

SEQ ID NO: 26 - protein, 5 aa, synthetic construct
GGGGG

SEQ ID NO: 27 - protein, 6 aa, synthetic construct
GGGGGG

SEQ ID NO: 28 - protein, 7 aa, synthetic construct
GGGGGGG

SEQ ID NO: 29 - protein, 6 aa, synthetic construct
GGGGGS

SEQ ID NO: 30 - DNA, 22 bp, Danio rerio
  source 1..22: mol_type = unassigned DNA
tgagcacggtagcattagtcga

SEQ ID NO: 31 - protein, 965 aa, Danio rerio
MCFLIPVVTNTRKTREVRCRRNPHNLRSIHVSTISQLSLSVGLWNCQSAVNKADFITSIATYSDYNLMALTETWLRPEDTATHATLSANFSFSHTPRQTGRGGGTGLLISKEWKFTLIPSLPTISSFEFHAVTIIHPFYINVVVIYRPPGKLGHFLDELDVLLSSFSNFATPLLVLGDFNIYVDKPQAADFQTLLASFDLKRAPTSATHKSGNQLDLIYTRHCFTDQTIVTPLQISDHFLLSLNIHITPEPPHTPTLVTFRRNLRSLSPNRLSTIVSDSLPPSRKLTALDSNSATNTLCSTLASCLDRLCPLASRPARASPPAPWLSDALREHRSKLRAAERIWRKTKNPAHLLTYQTLLSSFSAEVTSAKQTYYRLKINNATNPRLLFKTFSSLLYPPPPPASSTLTTDDFATFFCTKTAKISAQFAAPTTNTQDTTPTPHTLTSFSQLSESEVSKLVLSSHATTCPLDPIPSHLLQAISPAVIPTLTHIINTSLDSGLFPTTFKQARVTPLLKKPNLDHTLLENYRPVSLLPFMAKILEKVVFNQVLDFLTQNNLMDNKQSGFKKGHSTETALLSVVEDLRLAKADSKSSVLILLDLSAAFDTVNHQILLSTLESLGVAGTVIQWFRSYLSDRSFRVSWRGEVSNLQHLNTGVPQGSVLGPLLFSIYTSSLGPVIQRHGFSYHCYADDTQLYLSFHPDDPSVPARISACLLDISHWMKDHHLQLNLAKTEMLVVSANPTLHHNFSIQMDGATITASKMVKSLGVTIDDQLNFSDHISRTARSCRFALYNIRKIRPFLSEHAAQLLVQALVLSKLDYCNSLLAGLPANSIKPLQLLQNAAARVVFNEPKRAHVTPLLVRLHWLPVAARIKFKTLMFAYKVTSGLAPSYLHSLLQIYVPSRNLRSVNERRLVVPSQRGKKSLSRTLTLNLPSWWNELPNCIRTAESLAIFKKRLKTQLFSLHFTS

SEQ ID NO: 32 - DNA, 100 bp, Danio rerio
  source 1..100: mol_type = unassigned DNA
gtttcgcgtcgtccgcctcagtttcggcccttgttttgactcgggaggcgtgtccaaacgccaggtaaccactatatagtgagcacggtagcattagtcg

SEQ ID NO: 33 - skipped

SEQ ID NO: 34 - DNA, 1110 bp, Danio rerio
  source 1..1110: mol_type = unassigned DNA
gcaggagaagcactttagcagcatctagaacagcagcctgtaagtacatttaagatttgtttcagttgttgtgtatggtttgaggacttgtttccagctgtttgtgtaaagttgtaggacatttaaacttgctttcagttgtgtataatactagaagttgtttagccactgtttccttggttactataagagcttgtgtagcgaacgcagacgcggttcgcgtcgtccgcctcagtttcggcccttgttttgactcgggaggcgtgtccaaacgccaggtaaccactatatagtgagcacggtagcattagtcggcaggagaagcactttagcagcatctagaacagcagcctgtaagtacatttaagatttgtttcagttgttgtgtatggtttgaggacttgtttccagctgtttgtgtaaagttgtaggacatttaaacttgctttcagttgtgtataatactagaagttgtttagccactgtttccttggttactataagagcttgtgtagcgaacgcagacgcggttcgcgtcgtccgcctcagtttcggcccttgttttgactcgggaggcgtgtccaaacgccaggtaaccactatatagtgagcacggtagcattagtcggcaggagaagcactttagcagcatctagaacagcagcctgtaagtacatttaagatttgtttcagttgttgtgtatggtttgaggacttgtttccagctgtttgtgtaaagttgtaggacatttaaacttgctttcagttgtgtataatactagaagttgtttagccactgtttccttggttactataagagcttgtgtagcgaacgcagacgcggttcgcgtcgtccgcctcagtttcggcccttgttttgactctcgaggcgtgtccagctgaattcaatcagctagtgctttggggttatataaacaactagttcaccgcggcagcggtcgcggcagcctcgtgtgaagaccgacgagggtaaagaccatcgactctacctgcgcgactccaccgagcaaagacaccgacaaagcacttgagtactttactgtattgttttactttacacttattttttgttgtcagtgcacttttatt

SEQ ID NO: 35 - DNA, 233 bp, Danio rerio
  source 1..233: mol_type = unassigned DNA
taatctgcaattgcctctctgaatatcacactaactgtacccaaaaaaaaaaaaaaaaaaaaaaaaaataaaaawactactaatacttcccttcttagactttacagacctgaaacttgcctatagcacttattcattgttgctcttagttgtgtaaattgcttccttgtcctcatttgtaagtcgctttggataaaagcgtctgctaaatgactaaatgtaaatgtaaatgt

SEQ ID NO: 36 - protein, 965 aa, Danio rerio
MCFLIPVVTNTRKTREVRCRRNPHNLRSIHVSTISQLSLSVGLWNCQSAVNKADFITSIATYSDYNLMALTETWLRPEDTATHATLSANFSFSHTPRQTGRGGGTGLLISKEWKFTLIPSLPTISSFEFHAVTIIHPFYINVVVIYRPPGKLGHFLDELDVLLSSFSNFATPLLVLGDFNIYVDKPQAADFQTLLASFDLKRAPTSATHKSGNQLDLIYTRHCFTDQTIVTPLQISDHFLLSLNIHITPEPPHTPTLVTFRRNLRSLSPNRLSTIVSDSLPPSRKLTALDSNSATNTLCSTLASCLDRLCPLASRPARASPPAPWLSDALREHRSKLRAAERIWRKTKNPAHLLTYQTLLSSFSAEVTSAKQTYYRLKINNATNPRLLFKTFSSLLYPPPPPASSTLTTDDFATFFCTKTAKISAQFAAPTTNTQDTTPTPHTLTSFSQLSESEVSKLVLSSHATTCPLDPIPSHLLQAISPAVIPTLTHIINTSLDSGLFPTTFKQARVTPLLKKPNLDHTLLENYRPVSLLPFMAKILEKVVFNQVLDFLTQNNLMDNKQSGFKKGHSTETALLSVVEDLRLAKADSKSSVLILLDLSAAFDTVNHQILLSTLESLGVAGTVIQWFRSYLSDRSFRVSWRGEVSNLQHLNTGVPQGSVLGPLLFSIYTSSLGPVIQRHGFSYHCYADDTQLYLSFHPDDPSVPARISACLLDISHWMKDHHLQLNLAKTEMLVVSANPTLHHNFSIQMDGATITASKMVKSLGVTIDDQLNFSDHISRTARSCRFALYNIRKIRPFLSEHAAQLLVQALVLSKLDYCNSLLAGLPANSIKPLQLLQNAAARVVFNEPKRAHVTPLLVRLHWLPVAARIKFKALMFAYKVTSGLAPSYLLSLLQIYVPSRNLRSVNERRLVVPSQRGKKSLSRTLTLNLPSWWNELPNCIRTAESLAIFKKRLKTQLFSLHFTS

SEQ ID NO: 37 - DNA, 1110 bp, Danio rerio; identical to SEQ ID NO: 34
  source 1..1110: mol_type = unassigned DNA

SEQ ID NO: 38 - DNA, 233 bp, Danio rerio; identical to SEQ ID NO: 35
  source 1..233: mol_type = unassigned DNA

SEQ ID NO: 39 - protein, 1110 aa, Monodelphis domestica; identical to SEQ ID NO: 1

SEQ ID NO: 40 - DNA, 710 bp, Monodelphis domestica
  source 1..710: mol_type = unassigned DNA
gggtgtatggggtgctcagggaggggtagtatctctggtatggagggcttgtcgtgccctcctagggcagctctccagcctctgacccccacctgacacccagctctcacttgtggctcccagtagctgctagcatgtggcagcggccacaccccgggcaacggcttcgacaggccggctaaaccttgtgagggtagccatcgggtcatcgacccctggtgaaccagggctttgctcacccagcatgtgaagactgcttcggctgaacagacggaagaaaccaataagaaggttcaacggctgagagggcgacgcagcaaagcactgtggagtgcttagggcgtgttggagcacaaaggacaacacggccatccaatgcagctgaggaagtctccagatgtaacaatttttcgtgccactggacccaggcttccaacgccgagagagtgggactgtctctgtgcatcggcttttccacttaaatctctttcacgcacaagtatctttgtgcacactcatctatcctaaccccgtccaccctcttcaagacctgcggcgatgggggagtggcgacgcaacaggtggaggtgaccactggcagttgtagtcacgatcctgcacgtaggcggcccacggaccagtggtcgctcggccctgtgggcagcagggacgttcggcagcatcctgggcgactgagcagccctctctag

SEQ ID NO: 41 - DNA, 51 bp, Monodelphis domestica
  source 1..51: mol_type = unassigned DNA
tgaaactgcacaaagacaatagtcattctcgatcaccgagagactaccact

SEQ ID NO: 42 - protein, 1110 aa, Monodelphis domestica
MDSTAHPNQGRGLEKVSQTLPALQTPGQHTAAGGSSPLSGRNQRKNTKKLLLGAWNIRTLLDRENTPRPERRTALIGKELARYNIDIAALSETRLPEEGSLSEPTTGYTFFWKGRASNEDRIHGVGLAIKTSLLKQLPDLPVGISERLMKIRLPLSKDRYATIISAYAPTLTSTEETIEQFYSDLSAVLHSVPTNDKLILLGDFNARVGQDHERWKGVLGKHGVGKMNNNGLLLLSKCSEFELTITNTVFRMANKYKTTWMHPRSKQWHLIDYIIVRRRDIQDVKITRAMRGAECWTDHRLVRATLQMRIAPRHPKRAQTVRAFYNVSRLRDPSYLQTFQSCLDNKLSAKGPLTGSSTEKWNQFRDAVKETSKAVLGPKQRNHQDWFDENNTAIEDLLSKKNKAFMEWQNNPNSAPKKDRFKSLQATAQREIRKMQDRWWEKKAEEIQRFADMKNYKQFFSALKTVYGPLKPTTTPLLSSDGDTLIKDKKGISNRWKEHFSQLLNRPSSVDQSALDQIPQNRSIEQLDVPPSIEEVQKAIKQMSAGKAPGKDGIPTEVYKALNGKALQAFHIVLTSIWEEEDMPPELRDASIVALYKNKGSRAACDNYRGISLLSTAGKILARVILNRLLSSVSEQNLPESQCGFRPDRSTIDMVFTVRQMQEKCLEQNLSLYIVFIDLTKAFDTVNRDALWVILSKLGCPAKFVKLIQLFHVDMTGEVLSGGETSDRFNISNGVKQGCVLAPVLFNLFFTQVLRHAVMDLDLGVYIKYRLDGSLFDLRRLTAKTKTTERLILEALFADDCALMAHQENHLQTIVDRFSTATKLFGLTISLSKTEVLFQPAPGRPTNQPCITIDGTQLSNVNTFKYLGSTIANDGSLDHEINARIQKASQALGRLRCKVLQHRGVSTATKLKVYNAVVLSSLLYGCETWTLYRKHMKQLEQFHQRSLRSIMRIRWQDRITNQEVLDRANSTSIEVMVLKTQLRWSGHVIRMDPQRIPRQVFYGELSAGLRKQGRPKKRFKDQLKSNLKWAGITPKQLELAASDRSSWRTHINHAATTFEDERRRRLAAARERRHQATTAPPVTTGVPCPMCHKLCASAFGLQSHMRVHRR

SEQ ID NO: 43 - DNA, 710 bp, Monodelphis domestica; identical to SEQ ID NO: 40
  source 1..710: mol_type = unassigned DNA

SEQ ID NO: 44 - DNA, 51 bp, Monodelphis domestica; identical to SEQ ID NO: 41
  source 1..51: mol_type = unassigned DNA

SEQ ID NO: 45 - protein, 982 aa, Anolis carolinensis
MDEYQRSLSRPLLTIMSINIEGLSLAKEELLAKMSEDISCDILCIQETHRDITMRRPKILGMQLAVERPHRQYGSAIFVRSGVAISATSLTEVNNIEILSVELDSCTVSSLYKPPGADFYFTPPTSCHNHEAHFVVGDFNSHSCVWGYDEDDRNGEAVLTWADNSRMSLLHDSKLPPSFNSGRWKRGYNPDLIFVKESISHQCTKRVLNPIPNTQHRPICCVAYAAVRPKSVPFRRRYNFNKANWTKFTETLEAAISDIEPSIENYDLFVEAVKRSSRLSIPRGCRTSYLPGLNEESLNQLQEYLRLFQENPYSDGTIAAGQKLSTALANAKKDRWIELLENLDMSKSSRKAWQLLRRLDSDPLVNPGHANVTPDQIAHQLIQNGKTNCSRIKMKINRVPELETHQLSSPLNLKELREAIKRCKTGKAPGLDDLMMEQIKHLGPKAENWLLKFYNQCLAHKQIPRAWRKTKIIAILKPGKDASNARNYRPISLLCHLYKVYERMLLNRLGPVIEPKLIAQQAGFRPGKNCTGQILHLTEHIEEGYEKGCITGTVFVDLTAAYDTVQHRKMLHKVYHITRDFDFTKTVQTLLENRSFYVEFQGQKSRWRRQKNGLPQGSVLAPTLFNIFTNDQPQPPLTKSFIYADDLGLTTQAKDFETVEKQLTNALKDLSSYYKENHLKPNPAKTQVCAFHLRNREANRKLKVTWEGQELEHCFHPKYLGVTLDRTLTYRKHCMNTKHKVAARNNILRKLTGSAWGADPQVIRTSALALSFSTAEYACPVWHKSAHAKQVDIALNETCRIITGCLKPTPVDKLYKLAGIAPPDVRREVAANGERKKVEHCESHPLHDYHPPPTRLKSRKGFMRTTTPLDVPPATARVSLWAAKPGNSNWMAPQEGLPPGANQEWATWKSLNRLRSGVGRSKDNLARWHYLEESSTLCDCGAEQTTQHMYACPQCPASCTEEELFKATDNAVAVARFWSKTI

SEQ ID NO: 46 - DNA, 109 bp, Anolis carolinensis
  source 1..109: mol_type = unassigned DNA
gggggacacggaaagagcctccccgaagattgagtgaattcagtcgggcgtcccctgggcaacgtttcttgtaagcggccgatctttccaccccaaaagcattggatga

SEQ ID NO: 47 - DNA, 91 bp, Anolis carolinensis
  source 1..91: mol_type = unassigned DNA
tagttgcttgtgatttcttttcttttttattttatttccattatttgaaatgtatttgctgtaccaatgcttttgacacgaaataaataaa

SEQ ID NO: 48 - protein, 982 aa, Anolis carolinensis
MDEYQRSLSRPLLTIMSINIEGLSLAKEELLAKMSEDISCDILCIQETHRDITMRRPKILGMQLAVERPHRQYGSAIFVRSGVAISATSLTEVNNIEILSVELDSCTVSSLYKPPGADFYFTPPTSCHNHEAHFVVGDFNSHSCVWGYDEDDRNGEAVLTWADNSRMSLLHDSKLPPSFNSGRWKRGYNPDLIFVKESISHQCTKRVLNPIPNTQHRPICCVAYAAVRPKSVPFRRRYNFNKANWTKFTETLEAAISDIEPSIENYDLFVEAVKRSSRLSIPRGCRTSYLPGLNEESLNQLQEYLRLFQENPYSDGTIAAGQKLSTALANAKKDRWIELLENLDMSKSSRKAWQLLRRLDSDPLVNPGHANVTPDQIAHQLIQNGKTNCSRIKMKINRVPELETHQLSSPLNLKELREAIKRCKTGKAPGLDDLMMEQIKHLGAKAENWLLKFYNQCLAHKQIPRAWRKTKIIAILKPGKDASNARNYRPISLLCHLYKVYERMLLNRLGPVIEPKLIAQQAGFRPGKNCTGQILHLTEHIEEGYEKGCITGTVFVDLTAAYDTVQHRKMLHKVYHITRDFDFTKTVQTLLENRSFYVEFQGQKSRWRRQKNGLPQGSVLAPTLFNIFTNDQPQPPLTKSFIYADDLGLTTQAKDFETVEKQLTNALKDLSSYYKENHLKPNPAKTQVCAFHLRNREANRKLKVTWEGQELEHCFHPKYLGVTLDRTLTYRKHCMNTKHKVAARNNILRKLTGSAWGADPQVIRTSALALSFSTAEYACPVWHKSAHAKQVDIALNETCRIITGCLKPTPVDKLYKLAGIAPPDVRREVAANGERKKVEHCESHPLHGYHPPPTRLKSRKGFMRTTTPLDVPPAAARVSLWAAKPGNSNWMAPQEGLPPGANQEWATWKSLNRLRSGVGRSKDNLARWHYLEESSTLCDCGAEQTTQHMYACPQCPASCTEEELFKATDNAVAVARFWSKTI

SEQ ID NO: 49 - DNA, 109 bp, Anolis carolinensis; identical to SEQ ID NO: 46
  source 1..109: mol_type = unassigned DNA

SEQ ID NO: 50 - DNA, 91 bp, Anolis carolinensis
  source 1..91: mol_type = unassigned DNA
tagttgcttgtgatttcttttcttttttattttatttccattatttgaaatgtatttgttgtagcaatgcttttgacacgaaataaataaa

SEQ ID NO: 51 - protein, 979 aa, Parhyale hawaiensis
MLYTIFLVYGILCFFFVYFCIYLYITVYLCFLFFLFLLLCGDVESNPGPGRARGCRLLYCNIRGLHANLAELDFVSRGVDVVCCSETLVAGRRHDAELALAGFQSPFRRLCGSGPGFRGMAVYVRSGFCAYRQSVHECACHEIIVVRVCGRLNNYYLFSLYRSPATDDSLYDCLLTAMASIQSTDPKAAFVFVGDVNAHHRDWLGSASPTDCHGVAALDFCTLSSCVQLVRGSTHIAGNCLDLVMTDVPDLMTVTVGSPIGSSDHSHLVVSLDLNQVVPVVDTRRVVFVKSRANWVAITRAVRTLPWRQIIHSEDPVSELNDLTVSILERFVPKRTILVRSRDKPWFDDQCRLAFEAKQAAYRAWRRSRDRTLWQTYVDRRAEAKRVYEEAQRRLRQRSRESLLSIDHPHRWWSELKGSVFGAEPSLPPLVGPGGGIITDPLARAELLSAHFDGKQSRDVIALPHGCHPEPRLTSLAFRSGAIKVLLEGLDPYGGVDPVGMFPLFYKQLADVLAPKLAVIFRRLIRLGNFPRCWRTGNITPIPKGPVSPYVANYRPITLTPILSKVFEKLIAGKLGRFAEVTGLLPAGQFAYRIGLGCCDALLSVSHHLQSALDCRSEARLVQLDFSAAFDRVNHRGLLYKLESFGVGGRVLSIIRDFVSERTQSVSVDGVLSASVGVVSGVPQGSVLGPLLFVLYTSDMFSSLENTLINYADDSTLMAVIPAPRLRDAVAQSLNRDLSRISAWCSAWSMKLNASKTKSMIISRSRTLVPQHPQLEIDDTLLQESSSLEILGVVFDEKLTFEPHIRRLVSRASTKIGLLRKVNSVFGDSQVARRCFYAFLLPVLEYCSPVWASAADTHLRLLDRLVSSASRLCADNDLVNLSHRRRVAELCMFYKVYNNERHWLYSSLPALKVFGRETRAACGAHSFTLEAVRCRTNQFCRCFVPWSAKVWNLLPASAFGRIGLQAFKSAVNGFLLDFL

SEQ ID NO: 52 - DNA, 383 bp, Parhyale hawaiensis
  source 1..383: mol_type = unassigned DNA
gcgtggcctgcgcgttatcaggccgcccccattgtccggccaccggacgcctctttgtttgtatgagctggctttgggtttggggctctgggtagcccctgagttggaggaaatgccactctaaaggcggatgagacctcttttggtccaacggcatccctgagaggagatgaacaggctggtgccatagctgggtactcagtcttccgcagtctgctcctctctctcttccatcaggtagtaccacctgatgctcttttcctttcctttctcttcttcttcttttcggccaaagcctgtactggtgatggcagggtctggggcgcgtagatagctggccgctttcgtagctatcgttactgtcgatttgtttttgctttatt

SEQ ID NO: 53 - DNA, 99 bp, Parhyale hawaiensis
  source 1..99: mol_type = unassigned DNA
tagcagcgttttgggcttcccggtgttgcaagatgcttcatttgggttgtattacctgtccgtgggagctcaagcgtgaagttttaataataataataa

SEQ ID NO: 54 - DNA, 200 bp, Branchiostoma floridae
  source 1..200: mol_type = unassigned DNA
nnnnnnnnnnnnnnnnnnntnnnnncnngtnnnnannnnnnnnnnnnnncnnnnnnnnnnnnnnnntnnnnnnnnnnnnnnnnnnnntnnnnnnnnnnnnnntnnnnnnnnnnnnnnnnnnnncnnnnnnnnnnnnnnnnnngcnnnnnnnntnnnnnnnnnnnnnnnnnnnnnnnnntnnnnnnnnnnnannnnnnnnn

SEQ ID NO: 55 - protein, 1037 aa, Branchiostoma floridae
MWEKATSNVHLQSGTWITQNAYSVKINPGLSGVRLIGRSTTCTERTERTLNLLVCATLLLAGDVSPNPGPDTGGLPVWRKGIVYAFYNVVSLPRHLDEIQQLLLRNTRIHVLGLNETRLSDSIPDSSVDINGYTLYRTDRDRQGGGVGVYVKQTIASQRRCELEQEDLEVCCVEIKPEKARKTLLTCVYRPPTSGPDWRNSAESLVHKLNQTAEKENADVAIMGDFNSDLLTSTQAMSSVEFLMGLYQLVPVIREPTRITEKTESCIDNIFVSNPDRYKSSASVAWGPSDHNLILTCAKAGSEAGAAHRCEYRSYKLYTQQSFIDSLKSVRWDTVFDCTDVSEAWNAFKDIFLNVADEHAPLRTKTARENNRPAPWMTDTVKNMMGRRDAARRKAIRTKDVQDWDTYRSLRNQTTSIIRKEKKSHFATAVSEAKGDQSLMWKIINSFTGKSKSTKRVQKLLRADNTSMSDPGEMAQEFNDYFTSCASRLTDGMPDSEEDPLRHIPDSTTKFSFDCVEETEVLNELQKLKTKKATGLDKIPAKLLKDSAPVVAKPLAHIFNLSLASGEVPSDWKEAQITPVHKSGSCADVGNYRPVSVLSVTSKVMEKLVCNQVTRYLTRCKLLTTHQSGFRRHHSTATAVQKVVEDITSGYNCSKVTVALFLDLRKAFDSVNHEIMLSKLKKFGFDSDAMKWFTSYLSERLQCTCLQGQYSSKTRVSCGVPQGSVLGPLLFCLYVNDLPNVIQKCSIHMYADDTVLYYSAVSVKVCEETVSMDMKRVVKWLSENRLLLHPDKTKSMLFGLPQKLKHAGTTVNITDGVNVYEQVDSFTYLGITLDPALRWAAHVQKITKKLLSGLGAMGRARAFVTNEVLKTMYQTLLLAHLEYCATAWLPSLAQGNKTLMLQLDRLVNRAARLITGHKLRDHVTVDNLRAEAGIDSVRKRTEITTLVTVFKTIRGKAPAYLASLFKWEAPPTMSVRPTRSEVKRLRDYDPHLLWCPPARVIAFRNSLQSYGPFLWNSLPLKQRQLLSLRTFKKFIEN

SEQ ID NO: 56 - DNA, 13 bp, Branchiostoma floridae
  source 1..13: mol_type = unassigned DNA
agccctagtccct

SEQ ID NO: 57 - DNA, 171 bp, Branchiostoma floridae
  source 1..171: mol_type = unassigned DNA
aacttgatctggctgatgctccgtagtctggactttgatattggataattttattatgtattgtatatgtgctatgtgtactttgtacttttatgtccaggattacctgaaaagcaggccgatttccggcctgagatgtaattactggtaaaataaataaagtgaagtgaa

SEQ ID NO: 58 - protein, 986 aa, Gasterosteus aculeatus
MRRLLLLFLIMCLTPSPVPVRISSRRYYRPRARSALYRNLSSLSYPTRSTHVQHLVTGGLWNCQSATRKADFISGFAIQQSLDFLALTETWITPENTSTPAALSSAFSFSHTPRPTGRGGGTGLLISPKWSFSLYPLPPSTPLSFEFHAVTITHPVQLTIIVLYRPPGSLGHFLEELDILLSNFPENGPPLILLGDFNIQTEKSSDLLHLLSSFALSLSPSPPTHKAGNHLDYIFTRNCSTTNLSVTPLHVSDHFFISYSLPLSITNKPPSLTNSIPARRNIRSLSPSSLASSVLSALPSTDSFSLLHPNAAAETLLSTLSSSLDSLCPLTTRRTGKSPPAPWLSQPVRAMRATMRASERRWRKYKRPDDLLEFQSLLSSFSASISAAKSSFYQSKIESSFSNPKKLFSIFSNLLEPPTPPPPSTLLPGDFVNYFTKKIADIRSSFSNPPPTSRVPPTSPLSPSLSSFTALSPNQILTLVTSARPTTCPLDPIPSHLLQSIAPDLLPFLTCLINNALSSGCFPNSLKEARVNPLLKKPTLNPSEENNYRPVSLLPFLSKTLERAIFNQLSSYLHCNNLLDPHQSGFKAGHSTETALLAVSEQLHTARAASLSSVLILLDLSAAFDTVNHQILISSLQELGVTGSALSLLSSYLDGRTYRVTWRGSVSEPCPLTTGVPQGSVLGPLLFSLYTNSLGAVIRSHGFSYHSYADDTQLILSFPHSDTQVAARISACLTDISQWMSAHHLKINPDKTELLLFPGKDSLTQDLTVNFGNSVLTPTSTAKNLGVTLDSQLSLTPNITATTRSCRYTLYNIRRIRPLLTQKAAQVLIQALVISRLDYCNSLLAGLPATAIRPLQLIQNAAARLVFNLPKFSHTTPLLRSLHWLPVAARIQFKTLVLTYHAVNGSGPAYIQDMVKPYIPTRTLRSASAKLLVPPSLRAKHSTRSRLFAVLAPKWWNELSEDTRTAESLHIFRRKLKTHLFRLYLD

SEQ ID NO: 59 - DNA, 705 bp, Gasterosteus aculeatus
  source 1..705: mol_type = unassigned DNA
cagtgtgcatctcttctacaaggccaccagacatccaggcgacctgaaatcggactaagtatctctttctttaaaccaagctagtctgctaaatttgctgttgattctagtgtcttaagttaatttgattgctgatttgctttagtttttgtcgtctcacggccagttctctttgtttgaagttatcttggttgctagtttgctcaagctctttcagtcttttagtgccagtttgtgttctagattctactattaagttaactgccagtgtgccctcctaactctgctaggttttgacctgatctaagtctgttttgccttgttttctgaagcaaataagacttgtatccttaaactctcattttgtcaaaacaccacatggtgtttgttcactgattagggtgtcaagctattggtgctttgcattttgaggagtttctgtcgaggccaatcgaccttttgtctcctaaacgacgggggagcggccagcgcagccgcagccgacttccactaacgagggagtatttaactagaaatcgtgggaagcgttagcttatcctcgcagcacgaagacgagcagaacaaagaccagggagtctttcgtggagtcaagacgagagacaaagaccagggagtctttcgtggagtcaagacgagcagaacaaagacaagggagtctttcgtggcagtcttcggggcggctga

SEQ ID NO: 60 - DNA, 203 bp, Gasterosteus aculeatus
  source 1..203: mol_type = unassigned DNA
taaagactaacaaattgtagcacttaaattgtacttgtaacgtcactcatctatagcaaattgtaaattggcttatttgaggaaattgcactttcttgtttcttgttctcctgagtttgtaccctatggttgaatgcacttattgtacgtcgctttggataaaagcgtcagctaaatgacatgtaatgtaatgtaatgtaatg

SEQ ID NO: 61 - DNA, 200 bp, Branchiostoma floridae
  source 1..200: mol_type = unassigned DNA
ntnnnnnnnnnnntnnnnnnnnnnnnnnnnnnnnngnnnnnnnnnnnnnnnnnnnnnnnnnnnnnnnnnnnntnnntnnnnnnnnnnnnannnnnnnnnnnnnnnnnnnnnntnnnnnnnnnnnnnngngnnnnnntnnnnncnnnnnnnntnnnnnnnnnnnnnntnnnnnnnnnnnnnannnnnnnnnnnnnnnnnnn

SEQ ID NO: 62 - protein, 1129 aa, Branchiostoma floridae
MSGTPRVAFDSGKDLRNPIGQSPPALSRAAPGQLGTDPSRSACFIGCLELRVLLDKWIVCRAPDKQESKEKRRQKTQPIRIGSWNVRTMRTGLSDDLTVIEDIRKTAAIDRELYRLNIDIVALQETRLPDSGSLKEDSYTFFWQGKGMEETREHGVGFAVRNTLLHMIEPPTGGTERIITLRLSTHEGPVNLLCVYAPTLQATSEVKDQFYGQLDSAIKKIPVSEHIFILGDFNARVGTDQESWQTVLGHHGIGKMNENGQRLLELCCYHNLCVTNTFFQNKAIHKASWRHPRSQRWHQLDLVITRRTSLNSVCNTRAYHSADCDTDHSLIAARIKLRPKKLHHMKKKGQPKIDVSKTMLPDRNQKFLECLEGTLNNIQPQDAEHRWETLSKTIYSAAAQSYGKKERKNTDWFEAYISELEPVMDTKRKALVSYKQNPSSQNLQALKAARQEAQRASRRCANNYWLLLSERIQLASATGDIRRMYEGIKQATGKPIKKSAPLKAKSGEIITDKDKQMARWVEHYLDIYSTENSVSQDALDNIEDFSVLAELDADPTIEELSKAIDSMSNGKAPGEDNIPAEIIKSGKSVLLEPLHELLRLCWKEGKVPQSMRNSKIVTLYKNKGDRTDCNSYRGISLLSIVGKVFAKVVLTRLQVLADRVYPESQCGFRAERSTTDMIFSVRQLQEKCREQQRPLYIAFIDLTKAFDLVSRRGLFQLLRKIGCPPQLLDIIISFHEDMKGVVSFDGETSEPFAIRSGVKQGCVLAPTLFGIFFSLLLKSAFGHSTQGVHLHTRSDGKLFNLARLRAKTKVRSVLIRDMLFADDAALVAHVEDELQQLLNQFAHACSEFALTISIKKTVVMGQDVPQPPVVTIGSEVLEVTDHFTYLGSTVTSNLSLDKEIDRRIARAAGVMTKLGTRVWNNSHLTLNTKLEVYRSCVLSTLLYGSETWTTYAKQENRLESFHLRCLRRILGISWRDRVPNTTVLERSCSLSIHLLLCQRRLRWLGHVSRMKDGRIPKDILFGELATGKRPVGRPALRFRDVCKRDLKLTDIDPASWEQIAADRNRWRHTVKDGLAKGQERRTEHLESRRRKRKEKPQQGNPSAFICPNCGRDCHARIGLQSHSRRCQPP

SEQ ID NO: 63 - DNA, 764 bp, Branchiostoma floridae
  source 1..764: mol_type = unassigned DNA
tctgtaaatggctgtgtgatgcgtctagtgtgtaggcgtagcggtagtgtgcttgccacttctcgctttcagccctcaccgctggctagcggagctgcatgcagcactgcgaaaagagagccgagtgcgtaagtctctcctgccagtacatagcctctccacagcaagtccccattgcagtgcctcctcgtggctacagacggaaactgggcaccgtaaggccccaggctaaactgcagggagctcggaggaggtctggcccccagacgcacggcatgcatggcccaccggcgtgtggacacgccctgatgcctgcgaaccagacccccagctatgggcaaatagcacgggtagacggagctcgtcagccttggatggcagttcgtctaggggaaggaaaaccctgattcaaaaacctccgctgccttgcggctatacccagtcctgggaaaggctacgggagttaacccagagagaaaatccggagtggagtacgtgaggcggttggctgtcaaactctgtcatccttccggcaactcctgcagccaaaccaacgccaagtgtcacgcctcgcgttcccttggaccacgtcggtgaggtcgagaggggggtcctgttgtgtttttgggcagcgcaggtcctccataaacctgcccaggctagcgctctggagaggccactccagtcgcccccatcactgggggtgagaaacaaaccgggagacagcagtttacgggttataagtccttgctaaattgacgtaa

SEQ ID NO: 64 - DNA, 54 bp, Branchiostoma floridae
  source 1..54: mol_type = unassigned DNA
tgacctgatacagagcgctaccatcatctggaaagatggaaggatgcctactac

SEQ ID NO: 65 - DNA, 200 bp, Locusta migratoria
  source 1..200: mol_type = unassigned DNA
nnnnnnnnnnnnnnnnnnnnnnannnnnnnnnnnnnnnnannnnncnnnannnnnnnnnnnnnnnnnnnnnnnnnnnnnnnnnnnnnnnnnnnnnncnnnnnnnnnnnnnnannnnnnnnnnnnnantnntnnnnnnnnnnnnnnnnannnnnnnnnnnnngnnnnnnannnnntnnnnnnannnnnnnnnnnannnnnn

SEQ ID NO: 66 - protein, 965 aa, Locusta migratoria
MPRKNWNCGRRTGDEKRKMMFGCWNVQGISTKIDLLPAELDMFNIDVVVLSETKRKGKGEEELDNYVHIWSGVSKAVRAKAGVSIMIQKKWKKRITNWTFINERIITVEMTLFAREVVIIGVYAPTNDTKDKEKDAFWDTLRETIEKIPRRKELIIMGDMNGRVGIRESCKIVGKHGEAEYNDNGERLIDICAQFDLKITNTFFKHKDIHKYTWQQNTKELRSIIDYIIIRQTSSFKAADVRSYRGAQCGSDHYLVKMKSFWPWKNATNDTSNINKMNCSEKVQNVHFNIDSLQDESIRTFFKARMERTLDESFEGSTEEIYEYIKTKVKNVASEVLGIKENNPKRAAEWWSEEIETSVKEKRNAFVQWLNDKSEGTRSKYKEKKNEVEKKIRLAKNEAWERTCANVNSKLGFGRAKEAWSVLKALRQDTKGKSNLQLVTQKEWEEYFKKLLNEDRDEYLEEGTVEENEHHDDEILISESEVLQVLRTGKNGKSPGPGNINMEFLKYGGDKIVKLILQLFNKMLHGDSVPKEMKLGYISTIFKKGDRKICSNYRGICVTNTLMRIFGKIIKNKLEKNFRTQQEQCGFTAGRSCVDHIFTLRQILEKHREKSKNVGLIFIDLEKAYDTVPRKLLWRALHRANINTSLIKIIEQMYKDNICQVKIGNTLSQKFRTSKGLLQGCPMSPTLFKIYIDICLRTWSQKCNSMGLEIRDGVYLHHLLFADDQVVIAQDGEDANYMCNQLAIAYKNWGLKINYQKTEYLTNDPHELRIEGKKIKKVNTFCYLGSILETEGKSDSEINKRISSGRKVIGMLNSVLWSRNVMNRTKKIIYKSIFESAVLYGAETWTINQKHTKKLQALEMDFWRRSARISRKEKRRNTEVIKRMEIIERIDEVMDRKKLRWYGHVRRMEDTRIPKLVLEWQPEGRRRRGRPVTTWIKNVQLTMNRLGAEEEDTQDRHTWRNIVNN

SEQ ID NO: 67 - DNA, 268 bp, Locusta migratoria
  source 1..268: mol_type = unassigned DNA
cccgtgtggagtttgctggtcttccatcgggcacctccccaggtggcggataggggaatgctcaccagatatggtgggtaccggggaaataaaatacccggggtggaccaaaaccagcaactgctgccttgtagtatgatattggcttatcaaaggctaaaggaagaaaaccttgaatacaaattacctggtcctccaggttgggggttgtgcagtgggccagctcctcactcacataaaaatataaaatgctaaaaaacctaataat

SEQ ID NO: 68 - DNA, 104 bp, Locusta migratoria
  source 1..104: mol_type = unassigned DNA
tagtgtaaaaccttatgtactaggtgtattcatttctgggcgtattagtatgttggaggtaaacctctgtaatgaggacaatctcaataataaaataaaataaa

SEQ ID NO: 69 - protein, 4 aa, synthetic construct
YADD

SEQ ID NO: 70 - protein, 4 aa, synthetic construct
YMDD

SEQ ID NO: 71 - protein, 4 aa, synthetic construct
YVDD

SEQ ID NO: 72 - DNA, 26 bp, Homo sapiens
  source 1..26: mol_type = unassigned DNA
ctgacccttctctctctcccccagag

SEQ ID NO: 73 - DNA, 15 bp, Homo sapiens
  source 1..15: mol_type = unassigned DNA
tttctctcccacaag

SEQ ID NO: 74 - protein, 1381 aa, synthetic construct
MPAAKRVKLDGGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGG

SEQ ID NO: 75 - protein, 31 aa, synthetic construct
AGKRTADGSEFEKRTADGSEFESPKKKAKVE

SEQ ID NO: 76 - protein, 46 aa, synthetic construct
AEAAAKEAAAKEAAAKEAAAKALEAEAAAKEAAAKEAAAKEAAAKA

SEQ ID NO: 77 - protein, 673 aa, synthetic construct
TAPLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFNEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGKIGYCRLFIPGFAELAQPLYAATRPGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGWLTAGGKAIKNAPEILALLTAVWLPKRVAVMHCKGHQKDDAPTSTGNRRADEVAREVAIRPLSTQATIS

SEQ ID NO: 78 - protein, 670 aa, synthetic construct
TLQLEEEYRLFEPESTQKQEMDIWLKNFPQAWAETGGMGTAHCQAPVLIQLKATATPISIRQYPMPHEAYQGIKPHIRRMLDQGILKPCQSPWNTPLLPVKKPGTEDYRPVQDLREVNKRVEDIHPTVPNPYNLLSTLPPSHPWYTVLDLKDAFFCLRLHSESQLLFAFEWRDPEIGLSGQLTWTRLPQGFKNSPTLFNEALHSDLADFRVRYPALVLLQYVDDLLLAAATRTECLEGTKALLETLGNKGYRASAKKAQICLQEVTYLGYSLKDGQRWLTKARKEAILSIPVPKNSRQVREFLGKAGYCRLFIPGFAELAAPLYPLTRPGTLFQWGTEQQLAFEDIKKALLSSPALGLPDITKPFELFIDENSGFAKGVLVQKLGPWKRPVAYLSKKLDTVASGWPPCLRMVAAIAILVKDAGKLTLGQPLTILTSHPVEALVRQPPNKWLSNARMTHYQAMLLDAERVHFGPTVSLNPATLLPLPSGGNHHDCLQILAETHGTRPDLTDQPLPDADLTWYTDGSSFIRNGEREAGAAVTTESEVIWAAPLPPGTSAQRAELIALTQALKMAEGKKLTVYTDSRYAFATTHVHGEIYRRRGWLTSEGKEIKNKNEILALLEALFLPKRLSIIHCPGHQKGDSPQAKGNRLADDTAKKAATETHSSLTVLP

SEQ ID NO: 79 - protein, 671 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLL

SEQ ID NO: 80 - protein, 751 aa, synthetic construct
MDPLQLLQPLEAEIKGTKLKAHWNSGATITCVPQAFLEEEVPIKNIWIKTIHGEKEQPVYYLTFKIQGRKVEAEVISSPYDYILVSPSDIPWLMKKPLQLTTLVPLQEYEERLLKQTMLTGSYKEKLQSLFLKYDALWQHWENQVGHRRIKPHHIATGTVNPRPQKQYPINPKAKASIQTVINDLLKQGVLIQQNSIMNTPVYPVPKPDGKWRMVLDYREVNKTIPLIAAQNQHSAGILSSIFRGKYKTTLDLSNGFWAHSITPESYWLTAFTWLGQQYCWTRLPQGFLNSPALFNADVVDLLKEVPNVQVYVDDIYISHDDPREHLEQLEKVFSLLLNAGYVVSLKKSEIAQHEVEFLGFNITKEGRGLTETFKQKLLNITPPRDLKQLQSILGKLNFARNFIPNFSELVKPLYNIIATAPGKYITWTTDNSQQLQNIISMLNSAENLEERNPEVRLIMKVNTSPSAGYIRFYNEFAKRPIMYLNYVYTKAEVKFTNTEKLLTTIHKGLIKALDLGMGQEILVYSPIVSMTKIQKTPLPERKALPIRWITWMSYLEDPRIQFHYDKTLPELQQVPTVTDDIIAKIKHPSEFSMVFYTDGSAIKHPNVNKSHNAGMGIAQVQFKPEFTVINTWSIPLGDHTAQLAEVAAVEFACKKALKIDGPVLIVTDSFYVAESVNKELPYWQSNGFFNNKKKPLKHVSKWKSIADCIQLKPDIIIIHEKGHQPTASTFHTEGNNLADKLATQGSYVVN

SEQ ID NO: 81 - protein, 2133 aa, synthetic construct
MPAAKRVKLDGGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGAEAAAKEAAAKEAAAKEAAAKALEAEAAAKEAAAKEAAAKEAAAKAGGTAPLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFNEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGKIGYCRLFIPGFAELAQPLYAATRPGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGWLTAGGKAIKNAPEILALLTAVWLPKRVAVMHCKGHQKDDAPTSTGNRRADEVAREVAIRPLSTQATISAGKRTADGSEFEKRTADGSEFESPKKKAKVE

SEQ ID NO: 82 - protein, 2130 aa, synthetic construct
MPAAKRVKLDGGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGAEAAAKEAAAKEAAAKEAAAKALEAEAAAKEAAAKEAAAKEAAAKAGGTLQLEEEYRLFEPESTQKQEMDIWLKNFPQAWAETGGMGTAHCQAPVLIQLKATATPISIRQYPMPHEAYQGIKPHIRRMLDQGILKPCQSPWNTPLLPVKKPGTEDYRPVQDLREVNKRVEDIHPTVPNPYNLLSTLPPSHPWYTVLDLKDAFFCLRLHSESQLLFAFEWRDPEIGLSGQLTWTRLPQGFKNSPTLFNEALHSDLADFRVRYPALVLLQYVDDLLLAAATRTECLEGTKALLETLGNKGYRASAKKAQICLQEVTYLGYSLKDGQRWLTKARKEAILSIPVPKNSRQVREFLGKAGYCRLFIPGFAELAAPLYPLTRPGTLFQWGTEQQLAFEDIKKALLSSPALGLPDITKPFELFIDENSGFAKGVLVQKLGPWKRPVAYLSKKLDTVASGWPPCLRMVAAIAILVKDAGKLTLGQPLTILTSHPVEALVRQPPNKWLSNARMTHYQAMLLDAERVHFGPTVSLNPATLLPLPSGGNHHDCLQILAETHGTRPDLTDQPLPDADLTWYTDGSSFIRNGEREAGAAVTTESEVIWAAPLPPGTSAQRAELIALTQALKMAEGKKLTVYTDSRYAFATTHVHGEIYRRRGWLTSEGKEIKNKNEILALLEALFLPKRLSIIHCPGHQKGDSPQAKGNRLADDTAKKAATETHSSLTVLPAGKRTADGSEFEKRTADGSEFESPKKKAKVE

SEQ ID NO: 83 - protein, 2131 aa, synthetic construct
MPAAKRVKLDGGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGAEAAAKEAAAKEAAAKEAAAKALEAEAAAKEAAAKEAAAKEAAAKAGGTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLAGKRTADGSEFEKRTADGSEFESPKKKAKVE

SEQ ID NO: 84 - protein, 2211 aa, synthetic construct
MPAAKRVKLDGGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGAEAAAKEAAAKEAAAKEAAAKALEAEAAAKEAAAKEAAAKEAAAKAGGMDPLQLLQPLEAEIKGTKLKAHWNSGATITCVPQAFLEEEVPIKNIWIKTIHGEKEQPVYYLTFKIQGRKVEAEVISSPYDYILVSPSDIPWLMKKPLQLTTLVPLQEYEERLLKQTMLTGSYKEKLQSLFLKYDALWQHWENQVGHRRIKPHHIATGTVNPRPQKQYPINPKAKASIQTVINDLLKQGVLIQQNSIMNTPVYPVPKPDGKWRMVLDYREVNKTIPLIAAQNQHSAGILSSIFRGKYKTTLDLSNGFWAHSITPESYWLTAFTWLGQQYCWTRLPQGFLNSPALFNADVVDLLKEVPNVQVYVDDIYISHDDPREHLEQLEKVFSLLLNAGYVVSLKKSEIAQHEVEFLGFNITKEGRGLTETFKQKLLNITPPRDLKQLQSILGKLNFARNFIPNFSELVKPLYNIIATAPGKYITWTTDNSQQLQNIISMLNSAENLEERNPEVRLIMKVNTSPSAGYIRFYNEFAKRPIMYLNYVYTKAEVKFTNTEKLLTTIHKGLIKALDLGMGQEILVYSPIVSMTKIQKTPLPERKALPIRWITWMSYLEDPRIQFHYDKTLPELQQVPTVTDDIIAKIKHPSEFSMVFYTDGSAIKHPNVNKSHNAGMGIAQVQFKPEFTVINTWSIPLGDHTAQLAEVAAVEFACKKALKIDGPVLIVTDSFYVAESVNKELPYWQSNGFFNNKKKPLKHVSKWKSIADCIQLKPDIIIIHEKGHQPTASTFHTEGNNLADKLATQGSYVVNAGKRTADGSEFEKRTADGSEFESPKKKAKVE

SEQ ID NO: 85 - protein, 671 aa, synthetic construct
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 86 - protein, 672 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLI

SEQ ID NO: 87 - protein, 2138 aa, synthetic construct
MPAAKRVKLDGGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSSGGSSGSETPGTSESATPESSGGSSGGSSGGTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEAGKRTADGSEFEKRTADGSEFESPKKKAKVE

SEQ ID NO: 88 - DNA, 76 bp, synthetic construct
  source 1..76: mol_type = other DNA
gttttagagctagaaatagcaagttaaaataaggctagtccgttatcaacttgaaaaagtgggaccgagtcggtcc

SEQ ID NO: 89 - skipped

SEQ ID NO: 90 - protein, 45 aa, synthetic construct
TTTPAPRPPTPAPTIASQPLSLRPEACRPAAGGAVHTRGLDFACD

SEQ ID NO: 91 - protein, 24 aa, synthetic construct
IYIWAPLAGTCGVLLLSLVITLYC

SEQ ID NO: 92 - protein, 41 aa, synthetic construct
RSKRSRLLHSDYMNMTPRRPGPTRKHYQPYAPPRDFAAYRS

SEQ ID NO: 93 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - skipped

SEQ ID NO: 95 - protein, 18 aa, synthetic construct
EGRGSLLTCGDVEENPGP

SEQ ID NO: 96 - skipped

SEQ ID NO: 97 - protein, 6 aa, synthetic construct
DKTHTC

SEQ ID NO: 98 - protein, 7 aa, synthetic construct
DKTHTCA

SEQ ID NO: 99 - protein, 8 aa, synthetic construct
DKTHTCAA

SEQ ID NO: 100 - protein, 15 aa, synthetic construct
EPKSCDKTHTCPPCP

SEQ ID NO: 101 - protein, 12 aa, synthetic construct
EPKCCVECPPCP

SEQ ID NO: 102 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 103 - protein, 12 aa, synthetic construct
ESKYGPPCPSCP

SEQ ID NO: 104 - protein, 13 aa, synthetic construct
VPRDCGCKPCICT

SEQ ID NO: 105 - protein, 14 aa, synthetic construct
EPRGPTIKPCPPCK

SEQ ID NO: 106 - protein, 16 aa, synthetic construct
EPSGPISTINPCPPCK

SEQ ID NO: 107 - protein, 16 aa, synthetic construct
EPRIPKPSTPPGSSCP

SEQ ID NO: 108 - protein, 232 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 109 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 232 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCAA

SEQ ID NO: 111 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 112 - protein, 232 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCAA

SEQ ID NO: 113 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 114 - protein, 232 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTACLGCLVKDYFPEPVTVSWNSGALTSGVHTFPAVLQSSGLYSLSSVVTVPSSSLGTQTYICNVNHKPSNTKVDKKVEPKSSDKTHTCAA

SEQ ID NO: 115 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVCIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 116 - protein, 232 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVCTFPAVLQSSGLYSLSSVVTVPSSSLGTQTYICNVNHKPSNTKVDKKVEPKSSDKTHTCAA

SEQ ID NO: 117 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYCLSSTLTLSKADYEKHKVYACEVTHQGLSSPVTKSFNRGES

SEQ ID NO: 118 - protein, 232 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCAA

SEQ ID NO: 119 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

SEQ ID NO: 120 - protein, 232 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLCSVVTVPSSSLGTQTYICNVNHKPSNTKVDKKVEPKSSDKTHTCAA

SEQ ID NO: 121 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES